(19) United States
(12) Patent Application Publication
(10) Pub. No.: US 2009/0036495 A1
(43) Pub. Date: Feb. 5, 2009

(54) Combinations Comprising Alpha-2-Delta Ligands and Ep4 Receptor Antagonists

(75) Inventor: Laurent Pascal Audoly, Doylestown, PA (US)

Correspondence Address: PHARMACIA CORPORATION;GLOBAL PATENT DEPARTMENT, POST OFFICE BOX 1027, ST. LOUIS, MO, 63006, US

(73) Assignee: Pfizer, Inc.

(21) Appl. No.: 11/568,152

(22) PCT Filed: Apr. 8, 2005
(86) PCT No.: PCT/IB05/00935

§ 371 Date: Feb. 28, 2008

Related U.S. Application Data
(60) Provisional application No. 60/563,863, filed on Apr. 20, 2004.

(51) Int. Cl. A61K 31/4439; A61K 31/4184; A61K 31/44; A61K 31/197; A61K 31/401; A61P 25/02; A61P 29/00; A61P 25/00

(52) U.S. Cl. 514/339; 514/341; 514/394; 514/355; 514/561; 514/423

(57) ABSTRACT

The instant invention relates to a combination of an EP4-receptor antagonist and an alpha-2-delta ligand, and pharmaceutically acceptable salts thereof, pharmaceutical compositions thereof and their use in the treatment of pain, particularly inflammatory, neuropathic, visceral and nociceptive pain.

### FIELD OF THE INVENTION

This invention relates to a combination of an EP4-receptor antagonist and an alpha-2-delta ligand. The invention also relates to a the use of a combination of an EP4-receptor antagonist and an alpha-2-delta ligand for the manufacture of a medicament for the treatment of pain. It also relates to a method for treating pain through the use of effective amounts of a combination of an EP4-receptor antagonist and an alpha-2-delta ligand.

### BACKGROUND TO THE INVENTION

An alpha-2-delta receptor ligand is any molecule which binds to any sub-type of the human calcium channel alpha-2-delta sub-unit. The calcium channel alpha-2-delta sub-unit comprises a number of receptor sub-types which have been described in the literature (e.g. N. S. Gee, J. P. Brown, V. U. Dissanayake, J. Offord, R. Thurlow, and G. N. Woodruff, J-Biol-Chem 271 (10):5768-76, 1996, (type 1); Gong, J. Hang, W. Kohler, Z. Li, and T-Z. Su, J. Membr. Biol. 184 (1):35-43, 2001, (types 2 and 3); E. Marais, N. Klugbauer, and F. Hofmann, Mol. Pharmacol. 59 (5):1243-1248, 2001. (types 2 and 3); and N. Qin, S. Yagel, M. L. Momplaisir, E. E. Codd, and M. R. D'Andrea. Mol. Pharmacol. 62 (3):485-496, 2002, (type 4)). Alpha-2-delta receptor ligands may also be known as GABA analogs.

Alpha-2-delta ligands have been described for the treatment of a number of indications, including epilepsy and pain.

Prostaglandins are mediators of pain, fever and other symptoms associated with inflammation. Prostaglandin E₂ (PGE₂) is the predominant eicosanoid detected in inflammation conditions. In addition, it is also involved in various physiological and/or pathological conditions and such as hyperalgesia, uterine contraction, digestive peristalsis, awakeness, suppression of gastric acid secretion, blood pressure variation, platelet function, bone metabolism, angiogenesis or the like.

Four PGE₂ receptor subtypes (EP₁, EP₂, EP₃ and EP₄) displaying different pharmacological effects have been cloned. The EP₄ subtype, a Gs-coupled receptor which stimulates cAMP production, is distributed in a wide variety of tissue, suggesting a major role in PGE₂-mediated biological events.

### SUMMARY OF THE INVENTION

It has now been found that combination therapy with an EP4-receptor antagonist and an alpha-2-delta ligand, when administered simultaneously, sequentially or separately, results in improvement in the treatment of pain, particularly neuropathic, inflammatory, nociceptive or visceral pain. Advantageously, the EP4-receptor antagonist and alpha-2-delta ligand can interact in a synergistic manner to control pain. This synergy allows a reduction in the dose required of each compound, leading to a reduction in the side effects and enhancement of the clinical utility of the compounds.

Accordingly, the invention provides, as a first aspect, a combination of an EP4-receptor antagonist and an alpha-2-delta ligand.

The invention further provides a combination of an EP4-receptor antagonist and an alpha-2-delta ligand for the treatment of pain.

The invention further provides the use of a combination of an EP4-receptor antagonist and an alpha-2-delta ligand for the manufacture of a medicament for the treatment of pain.

The invention further provides a method for treating pain through the use of effective amounts of a combination of an EP4-receptor antagonist and an alpha-2-delta ligand.

The best known alpha-2-delta ligand, gabapentin (Neurontin®), 1-(aminomethyl)-cyclohexylacetic acid, was first described in the patent literature in the patent family comprising U.S. Pat. No. 4,024,175. The compound is approved for the treatment of epilepsy and neuropathic pain.

A second alpha-2-delta ligand, pregabalin, (S)-(+)-4-amino-3-(2-methylpropyl)butanoic acid, is described in European patent application publication number EP0641330 as an anti-convulsant treatment useful in the treatment of epilepsy and in EP0934061 for the treatment of pain.

Further suitable alpha-2-delta ligands are described in the following documents.

International Patent Application Publication No. WO-A-01/28978, describes a series of novel bicyclic amino acids, their pharmaceutically acceptable salts, and their prodrugs of formula:

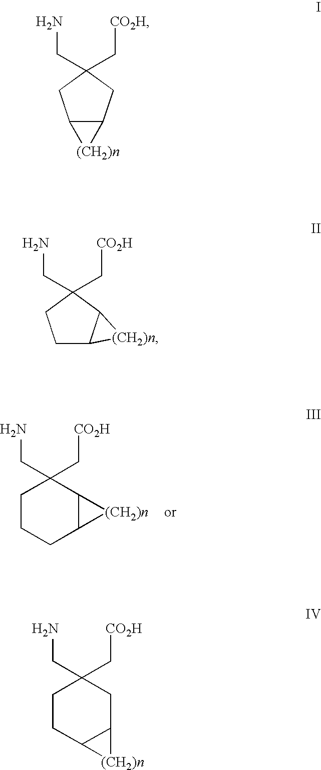

wherein n is an integer of from 1 to 4. Where there are stereocentres, each center may be independently R or S, preferred compounds being those of Formulae I-IV above in which n is an integer of from 2 to 4.

WO-A-02/85839 describes alpha-2-delta ligands of the following formulae:

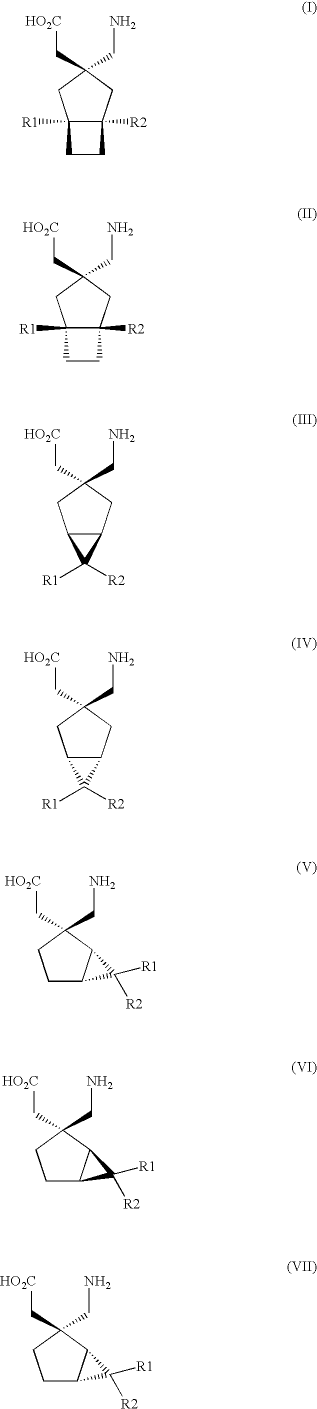

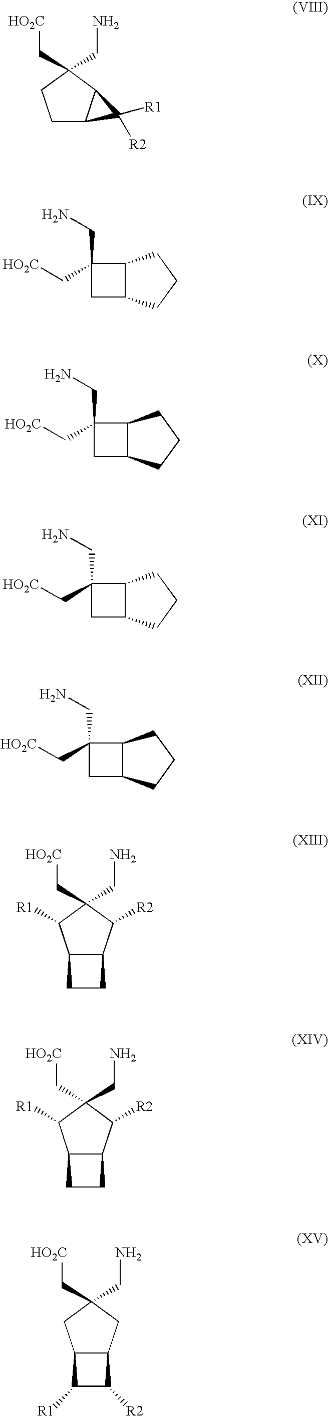

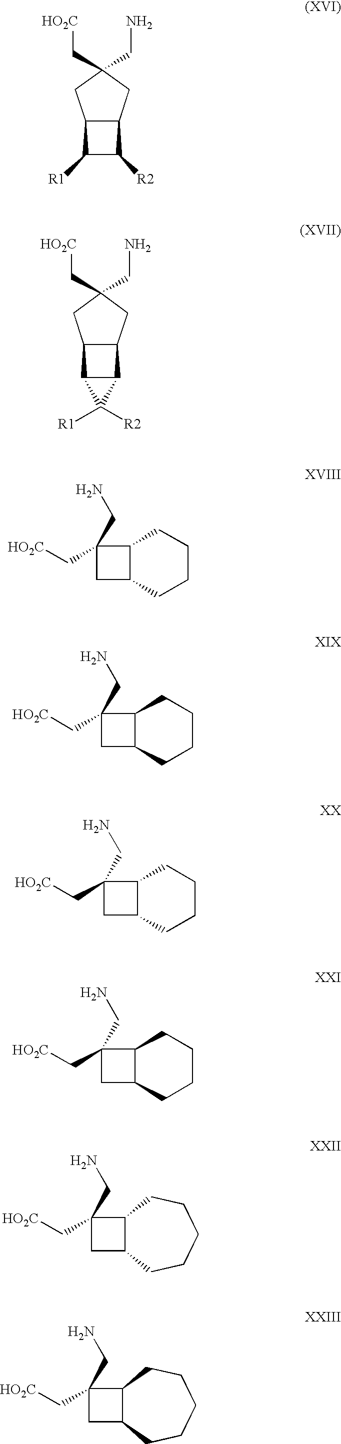

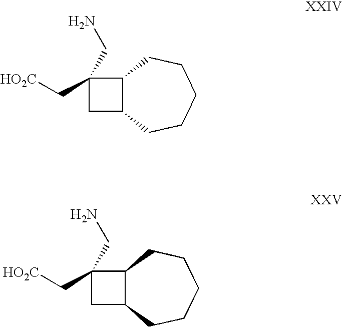

wherein R¹ and R² are each independently selected from H, straight or branched alkyl of 1-6 carbon atoms, cycloalkyl of from 3-6 carbon atoms, phenyl and benzyl, subject to the proviso that, except in the case of a tricyclooctane compound of formula (XVII), R¹ and R² are not simultaneously hydrogen; for use in the treatment of a number of indications, including pain.

International Patent Application Publication No. WO-A-03/082807, describes compounds of the formula I, below:

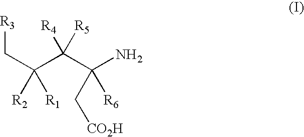

wherein R₁ is hydrogen or (C₁-C₆)alkyl optionally substituted with from one to five fluorine atoms;
R₂ is hydrogen or (C₁-C₆)alkyl optionally substituted with from one to five fluorine atoms; or
R₁ and R₂, together with the carbon to which they are attached, form a three to six membered cycloalkyl ring;
R₃ is (C₁-C₆)alkyl, (C₃-C₆)cycloalkyl, (C₃-C₆)cycloalkyl-(C₁-C₃)alkyl, phenyl, phenyl-(C₁-C₃)alkyl, pyridyl, pyridyl-(C₁-C₃)alkyl, phenyl-N(H)—, or pyridyl-N(H)—, wherein each of the foregoing alkyl moieties can be optionally substituted with from one to five fluorine atoms, preferably with from zero to three fluorine atoms, and wherein said phenyl and said pyridyl and the phenyl and pyridyl moieties of said phenyl-(C₁-C₃)alkyl and said pyridyl-(C₁-C₃)alkyl, respectively, can be optionally substituted with from one to three substituents, preferably with from zero to two substituents, independently selected from chloro, fluoro, amino, nitro, cyano, (C₁-C₃)alkylamino, (C₁-C₃)alkyl optionally substituted with from one to three fluorine atoms and (C₁-C₃)alkoxy optionally substituted with from one to three fluorine atoms;
R₄ is hydrogen or (C₁-C₆)alkyl optionally substituted with from one to five fluorine atoms;
R₅ is hydrogen or (C₁-C₆)alkyl optionally substituted with from one to five fluorine atoms; and
R₆ is hydrogen or (C₁-C₆)alkyl;
or a pharmaceutically acceptable salt thereof.

International Patent Application No. WO-A-2004/039367 describes compounds of the formula (I), below:

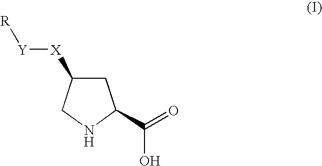

wherein
either X is O, S, NH or CH₂ and Y is CH₂ or a direct bond, or Y is O, S or NH and X is CH₂; and
R is a 3-12 membered cycloalkyl, 4-12 membered heterocycloalkyl, aryl or heteroaryl, where any ring may be optionally substituted with one or more substituents independently selected from halogen, hydroxy, cyano, nitro, amino, hydroxycarbonyl,
C₁-C₆ alkyl, C₁-C₆ alkenyl, C₁-C₆ alkynyl,
C₁-C₆ alkoxy, hydroxyC₁-C₆ alkyl, C₁-C₆ alkoxyC₁-C₆ alkyl, perfluoro C₁-C₆ alkyl, perfluoroC₁-C₆ alkoxy,
C₁-C₆ alkylamino, di-C₁-C₆ alkylamino, aminoC₁-C₆ alkyl, C₁-C₆ alkylaminoC₁-C₆ alkyl, di-C₁-C₆ alkylaminoC₁-C₆ alkyl,
C₁-C₆acyl, C₁-C₆acyloxy, C₁-C₆acyloxyC₁-C₆ alkyl, C₁-C₆ acylamino,
C₁-C₆ alkylthio, C₁-C₆ alkylthiocarbonyl, C₁-C₆ alkylthioxo, C₁-C₆ alkoxycarbonyl,
C₁-C₆ alkylsulfonyl, C₁-C₆ alkylsulfonylamino, aminosulfonyl, C₁-C₆ alkylaminosulfonyl, di-C₁-C₆ alkylaminosulfonyl, 3-8 membered cycloalkyl, 4-8 membered heterocycloalkyl, phenyl and monocyclic heteroaryl;
or a pharmaceutically acceptable salt thereof.

Examples of alpha-2-delta ligands for use in the present invention are those compounds, or pharmaceutically acceptable salts thereof, generally or specifically disclosed in U.S. Pat. No. 4,024,175, particularly gabapentin, EP641330, particularly pregabalin, U.S. Pat. No. 5,563,175, WO-A-97/33858, WO-A-97/33859, WO-A-99/31057, WO-A-99/31074, WO-A-97/29101, WO-A-02/085839, particularly [(1R,5R,6S)-6-(aminomethyl)bicyclo[3.2.0]hept-6-yl]acetic acid, WO-A-99/31075, particularly 3-(1-aminomethyl-cyclohexylmethyl)-4H-[1,2,4]oxadiazol-5-one and C-[1-(1H-tetrazol-5-ylmethyl)-cycloheptyl]-methylamine, WO-A-99/21824, particularly (3S,4S)-(1-aminomethyl-3,4-dimethyl-cyclopentyl)-acetic acid, WO-A-01/90052, WO-A-01/28978, particularly (1α,3α,5α)(3-aminomethyl-bicyclo[3.2.0]hept-3-yl)-acetic acid, EP0641330, WO-A-98/17627, WO-A-00/76958, particularly (3S,5R)-3-aminomethyl-5-methyl-octanoic acid, WO-A-03/082807, particularly (3S,5R)-3-amino-5-methyl-heptanoic acid, (3S,5R)-3-amino-5-methyl-nonanoic acid and (3S,5R)-3-amino-5-methyl-octanoic acid, WO-A-2004/039367, particularly (2S,4S)-4-(3-fluoro-phenoxymethyl)-pyrrolidine-2-carboxylic acid, (2S,4S)-4-(2,3-difluoro-benzyl)-pyrrolidine-2-carboxylic acid, (2S,4S)-4-(3-chlorophenoxy)proline and (2S,4S)-4-(3-fluorobenzyl)proline, EP1178034, EP1201240, WO-A-99/31074, WO-A-03/000642, WO-A-02/22568, WO-A-02/30871, WO-A-02/30881 WO-A-02/100392, WO-A-02/100347, WO-A-02/42414, WO-A-02/32736 and WO-A-02/28881, all of which are incorporated herein by reference.

Preferred alpha-2-delta ligands for use in the combination of the present invention include: gabapentin, pregabalin, [(1R,5R,6S)-6-(aminomethyl)bicyclo[3.2.0]hept-6-yl]acetic acid, 3-(1-aminomethyl-cyclohexylmethyl)-4H-[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)-acetic acid, (1α,3α,5α)(3-amino-methyl-bicyclo[3.2.0]hept-3-yl)-acetic acid, (3S,5R)-3-aminomethyl-5-methyl-octanoic acid, (3S,5R)-3-amino-5-methyl-heptanoic acid, (3S,5R)-3-amino-5-methyl-nonanoic acid, (3S,5R)-3-amino-5-methyl-octanoic acid, (2S,4S)-4-(3-chlorophenoxy)proline and (2S,4S)-4-(3-fluorobenzyl)proline or pharmaceutically acceptable salts thereof.

Further preferred alpha-2-delta ligands for use in the combination of the present invention are (3S,5R)-3-amino-5-methyloctanoic acid, (3S,5R)-3-amino-5-methylnonanoic acid, (3R,4R,5R)-3-amino-4,5-dimethylheptanoic acid and (3R,4R,5R)-3-amino-4,5-dimethyloctanoic acid, and the pharmaceutically acceptable salts thereof.

Particularly preferred alpha-2-delta ligands for use in the combination of the present invention are selected from gabapentin, pregabalin, (1α,3α,5α)(3-amino-methyl-bicyclo[3.2.0]hept-3-yl)-acetic acid, (2S,4S)-4-(3-chlorophenoxy)proline and (2S,4S)-4-(3-fluorobenzyl)proline or pharmaceutically acceptable salts thereof.

Suitable EP4-receptor antagonists are described in the following documents.

International Patent Application Publication No. WO-A-02/32900, describes a series of novel aryl or heteroaryl fused imidazole compounds and their pharmaceutically acceptable salts, of formula:

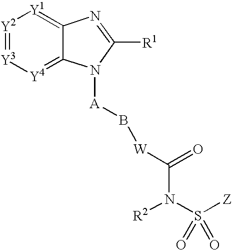

wherein Y¹, Y², Y³ and Y⁴ are independently selected from N, CH or C(L);
R¹ is H, C₁₋₈ alkyl, C₂₋₈ alkenyl, C₂₋₈ alkynyl, C₃₋₇ cycloalkyl, C₁₋₈ alkoxy, halo-substituted C₁₋₈ alkoxy, C₁₋₈ alkyl-S(O)m-, Q¹-, pyrrolidinyl, piperidyl, oxopyrrolidinyl, oxopiperidyl, amino, mono- or di-(C₁₋₈ alkyl)amino, C₁₋₄alkyl-C(═O)—N(R³)— or C₁₋₄alkyl-S(O)ₘ—N(R³)—, wherein said C₁₋₈ alkyl, C₂₋₈ alkenyl and C₂₋₈ alkynyl are optionally substituted with halo, C₁₋₃ alkyl, hydroxy, oxo, C₁₋₄ alkoxy-, C₁₋₄ alkyl-S(O)ₘ—, C₃₋₇ cycloalkyl-, cyano, indanyl, 1,2,3,4-tetrahydronaphtyl, 1,2-dihydronaphtyl, pyrrolidinyl, piperidyl, oxopyrrolidinyl, oxopiperidyl, Q¹-, Q¹-C(═O)—, Q¹-O—, Q¹-S(O)ₘ—, Q¹-C₁₋₄alkyl-O—, Q¹-C₁₋₄alkyl-S(O)ₘ—, Q¹-C₁₋₄alkyl-C(O)—N(R³)—, Q¹-C₁₋₄alkyl-N(R³)— or C₁₋₄alkyl-C(O)—N(R³)—;
Q¹ is a 5-12 membered monocyclic or bicyclic aromatic ring optionally containing up to 4 heteroatoms selected from O, N and S, and is optionally substituted with halo, C₁₋₄ alkyl, halo-substituted C₁₋₄ alkyl, hydroxy, C₁₋₄ alkoxy, halo-substituted C₁₋₄ alkoxy, C₁₋₄ alkylthio, nitro, amino, mono- or di-(C₁₋₄alkyl)amino, cyano, HO—C₁₋₄ alkyl, C₁₋₄ alkoxy-C₁₋₄alkyl, C₁₋₄ alkylsulfonyl, aminosulfonyl, C₁₋₄alkylC(═O)—, HO(O═)C—, C₁₋₄alkyl-O(O═)C—, R³N(R⁴)C(═O)—, C₁₋₄ alkylsulfonylamino, C₃₋₇ cycloalkyl, R³C(═O)N(R⁴)— or NH₂(HN═)C—;
A is a 5-6 membered monocyclic aromatic ring optionally containing up to 3 heteroatoms selected from O, N and S, wherein said 5-6 membered monocyclic aromatic ring is optionally substituted with up to 3 substituents selected from halo, C₁₋₄ alkyl, halo-substituted C₁₋₄ alkyl, hydroxy, C₁₋₄ alkoxy, halo-substituted C₁₋₄ alkoxy, C₁₋₄alkylthio, nitro, amino, mono- or di-(C₁₋₄ alkyl)amino, cyano, HO—C₁₋₄ alkyl, C₁₋₄ alkoxy-C₁₋₄alkyl, C₁₋₄ alkylsulfonyl, aminosulfonyl, acetyl, R³N(R⁴)C(═O)—, HO(O═)C—, C₁₋₄alkyl-O(O═)C—, C₁₋₄ alkylsulfonylamino, C₃₋₇ cycloalkyl, R³C(═O)N(R⁴)— and NH₂(HN═)C—;
B is halo-substituted C₁₋₆ alkylene, C₃₋₇ cycloalkylene, C₂₋₆ alkenylene, C₂₋₆ alkynylene, —O—C₁₋₅ alkylene, C₁₋₂ alkylene-O—C₁₋₂ alkylene or C₁₋₆ alkylene optionally substituted with an oxo group or C₁₋₃ alkyl;
W is NH, N—C₁₋₄ alkyl, O, S, N—OR⁵ or a covalent bond;
R² is H, C₁₋₄ alkyl, OH or C₁₋₄ alkoxy;
Z is a 5-12 membered monocyclic or bicyclic aromatic ring optionally containing up to 3 heteroatoms selected from O, N and S, wherein said 5-12 membered monocyclic or bicyclic aromatic ring is optionally substituted with halo, C₁₋₄ alkyl, halo-substituted C₁₋₄ alkyl, C₁₋₄ alkenyl, C₁₋₄ alkynyl, hydroxy, C₁₋₄ alkoxy, halo-substituted C₁₋₄ alkoxy, C₁₋₄ alkylthio, nitro, amino, mono- or di-(C₁₋₄ alkyl)amino, cyano, HO—C₁₋₄ alkyl, C₁₋₄ alkoxy-C₁₋₄alkyl, C₁₋₄ alkylsulfonyl, aminosulfonyl, C₁₋₄alkylC(═O)—, R³C(═O)N(R⁴)—, HO(O═)C—, C₁₋₄alkyl-O(O═)C—, C₁₋₄ alkylsulfonylamino, C₃₋₇ cycloalkyl, NH₂(HN═)C—, Q²-S(O)ₘ—, Q²-O—, Q²-N(R³)— or Q²-;
L is halo, C₁₋₄ alkyl, halo-substituted C₁₋₄ alkyl, hydroxy, C₁₋₄ alkoxy, halo-substituted C₁₋₄ alkoxy, C₁₋₄ alkylthio, nitro, amino, mono- or di-(C₁₋₄ alkyl)amino, cyano, HO—C₁₋₄ alkyl, C₁₋₄ alkoxy-C₁₋₄alkyl, C₁₋₄ alkylsulfonyl, aminosulfonyl, C₁₋₄alkylC(═O)—, HO(O═)C—, C₁₋₄alkyl-O(O═)C—, C₁₋₄ alkylsulfonylamino, C₃₋₇ cycloalkyl, R³C(═O)N(R⁴)—, NH₂(HN═)C—, R³N(R⁴)C(═O)—, R³N(R⁴)S(O)ₘ—, Q²-, Q²-C(═O)—, Q²-O—, Q²-C₁₋₄alkyl-O—, or two adjacent L groups are optionally joined together to form an alkylene chain having 3 or 4 members in which one or two (non-adjacent) carbon atoms are optionally replaced by oxygen atoms;
m is 0, 1 or 2;
R³ and R⁴ are independently selected from H and C₁₋₄ alkyl;
R⁵ is H, C₁₋₄ alkyl, C₁₋₄ alkyl-(O═)C— or C₁₋₄ alkyl-O—(O═)C—; and
Q² is a 5-12 membered monocyclic or bicyclic aromatic ring, or a 5-12 membered tricyclic ring optionally containing up to 3 heteroatoms selected from O, N and S, wherein said 5-12 membered monocyclic or bicyclic aromatic ring is optionally substituted with halo, C₁₋₄ alkyl, halo-substituted C₁₋₄ alkyl, C₁₋₄ alkenyl, C₁₋₄ alkynyl, hydroxy, C₁₋₄ alkoxy, halo-substituted C₁₋₁₄ alkoxy, C₁₋₄ alkylthio, nitro, amino, mono- or di-(C₁₋₄ alkyl)amino, cyano, HO—C₁₋₄ alkyl, C₁₋₄ alkoxy-C₁₋₄alkyl, C₁₋₄ alkylsulfonyl, aminosulfonyl, C₁₋₄alkyl-(O═)C—, R³(R⁴)C(═O)N—, HO(O═)C—, C₁₋₄ alkyl-O(O═)C—, C₁₋₄ alkylsulfonylamino, C₃₋₇ cycloalkyl, C₁₋₄ alkyl-C(═O)NH— or NH₂(HN═)C—.

International Patent Application No. WO-A-03/087061, describes a series of novel pyrazole compounds and their pharmaceutically acceptable salts, of formula:

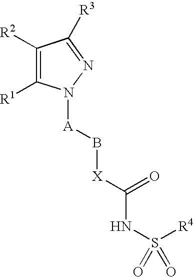

wherein R¹ represents a hydrogen atom, an alkyl group having from 1 to 6 carbon atoms, an amino group, mono- or di-alkylamino groups, the alkyl group(s) having from 1 to 6 carbon atoms, an aryl group or a heteroaryl group;
R² represents a hydrogen atom, a halogen atom, an alkyl group having from 1 to 6 carbon atoms, a cycloalkyl group having from 3 to 8 carbon atoms, a cycloalkenyl group having from 3 to 10 carbon atoms, an aralkyl group, an aryl group, or a heteroaryl group;
R³ represents an alkyl group having from 1 to 6 carbon atoms, a haloalkyl group having from 1 to 6 carbon atoms, a hydroxyalkyl group having from 1 to 6 carbon atoms, an aryl group or a heteroaryl group;
R⁴ represents an aryl group, or a heteroaryl group;
A represents an aryl¹ group having from 6 to 10 carbon atoms or an heteroaryl¹ group having from 5 to 7 atoms, wherein 1 to 4 of said atoms of the heteroaryl¹ group are independently selected from the group consisting of sulfur atoms, oxygen atoms and nitrogen atoms;
B represents an alkylene group having from 1 to 6 carbon atoms;
X represents NH, N[(C₁-C₆)alkyl], oxygen or sulfur;
said aryl groups have 6 to 14 carbon atoms;
said heteroaryl groups are 5- to 14-membered aromatic heterocyclic groups containing from 1 to 4 heteroatoms selected from the group consisting of sulfur atoms, oxygen atoms and nitrogen atoms;
said aryl groups and said heteroaryl groups are unsubstituted or are substituted by at least one substituent selected from the group consisting of substituents α, defined below;
said aralkyl groups are alkyl groups having from 1 to 6 carbon atoms and which are substituted by at least one aryl group as defined above;
said substituents α are selected from the group consisting of alkyl group having from 1 to 6 carbon atoms, an aryl group defined above, a heteroaryl group defined above, hydroxy group, halogen atoms, alkoxy group having from 1 to 6 carbon atoms, alkylthio group having from 1 to 6 carbon atoms, alkanoyl group having from 1 to 6 carbon atoms, alkanoylamino group having from 1 to 6 carbon atoms, alkanoylaminoalkyl group having from 1 to 6 carbon atoms in the alkanoyl and alkyl part, amino group, mono- or di-alkylamino group having from 1 to 6 carbon atoms, haloalkyl group having from 1 to 6 carbon atoms, haloalkoxy group having from 1 to 6 carbon atoms, carbamoyl group, cyano group, a hydroxyalkyl group having from 1 to 6 carbon atoms, alkylsufinyl group having from 1 to 6 carbon atoms, alkylsulfonyl group having from 1 to 6 carbon atoms, aminoalkoxy group having from 1 to 6 carbon atoms, mono- or di-alkylaminoalkoxy group, the alkyl group(s) having from 1 to 6 carbon atoms in the alkyl and alkoxy part, alkylsulfonylamino group having from 1 to 6 carbon atoms and aminosulfonyl group;
with the proviso that said aryl group and said heteroaryl group in said substituents α are not substituted by an aryl group or an heteroaryl group: or a pharmaceutically acceptable ester of such compound, or a pharmaceutically acceptable salt thereof.

International Patent Application No. WO-A-03/086390, describes a series of novel imidazole compounds and their pharmaceutically acceptable salts, of formula:

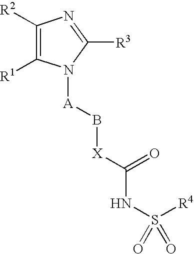

wherein either R¹ represents a hydrogen atom, an alkyl group having from 1 to 6 carbon atoms, an aryl group or a heteroaryl group; and
R² represents a hydrogen atom, a halogen atom, an alkyl group having from 1 to 6 carbon atoms, a cycloalkyl group having from 3 to 8 carbon atoms, a cycloalkenyl group having from 3 to 10 carbon atoms, an aralkyl group, an aryl group, or a heteroaryl group; or R¹ and R² groups are joined together to form an alkylene chain having 3 to 6 carbon atoms;
R³ represents a hydrogen atom, a halogen atom, an alkyl group having from 1 to 6 carbon atoms, an amino group, mono- or di-alkylamino groups, with alkyl group(s) having from 1 to 6 carbon atoms, a haloalkyl group having from 1 to 6 carbon atoms, a cycloalkyl group having from 3 to 8 carbon atoms, a cycloalkenyl group having from 3 to 10 carbon atoms, an aralkyl group, an aryl group or a heteroaryl group;
R⁴ represents an aryl group, or a heteroaryl group;
A represents an aryl¹ group having from 6 to 10 carbon atoms or an heteroaryl¹ group having from 5 to 7 atoms, wherein 1 to 4 of said atoms of the heteroaryl¹ group are independently selected from the group consisting of sulfur atoms, oxygen atoms and nitrogen atoms;
B represents an alkylene group having from 1 to 6 carbon atoms;
X represents NH, N[(C₁-C₆)alkyl], oxygen or sulfur;
said aryl groups have from 6 to 14 carbon atoms;
said heteroaryl groups are 5- to 14-membered aromatic heterocyclic groups containing from 1 to 4 heteroatoms selected from the group consisting of sulfur atoms, oxygen atoms and nitrogen atoms;
said aryl groups and said heteroaryl groups are unsubstituted or are substituted by at least one substituent selected from the group consisting of substituents α, defined below;
said aralkyl groups are alkyl groups having from 1 to 6 carbon atoms and which are substituted by at least one aryl group as defined above;
said substituents α are selected from the group consisting of alkyl group having from 1 to 6 carbon atoms, an aryl group defined above, a heteroaryl group defined above, hydroxy groups, halogen atom, alkoxy group having from 1 to 6 carbon atoms, alkylthio group having from 1 to 6 carbon atoms, alkanoyl group having from 1 to 6 carbon atoms, alkanoylamino group having from 1 to 6 carbon atoms, alkanoylaminoalkyl group having from 1 to 6 carbon atoms in the alkanoyl and alkyl part, amino group, mono- or di-alkylamino group having from 1 to 6 carbon atoms, haloalkyl group having from 1 to 6 carbon atoms, haloalkoxy group having from 1 to 6 carbon atoms, carbamoyl group, cyano group, a hydroxyalkyl group having from 1 to 6 carbon atoms, alkylsufinyl group having from 1 to 6 carbon atoms, alkylsulfonyl group having from 1 to 6 carbon atoms, aminoalkoxy group having from 1 to 6 carbon atoms, mono- or di-alkylaminoalkoxy group, with alkyl group(s) having from 1 to 6 carbon atoms in the alkyl and alkoxy part, alkylsulfonylamino group having from 1 to 6 carbon atoms and aminosulfonyl group;
with the proviso that said aryl groups and said heteroaryl groups in said substituents α are not substituted by an aryl group or an heteroaryl group.

U.S. Application No. US60/500,131 (not published at the filing date of the present invention), describes a series of novel aryl or heteroaryl fused imidazole compounds and their pharmaceutically acceptable salts, of formula:

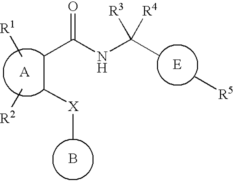

wherein A represents a phenyl group or a pyridyl group;
B represents an aryl group or a heteroaryl group;
E represents a phenylene group;
R¹ and R² independently represent a hydrogen atom, a halogen atom, an alkyl group having from 1 to 4 carbon atoms, an alkoxy group having from 1 to 4 carbon atoms, a haloalkyl group having from 1 to 4 carbon atoms, a haloalkoxy group having from 1 to 4 carbon atoms, a cyano group or an aminocarbonyl group;
R³ and R⁴ independently represent a hydrogen atom or an alkyl group having from 1 to 4 carbon atoms; or R³ and R⁴ groups may be joined together to form an alkylene chain having 3 to 6 carbon atoms;
R⁵ represents

### —CO₂H,

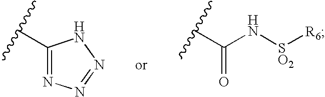

R⁶ represents an alkyl group having from 1 to 6 carbon atoms, a cycloalkyl group having from 3 to 7 ring atoms, an aryl group or a heteroaryl group;
X represents a methylene group, an oxygen atom or a sulfur atom;
said aryl groups have from 6 to 10 carbon atoms;
said heteroaryl groups are 5- to 10-membered aromatic heterocyclic groups containing from 1 to 3 heteroatoms selected from the group consisting of sulfur atoms, oxygen atoms and nitrogen atoms;
said aryl groups and said heteroaryl groups referred to in the definitions of B are unsubstituted or are substituted by at least one substituent selected from the group consisting of substituents α;
said phenylene groups referred to in the definitions of E are unsubstituted or are substituted by at least one substituent selected from the group consisting of substituents α;
said aryl groups and said heteroaryl groups referred to in the definitions of R⁶ and α are unsubstituted or are substituted by at least one substituent selected from the group consisting of substituents β;
said substituents α are selected from the group consisting of halogen atoms, alkyl groups having from 1 to 4 carbon atoms, alkoxy groups having from 1 to 4 carbon atoms, haloalkyl groups having from 1 to 4 carbon atoms, haloalkoxy groups having from 1 to 4 carbon atoms, cyano groups, alkynyl groups having from 2 to 6 carbon atoms, alkanoyl groups having from 1 to 5 carbon atoms, cycloalkyl groups having from 3 to 7 ring atoms, heteroaryl groups, aryl groups, aralkoxy groups having from 7 to 10 carbon atoms, arylcarbonyl groups, two adjacent α groups are optionally joined together to form an alkylene or an alkenylene chain having 3 or 4 carbon atoms, aminocarbonyl groups, alkenyl groups having from 2 to 5 carbon atoms, alkylthio groups having from 1 to 4 carbon atoms, aminosulfinyl groups, aminosulfonyl groups, hydroxy groups, hydroxyalkyl groups having from 1 to 4 carbon atoms, nitro groups, amino groups, carboxy groups, alkoxycarbonyl groups having from 2 to 5 carbon atoms, alkoxyalkyl groups having from 1 to 4 carbon atoms, alkylsulfonyl groups having from 1 to 4 carbon atoms, alkanoylamino groups having from 1 to 4 carbon atoms, alkanoyl(alkyl)amino groups having from 1 to 6 carbon atoms, alkanoylaminoalkyl groups having from 1 to 6 carbon atoms in the alkanoyl and alkyl part, alkanoyl(alkyl)aminoalkyl groups having from 1 to 6 carbon atoms in the alkanoyl and each alkyl part, alkylsulfonylamino groups having from 1 to 4 carbon atoms, mono- or di-alkylaminocarbonyl groups having from 1 to 6 carbon atoms, mono- or di-alkylaminosulfinyl groups having from 1 to 6 carbon atoms, mono- or di-alkylaminosulfonyl groups having from 1 to 6 carbon atoms, aminoalkyl groups having from 1 to 4 carbon atoms, mono- or di-alkylamino groups having from 1 to 6 carbon atoms, mono- or di-alkylaminoalkyl groups having from 1 to 6 carbon atoms in the alkyl part, aralkyl groups having from 7 to 10 carbon atoms, heteroarylalkyl groups having from 1 to 4 carbon atoms in the alkyl part, heteroarylalkoxy groups having from 1 to 4 carbon atoms in the alkoxy part or alkylsulfonylamino groups having from 1 to 4 carbon atoms;
said substituents β are selected from the group consisting of halogen atoms, alkyl groups having from 1 to 4 carbon atoms, alkoxy groups having from 1 to 4 carbon atoms, haloalkyl groups having from 1 to 4 carbon atoms or haloalkoxy groups having from 1 to 4 carbon atoms or cyano groups.

U.S. Application No. US60/568,088 (not published at the filing date of the present invention), describes a series of novel methyl aryl or heteroaryl amide compounds, and their pharmaceutically acceptable salts, of formula:

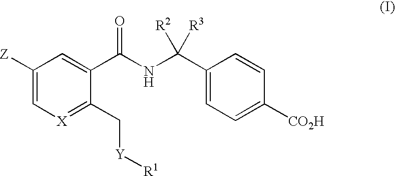

wherein
X represents —CH— or a nitrogen atom;
Y represents —NR⁴, an oxygen atom or a sulfur atom;
R⁴ represents a hydrogen atom or an alkyl group having from 1 to 3 carbon atoms;
Z represents a hydrogen atom or a halogen atom;
R¹ represents an alkyl group having from 1 to 6 carbon atoms optionally substituted with an alkoxy group having from 1 to 6 carbon atoms or a cycloalkyl group having from 3 to 7 carbon atoms; a cycloalkyl group having from 3 to 7 carbon atoms optionally substituted with an alkyl group having from 1 to 3 carbon atoms; a phenyl group optionally substituted with one or more substituents α; or a group Het¹ optionally substituted with one or more substituents α;
Het¹ represents a heterocyclic group having from 4 to 7 ring atoms which contains either from 1 to 4 ring nitrogen heteroatoms or from 0 to 2 nitrogen ring heteroatoms and 1 oxygen or 1 sulfur ring heteroatom;
R² and R³ independently represent a hydrogen atom or an alkyl group having from 1 to 3 carbon atoms; or R² and R³ groups together form an alkylene chain having from 3 to 6 carbon atoms; and
said substituent α is selected from the group consisting of halogen atoms, alkyl groups having from 1 to 4 carbon atoms, haloalkyl groups having from 1 to 4 carbon atoms, hydroxy groups, alkoxy groups having from 1 to 4 carbon atoms, haloalkoxy groups having from 1 to 4 carbon atoms, cyano groups, hydroxy alkyl groups having from 1 to 4 carbon atoms, alkoxyalkyl groups having from 1 to 4 carbon atoms in alkoxy and alkyl groups, alkylsulfonyl groups having from 1 to 4 carbon atoms, alkanoyl groups having from 2 to 5 carbon atoms, alkenyl groups having from 2 to 4 carbon atoms, alkynyl groups having from 2 to 4 carbon atoms, alkylthio groups having from 1 to 4 carbon atoms, nitro groups, amino groups, mono- or di-alkylamino groups having from 1 to 4 carbon atoms, aminosulfonyl groups, alkoxycarbonyl groups having from 1 to 4 carbon atoms, alkylsulfonylamino groups having from 1 to 4 carbon atoms, cycloalkyl groups having from 3 to 7 carbon atoms and a mono- or di-alkylaminocarbonyl groups having from 1 to 6 carbon atoms.

U.S. Application No. US60/568,088 (not published at the filing date of the present invention), describes a series of novel ortho substituted aryl or heteroaryl amide compounds, and their pharmaceutically acceptable salts, of formula:

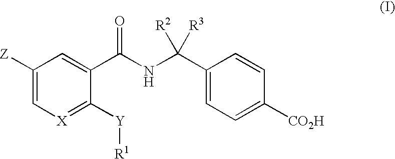

wherein
X represents a —CH— or a nitrogen atom;
Y represents NR⁴, an oxygen atom or a sulfur atom;
R⁴ represents a hydrogen atom or an alkyl group having from 1 to 3 carbon atoms;
Z represents a hydrogen atom or a halogen atom;
R¹ represents an alkyl group having from 1 to 6 carbon atoms optionally substituted with 1 to 2 groups independently selected from an alkoxy group having from 1 to 6 carbon atoms, a trifluoromethyl group, an alkanoyl group having from 2 to 5 carbon atoms, a cycloalkyl group having from 3 to 7 carbon atoms, a phenyl group, a phenoxy group, a heterocyclic group and a heteroaryl group; a cycloalkyl group having from 3 to 7 carbon atoms optionally substituted with an alkyl group having from 1 to 3 carbon atoms; or a heterocyclic group;
R² and R³ independently represent a hydrogen atom or an alkyl group having from 1 to 3 carbon atoms; or R² and R³ groups together form an alkylene chain having from 3 to 6 carbon atoms;
said heteroaryl group is a 4 to 7-membered aromatic ring system having either from 1 to 4 ring nitrogen heteroatoms or 0 to 2 nitrogen ring heteroatoms and 1 oxygen or 1 sulfur ring heteroatom;
said heterocyclic group is a 4 to 7-membered saturated ring system having either from 1 to 4 ring nitrogen heteroatoms or 0 to 2 nitrogen ring heteroatoms and 1 oxygen or 1 sulphur ring heteroatom;
said phenyl groups, phenoxy group and said heteroaryl groups referred to in the definitions of R¹ are unsubstituted or are substituted by at least one substituent selected from the group consisting of substituents α;
said substituent α is selected from the group consisting of halogen atoms, alkyl groups having from 1 to 4 carbon atoms, haloalkyl groups having from 1 to 4 carbon atoms, hydroxy groups, alkoxy groups having from 1 to 4 carbon atoms, haloalkoxy groups having from 1 to 4 carbon atoms, cyano groups, hydroxy alkyl groups having from 1 to 4 carbon atoms, alkoxyalkyl groups having from 1 to 4 carbon atoms in alkoxy and alky groups, alkylsulfonyl groups having from 1 to 4 carbon atoms, alkanoyl groups having from 2 to 5 carbon atoms, alkenyl groups having from 2 to 4 carbon atoms, alkynyl groups having from 2 to 4 carbon atoms, alkylthio groups having from 1 to 4 carbon atoms, nitro groups, amino groups, mono- or di-alkylamino groups having from 1 to 4 carbon atoms, aminosulfonyl groups, alkoxycarbonyl groups having from 1 to 4 carbon atoms, alkylsulfonylamino groups having from 1 to 4 carbon atoms, cycloalkyl groups having from 3 to 7 carbon atoms and a mono- or di-alkylaminocarbonyl groups having from 1 to 6 carbon atoms.

Examples of EP4-receptor antagonists for use with the present invention are those compounds, or pharmaceutically acceptable salts thereof, generally or specifically disclosed in WO-A-02/32900, particularly 2-ethyl-4,6-dimethyl-1-(4-{2-[({[(4-methylphenyl)sulfonyl]amino}carbonyl)amino]ethyl}phenyl)-1H-imidazo[4,5-c]pyridine and 4-(6-chloro-2-ethyl-5-trifluoromethyl-1H-benzimidazol-1-yl)phenethyl-(4-methylphenyl)sulfonylcarbamate, WO-A-03/087061, particularly 2-fluoro-N-{[(2-{4-[5-methyl-4-phenyl-3-(trifluoromethyl)-1H-pyrazol-1-yl]phenyl}ethyl)amino]carbonyl}benzenesulfonamide, WO-A-03/086390, particularly 2-[4-(2-isopropyl-4-phenyl-1H-imidazol-1-yl)phenyl]ethyl (2-chlorophenyl)sulfonylcarbamate, US60/500,131, particularly 4-[(1S)-1-({[5-chloro-2-(3-fluorophenoxy)pyridin-3-yl]carbonyl}amino)ethyl]benzoic acid, WO-A-03/016254, particularly 4-[2-[[2-(naphthalen-1-yl)propanoyl]amino]-4-methylthiomethylphenyl]butanoic acid, WO-A-02/20462, WO-A-02/16311, WO-A-01/62708, WO-A-00/15608, EP0985663, WO-A-00/03980, particularly methyl 7-[(1R,2R,3R)-3-hydroxy-2-[(E)-(3S)-3-hydroxy-4-(m-methoxymethylphenyl)-1-butenyl]-5-oxocyclopentyl]-5-thiaheptanoate, U.S. 60/568,088, U.S. 60/567,931, EP0855389, WO-A-02/50031, WO-A-02/50032, WO-A-02/50033, GB2330307, WO-A-01/10426, WO-A-00/18744, WO-A-00/16760, WO-A-98/55468, WO-A-00/21532, WO-A-00/24393, WO-A-99/47497, WO-A-01/72302, WO-A-00/18405 and WO-A-01/42281, all of which are incorporated by reference.

Suitable EP4-receptor antagonists for use in the present invention are compounds selected from:
2-ethyl-4,6-dimethyl-1-(4-{2-[({[(4-methylphenyl)sulfonyl]amino}carbonyl)amino]ethyl}phenyl)-1H-imidazo[4,5-c]pyridine;
4-(6-chloro-2-ethyl-5-trifluoromethyl-1H-benzimidazol-1-yl)phenethyl-(4-methylphenyl)sulfonylcarbamate;
5-acetyl-2-ethyl-3-(4-{2-[({[(4-methylphenyl)sulfonyl]amino}carbonyl)amino]ethyl}phenyl)benzimidazole;
N-{[(2-{4-[2-ethyl-5-(1-hydroxy-1-methylethyl)-1H-benzimidazol-1-yl]phenyl}ethyl)amino]carbonyl}-4-methylbenzenesulfonamide;
2-{4-[6-chloro-2-ethyl-5-(trifluoromethyl)-1H-benzimidazol-1-yl]phenyl}ethyl (5-methyl-2-pyridinyl)sulfonylcarbamate;
2-{4-[6-chloro-2-(4-pyridinyl)-5-(trifluoromethyl)-1H-benzimidazol-1-yl]phenyl}ethyl (4-methylphenyl)sulfonylcarbamate;
2-{4-[5,7-dimethyl-2-(methylamino)-3H-imidazo[4,5-b]pyridin-3-yl]phenyl}ethyl (4-methylphenyl)sulfonylcarbamate;
N-{[(2-{4-[5,7-dimethyl-2-(methylamino)-3H-imidazo[4,5-b]pyridin-3-yl]phenyl}ethyl)amino]carbonyl}-4-methyl benzenesulfonamide;
2-{5-[6-chloro-2-ethyl-5-(trifluoromethyl)-1H-benzimidazol-1-yl]-2-pyridinyl}ethyl (4-methylphenyl)sulfonylcarbamate;
2-{4-[2-(1,1-dimethylethyl)-4,6-dimethyl-1H-imidazo[4,5-c]pyridin-1-yl]phenyl}ethyl (4-methyl phenyl)sulfonylcarbamate;
6-chloro-2-ethyl-1-(4-{2-[methyl({[(4-methylphenyl)sulfonyl]amino}carbonyl)amino]ethyl}phenyl)-1H-benzimidazole-5-carboxamide;
4-[(1S)-1-({[5-chloro-2-(3-fluorophenoxy)pyridin-3-yl]carbonyl}amino)ethyl]benzoic acid;
4-((1S)-1-{[5-chloro-2-(3-fluorophenoxy)benzoyl]amino}ethyl)benzoic acid;
4-[(1S)-1-({[5-chloro-2-(3,4-difluorophenoxy)pyridin-3-yl]carbonyl}amino)ethyl]benzoic acid;
4-[(1S)-1-({[5-chloro-2-(4-fluorophenoxy)pyridin-3-yl]carbonyl}amino)ethyl]benzoic acid;
4-((1S)-1-{[5-chloro-2-(4-fluorophenoxy)benzoyl]amino}ethyl)benzoic acid;
4-[(1S)-1-({[5-chloro-2-(3-chlorophenoxy)pyridin-3-yl]carbonyl}amino)ethyl]benzoic acid;
4-[(1S)-1-({[5-chloro-2-(3-cyanophenoxy)pyridin-3-yl]carbonyl}amino)ethyl]benzoic acid;
4-[(1S)-1-({[5-chloro-2-(2,6-difluorophenoxy)pyridin-3-yl]carbonyl}amino)ethyl]benzoic acid;
4-((1S)-1-{[5-chloro-2-(3-chlorophenoxy)benzoyl]amino}ethyl)benzoic acid;
4-[(1S)-1-({[5-chloro-2-(2-chloro-4-fluorophenoxy)pyridin-3-yl]carbonyl}amino)ethyl]benzoic acid;
2-fluoro-N-{[(2-{4-[5-methyl-4-phenyl-3-(trifluoromethyl)-1H-pyrazol-1-yl]phenyl}ethyl)amino]carbonyl}benzenesulfonamide;
2,4-difluoro-N-{[(2-{4-[5-methyl-4-phenyl-3-(trifluoromethyl)-1H-pyrazol-1-yl]phenyl}ethyl)amino]carbonyl}benzenesulfonamide;
N-[({2-[4-(3,5-dimethyl-4-phenyl-1H-pyrazol-1-yl)phenyl]ethyl}amino)carbonyl]-4-methylbenzenesulfonamide;
2-[4-(3,5-dimethyl-4-phenyl-1H-pyrazol-1-yl)phenyl]ethyl[(4-methyl phenyl)sulfonyl]carbamate;
N-[({2-[4-(3,5-dimethyl-4-phenyl-1H-pyrazol-1-yl)phenyl]ethyl}amino)carbonyl]-2-fluorobenzenesulfonamide;
N-[({2-[4-(3,5-dimethyl-4-phenyl-1H-pyrazol-1-yl)phenyl]ethyl}amino)carbonyl]-4-methoxybenzenesulfonamide;
N-[({2-[4-(3,5-dimethyl-4-phenyl-1H-pyrazol-1-yl)phenyl]ethyl}amino)carbonyl]-3,4-dimethoxybenzenesulfonamide;
N-{[(2-{4-[4-(4-ethoxyphenyl)-3,5-dimethyl-1H-pyrazol-1-yl]phenyl}ethyl)amino]carbonyl}-4-methylbenzenesulfonamide;
N-[({2-[4-(3,5-dimethyl-4-phenyl-1H-pyrazol-1-yl)phenyl]ethyl}amino)carbonyl]-2,4-difluorobenzenesulfonamide;
2-{4-[4-(4-fluorophenyl)-3,5-dimethyl-1H-pyrazol-1-yl]phenyl}ethyl[(4-methyl phenyl)sulfonyl]carbamate;
2-[4-(2-isopropyl-4-phenyl-1H-imidazol-1-yl)phenyl]ethyl (2-chlorophenyl)sulfonylcarbamate;
2-[4-(2-ethyl-4-phenyl-1H-imidazole-1-yl)phenyl]ethyl (4-methylphenyl)sulfonylcarbamate;
2-[4-(2-butyl-4-phenyl-1H-imidazol-1-yl)phenyl]ethyl (2-chlorophenyl)sulfonylcarbamate;
2-[4-(2-isobutyl-4-phenyl-1H-imidazol-1-yl)phenyl]ethyl (2-chlorophenyl)sulfonylcarbamate;
4-chloro-N-[({2-[4-(2-ethyl-4-phenyl-1H-imidazol-1-yl)phenyl]ethyl}amino)carbonyl]benzenesulfonamide;
2-[4-(2-amino-4,5-diphenyl-1H-imidazol-1-yl)phenyl]ethyl (4-methylphenyl)sulfonylcarbamate;
N-[({2-[4-(2-ethyl-4-phenyl-1H-imidazol-1-yl)phenyl]ethyl}amino)carbonyl]-4-methylbenzenesulfonamide;
2-chloro-N-[({2-[4-(2-ethyl-4-phenyl-1H-imidazol-1-yl)phenyl]ethyl}amino)carbonyl]benzenesulfonamide;
2-[4-(2-tert-butyl-4-phenyl-1H-imidazol-1-yl)phenyl]ethyl (2-chlorophenyl)sulfonylcarbamate;
4-chloro-N-[({2-[4-(2-isopropyl-4-phenyl-1H-imidazol-1-yl)phenyl]ethyl}amino)carbonyl]benzenesulfonamide;
   and their pharmaceutically acceptable salts.

Preferred EP4 receptor antagonists for use with the present invention are selected from:
2-ethyl-4,6-dimethyl-1-(4-{2-[({[(4-methylphenyl)sulfonyl]amino}carbonyl)amino]ethyl}phenyl)-1H-imidazo[4,5-c]pyridine;
4-(6-chloro-2-ethyl-5-trifluoromethyl-1H-benzimidazol-1-yl)phenethyl-(4-methylphenyl)sulfonylcarbamate;
5-acetyl-2-ethyl-3-(4-{2-[({[(4-methylphenyl)sulfonyl]amino}carbonyl)amino]ethyl}phenyl)benzimidazole;
N-{[(2-{4-[2-ethyl-5-(1-hydroxy-1-methylethyl)-1H-benzimidazol-1-yl]phenyl}ethyl)amino]carbonyl}-4-methylbenzenesulfonamide;
2-{4-[6-chloro-2-ethyl-5-(trifluoromethyl)-1H-benzimidazol-1-yl]phenyl}ethyl (5-methyl-2-pyridinyl)sulfonylcarbamate;
2-{4-[6-chloro-2-(4-pyridinyl)-5-(trifluoromethyl)-1H-benzimidazol-1-yl]phenyl}ethyl (4-methylphenyl)sulfonylcarbamate;
4-[(1S)-1-({[5-chloro-2-(3-fluorophenoxy)pyridin-3-yl]carbonyl}amino)ethyl]benzoic acid;
4-((1S)-1-{[5-chloro-2-(3-fluorophenoxy)benzoyl]amino}ethyl)benzoic acid;
4-[(1S)-1-({[5-chloro-2-(3,4-difluorophenoxy)pyridin-3-yl]carbonyl}amino)ethyl]benzoic acid;
4-[(1S)-1-({[5-chloro-2-(4-fluorophenoxy)pyridin-3-yl]carbonyl}amino)ethyl]benzoic acid;
4-((1S)-1-{[5-chloro-2-(4-fluorophenoxy)benzoyl]amino}ethyl)benzoic acid;
2-fluoro-N-{[(2-{4-[5-methyl-4-phenyl-3-(trifluoromethyl)-1H-pyrazol-1-yl]phenyl}ethyl)amino]carbonyl}benzenesulfonamide;
2,4-difluoro-N-{[(2-{4-[5-methyl-4-phenyl-3-(trifluoromethyl)-1H-pyrazol-1-yl]phenyl}ethyl)amino]carbonyl}benzenesulfonamide;
N-[({2-[4-(3,5-dimethyl-4-phenyl-1H-pyrazol-1-yl)phenyl]ethyl}amino)carbonyl]-4-methylbenzenesulfonamide;
2-[4-(3,5-dimethyl-4-phenyl-1H-pyrazol-1-yl)phenyl]ethyl [(4-methyl phenyl)sulfonyl]carbamate;
N-[({2-[4-(3,5-dimethyl-4-phenyl-1H-pyrazol-1-yl)phenyl]ethyl}amino)carbonyl]-2-fluorobenzenesulfonamide;
2-[4-(2-isopropyl-4-phenyl-1H-imidazol-1-yl)phenyl]ethyl (2-chlorophenyl)sulfonylcarbamate;
2-[4-(2-ethyl-4-phenyl-1H-imidazole-1-yl)phenyl]ethyl (4-methyl phenyl)sulfonylcarbamate;
2-[4-(2-butyl-4-phenyl-1H-imidazol-1-yl)phenyl]ethyl (2-chlorophenyl)sulfonylcarbamate;
2-[4-(2-isobutyl-4-phenyl-1H-imidazol-1-yl)phenyl]ethyl (2-chlorophenyl)sulfonylcarbamate;
4-chloro-N-[({2-[4-(2-ethyl-4-phenyl-1H-imidazol-1-yl)phenyl]ethyl}amino)carbonyl]benzenesulfonamide;
   and their pharmaceutically acceptable salts.

Particularly preferred EP4-receptor antagonists for use in the present invention are selected from:
2-ethyl-4,6-dimethyl-1-(4-{2-[({[(4-methylphenyl)sulfonyl]amino}carbonyl)amino]ethyl}phenyl)-1H-imidazo[4,5-c]pyridine;
4-(6-chloro-2-ethyl-5-trifluoromethyl-1H-benzimidazol-1-yl)phenethyl-(4-methylphenyl)sulfonylcarbamate;
4-[(1S)-1-({[5-chloro-2-(3-fluorophenoxy)pyridin-3-yl]carbonyl}amino)ethyl]benzoic acid;
2-fluoro-N-{[(2-{4-[5-methyl-4-phenyl-3-(trifluoromethyl)-1H-pyrazol-1-yl]phenyl}ethyl)amino]carbonyl}benzenesulfonamide;
2-[4-(2-isopropyl-4-phenyl-1H-imidazol-1-yl)phenyl]ethyl (2-chlorophenyl)sulfonylcarbamate;
   and their pharmaceutically acceptable salts.

Preferably, the EP4-receptor antagonist is selected from those compounds, or pharmaceutically acceptable salts thereof, generally or specifically disclosed in WO-A-02/32900, particularly 2-ethyl-4,6-dimethyl-1-(4-{2-[({[(4-methylphenyl)sulfonyl]amino}carbonyl)amino]ethyl}phenyl)-1H-imidazo[4,5-c]pyridine and 4-(6-chloro-2-ethyl-5-trifluoromethyl-1H-benzimidazol-1-yl)phenethyl-(4-methylphenyl)sulfonylcarbamate, WO-A-03/087061, particularly 2-fluoro-N-{[(2-{4-[5-methyl-4-phenyl-3-(trifluoromethyl)-1H-pyrazol-1-yl]phenyl}ethyl)amino]carbonyl}benzenesulfonamide, WO-A-03/086390, particularly 2-[4-(2-isopropyl-4-phenyl-1H-imidazol-1-yl)phenyl]ethyl (2-chlorophenyl)sulfonylcarbamate, US60/500,131, particularly 4-[(1S)-1-({[5-chloro-2-(3-fluorophenoxy)pyridin-3-yl]carbonyl}amino)ethyl]benzoic acid, WO-A-03/016254, particularly 4-[2-[[2-(naphthalen-1-yl)propanoyl]amino]-4-methylthiomethylphenyl]butanoic acid, WO-A-02/20462, WO-A-02/16311, WO-A-01/62708, WO-A-00/15608, EP0985663, WO-A-00/03980, particularly methyl 7-[(1R,2R,3R)-3-hydroxy-2-[(E)-(3S)-3-hydroxy-4-(m-methoxymethylphenyl)-1-butenyl]-5-oxocyclopentyl]-5-thiaheptanoate, U.S. 60/568,088, U.S. 60/567,931, EP0855389, WO-A-02/50031, WO-A-02/50032, WO-A-02/50033, GB2330307, WO-A-01/10426, WO-A-00/18744, WO-A-00/16760, WO-A-98/55468, WO-A-00/21532, WO-A-00/24393, WO-A-99/47497, WO-A-01/72302, WO-A-00/18405 and WO-A-01/42281, as described above.

As a further aspect of the present invention, there is provided a combination comprising 2-ethyl-4,6-dimethyl-1-(4-{2-[({[(4-methylphenyl)sulfonyl]amino}carbonyl)amino]ethyl}phenyl)-1H-imidazo[4,5-c]pyridine, or a pharmaceutically acceptable salt thereof, and an alpha-2-delta ligand selected from gabapentin, pregabalin, [(1R,5R,6S)-6-(aminomethyl)bicyclo[3.2.0]hept-6-yl]acetic acid, 3-(1-aminomethyl-cyclohexylmethyl)-4H-[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)-acetic acid, (1α,3α,5α)(3-amino-methyl-bicyclo[3.2.0]hept-3-yl)-acetic acid, (3S,5R)-3-aminomethyl-5-methyl-octanoic acid, (3S,5R)-3-amino-5-methyl-heptanoic acid, (3S,5R)-3-amino-5-methyl-nonanoic acid, (3S,5R)-3-Amino-5-methyl-octanoic acid, (2S,4S)-4-(3-chlorophenoxy)proline and (2S,4S)-4-(3-fluorobenzyl)proline, or a pharmaceutically acceptable salt thereof.

As a further aspect of the present invention, there is provided a combination comprising 4-[(1S)-1-({[5-chloro-2-(3-fluorophenoxy)pyridin-3-yl]carbonyl}amino)ethyl]benzoic acid, or a pharmaceutically acceptable salt thereof, and an alpha-2-delta ligand selected from gabapentin, pregabalin, [(1R,5R,6S)-6-(aminomethyl)bicyclo[3.2.0]hept-6-yl]acetic acid, 3-(1-aminomethyl-cyclohexylmethyl)-4H-[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)-acetic acid, (1α,3α,5α)(3-amino-methyl-bicyclo[3.2.0]hept-3-yl)-acetic acid, (3S,5R)-3-aminomethyl-5-methyl-octanoic acid, (3S,5R)-3-amino-5-methyl-heptanoic acid, (3S,5R)-3-amino-5-methyl-nonanoic acid, (3S,5R)-3-amino-5-methyl-octanoic acid, (2S,4S)-4-(3-chlorophenoxy)proline and (2S,4S)-4-(3-fluorobenzyl)proline, or a pharmaceutically acceptable salt thereof.

As a yet further preferred aspect of the present invention, the combination is selected from:
2-ethyl-4,6-dimethyl-1-(4-{2-[({[(4-methylphenyl)sulfonyl]amino}carbonyl)amino]ethyl}phenyl)-1H-imidazo[4,5-c]pyridine and gabapentin;
2-ethyl-4,6-dimethyl-1-(4-{2-[({[(4-methylphenyl)sulfonyl]amino}carbonyl)amino]ethyl}phenyl)-1H-imidazo[4,5-c]pyridine and pregabalin;
2-ethyl-4,6-dimethyl-1-(4-{2-[({[(4-methylphenyl)sulfonyl]amino}carbonyl)amino]ethyl}phenyl)-1H-imidazo[4,5-c]pyridine and (1α,3α,5α)(3-amino-methyl-bicyclo[3.2.0]hept-3-yl)-acetic acid;
2-ethyl-4,6-dimethyl-1-(4-{2-[({[(4-methylphenyl)sulfonyl]amino}carbonyl)amino]ethyl}phenyl)-1H-imidazo[4,5-c]pyridine and [(1R,5R,6S)-6-(aminomethyl)bicyclo[3.2.0]hept-6-yl]acetic acid;
2-ethyl-4,6-dimethyl-1-(4-{2-[({[(4-methylphenyl)sulfonyl]amino}carbonyl)amino]ethyl}phenyl)-1H-imidazo[4,5-c]pyridine and (3S,4S)-(1-aminomethyl-3,4-dimethyl-cyclopentyl)-acetic acid;
2-ethyl-4,6-dimethyl-1-(4-{2-[({[(4-methylphenyl)sulfonyl]amino}carbonyl)amino]ethyl}phenyl)-1H-imidazo[4,5-c]pyridine and (2S,4S)-4-(3-fluorobenzyl)proline;
4-(6-chloro-2-ethyl-5-trifluoromethyl-1H-benzimidazol-1-yl)phenethyl-(4-methylphenyl)sulfonylcarbamate and gabapentin;
4-(6-chloro-2-ethyl-5-trifluoromethyl-1H-benzimidazol-1-yl)phenethyl-(4-methylphenyl)sulfonylcarbamate and pregabalin;
4-(6-chloro-2-ethyl-5-trifluoromethyl-1H-benzimidazol-1-yl)phenethyl-(4-methylphenyl)sulfonylcarbamate and (1α,3α,5α)(3-amino-methyl-bicyclo[3.2.0]hept-3-yl)-acetic acid;
4-(6-chloro-2-ethyl-5-trifluoromethyl-1H-benzimidazol-1-yl)phenethyl-(4-methylphenyl)sulfonylcarbamate and [(1R,5R,6S)-6-(aminomethyl)bicyclo[3.2.0]hept-6-yl]acetic acid;
4-(6-chloro-2-ethyl-5-trifluoromethyl-1H-benzimidazol-1-yl)phenethyl-(4-methyl phenyl)sulfonylcarbamate and (3S,4S)-(1-aminomethyl-3,4-dimethyl-cyclopentyl)-acetic acid;
4-(6-chloro-2-ethyl-5-trifluoromethyl-1H-benzimidazol-1-yl)phenethyl-(4-methylphenyl)sulfonylcarbamate and (2S,4S)-4-(3-chlorophenoxy)proline;
4-(6-chloro-2-ethyl-5-trifluoromethyl-1H-benzimidazol-1-yl)phenethyl-(4-methylphenyl)sulfonylcarbamate and (2S,4S)-4-(3-fluorobenzyl)proline;
4-[(1S)-1-({[5-chloro-2-(3-fluorophenoxy)pyridin-3-yl]carbonyl}amino)ethyl]benzoic acid and gabapentin;
4-[(1S)-1-({[5-chloro-2-(3-fluorophenoxy)pyridin-3-yl]carbonyl}amino)ethyl]benzoic acid and pregabalin;
4-[(1S)-1-({[5-chloro-2-(3-fluorophenoxy)pyridin-3-yl]carbonyl}amino)ethyl]benzoic acid and (1α,3α,5α)(3-amino-methyl-bicyclo[3.2.0]hept-3-yl)-acetic acid;
4-[(1S)-1-({[5-chloro-2-(3-fluorophenoxy)pyridin-3-yl]carbonyl}amino)ethyl]benzoic acid and [(1R,5R,6S)-6-(aminomethyl)bicyclo[3.2.0]hept-6-yl]acetic acid;
4-[(1S)-1-({[5-chloro-2-(3-fluorophenoxy)pyridin-3-yl]carbonyl}amino)ethyl]benzoic acid and (3S,4S)-(1-aminomethyl-3,4-dimethyl-cyclopentyl)-acetic acid;
4-[(1S)-1-({[5-chloro-2-(3-fluorophenoxy)pyridin-3-yl]carbonyl}amino)ethyl]benzoic acid and (2S,4S)-4-(3-chlorophenoxy)proline;
4-[(1S)-1-({[5-chloro-2-(3-fluorophenoxy)pyridin-3-yl]carbonyl}amino)ethyl]benzoic acid and (2S,4S)-4-(3-fluorobenzyl)proline;
2-fluoro-N-{[(2-{4-[5-methyl-4-phenyl-3-(trifluoromethyl)-1H-pyrazol-1-yl]phenyl}ethyl)amino]carbonyl}benzenesulfonamide and gabapentin;
2-fluoro-N-{[(2-{4-[5-methyl-4-phenyl-3-(trifluoromethyl)-1H-pyrazol-1-yl]phenyl}ethyl)amino]carbonyl}benzenesulfonamide and pregabalin;
2-fluoro-N-{[(2-{4-[5-methyl-4-phenyl-3-(trifluoromethyl)-1H-pyrazol-1-yl]phenyl}ethyl)amino]carbonyl}benzenesulfonamide and (1α,3α,5α)(3-amino-methyl-bicyclo[3.2.0]hept-3-yl)-acetic acid;
2-fluoro-N-{[(2-{4-[5-methyl-4-phenyl-3-(trifluoromethyl)-1H-pyrazol-1-yl]phenyl}ethyl)amino]carbonyl}benzenesulfonamide and [(1R,5R,6S)-6-(aminomethyl)bicyclo[3.2.0]hept-6-yl]acetic acid;
2-fluoro-N-{[(2-{4-[5-methyl-4-phenyl-3-(trifluoromethyl)-1H-pyrazol-1-yl]phenyl}ethyl)amino]carbonyl}benzenesulfonamide and (3S,4S)-(1-aminomethyl-3,4-dimethyl-cyclopentyl)-acetic acid;
2-fluoro-N-{[(2-{4-[5-methyl-4-phenyl-3-(trifluoromethyl)-1H-pyrazol-1-yl]phenyl}ethyl)amino]carbonyl}benzenesulfonamide and (2S,4S)-4-(3-chlorophenoxy)proline;
2-fluoro-N-{[(2-{4-[5-methyl-4-phenyl-3-(trifluoromethyl)-1H-pyrazol-1-yl]phenyl}ethyl)amino]carbonyl}benzenesulfonamide and (2S,4S)-4-(3-fluorobenzyl)proline;
2-[4-(2-isopropyl-4-phenyl-1H-imidazol-1-yl)phenyl]ethyl (2-chlorophenyl)sulfonylcarbamate and gabapentin;
2-[4-(2-isopropyl-4-phenyl-1H-imidazol-1-yl)phenyl]ethyl (2-chlorophenyl)sulfonylcarbamate and pregabalin;
2-[4-(2-isopropyl-4-phenyl-1H-imidazol-1-yl)phenyl]ethyl (2-chlorophenyl)sulfonylcarbamate and (1α,3α,5α)(3-amino-methyl-bicyclo[3.2.0]hept-3-yl)-acetic acid;
2-[4-(2-isopropyl-4-phenyl-1H-imidazol-1-yl)phenyl]ethyl (2-chlorophenyl)sulfonylcarbamate and [(1R,5R,6S)-6-(aminomethyl)bicyclo[3.2.0]hept-6-yl]acetic acid;
2-[4-(2-isopropyl-4-phenyl-1H-imidazol-1-yl)phenyl]ethyl (2-chlorophenyl)sulfonylcarbamate and (3S,4S)-(1-aminomethyl-3,4-dimethyl-cyclopentyl)-acetic acid; and
2-[4-(2-isopropyl-4-phenyl-1H-imidazol-1-yl)phenyl]ethyl (2-chlorophenyl)sulfonylcarbamate and (2S,4S)-4-(3-chlorophenoxy)proline;
2-[4-(2-isopropyl-4-phenyl-1H-imidazol-1-yl)phenyl]ethyl (2-chlorophenyl)sulfonylcarbamate and (2S,4S)-4-(3-fluorobenzyl)proline;
   or pharmaceutically acceptable salts or solvates of either or both components of any such combination.

Particularly preferred combinations of the invention include those in which each variable of the combination is selected from the suitable parameters for each variable. Even more preferable combinations of the invention include those where each variable of the combination is selected from the more suitable, most suitable, preferred or more preferred parameters for each variable.

The compounds of the combination of the present combination invention can exist in unsolvated forms as well as solvated forms, including hydrated forms. In general, the solvated forms, including hydrated forms, which may contain isotopic substitutions (e.g. D₂O), are equivalent to unsolvated forms and are encompassed within the scope of the present invention.

Certain of the compounds of the combination of the present invention possess one or more chiral centers and each center may exist in the R or S configuration. The present invention includes all individual enantiomeric and epimeric forms as well as the appropriate mixtures thereof. Separation of diastereoisomers or cis and trans isomers may be achieved by conventional techniques, e.g. by fractional crystallisation, chromatography or H.P.L.C. of a stereoisomeric mixture of a compound of the invention or a suitable salt or derivative thereof.

Pharmaceutically acceptable salts of EP4-receptor antagonists and alpha-2-delta ligands include the acid addition and base salts thereof.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloridee, hydrobromide, hydroiodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

For a review on suitable salts, see “Handbook of Pharmaceutical Salts: Properties, Selection, and Use” by Stahl and Wermuth (Wiley-VCH, Weinheim, Germany, 2002).

A pharmaceutically acceptable salt of an EP4-receptor antagonist or alpha-2-delta ligand may be readily prepared by mixing together solutions of the EP4-receptor antagonist or alpha-2-delta ligand and the desired acid or base, as appropriate. The salt may precipitate from solution and be collected by filtration or may be recovered by evaporation of the solvent. The degree of ionisation in the salt may vary from completely ionised to almost non-ionised.

The compounds of the combination of the invention may exist in both unsolvated and solvated forms. The term ‘solvate’ is used herein to describe a molecular complex comprising the compound of the combination of the invention and one or more pharmaceutically acceptable solvent molecules, for example, ethanol. The term ‘hydrate’ is employed when said solvent is water. Included within the scope of the invention are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionised, partially ionised, or non-ionised. For a review of such complexes, see J Pharm Sci, 64 (8), 1269-1288 by Haleblian (August 1975).

Hereinafter all references to an EP4-receptor antagonist or alpha-2-delta ligand include references to salts, solvates and complexes thereof and to solvates and complexes of salts thereof.

The term ‘EP4-receptor antagonist’ includes EP4-receptor antagonists as hereinbefore defined, polymorphs, prodrugs, and isomers thereof (including optical, geometric and tautomeric isomers) as hereinafter defined and isotopically-labeled EP4-receptor antagonists.

The term ‘alpha-2-delta ligand’ includes alpha-2-delta ligands as hereinbefore defined, polymorphs, prodrugs, and isomers thereof (including optical, geometric and tautomeric isomers) as hereinafter defined and isotopically-labeled alpha-2-delta ligands.

As stated, the invention includes all polymorphs of the compounds of formula (I) as hereinbefore defined.

A number of the alpha-2-delta ligands of the combination of the present invention are amino acids. Since amino acids are amphoteric, pharmacologically compatible salts can be salts of appropriate non-toxic inorganic or organic acids or bases. Salts with quaternary ammonium ions can also be prepared with, for example, the tetramethyl-ammonium ion. The alpha-2-delta ligands of the combination of the invention may also be formed as a zwitterion.

A suitable salt for amino acid compounds of the present invention is the hydrochloride salt.

Prodrugs of the above compounds of the combination of the invention are included in the scope of the instant invention. The chemically modified drug, or prodrug, should have a different pharmacokinetic profile to the parent, enabling easier absorption across the mucosal epithelium, better salt formulation and/or solubility, improved systemic stability (for an increase in plasma half-life, for example). These chemical modifications may be

(1) Ester or amide derivatives which may be cleaved by, for example, esterases or lipases. For ester derivatives, the ester is derived from the carboxylic acid moiety of the drug molecule by known means. For amide derivatives, the amide may be derived from the carboxylic acid moiety or the amine moiety of the drug molecule by known means.
(2) Peptides which may be recognized by specific or nonspecific proteinases. A peptide may be coupled to the drug molecule via amide bond formation with the amine or carboxylic acid moiety of the drug molecule by known means.
(3) Derivatives that accumulate at a site of action through membrane selection of a prodrug form or modified prodrug form.
Any combination of 1 to 3.
Aminoacyl-glycolic and -lactic esters are known as prodrugs of amino acids (Wermuth C. G., Chemistry and Industry, 1980:433-435). The carbonyl group of the amino acids can be esterified by known means. Prodrugs and soft drugs are known in the art (Palomino E., Drugs of the Future, 1990; 15(4):361-368). The last two citations are hereby incorporated by reference.

The combination of the present invention is useful for the general treatment of pain, particularly inflammatory, neuropathic, visceral or nociceptive pain.

As a further aspect of the invention, there is provided the use of an EP4-receptor antagonist and an alpha-2-delta ligand in the manufacture of a medicament for the curative, prophylactic or palliative treatment of pain, particularly inflammatory, neuropathic, visceral or nociceptive pain.

As an alternative feature, the invention provides the use of a synergistic effective amount of an EP4-receptor antagonist and an alpha-2-delta ligand in the manufacture of a medicament for the curative, prophylactic or palliative treatment of pain, particularly inflammatory, neuropathic, visceral or nociceptive pain.

As an alternative aspect, there is provided a method for the curative, prophylactic or palliative treatment of pain, particularly inflammatory, neuropathic, visceral or nociceptive pain, comprising simultaneous, sequential or separate administration of a therapeutically effective amount of an alpha-2-delta ligand and an EP4-receptor antagonist, to a mammal in need of said treatment.

As an alternative feature, there is provided a method for the curative, prophylactic or palliative treatment of pain, particularly inflammatory, neuropathic, visceral or nociceptive pain, comprising simultaneous, sequential or separate administration of a therapeutically synergistic amount of an alpha-2-delta ligand and EP4-receptor antagonist, to a mammal in need of said treatment.

Physiological pain is an important protective mechanism designed to warn of danger from potentially injurious stimuli from the external environment. The system operates through a specific set of primary sensory neurones and is exclusively activated by noxious stimuli via peripheral transducing mechanisms (Millan 1999 Prog. Neurobio. 57: 1-164 for an integrative Review). These sensory fibres are known as nociceptors and are characterised by small diameter axons with slow conduction velocities. Nociceptors encode the intensity, duration and quality of noxious stimulus and by virtue of their topographically organised projection to the spinal cord, the location of the stimulus. The nociceptors are found on nociceptive nerve fibres of which there are two main types, A-delta fibres (myelinated) and C fibres (non-myelinated). The activity generated by nociceptor input is transferred after complex processing in the dorsal horn, either directly or via brain stem relay nuclei to the ventrobasal thalamus and then on to the cortex, where the sensation of pain is generated.

Intense acute pain and chronic pain may involve the same pathways driven by pathophysiological processes and as such cease to provide a protective mechanism and instead contribute to debilitating symptoms associated with a wide range of disease states. Pain is a feature of many trauma and disease states. When a substantial injury, via disease or trauma, to body tissue occurs the characteristics of nociceptor activation are altered. There is sensitisation in the periphery, locally around the injury and centrally where the nociceptors terminate. This leads to hypersensitivity at the site of damage and in nearby normal tissue. In acute pain these mechanisms can be useful and allow for the repair processes to take place and the hypersensitivity returns to normal once the injury has healed. However, in many chronic pain states, the hypersensitivity far outlasts the healing process and is normally due to nervous system injury. This injury often leads to maladaptation of the afferent fibres (Woolf & Salter 2000 Science 288: 1765-1768). Clinical pain is present when discomfort and abnormal sensitivity feature among the patient's symptoms. Patients tend to be quite heterogeneous and may present with various pain symptoms. There are a number of typical pain subtypes: 1) spontaneous pain which may be dull, burning, or stabbing; 2) pain responses to noxious stimuli are exaggerated (hyperalgesia); 3) pain is produced by normally innocuous stimuli (allodynia) (Meyer et al., 1994 Textbook of Pain 13-44). Although patients with back pain, arthritis pain, CNS trauma, or neuropathic pain may have similar symptoms, the underlying mechanisms are different and, therefore, may require different treatment strategies. Therefore pain can be divided into a number of different areas because of differing pathophysiology, these include nociceptive, inflammatory, neuropathic pain etc. It should be noted that some types of pain have multiple aetiologies and thus can be classified in more than one area, e.g. Back pain, Cancer pain have both nociceptive and neuropathic components.

Nociceptive pain is induced by tissue injury or by intense stimuli with the potential to cause injury. Pain afferents are activated by transduction of stimuli by nociceptors at the site of injury and sensitise the spinal cord at the level of their termination. This is then relayed up the spinal tracts to the brain where pain is perceived (Meyer et al., 1994 Textbook of Pain 13-44). The activation of nociceptors activates two types of afferent nerve fibres. Myelinated A-delta fibres transmitted rapidly and are responsible for the sharp and stabbing pain sensations, whilst unmyelinated C fibres transmit at a slower rate and convey the dull or aching pain. Moderate to severe acute nociceptive pain is a prominent feature of, but is not limited to pain from strains/sprains, post-operative pain (pain following any type of surgical procedure), posttraumatic pain, burns, myocardial infarction, acute pancreatitis, and renal colic. Also cancer related acute pain syndromes commonly due to therapeutic interactions such as chemotherapy toxicity, immunotherapy, hormonal therapy and radiotherapy. Moderate to severe acute nociceptive pain is a prominent feature of, but is not limited to, cancer pain which may be tumour related pain, (e.g. bone pain, headache and facial pain, viscera pain) or associated with cancer therapy (e.g. postchemotherapy syndromes, chronic postsurgical pain syndromes, post radiation syndromes), back pain which may be due to herniated or ruptured intervertabral discs or abnormalities of the lumber facet joints, sacroiliac joints, paraspinal muscles or the posterior longitudinal ligament.

Neuropathic pain is defined as pain initiated or caused by a primary lesion or dysfunction in the nervous system (IASP definition). Nerve damage can be caused by trauma and disease and thus the term ‘neuropathic pain’ encompasses many disorders with diverse aetiologies. These include but are not limited to, Diabetic neuropathy, Post herpetic neuralgia, Back pain, Cancer neuropathy, HIV neuropathy, Phantom limb pain, Carpal Tunnel Syndrome, chronic alcoholism, hypothyroidism, trigeminal neuralgia, uremia, or vitamin deficiencies. Neuropathic pain is pathological as it has no protective role. It is often present well after the original cause has dissipated, commonly lasting for years, significantly decreasing a patients quality of life (Woolf and Mannion 1999 Lancet 353: 1959-1964). The symptoms of neuropathic pain are difficult to treat, as they are often heterogeneous even between patients with the same disease (Woolf & Decosterd 1999 Pain Supp. 6: S141-S147; Woolf and Mannion 1999 Lancet 353: 1959-1964). They include spontaneous pain, which can be continuous, or paroxysmal and abnormal evoked pain, such as hyperalgesia (increased sensitivity to a noxious stimulus) and allodynia (sensitivity to a normally innocuous stimulus).

The inflammatory process is a complex series of biochemical and cellular events activated in response to tissue injury or the presence of foreign substances, which result in swelling and pain (Levine and Taiwo 1994: Textbook of Pain 45-56). Arthritic pain makes up the majority of the inflammatory pain population. Rheumatoid disease is one of the commonest chronic inflammatory conditions in developed countries and rheumatoid arthritis is a common cause of disability. The exact aetiology of RA is unknown, but current hypotheses suggest that both genetic and microbiological factors may be important (Grennan & Jayson 1994 Textbook of Pain 397-407). It has been estimated that almost 16 million Americans have symptomatic osteoarthritis (OA) or degenerative joint disease, most of whom are over 60 years of age, and this is expected to increase to 40 million as the age of the population increases, making this a public health problem of enormous magnitude (Houge & Mersfelder 2002 Ann Pharmacother. 36: 679-686; McCarthy et al., 1994 Textbook of Pain 387-395). Most patients with OA seek medical attention because of pain. Arthritis has a significant impact on psychosocial and physical function and is known to be the leading cause of disability in later life. Other types of inflammatory pain include but are not limited to inflammatory bowel diseases (IBD);

Other types of pain include but are not limited to;

Musculo-skeletal disorders including but not limited to myalgia, fibromyalgia, spondylitis, sero-negative (non-rheumatoid) arthropathies, non-articular rheumatism, dystrophinopathy, Glycogenolysis, polymyositis, pyomyositis.

Central pain or ‘thalamic pain’ as defined by pain caused by lesion or dysfunction of the nervous system including but not limited to central post-stroke pain, multiple sclerosis, spinal cord injury, Parkinson's disease and epilepsy.

Heart and vascular pain including but not limited to angina, myocardical infarction, mitral stenosis, pericarditis, Raynaud's phenomenon, scleredoma, scleredoma, skeletal muscle ischemia.

Visceral pain, and gastrointestinal disorders. The viscera encompasses the organs of the abdominal cavity. These organs include the sex organs, spleen and part of the digestive system. Pain associated with the viscera can be divided into digestive visceral pain and non-digestive visceral pain. Commonly encountered gastrointestinal (GI) disorders include the functional bowel disorders (FBD) and the inflammatory bowel diseases (IBD). These GI disorders include a wide range of disease states that are currently only moderately controlled, including—for FBD, gastro-esophageal reflux, dyspepsia, the irritable bowel syndrome (IBS) and functional abdominal pain syndrome (FAPS), and—for IBD, Crohn's disease, ileitis, and ulcerative colitis, which all regularly produce visceral pain. Other types of visceral pain include the pain associated with dysmenorrhea, pelvic pain, cystitis and pancreatitis.

Head pain including but not limited to migraine, migraine with aura, migraine without aura cluster headache, tension-type headache.

Orofacial pain including but not limited to dental pain, temporomandibular myofascial pain.

The invention also relates to therapeutic use of the present combinations as agents for treating or relieving the symptoms of neurodegenerative disorders. Such neurodegenerative disorders include, for example, Alzheimer's disease, Huntington's disease, Parkinson's disease, and Amyotrophic Lateral Sclerosis. The present invention also covers treating neurodegenerative disorders termed acute brain injury. These include but are not limited to: stroke, head trauma, and asphyxia. Stroke refers to a cerebral vascular disease and may also be referred to as a cerebral vascular accident (CVA) and includes acute thromboembolic stroke. Stroke includes both focal and global ischemia. Also, included are transient cerebral ischemic attacks and other cerebral vascular problems accompanied by cerebral ischemia. These vascular disorders may occur in a patient undergoing carotid endarterectomy specifically or other cerebrovascular or vascular surgical procedures in general, or diagnostic vascular procedures including cerebral angiography and the like. Other incidents are head trauma, spinal cord trauma, or injury from general anoxia, hypoxia, hypoglycemia, hypotension as well as similar injuries seen during procedures from embole, hyperfusion, and hypoxia. The instant invention would be useful in a range of incidents, for example, during cardiac bypass surgery, in incidents of intracranial hemorrhage, in perinatal asphyxia, in cardiac arrest, and status epilepticus.

A skilled physician will be able to determine the appropriate situation in which subjects are susceptible to or at risk of, for example, stroke as well as suffering from stroke for administration by methods of the present invention.

The combinations of the present invention are also expected to be useful in the treatment of depression. Depression can be the result of organic disease, secondary to stress associated with personal loss, or idiopathic in origin. There is a strong tendency for familial occurrence of some forms of depression suggesting a mechanistic cause for at least some forms of depression. The diagnosis of depression is made primarily by quantification of alterations in patients' mood. These evaluations of mood are generally performed by a physician or quantified by a neuropsychologist using validated rating scales, such as the Hamilton Depression Rating Scale or the Brief Psychiatric Rating Scale. Numerous other scales have been developed to quantify and measure the degree of mood alterations in patients with depression, such as insomnia, difficulty with concentration, lack of energy, feelings of worthlessness, and guilt. The standards for diagnosis of depression as well as all psychiatric diagnoses are collected in the Diagnostic and Statistical Manual of Mental Disorders (Fourth Edition) referred to as the DSM-IV-R manual published by the American Psychiatric Association, 1994.

As a yet further aspect, there is provided the use of a combination of an alpha-2-delta ligand and an EP4-receptor antagonist in the manufacture of a medicament for the treatment of a disease selected from epilepsy, faintness attacks, hypokinesia, cranial disorders, neurodegenerative disorders, depression, anxiety, panic, pain, irritable bowel syndrome, sleep disorders, osteoarthritis, rheumatoid arthritis, neuropathological disorders, visceral pain, functional bowel disorders, inflammatory bowel diseases, pain associated with dysmenorrhea, pelvic pain, cystitis and pancreatitis.

As a alternative aspect, there is provided a method for treating a disease selected from epilepsy, faintness attacks, hypokinesia, cranial disorders, neurodegenerative disorders, depression, anxiety, panic, pain, irritable bowel syndrome, sleep disorders, osteoarthritis, rheumatoid arthritis, neuropathological disorders, visceral pain, functional bowel disorders, inflammatory bowel diseases, pain associated with dysmenorrhea, pelvic pain, cystitis and pancreatitis comprising administering a therapeutically effective amount of a combination of an alpha-2-delta ligand and an EP4-receptor antagonist to a mammal in need of said treatment.

### DETAILED DESCRIPTION OF THE INVENTION

The compounds of the combination of the present invention are prepared by methods well known to those skilled in the art. Specifically, the patents, patent applications and publications, mentioned hereinabove, each of which is hereby incorporated herein by reference, exemplify compounds which can be used in the combinations, pharmaceutical compositions, methods and kits in accord with the present invention, and refer to methods of preparing those compounds.

The following reaction schemes illustrate the preparation of EP4-receptor antagonists described in U.S. Patent Application No. US 60/500,0131. Unless otherwise indicated R¹ through R⁶ and A, B, E and X in the reaction Schemes and discussion that follow are defined as above. The term “protecting group”, as used hereinafter, means a hydroxy or amino protecting group which is selected from typical hydroxy or amino protecting groups described in Protective Groups in Organic Synthesis edited by T. W. Greene et al. (John Wiley & Sons, 1991);

### Scheme 1:

This illustrates the preparation of compounds of formula (Ia) wherein R⁵ represents —CO₂H.

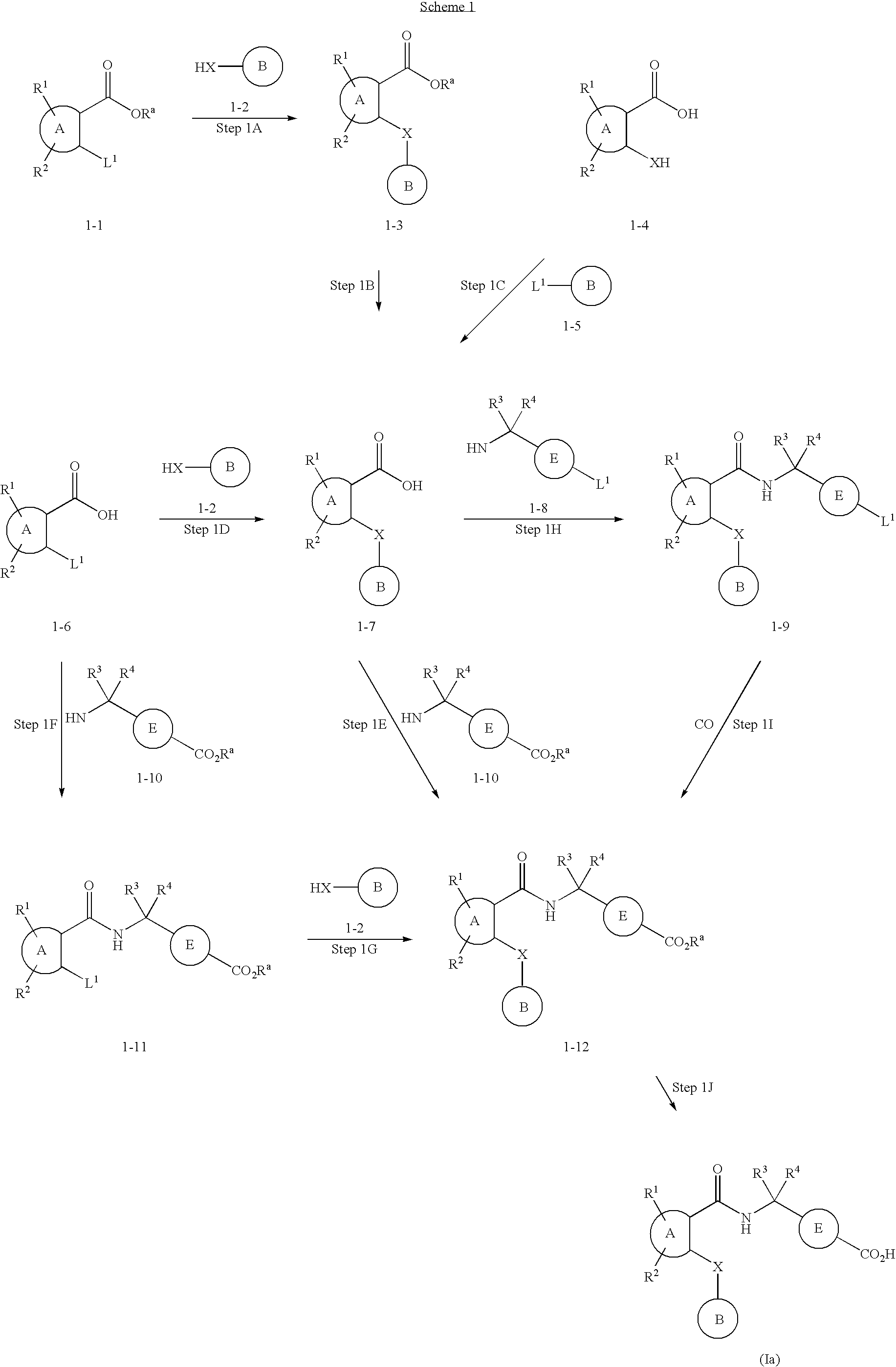

In the above formula, L¹ represents a halogen atom such as, chlorine, bromine or iodine; an alkanesulfonyloxy group such as, a methanesulfonyl group; an arylsulfonyloxy group such as, a p-toluenesulfonyloxy group; a haloalkanesulfonyloxy group such as, a trifluoromethanesulfonyloxy group; or a boronic acid group; R^{a} represents an alkyl groups having from 1 to 6 carbon atoms or an aralkyl group having from 7 to 12 carbon atoms; and all other symbols are as already defined.

### Step 1A

In this Step, a compound of formula 1-3 may be prepared by the coupling reaction of an ester compound of formula 1-1 with a cyclic compound of formula 1-2 in an inert solvent.

The coupling reaction may be carried out in the absence or presence of a base in a reaction inert solvent or without solvent. A preferred base is selected from, for example, an alkali or alkaline earth metal hydroxide, alkoxide, carbonate, or hydride, such as sodium hydroxide, potassium hydroxide, sodium methoxide, sodium ethoxide, potassium tert-butoxide, sodium carbonate, cesium carbonate or potassium carbonate, 2-tert-butylimino-2-diethylamino-1,3-dimethylperhydro-1,3,2-diazaphosphorine (BEMP), tert-butylimino-tri(pyrrolidino)phosphorane (BTPP), cesium fluoride (CsF), potassium fluoride, sodium hydride or potassium hydride, or an amine such as triethylamine, tributylamine, diisopropylethylamine, 2,6-lutidine, pyridine or dimethylaminopyridine. Preferred reaction inert solvents include, for example, acetone, benzene, toluene, xylene, o-dichlorobenzene, nitrobenzene, nitromethane, pyridine, dichloromethane, dichloroethane, tetrahydrofuran, dimethylformamide (DMF), dimethylacetamide (DMA), dioxane, dimethylsulfoxide (DMSO), acetonitrile, sulfolane, N-methylpyrrolidinone (NMP), methyl ethyl ketone (2-butanone), tetrahydrofuran (THF), dimethoxyethane (DME) or mixtures thereof. Reaction temperatures are generally in the range of 0 to 200° C., preferably in the range of room temperature to 150° C. Reaction times are, in general, from 1 minute to a day, preferably from 1 hour to 10 hours. If desired, the reaction may be conducted in the presence of metal catalyst such as copper (e.g. copper bronze or cuprous iodide) and nickel.

When L¹ represents a boronic acid group, the reaction may be carried out in the presence of a suitable catalyst to form the compound of formula 1-3 by any synthetic procedure applicable to structure-related compounds known to those skilled in the literature (e.g., Lam, P. Y. S.; Clark, C. G.; Saubern, S; Adams, J; Winters, M. P.; Chan, D. M. T.; Combs, A., Tetrahedron Lett., 1998, 39, 2941-2944., Kiyomori, A.; Marcoux, J.; Buchwald, S. L., Tetrahedron Lett., 1999, 40, 2657-2660., Lam, P. Y. S.; Deudon, S.; Averill, K. M.; Li, R.; He, M. Y.; DeShong, P.; Clark, C. G., J. Am. Chem. Soc., 2000, 122, 7600-7601., Collman, J. P.; Zhong, M., Org. Lett., 2000, 2, 1233-1236). A preferred reaction catalyst is selected from, for example, tetrakis(triphenylphosphine)-palladium, bis(triphenylphosphine)palladium(II) chloride, copper(0), copper(I) acetate, copper(I) bromide, copper(I) chloride, copper(I) iodide, copper(I) oxide, copper(II) trifluoromethanesulfonate, copper(II) acetate, copper(II) bromide, copper(II) chloride, copper(II) iodide, copper(II) oxide, or copper(II) trifluoromethanesulfonate.

### Step 1B

In this Step, an acid compound of formula 1-7 may be prepared by hydrolysis of the ester compound of formula 1-3 in a solvent.

The hydrolysis may be carried out by conventional procedures. In a typical procedure, the hydrolysis carried out under the basic condition, e.g. in the presence of sodium hydroxide, potassium hydroxide or lithium hydroxide. Suitable solvents include, for example, alcohols such as methanol, ethanol, propanol, butanol, 2-methoxyethanol, and ethylene gylcol; ethers such as tetrahydrofuran (THF), 1,2-dimethoxyethane (DME), and 1,4-dioxane; amides such as N,N-dimethylformamide (DMF) and hexamethylphospholictriamide; and sulfoxides such as dimethyl sulfoxide (DMSO). This reaction may be carried out at a temperature in the range from −20 to 100° C., usually from 20° C. to 65° C. for 30 minutes to 24 hours, usually 60 minutes to 10 hour.

The hydrolysis may also be carried out under the acidic condition, e.g. in the presence of e.g. in the presence of hydrogen halides, such as hydrogen chloride and hydrogen bromide; sulfonic acids, such as p-toluenesulfonic acid and benzenesulfonic acid; pyridium p-toluenesulfonate; and carboxylic acid, such as acetic acid and trifluoroacetic acid. Suitable solvents include, for example, alcohols such as methanol, ethanol, propanol, butanol, 2-methoxyethanol, and ethylene gylcol; ethers such as tetrahydrofuran (THF), 1,2-dimethoxyethane (DME), and 1,4-dioxane; amides such as N,N-dimethylformamide (DMF) and hexamethylphospholictriamide; and sulfoxides such as dimethyl sulfoxide (DMSO). This reaction may be carried out at a temperature in the range from −20 to 100° C., usually from 20° C. to 65° C. for 30 minutes to 24 hours, usually 60 minutes to 10 hour.

### Step 1C

In this Step, the acid compound of formula 1-7 may also be prepared by coupling reaction of an acid compound of formula 1-4 with a cyclic compound of formula 1-5. This reaction is essentially the same as and may be carried out in the same manner as and using the same reagents and reaction conditions as Step 1A in Scheme 1.

### Step 1D

In this Step, the acid compound of formula 1-7 may also be prepared by coupling reaction of an acid compound of formula 1-6 with the cyclic compound of formula 1-2. This reaction is essentially the same as and may be carried out in the same manner as and using the same reagents and reaction conditions as Step 1A in Scheme 1.

In this Step, an amide compound of formula 1-12 may be prepared by the coupling reaction of an amine compound of formula 1-10 with the acid compound of formula 1-7 in the presence or absence of a coupling reagent in an inert solvent. If desired, this reaction may be carried out in the presence or absence of an additive such as 1-hydroroxybenzotriazole or 1-hydroxyazabenzotriazole.

The reaction is normally and preferably effected in the presence of a solvent. There is no particular restriction on the nature of the solvent to be employed, provided that it has no adverse effect on the reaction or on the reagents involved and that it can dissolve the reagents, at least to some extent. Examples of suitable solvents include: acetone, nitromethane, DMF, sulfolane, DMSO, NMP, 2-butanone, acetonitrile; halogenated hydrocarbons, such as dichloromethane, dichloroethane, chloroform; and ethers, such as tetrahydrofuran and dioxane.

The reaction can take place over a wide range of temperatures, and the precise reaction temperature is not critical to the invention. The preferred reaction temperature will depend upon such factors as the nature of the solvent, and the starting material or reagent used. However, in general, we find it convenient to carry out the reaction at a temperature of from −20° C. to 100° C., more preferably from about 0° C. to 60° C. The time required for the reaction may also vary widely, depending on many factors, notably the reaction temperature and the nature of the reagents and solvent employed. However, provided that the reaction is effected under the preferred conditions outlined above, a period of 5 minutes to 1 week, more preferably 30 minutes to 24 hours, will usually suffice.

Suitable coupling reagents are those typically used in peptide synthesis including, for example, diimides (e.g., dicyclohexylcarbodiimide (DCC), water soluble carbodiimide (WSC)), 2-ethoxy-N-ethoxycarbonyl-1,2-dihydroquinoline, 2-bromo-1-ethyl pyridinium tetrafluoroborate (BE P), 2-chloro-1,3-dimethyl imidazolinium chloride, benzotriazol-1-yloxy-tris(dimethylamino)phosphonium hexafluorophosphate (BOP), diethyl azodicarboxylate-triphenylphosphine, diethylcyanophosphate, diethylphosphorylazide, 2-chloro-1-methylpyridinium iodide, N,N′-carnbonyldiimidazole, benzotriazole-1-yl diethyl phosphate, ethyl chloroformate or isobutyl chloroformate. If desired, the reaction may be carried out in the presence of a base such as, N,N-diisopropylethylamine, N-methylmorpholine and triethylamine. The amide compound of formula 1-12 may be formed via an acylhalide, which may be obtained by the reaction with halogenating agents such as oxalylchloride, phosphorus oxychloride and thionyl chloride. The resulting acylhalide may be converted to the corresponding amide compound by treating with the amine compound of formula 1-10 under the similar conditions as described in this Step.

### Step 1F

In this Step, an amide compound of formula 1-11 may be prepared by coupling reaction of the acid compound of formula 1-6 with the amine compound of formula 1-10. This reaction is essentially the same as and may be carried out in the same manner as and using the same reagents and reaction conditions as Step 1E in Scheme 1.

### Step 1G

In this Step, the amide compound of formula 1-12 may also be prepared by coupling reaction of the compound of formula 1-11 with the cyclic compound of formula 1-2. This reaction is essentially the same as and may be carried out in the same manner as and using the same reagents and reaction conditions as Step 1A in Scheme 1.

### Step 1H

In this Step, an amide compound of formula 1-9 may be prepared by coupling reaction of the acid compound of formula 1-7 with an amino compound of formula 1-8. This reaction is essentially the same as and may be carried out in the same manner as and using the same reagents and reaction conditions as Step 1E in Scheme 1.

### Step 1I

In this Step, the amide compound of formula 1-12 may also be prepared by reacting the amide compound of formula 1-9 with carbon monoxide and alcohol (e.g. methanol or ethanol) in the presence of a catalyst and/or base in an inert solvent. Example of suitable catalysts include: palladium reagents, such as palladium acetate and palladium dibenzylacetone. Example of suitable bases include: N,N-diisopropylethylamine, N-methylmorpholine and triethylamine. If desired, this reaction may be carried out in the presence or absence of an additive such as 1,1′-bis(diphenylphosphino)ferrocene, triphenylphosphine or 1,3-bis-(diphenylphosphino)propane (DPPP).

The reaction is normally and preferably effected in the presence of a solvent. There is no particular restriction on the nature of the solvent to be employed, provided that it has no adverse effect on the reaction or on the reagents involved and that it can dissolve the reagents, at least to some extent. Examples of suitable solvents include: acetone, nitromethane, DMF, sulfolane, DMSO, NMP, 2-butanone, acetonitrile; halogenated hydrocarbons, such as dichloromethane, dichloroethane, chloroform; and ethers, such as tetrahydrofuran and dioxane.

The reaction can take place over a wide range of temperatures, and the precise reaction temperature is not critical to the invention. The preferred reaction temperature will depend upon such factors as the nature of the solvent, and the starting material or reagent used. However, in general, we find it convenient to carry out the reaction at a temperature of from −20° C. to 150° C., more preferably from about 50° C. to 80° C. The time required for the reaction may also vary widely, depending on many factors, notably the reaction temperature and the nature of the reagents and solvent employed. However, provided that the reaction is effected under the preferred conditions outlined above, a period of 30 minutes to 24 hours, more preferably 1 hour to 10 hours, will usually suffice.

### Step 1J

In this Step, an acid compound of formula Ia may be prepared by hydrolysis of the ester compound of formula 1-12.

This reaction is essentially the same as and may be carried out in the same manner as and using the same reagents and reaction conditions as Step 1B in Scheme 1.

### Scheme 2:

This illustrates the preparation of compounds of formula (Ib) wherein R⁵ represents —CO₂H; and X represents a group of formula:

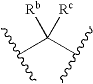

wherein R^{b} and R^{c} independently represents a hydrogen atom or an alkyl group having from 1 to 3 carbon atoms.

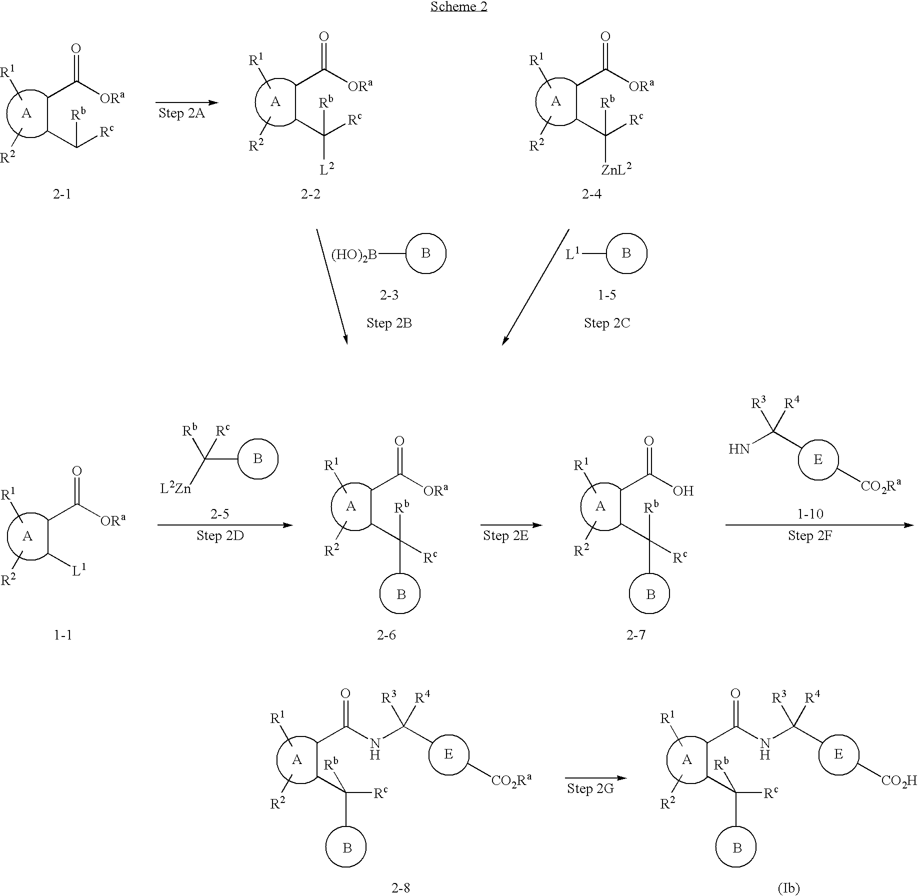

In the above formula, L² represents a halogen atom such as, chlorine, bromine or iodine; and all other symbols are as already defined.

### Step 2A

In this Step, a 2-alkyl cyclic ester compound of formula 2-1 may be converted to compound with a leaving group L² of formula 2-2 under conditions known to those skilled in the art.

The halogenated compound 2-2 may be generally prepared by halogenation with a halogenating reagent in a reaction-inert solvent. Examples of suitable solvents include: such as aqueous or non-aqueous organic solvents such as tetrahydrofuran, dioxane, dimethylformamide, acetonitrile; alcohols, such as methanol or ethanol; halogenated hydrocarbons, such as dichloromethane, dichloroethane or chloroform; and acetic acid. Suitable halogenating reagents include, for example, bromine, chlorine, iodine, N-chlorosuccimide, N-bromosuccimide, 1,3-dibromo-5,5-dimethylhydantoin, bis(dimethylacetamide)hydrogen tribromide, tetrabutylammonium tribromide, bromodimethylsulfonium bromide, hydrogen bromide-hydrogen peroxide, nitrodibromoacetonitrile or copper(II) bromide. The reaction can be carried out at a temperature of from 0° C. to 200° C., more preferably from 20° C. to 120° C. Reaction times are, in general, from 5 minutes to 48 hours, more preferably 30 minutes to 24 hours, will usually suffice.

### Step 2B

In this Step, a compound of formula 2-5 may be prepared by the coupling reaction of the halogenated compound of formula 2-2 with a boronic acid compound of formula 2-3 in an inert solvent.

Examples of suitable solvents include: aromatic hydrocarbons, such as benzene, toluene, xylene, nitrobenzene, and pyridine; halogenated hydrocarbons, such as methylene chloride, chloroform, carbon tetrachloride and dichloroethane; ethers, such as diethyl ether, diisopropyl ether, DME, tetrahydrofuran and dioxane; ethyl acetate, acetonitrile, N,N-dimethylformamide, dimethylsulfoxide and water.

The reaction can be carried out at a temperature of from −100° C. to 250° C., more preferably from 0° C. to the reflux temperature. Reaction times are, in general, from 1 minute to 10 day, more preferably from 20 minutes to 5 days, will usually suffice. from 1 minute to a day, preferably from 1 hour to 10 hours.

This reaction may be carried out in the presence a suitable catalyst. There is likewise no particular restriction on the nature of the catalysts used, and any catalysts commonly used in reactions of this type may equally be used here. Examples of such catalysts include: tetrakis(triphenylphosphine)-palladium, bis(triphenylphosphine)palladium(II) chloride, copper(0), copper(I) acetate, copper(I) bromide, copper(I) chloride, copper(I) iodide, copper(I) oxide, copper(II) trifluoromethanesulfonate, copper(II) acetate, copper(II) bromide, copper(II) chloride, copper(II) iodide, copper(II) oxide, copper(II) trifluoromethanesulfonate palladium(II) acetate, palladium(II) chloride, bisacetonitriledichloropalladium(0), bis(dibenzylideneacetone)palladium(0), tris(dibenzylideneacetone)dipalladium(0) or [1,1′-bis(diphenylphosphino)ferrocene]palladium(II) dichloride.

This reaction may be carried out in the presence of a suitable additive agent. Examples of such additive agents include: tiphenylphosphine, tri-tert-butylphosphine, 1,1′-bis(diphenylphosphino)ferrocene, tri-2-furylphosphine, tri-o-tolylphosphine, 2-(dichlorohexylphosphino)biphenyl or triphenylarsine.

This reaction may be carried out in the presence or absence of a base. There is likewise no particular restriction on the nature of the bases used, and any base commonly used in reactions of this type may equally be used here. Examples of such bases include: lithium hydroxide, sodium hydroxide, potassium hydroxide, barium hydroxide, sodium carbonate, potassium carbonate, sodium bicarbonate, cesium carbonate, thallium(I) carbonate, sodium ethoxide, potassium tert-butoxide, potassium acetate, cesium fluoride, tetrabutylammonium fluoride, tetrabutylammonium chloride, tetrabutylammonium iodide, pyridine, 1,8-diazabicyclo[5.4.0]undecan, picoline, 4-(N,N-dimethylamino)pyridine, triethylamine, tributylamine, diisopropylethylamine, N-methylmorphorine and N-methylpiperidine.

This reaction may be carried out in the presence or absence of a dehydrating reagent. There is likewise no particular restriction on the nature of the dehydrating reagents used, and any dehydrating reagents commonly used in reactions of this type may equally be used here. Examples of such dehydrating reagents include: molecular sieves.

### Step 2C

In this Step, the compound of formula 2-7 may be prepared by the coupling reaction of a zinc compound of formula 2-4 with the compound of formula 1-5 in an inert solvent.

Examples of suitable solvents include: aromatic hydrocarbons, such as benzene, toluene, xylene, nitrobenzene, and pyridine; halogenated hydrocarbons, such as methylene chloride, chloroform, carbon tetrachloride and dichloroethane; ethers, such as diethyl ether, diisopropyl ether, tetrahydrofuran and dioxane; ethyl acetate, acetonitrile, N,N-dimethylformamide, dimethylsulfoxide. This reaction may be carried out in the presence a suitable catalyst. Example of suitable catalysts include: dichlorobis[triphenylphosphine]nickel, tetrakis(triphenylphosphine)-palladium, bis(triphenylphosphine)palladium(II) chloride, copper(0), copper(I) acetate, copper(I) bromide, copper(I) chloride, copper(I) iodide, copper(I) oxide, copper(II) trifluoromethanesulfonate, copper(II) acetate, copper(II) bromide, copper(II) chloride, copper(II) iodide, copper(II) oxide, copper(II) trifluoromethanesulfonate palladium(II) acetate, palladium(II) chloride, bisacetonitriledichloropalladium(0), bis(dibenzylideneacetone)palladium(0), tris(dibenzylideneacetone)dipalladium(0) or [1,1′-bis(diphenylphosphino)ferrocene]palladium(II) dichloride.

This reaction can be carried out at temperature of −50° C. to 150° C., preferably from about −10° C. to 80° C. for 5 minutes to 48 hours, preferably 30 minutes to 24 hours.

### Step 2D

In this Step, the compound of formula 2-7 may be prepared by the coupling reaction of a zinc compound of formula 2-6 with the compound of formula 1-1 in an inert solvent.

This reaction is essentially the same as and may be carried out in the same manner as and using the same reagents and reaction conditions as Step 2C in Scheme 2.

### Step 2E

In this Step, an acid compound of formula 2-8 may be prepared by hydrolysis of the ester compound of formula 2-7.

This reaction is essentially the same as and may be carried out in the same manner as and using the same reagents and reaction conditions as Step 1B in Scheme 1.

### Step 2F

In this Step, an amide compound of formula 2-9 may be prepared by coupling reaction of the acid compound of formula 2-8 with the amino compound of formula 1-10. This reaction is essentially the same as and may be carried out in the same manner as and using the same reagents and reaction conditions as Step 1E in Scheme 1.

### Step 2F

In this Step, an acid compound of formula Ib may be prepared by hydrolysis of the ester compound of formula 2-9.

This reaction is essentially the same as and may be carried out in the same manner as and using the same reagents and reaction conditions as Step 1B in Scheme 1.

### Scheme 3:

This illustrates the preparation of compounds of formula (Ic) wherein R⁵ represents

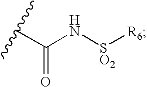

and R⁶ represents an alkyl group having from 1 to 6 carbon atoms, a cycloalkyl group having from 3 to 7 ring atoms, an aryl group or a heteroaryl group.

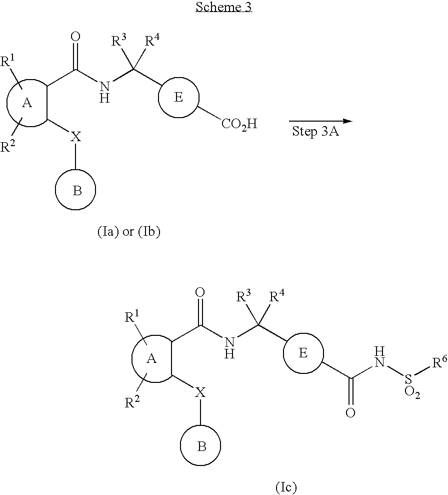

In the above formula all symbols are as already defined.

### Step 3A

In this Step, the desired compound of formula Ic may be prepared by the coupling of the compound of formula Ia or Ib, prepared as described in Step 1J in Scheme 1 and Step 2F in Scheme 2 respectively, with a compound of formula R⁶SO₂NH₂ in an inert solvent.

This reaction is essentially the same as and may be carried out in the same manner as and using the same reagents and reaction conditions as Step 1E in Scheme 1.

### Scheme 4:

This illustrates the preparation of compounds of formula (Id) wherein R⁵ represents

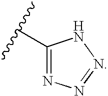

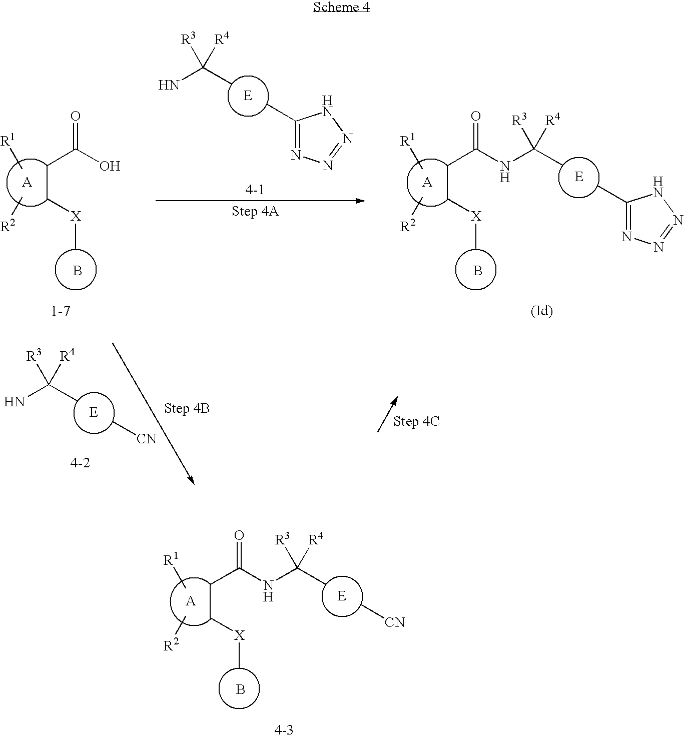

In the above formula all symbols are as already defined.

### Step 4A

In this Step, a tetrazole compound of formula Id may be prepared by the coupling of the acid compound of formula 1-7 with an amino compound of formula 4-1. This reaction is essentially the same as and may be carried out in the same manner as and using the same reagents and reaction conditions as Step 1E in Scheme 1.

### Step 4B

In this Step, an amide compound of formula 4-3 may be prepared by the coupling of the acid compound of formula 1-7 with an amino compound of formula 4-2. This reaction is essentially the same as and may be carried out in the same manner as and using the same reagents and reaction conditions as Step 1E in Scheme 1.

### Step 4C

In this Step, the tetrazole compound of formula Id may also be prepared by converting a nitrile group of the compound of formula 4-3 into the tetrazole group in a inert solvent toluene; DMF, DMSO, 2-methoxyethanol, water and THF. Examples of suitable tetrazole forming reagents include: sodium azide, lithium azide, trialkyltinazide(alkyl is typically methyl or butyl) and trimethylsilylazide. This reaction may be carried out in the presence or absence of a catalyst. Example of suitable catalysts include dialkyltin oxide (alkyl is typically methyl or butyl), alkylamino hydrochloride, alkylamino hydrobromide or lithium chloride. If desired, this reaction may be carried out in the presence or absence of an acid or a base. Examples of suitable bases include: trimethyl amine, triethyl amine and N,N-diisopropyl ethyl amine. Examples of suitable acids include: ammonium chloride, hydrogen chloride, aluminum chloride or zinc bromide. This reaction may be carried out at temperature of 50° C. to 200° C., preferably from about 80° C. to 150° C. for 5 minutes to 48 hours, preferably 30 minutes to 30 hours. If desired, this reaction may be carried out in a sealable tube.

The starting materials in the aforementioned general syntheses may be commercially available or obtained by conventional methods known to those skilled in the art.

In the above Schemes from 1 to 4, examples of suitable solvents include a mixture of any two or more of those solvents described in each Step.

The compounds of formula (I), and the intermediates above-mentioned preparation methods can be isolated and purified by conventional procedures, such as recrystallization or chromatographic purification.

The following reaction schemes illustrate the preparation of EP4-receptor antagonists described in U.S. Patent Application No. US 60/568,088. Unless otherwise indicated R¹ through R³ and X, Y, and Z in the reaction schemes and discussion that follow are defined as above. The term “protecting group”, as used hereinafter, means a hydroxy or amino protecting group which is selected from typical hydroxy or amino protecting groups described in Protective Groups in Organic Synthesis edited by T. W. Greene et al. (John Wiley & Sons, 1999);

The following reaction schemes illustrate the preparation of compounds of formula (I).

### Scheme 1:

This illustrates the preparation of compounds of formula (I).

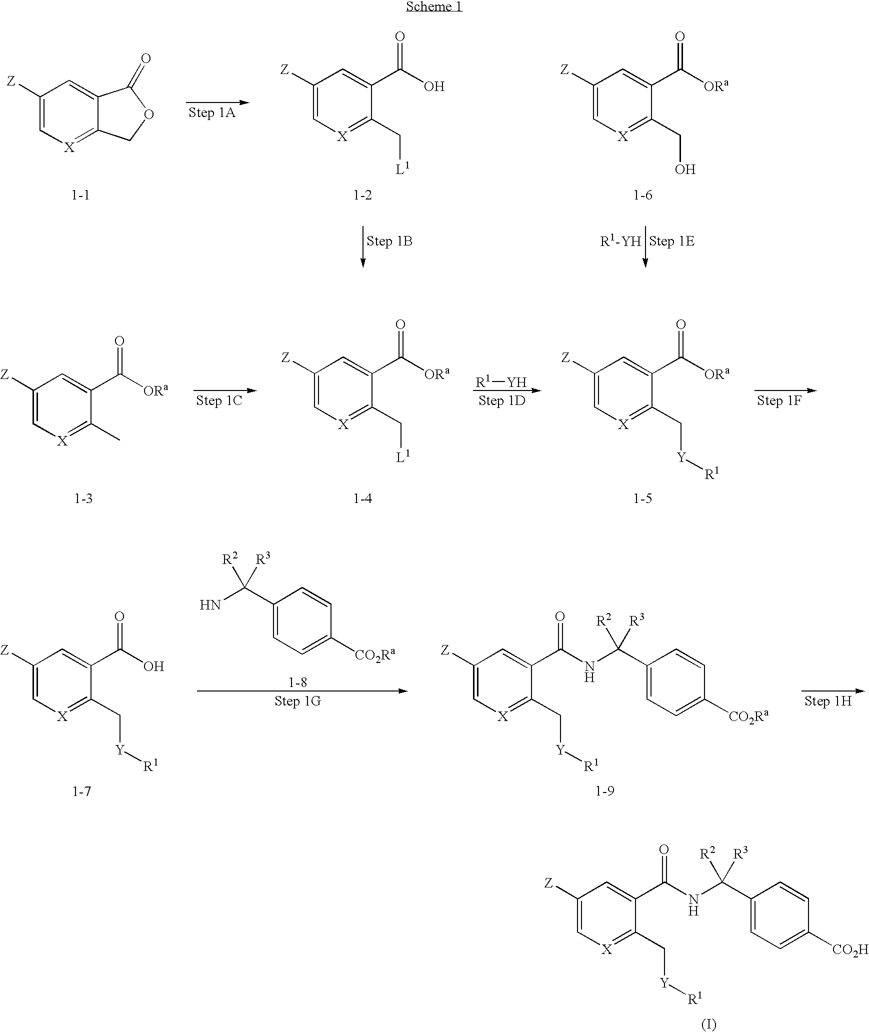

In the above formula, R^{a} represents an alkyl group having from 1 to 4 carbon atoms. L¹ represents a leaving group. Examples of suitable leaving groups include: halogen atoms, such as chlorine, bromine and iodine; sulfonic esters such as TfO (triflates), MsO (mesylates), TsO (tosylates); and the like.

### Step 1A

In this step, a compound of the formula 1-2 in which L¹ represents a halogen atom can be prepared by the halogenating the compound of the formula 1-1 under halogenation conditions with a halogenating reagent in a reaction-inert solvent.

Examples of suitable solvents include: acetic acid, water, acetonitrile, and dichloromethane. Preferred halogenating agents include: chlorinating agents; such as hydrogen chloride, chlorine, and acetyl chloride, brominating agents, such as hydrogen bromide, bromine, and boron tribromide, iodinating agents; hydrogen iodide, trimethylsilyl iodide, sodium iodide-boron tribromide. The reaction can be carried out at a temperature of from 0° C. to 200° C., more preferably from 20° C. to 120° C. Reaction times are, in general, from 5 minutes to 24 hours, more preferably 30 minutes to 10 hours, will usually suffice.

### Step 1B

In this Step, an ester compound of formula 1-4 can be prepared by the esterification of the acid compound of formula 1-2.

The esterification may be carried out by a number of standard procedures known to those skilled in the art (e.g., Protective Groups in Organic Synthesis, Third edition. ed. T. W. Green and P. G. M. Wuts, Wiley-Interscience., pp 373-377). Typical esterification can be carried out in the presence of an acid catalyst, e.g. sulfuric acid, p-toluenesulfonic acid, camphorsulfonic acid and benzenesulfonic acid, in a suitable reaction-inert solvent, e.g. methanol or ethanol. Typical esterification can also be carried out with a suitable C₁₋₆ alkylhalide or benzylhalide in the presence of a base, K₂CO₃, Cs₂CO₃, NaHCO₃ and DBU, in a suitable reaction-inert solvent, e.g. ethers such as tetrahydrofuran, 1,2-dimethoxyethane, diethyl ether, diisopropyl ether, diphenyl ether, DMF, DMSO, R′OH and 1,4-dioxane. The esterification also carried out with trimethylsilyldiazomethane in a suitable reaction-inert solvent, e.g. methanol, benzene and toluene. The esterification also carried out with diazomethane in a suitable reaction-inert solvent, e.g. diethyl ether. Alternatively, the esterification may be carried out with R′OH, in the presence of a coupling agent, e.g. DCC, WSC, diisoproopylcyanophosphonate (DIPC), BOPCl and 2,4,6-trichlorobenzoic acid chloride, and a tertiaryamine, e.g. i-Pr₂Net or Et₃N, in a suitable solvent, e.g. DMF, THF, diethyl ether, DME, dichloromethane and DCE.

### Step 1C

Alternatively, in this step, the compound of the formula 1-4 in which L¹ represents a halogen atom can also be prepared by the halogenating the compound of a formula 1-3 under halogenation conditions with a halogenating reagent in a reaction-inert solvent.

Examples of suitable solvents include: tetrahydrofuran, 1,4-dioxane, N,N-dimethylformamide, acetonitrile; alcohols, such as methanol or ethanol; halogenated hydrocarbons, such as dichloromethane, 1,2-dichloroethane, chloroform or carbon tetrachloride and acetic acid. Suitable halogenating reagents include, for example, bromine, chlorine, iodine, N-chlorosuccimide, N-bromosuccimide, 1,3-dibromo-5,5-dimethylhydantoin, bis(dimethylacetamide)hydrogen tribromide, tetrabutylammonium tribromide, bromodimethylsulfonium bromide, hydrogen bromide-hydrogen peroxide, nitrodibromoacetonitrile or copper(II) bromide. The reaction can be carried out at a temperature of from 0° C. to 200° C., more preferably from 20° C. to 120° C. Reaction times are, in general, from 5 minutes to 48 hours, more preferably 30 minutes to 24 hours, will usually suffice.

### Step 1D

In this step, a compound of formula 1-5 can be prepared by the alkylation of the compound of formula 1-4 with a compound of formula R¹—YH in the presence of a base in a reaction-inert solvent. Examples of suitable solvents include: tetrahydrofuran, N,N-dimethylformamide, dimethylsulfoxide, diethylether, toluene, ethylene glycol dimethylether generally or 1,4-dioxane. Examples of suitable bases include: alkyl lithiums, such as n-butyllithium, sec-butyllithium or tert-butyllithium; aryllithiums, such as phenyllithium or lithium naphtilide; methalamide such as sodium amide or lithium diisopropylamide; and alkali metal, such as potassium hydride or sodium hydride. This reaction may be carried out at a temperature in the range from −50° C. to 200° C., usually from 0° C. to 80° C. for 5 minutes to 72 hours, usually 30 minutes to 24 hours.

### Step 1E

Alternatively, in this step, the compound of formula 1-5 can also be prepared by Mitsunobu reaction of a compound of formula 1-6 with a compound of formula R¹—YH in the presence of dialkyl azodicarboxylate in a reaction-inert solvent. The compound of formula 1-6 may be treated with a compound of formula R¹—YH in the presence of dialkyl azodicarboxylate such as diethyl azodicarboxylate (DEAD) and phosphine reagent such as triphenylphosphine. Preferably, this reaction may be carried out in a reaction-inert solvent. Preferred reaction inert solvents include, but are not limited to, tetrahydrofuran (THF), diethyl ether, dimethylformamide (DMF), benzene, toluene, xylene, o-dichlorobenzene, nitrobenzene, dichloromethane, 1,2-dichloroethane, dimethoxyethane (DME), or mixtures thereof. This reaction may be carried out at a temperature in the range from −50° C. to 200° C., usually from 0° C. to 80° C. for 5 minutes to 72 hours, usually 30 minutes to 24 hours.

### Step 1F

In this step, an acid compound of formula 1-7 may be prepared by hydrolysis of the ester compound of formula 1-5 in a solvent.

The hydrolysis may be carried out by conventional procedures. In a typical procedure, the hydrolysis carried out under the basic condition, e.g. in the presence of sodium hydroxide, potassium hydroxide or lithium hydroxide. Suitable solvents include, for example, alcohols such as methanol, ethanol, propanol, butanol, 2-methoxyethanol, and ethylene glycol; ethers such as tetrahydrofuran (THF), 1,2-dimethoxyethane (DME), and 1,4-dioxane; amides such as N,N-dimethylformamide (DMF) and hexamethylphospholictriamide; and sulfoxides such as dimethyl sulfoxide (DMSO). This reaction may be carried out at a temperature in the range from −20° C. to 100° C., usually from 20° C. to 75° C. for 30 minutes to 48 hours, usually 60 minutes to 30 hours.

The hydrolysis may also be carried out under the acidic condition, e.g. in the presence of hydrogen halides, such as hydrogen chloride and hydrogen bromide; sulfonic acids, such as p-toluenesulfonic acid and benzenesulfonic acid; pyridium p-toluenesulfonate; and carboxylic acid, such as acetic acid and trifluoroacetic acid. Suitable solvents include, for example, alcohols such as methanol, ethanol, propanol, butanol, 2-methoxyethanol, and ethylene glycol; ethers such as tetrahydrofuran (THF), 1,2-dimethoxyethane (DME), and 1,4-dioxane; halogenated hydrocarbons, such as dichloromethane, 1,2-dichloroethane, amides such as N,N-dimethylformamide (DMF) and hexamethylphospholictriamide; and sulfoxides such as dimethyl sulfoxide (DMSO). This reaction may be carried out at a temperature in the range from −20° C. to 100° C., usually from 0° C. to 65° C. for 30 minutes to 24 hours, usually 60 minutes to 10 hours.

### Step 1G

In this step, an amide compound of formula 1-9 may be prepared by the coupling reaction of an amine compound of formula 1-8 with the acid compound of formula 1-7 in the presence or absence of a coupling reagent in an inert solvent. If desired, this reaction may be carried out in the presence or absence of an additive such as 1-hydroroxybenzotriazole (HOBt) or 1-hydroxyazabenzotriazole. Examples of suitable solvents include: acetone, nitromethane, N,N-dimethylformamide (DMF), sulfolane, dimethyl sulfoxide (DMSO), 1-methyl-2-pirrolidinone (NMP), 2-butanone, acetonitrile; halogenated hydrocarbons, such as dichloromethane, 1,2-dichloroethane, chloroform; and ethers, such as tetrahydrofuran and 1,4-dioxane. This reaction may be carried out at a temperature in the range from −20° C. to 100° C., more preferably from about 0° C. to 60° C. for 5 minutes to 1 week, more preferably 30 minutes to 24 hours, will usually suffice. Suitable coupling reagents are those typically used in peptide synthesis including, for example, diimides (e.g., dicyclohexylcarbodiimide (DCC), water soluble carbodiimide (WSC)), O-benzotriazol-1-yl-N,N,N′,N′-tetramethyluronium hexafluorophosphate (HBTU), 2-ethoxy-N-ethoxycarbonyl-1,2-dihydroquinoline, 2-bromo-1-ethylpyridinium tetrafluoroborate (BEP), 2-chloro-1,3-dimethylimidazolinium chloride, benzotriazol-1-yloxy-tris(dimethylamino)phosphonium hexafluorophosphate (BOP), diethyl azodicarboxylate-triphenylphosphine, diethylcyanophosphate, diethylphosphorylazide, 2-chloro-1-methylpyridinium iodide, N,N′-carnbonyldiimidazole, benzotriazole-1-yl diethyl phosphate, ethyl chloroformate or isobutyl chloroformate. If desired, the reaction may be carried out in the presence of a base such as, N,N-diisopropylethylamine, N-methylmorpholine, 4-(dimethylamino)pyridine and triethylamine. The amide compound of formula (I) may be formed via an acylhalide, which may be obtained by the reaction with halogenating agents such as oxalylchloride, phosphorus oxychloride and thionyl chloride. The resulting acylhalide may be converted to the corresponding amide compound by treating with the amine compound of formula 1-13 under the similar conditions as described in this step.

### Step 1H

In this Step, the compound of formula (I) may be prepared by hydrolysis of the ester compound of formula 1-9. This reaction is essentially the same as and may be carried out in the same manner as and using the same reagents and reaction conditions as Step 1F in Scheme 1.

### Scheme 2:

This illustrates the preparation of compounds of formula (Ia) wherein X represents a nitrogen atom; and Y represents an oxygen atom.

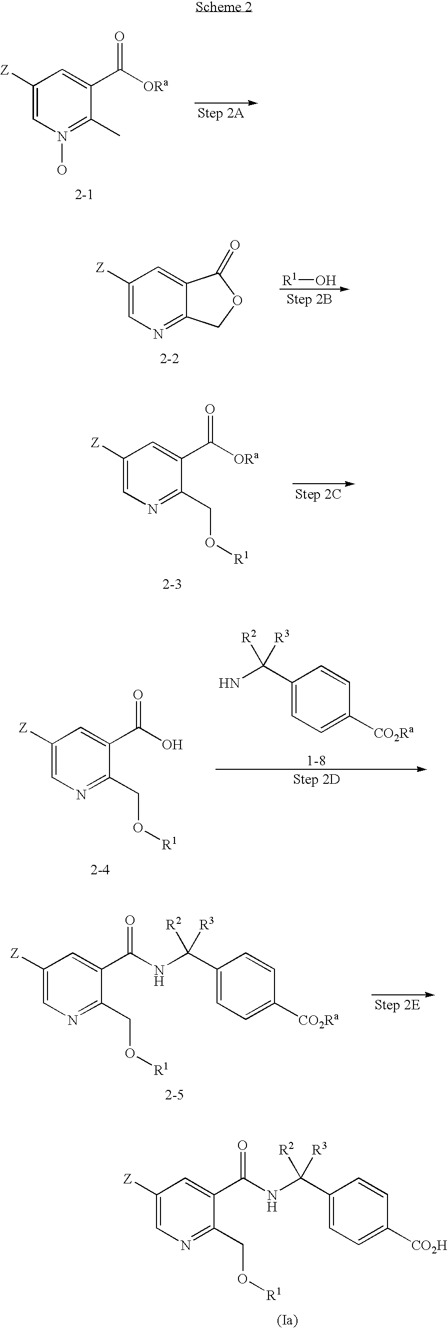

In the above formula, and R^{a} is defined in Scheme 1.

### Step 2A

In this Step, a lactone compound of formula 2-2 may be prepared by rearrangement of a compound of formula 2-1 followed by cyclization in a reaction-inert solvent.

Firstly, the compound 2-1 may be treated with an reagent in a reaction-inert solvent. Examples of suitable solvents include: such as dichloromethane and dimethylformamide. Examples of suitable reagents include: such as trifluoroacetic anhydride and acetic anhydride. The reaction can be carried out at a temperature of from −50° C. to 100° C., more preferably from −0° C. to 40° C. Reaction times are, in general, from 5 minutes to 48 hours, more preferably 30 minutes to 24 hours, will usually suffice.

Secondly, the obtained alcohol compound may be treated with a base or a acid in a reaction-inert solvent. Examples of suitable solvents include: such as methanol, benzene, toluene, and acetic acid. Example of such bases include: an alkali or alkaline earth metal hydroxide, alkoxide, carbonate, halide or hydride, such as sodium hydroxide, potassium hydroxide, sodium methoxide, sodium ethoxide, potassium tert-butoxide, sodium carbonate, potassium carbonate, sodium hydride or potassium hydride, or an amine such as triethylamine, tributylamine, diisopropylethylamine, pyridine or dimethylaminopyridine. Example of such acids include: hydrogen halides, such as hydrogen chloride and hydrogen bromide; sulfonic acids, such as p-toluenesulfonic acid and benzenesulfonic acid; pyridium p-toluenesulfonate; and carboxylic acid, such as acetic acid and trifluoroacetic acid. The reaction can be carried out at a temperature of from 0° C. to 200° C., more preferably from room temperature to 100° C. Reaction times are, in general, from 5 minutes to 48 hours, more preferably 30 minutes to 24 hours, will usually suffice.

### Step 2B

In this Step, a compound of formula 2-3 may be prepared by the reaction of the lactone compound of formula 2-2 with an alcohol compound of formula R¹—OH in the absence or the presence of a base in an inert solvent.

Examples of suitable solvents include: alcohols, such as methanol or ethanol; halogenated hydrocarbons, such as dichloromethane, 1,2-dichloroethane, chloroform or carbon tetrachloride and acetic acid; aromatic hydrocarbons, such as benzene, toluene, xylene, nitrobenzene, and pyridine; halogenated hydrocarbons, such as methylene chloride, chloroform, carbon tetrachloride and dichloroethane; ethers, such as diethyl ether, diisopropyl ether, DME, tetrahydrofuran and dioxane; ethyl acetate, acetonitrile, N,N-dimethylformamide, dimethylsulfoxide and water. Example of such bases include: an alkali or alkaline earth metal hydroxide, alkoxide, carbonate, halide or hydride, such as sodium hydroxide, potassium hydroxide, sodium methoxide, sodium ethoxide, potassium tert-butoxide, sodium carbonate, potassium carbonate, potassium fluoride, sodium hydride or potassium hydride, or an amine such as triethylamine, tributylamine, diisopropylethylamine, pyridine or dimethylaminopyridine in the presence or absence of a reaction-inert solvent.

The reaction can be carried out at a temperature of from −100° C. to 250° C., more preferably from 0° C. to the reflux temperature. Reaction times are, in general, from 1 minute to 10 day, more preferably from 20 minutes to 5 days, will usually suffice. from 1 minute to a day, preferably from 1 hour to 10 hours.

### Step 2C

In this step, an acid compound of formula 2-4 may be prepared by hydrolysis of the compound of formula 2-3. This reaction is essentially the same as and may be carried out in the same manner as and using the same reagents and reaction conditions as Step 1F in Scheme 1.

### Step 2D

In this Step, the compound of formula 2-5 may be prepared by the coupling reaction of the compound of formula 2-4 with the compound of formula 1-8 in an inert solvent.

This reaction is essentially the same as and may be carried out in the same manner as and using the same reagents and reaction conditions as Step 1G in Scheme 1.

### Step 2E

In this Step, the compound of formula (Ia) may be prepared by hydrolysis of the ester compound of formula 2-5.

This reaction is essentially the same as and may be carried out in the same manner as and using the same reagents and reaction conditions as Step 1F in Scheme 1.

In the above Schemes from 1 and 2, examples of suitable solvents include a mixture of any two or more of those solvents described in each step.

The starting materials in the aforementioned general syntheses are commercially available or may be obtained by conventional methods known to those skilled in the art.

The compounds of formula (I), and the intermediates above-mentioned preparation methods can be isolated and purified by conventional procedures, such as recrystallization or chromatographic purification.

The various general methods described above may be useful for the introduction of the desired groups at any stage in the stepwise formation of the required compound, and it will be appreciated that these general methods can be combined in different ways in such multi-stage processes. The sequence of the reactions in multi-stage processes should of course be chosen so that the reaction conditions used do not affect groups in the molecule which are desired in the final product.

The following reaction schemes illustrate the preparation of EP4-receptor antagonists described in U.S. Patent Application No. US 60/567,931. Unless otherwise indicated R¹ through R³ and X, Y, and Z in the reaction schemes and discussion that follow are defined as above. The term “protecting group”, as used hereinafter, means a hydroxy or amino protecting group which is selected from typical hydroxy or amino protecting groups described in Protective Groups in Organic Synthesis edited by T. W. Greene et al. (John Wiley & Sons, 1999);

### Scheme 1:

This illustrates the preparation of compounds of formula (I).

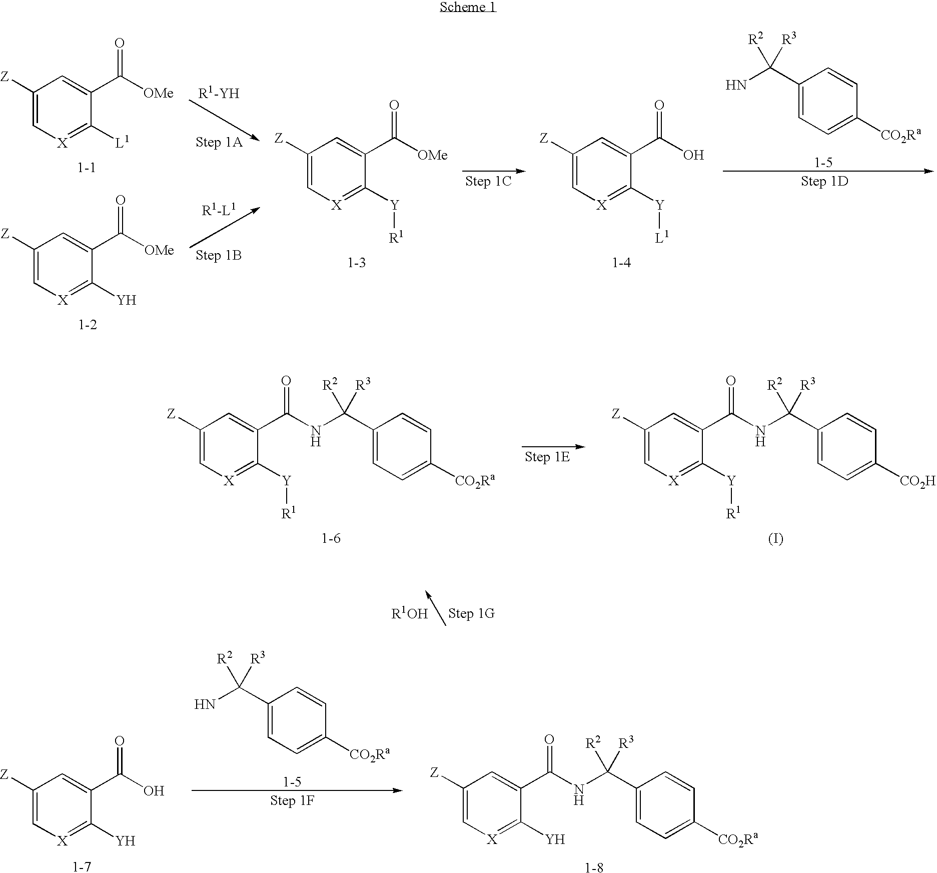

In the above formula, R^{a} represents an alkyl group having from 1 to 4 carbon atoms. L¹ represents a leaving group. Examples of suitable leaving groups include: halogen atoms, such as chlorine, bromine and iodine; sulfonic esters such as TfO (triflates), MsO (mesylates), TsO (tosylates); or a boronic acid group.

### Step 1A

In this Step, a compound of formula 1-3 may be prepared by the coupling reaction of an ester compound of formula 1-1 with a compound of formula R¹—YH in an inert solvent.

The coupling reaction may be carried out in the absence or presence of a base in a reaction inert solvent or without solvent. A preferred base is selected from, for example, an alkali or alkaline earth metal hydroxide, alkoxide, carbonate, or hydride, such as sodium hydroxide, potassium hydroxide, sodium methoxide, sodium ethoxide, potassium tert-butoxide, sodium carbonate, cesium carbonate or potassium carbonate, 2-tert-butylimino-2-diethylamino-1,3-dimethyl-perhydro-1,3,2-diazaphosphorine (BEMP), tert-butylimino-tri(pyrrolidino)phosphorane (BTPP), cesium fluoride (CsF), potassium fluoride, sodium hydride or potassium hydride, or an amine such as triethylamine, tributylamine, diisopropylethylamine, 2,6-lutidine, pyridine or dimethylaminopyridine. Preferred reaction inert solvents include, for example, acetone, benzene, toluene, xylene, o-dichlorobenzene, nitrobenzene, nitromethane, pyridine, dichloromethane, dichloroethane, tetrahydrofuran, dimethylformamide (DMF), dimethylacetamide (DMA), dioxane, dimethylsulfoxide (DMSO), acetonitrile, sulfolane, N-methylpyrrolidinone (NMP), methyl ethyl ketone (2-butanone), tetrahydrofuran (THF), dimethoxyethane (DME) or mixtures thereof. Reaction temperatures are generally in the range of 0 to 200° C., preferably in the range of room temperature to 150° C. Reaction times are, in general, from 1 minute to a day, preferably from 1 hour to 10 hours. If desired, the reaction may be conducted in the presence of metal catalyst such as copper (e.g. copper bronze or cuprous iodide) and nickel.

When L¹ represents a boronic acid group, the reaction may be carried out in the presence of a suitable catalyst to form the compound of formula 1-3 by any synthetic procedure applicable to structure-related compounds known to those skilled in the literature (e.g., Lam, P. Y. S.; Clark, C. G.; Saubern, S; Adams, J; Winters, M. P.; Chan, D. M. T.; Combs, A., Tetrahedron Lett., 1998, 39, 2941-2944, Kiyomori, A.; Marcoux, J.; Buchwald, S. L., Tetrahedron Lett., 1999, 40, 2657-2660., Lam, P. Y. S.; Deudon, S.; Averill, K. M.; Li, R.; He, M. Y.; DeShong, P.; Clark, C. G., J. Am. Chem. Soc., 2000, 122, 7600-7601., Collman, J. P.; Zhong, M., Org. Lett., 2000, 2, 1233-1236). A preferred reaction catalyst is selected from, for example, tetrakis(triphenylphosphine)-palladium, bis(triphenylphosphine)palladium(II) chloride, copper(0), copper(I) acetate, copper(I) bromide, copper(I) chloride, copper(I) iodide, copper(I) oxide, copper(II) trifluoromethanesulfonate, copper(II) acetate, copper(II) bromide, copper(II) chloride, copper(II) iodide, copper(II) oxide, or copper(II) trifluoromethanesulfonate.

### Step 1B

In this Step, the ester compound of formula 1-3 may also be prepared by coupling reaction of an ester compound of formula 1-2 with a compound of formula R¹-L¹. This reaction is essentially the same as and may be carried out in the same manner as and using the same reagents and reaction conditions as Step 1A in Scheme 1.

### Step 1C

In this step, an acid compound of formula 1-4 may be prepared by hydrolysis of the ester compound of formula 1-3 in a solvent.

The hydrolysis may be carried out by conventional procedures. In a typical procedure, the hydrolysis carried out under the basic condition, e.g. in the presence of sodium hydroxide, potassium hydroxide or lithium hydroxide. Suitable solvents include, for example, alcohols such as methanol, ethanol, propanol, butanol, 2-methoxyethanol, and ethylene glycol; ethers such as tetrahydrofuran (THF), 1,2-dimethoxyethane (DME), and 1,4-dioxane; amides such as N,N-dimethylformamide (DMF) and hexamethylphospholictriamide; and sulfoxides such as dimethyl sulfoxide (DMSO). This reaction may be carried out at a temperature in the range from −20° C. to 100° C., usually from 20° C. to 75° C. for 30 minutes to 48 hours, usually 60 minutes to 30 hours.

The hydrolysis may also be carried out under the acidic condition, e.g. in the presence of hydrogen halides, such as hydrogen chloride and hydrogen bromide; sulfonic acids, such as p-toluenesulfonic acid and benzenesulfonic acid; pyridium p-toluenesulfonate; and carboxylic acid, such as acetic acid and trifluoroacetic acid. Suitable solvents include, for example, alcohols such as methanol, ethanol, propanol, butanol, 2-methoxyethanol, and ethylene glycol; ethers such as tetrahydrofuran (THF), 1,2-dimethoxyethane (DME), and 1,4-dioxane; halogenated hydrocarbons, such as dichloromethane, 1,2-dichloroethane, amides such as N,N-dimethylformamide (DMF) and hexamethylphospholictriamide; and sulfoxides such as dimethyl sulfoxide (DMSO). This reaction may be carried out at a temperature in the range from −20° C. to 100° C., usually from 0° C. to 65° C. for 30 minutes to 24 hours, usually 60 minutes to 10 hours.

### Step 1D

In this step, an amide compound of formula 1-6 may be prepared by the coupling reaction of an amine compound of formula 1-5 with the acid compound of formula 1-4 in the presence or absence of a coupling reagent in an inert solvent. If desired, this reaction may be carried out in the presence or absence of an additive such as 1-hydroroxybenzotriazole (HOBt) or 1-hydroxyazabenzotriazole. Examples of suitable solvents include: acetone, nitromethane, N,N-dimethylformamide (DMF), sulfolane, dimethyl sulfoxide (DMSO), 1-methyl-2-pirrolidinone (NMP), 2-butanone, acetonitrile; halogenated hydrocarbons, such as dichloromethane, 1,2-dichloroethane, chloroform; and ethers, such as tetrahydrofuran and 1,4-dioxane. This reaction may be carried out at a temperature in the range from −20° C. to 100° C., more preferably from about 0° C. to 60° C. for 5 minutes to 1 week, more preferably 30 minutes to 24 hours, will usually suffice. Suitable coupling reagents are those typically used in peptide synthesis including, for example, diimides (e.g., dicyclohexylcarbodiimide (DCC), water soluble carbodiimide (WSC)), O-benzotriazol-1-yl-N,N,N′,N′-tetramethyluronium hexafluorophosphate (HBTU), 2-ethoxy-N-ethoxycarbonyl-1,2-dihydroquinoline, 2-bromo-1-ethylpyridinium tetrafluoroborate (BEP), 2-chloro-1,3-dimethylimidazolinium chloride, benzotriazol-1-yloxy-tris(dimethylamino)phosphonium hexafluorophosphate (BOP), diethyl azodicarboxylate-triphenylphosphine, diethylcyanophosphate, diethylphosphorylazide, 2-chloro-1-methylpyridinium iodide, N,N′-carnbonyldiimidazole, benzotriazole-1-yl diethyl phosphate, ethyl chloroformate or isobutyl chloroformate. If desired, the reaction may be carried out in the presence of a base such as, N,N-diisopropylethylamine, N-methylmorpholine, 4-(dimethylamino)pyridine and triethylamine. The amide compound of formula (I) may be formed via an acylhalide, which may be obtained by the reaction with halogenating agents such as oxalylchloride, phosphorus oxychloride and thionyl chloride. The resulting acylhalide may be converted to the corresponding amide compound by treating with the amine compound of formula 1-13 under the similar conditions as described in this step.

### Step 1E

In this Step, the compound of formula (I) may be prepared by hydrolysis of the ester compound of formula 1-6. This reaction is essentially the same as and may be carried out in the same manner as and using the same reagents and reaction conditions as Step 1C in Scheme 1.

### Step 1F

In this Step, a compound of formula 1-8 may be prepared by the coupling reaction of an acid compound of formula 1-7 with the amine compound of formula 1-5 in an inert solvent.

This reaction is essentially the same as and may be carried out in the same manner as and using the same reagents and reaction conditions as Step 1D in Scheme 1.

### Step 1G

Alternatively, in this step, the compound of formula 1-6 can also be prepared by Mitsunobu reaction of the compound of formula 1-8 with a compound of formula R¹—OH in the presence of dialkyl azodicarboxylate in a reaction-inert solvent. The compound of formula 1-6 may be treated with a compound of formula R¹—OH in the presence of dialkyl azodicarboxylate such as diethyl azodicarboxylate (DEAD) and phosphine reagent such as triphenylphosphine. Preferably, this reaction may be carried out in a reaction-inert solvent. Preferred reaction inert solvents include, but are not limited to, tetrahydrofuran (THF), diethyl ether, dimethylformamide (DMF), benzene, toluene, xylene, o-dichlorobenzene, nitrobenzene, dichloromethane, 1,2-dichloroethane, dimethoxyethane (DME), or mixtures thereof. This reaction may be carried out at a temperature in the range from −50° C. to 200° C., usually from 0° C. to 80° C. for 5 minutes to 72 hours, usually 30 minutes to 24 hours.

In the above Schemes from 1, examples of suitable solvents include a mixture of any two or more of those solvents described in each step.

The starting materials in the aforementioned general syntheses are commercially available or may be obtained by conventional methods known to those skilled in the art.

The compounds of formula (I), and the intermediates above-mentioned preparation methods can be isolated and purified by conventional procedures, such as recrystallization or chromatographic purification.

The various general methods described above may be useful for the introduction of the desired groups at any stage in the stepwise formation of the required compound, and it will be appreciated that these general methods can be combined in different ways in such multi-stage processes. The sequence of the reactions in multi-stage processes should of course be chosen so that the reaction conditions used do not affect groups in the molecule which are desired in the final product.

The following experimental procedures illustrate the preparation of certain preferred alpha-2-delta ligands described above.

### (S)-3-((E)-2-Methyl-pent-2-enoyl)-4-phenyl-oxazolidin-2-one

A 20 L jacketed reactor was fitted with a reflux condenser and a nitrogen inlet. To the flask was charged 1006 g (8.81 mol) of (E)-2-methyl-2-pentenoic acid, 1250 g (7.661 mol) of (S)-(+)-4-phenyl-oxazolidin-2-one, 2179 g (8.81 mol) of 2-ethoxy-1-ethoxycarbonyl-1,2-dihydroquinoline (EEDQ), 81 g (1.915 mol) of lithium chloride, and 12.5 L of ethyl acetate (EtOAc). The reaction was heated to 75° C. for 20 hours and then cooled to room temperature. The reaction solution was extracted 3× with 4 L aliquots of 1 N HCl and 1× with 4 L of 0.2N NaOH. The 20 L reactor was fitted with a distillation head. The organic layer was distilled to remove, in succession: 6.5 L of EtOAc, after which 8 L of heptane was added back to the reactor; 4 L of EtOAc/heptane, after which 4 L of heptane was added to the reactor; and 4 L of EtOAc/heptane, after which 8 L of heptane was added to the reactor. After an additional 2 L of EtOAc/heptane was removed by distillation, the reaction mixture was cooled to an internal temperature of 40° C., and the reactor contents were charged to a filter and filtered under 5 psig of nitrogen washing with 8 L of heptane. The solids were dried under 5 psig of nitrogen overnight to give 1772 g of the titled compound: ¹H-NMR (DMSO) 7.363-7.243 (m, 5H), 6.137-6.096 (m, 1H), 5.434-5.394 (m, 1H), 4.721-4.678 (t, 1H, J=8.578), 4.109-4.069 (m, 1H), 2.119-2.044 (m, 2H), 1.703-1.700 (d, 3H, J=1.364), 0.945-0.907 (t, 3H, J=7.603); Anal Calc'd for C₁₅H₁₇N₁O₃: C, 69.48; H, 6.61; N, 5.40. Found: C, 68.66; H, 6.60; N, 5.60; MS (ion Mode: APCl) m/z=260 [M+1]⁺.

### (4S,5R)-3-((E)-2-Methyl-pent-2-enoyl)-4,5-diphenyl-oxazolidin-2-one

To a solution of (E)-2-methyl-2-pentenoic acid (5.3 g, 47 mmol) in 250 mL of THF at 0° C. was added 16.3 mL (117 mmol) of triethylamine, then 5.8 mL (47 mmol) of pivaloyl chloride resulting in a thick suspension. The mixture was stirred for 1 hour at 0° C. at which time 2.0 g (47 mmol) of lithium chloride was added in one portion, followed by 10 g (42 mmol) of (4S,5R)-4,5-diphenyl-2-oxazolidinone in four batches. Stirring was maintained throughout the solid additions. The reaction mixture was stirred for 1 hour at 0° C., and for 1 hour at ambient temperature, and was vacuum filtered through a coarse frit and concentrated. The residue was partitioned between EtOAc/water, and the organics were dried over MgSO₄ and concentrated. To the residue was added 200 mL of MTBE and the mixture was warmed cautiously with swirling. The warm slurry was filtered to provide 13.0 g (83% yield) of the titled compound as a colorless solid: ¹H NMR (CDCl₃) δ 7.12 (m, 3H), 7.08 (m, 3H), 6.93 (m, 2H), 6.86 (m, 2H), 6.14 (m, 1H), 5.90 (d, J=7.8 Hz, 1H), 5.69 (d, J=7.8 Hz, 1H), 2.23 (pent, J=7.6 Hz, 2H), 1.92 (s, 3H), 1.07 (t, J=7.6 Hz, 3H). The titled acylated oxazolidinone may be used in the next step instead of (S)-3-((E)-2-Methyl-pent-2-enoyl)-4-phenyl-oxazolidin-2-one.

### (2R,3R,4S)-3-(2,3-Dimethyl-pentanoyl)-4-phenyl-oxazolidin-2-one

A 20 L jacketed reactor was fit with a gas inlet and a 2 L dripping funnel. A nitrogen sweep was begun over the reactor and maintained throughout the process. To the reactor was charged 392 g (9.26 mol) of lithium chloride, 1332 g (6.479 mol) of copper bromide dimethylsulfide complex and 11 L of tetrahydrofuran. The reaction was stirred for 30 minutes at room temperature and then cooled to −15° C. To the reaction mixture was added 4.268 L (12.80 mol) of 3.0M methyl magnesium chloride at a rate such that the reaction temperature did not exceed −10° C. Upon completion of the addition, the cuprate solution was allowed to stir at −5° C. overnight. To the cuprate solution was added 500 g (3.09 mol) of (S)-3-((E)-2-methyl-pent-2-enoyl)-4-phenyl-oxazolidin-2-one as a solid. The reaction was stirred at −3° C. for 2 hours. The reaction solution was charged to a 22 L round bottom flask containing 800 mL of acetic acid and 2 L of tetrahydrofuran at a rate such that the temperature of the quench solution did not exceed 25° C. To the quenched solution was added 6 L water. The resulting emulsion was filtered and the layers were separated. The organic layer was extracted with 9 L of 4.8 M NH₄OH followed by 9 L of saturated NH₄Cl. The organic layer was clarified through a plug of magnesol. The organic layer was concentrated to give 822 g of a crude solid. The crude solid was recrystallized from 8 L of 20% H₂O in MeOH, filtered and dried in a vacuum oven to give 550 g of a white solid. The white solid was recrystallized from 5 L of 20% H₂O in MeOH, filtered and dried in a vacuum oven to give 475 g of the titled compound: ¹H-NMR (DMSO) 7.338-7.224 (m, 5H), 5.431-5.399 (q, 1H, J=4.288), 4.696-4.652 (t, 1H, J=8.773), 4.120-4.087 (m, 1H), 3.622-3.556 (m, 1H), 1.648-1.584 (m, 1H), 1.047-0.968 (m, 1H), 0.900-0.883 (d, 3H, J=6.823), 0.738-0.721 (d, 3H, J=6.628), 0.693-0.656 (t, 3H, J=7.408); Anal Calc'd for C₁₆H₂₁N₁O₃: C, 69.79; H, 7.69; N, 5.09. Found: C, 69.81; H, 7.61; N, 5.07; MS (ion Mode: APCl) m/z=276 [M+1]⁺.

### (2R,3R)-2,3-Dimethyl-pentanoic acid

A 20 L jacketed flask was fit with a gas inlet. A nitrogen purge was begun over the reactor and maintained throughout the process. To the flask was charged 450 g (1.634 mol) of (2R,3R,4S)-3-(2,3-dimethyl-pentanoyl)-4-phenyl-oxazolidin-2-one and 3.375 L tetrahydrofuran. The contents of the reactor were stirred at 15° C. In a separate 3 L round bottom flask, placed in an ice bath, was charged 500 mL of water, 137 g (3.269 mol) of LiOH—H₂O and 942 mL (9.81 mol) of 30% wt/wt H₂O₂. The contents of the 3 L round bottom flask were stirred for 3 minutes and then poured into the 20 L jacketed reactor at a rate such that the temperature did not exceed 25° C. The reaction was stirred at 15° C. for 2 hours and then raised to 25° C. and stirred for an additional 2 hours. The jacket temperature of the reactor was set to −20° C. To the reaction was added 1.66 L of saturated NaHSO₃ at a rate such that the temperature of the reaction did not exceed 25° C. The layers were separated. The aqueous layer was extracted 2× with 1 L aliquots of MTBE. The organic phases were combined and concentrated to give a solid/oil mixture. The solid/oil mixture was slurried in 1.7 L of hexane. The slurry was filtered and the collected solids were washed with 1.7 L of hexane. The hexane filtrates were extracted 2× with 1.35 L aliquots of 1 N NaOH. The aqueous extracts were combined and extracted with 800 mL of dichloromethane. The aqueous layer was then acidified with 240 mL of concentrated hydrochloric acid. The aqueous solution was extracted 2× with 1 L aliquots of dichloromethane. The organic extracts were combined, dried over MgSO₄ and concentrated to give 201 g of the titled compound: ¹H-NMR (DMSO) 11.925 (bs, 1H), 2.204-2.135 (m, 1H), 1.556-1.490 (m, 1H), 1.382-1.300 (m, 1H), 1.111-1.000 (m, 1H), 0.952-0.934 (d, 3H, J=7.018), 0.809-0.767 (m, 6H); Gas Chromatogram 9.308 minutes, 98.91% area; Anal Calc'd for C₇H₁₄O₂: C, 64.58; H, 10.84; N, O. Found: C, 64.39; H, 10.77; N, 0.18; MS (ion Mode: APCl) m/z=131 [M+1]⁺.

### (4R,5R)-4,5-Dimethyl-3-oxo-heptanoic acid ethyl ester

To a 1 L round bottom flask equipped with a nitrogen inlet was charged 22 g (230 mmol) of magnesium chloride, 39 g (230 mmol) of potassium ethyl malonate and 200 mL of dimethylformamide. The contents of the flask were stirred at 50° C. for 1 hour and then cooled to 35° C. In a separate 500 mL, nitrogen inerted flask was added 200 mL of dimethylformamide, 28.6 g (177 mmol) of carbonyl diimidazole and 20 g of (2R,3R)-2,3-dimethyl-pentanoic acid was dripped in over 30 minutes. When the gas evolution had ceased, the contents of the 500 mL flask were added to the 1 L flask. The reaction was stirred for 2 days at 35° C. The reaction was cooled to room temperature and diluted with 800 mL of 1N HCl. The aqueous solution was extracted 3× with 1 L aliquots of MTBE. The organic extracts were combined and extracted with 200 mL of saturated NaHCO₃. The organic layer was dried over MgSO₄ and concentrated to give 31.74 g of the titled compound: ¹H-NMR (CDCl₃) 4.180-4.120 (m, 2H), 3.454 (s, 2H), 2.522-2.453 (q, 1H, J=7.018), 1.738-1.673 (m, 1H), 1.418-1.328 (m, 1H), 1.270-1.217 (m, 3H), 1.113-1.010 (m, 4H), 0.889-0.815 (m, 5H); MS (ion Mode: APCl) m/z=201 [M+1]⁺.

### (4R,5R)-3-Methoxyimino-4,5-dimethyl-heptanoic acid ethyl ester

(4R,5R)-4,5-Dimethyl-3-oxo-heptanoic acid ethyl ester (21.23 g, 106 mmol) was dissolved in 200 mL of EtOH and added to 10.6 g (127 mmol) of methoxylamine-HCl and 10.6 g (127 mmol) of sodium acetate solids. The slurry was stirred at room temperature for 48 hours. MTBE (200 mL) and 100 mL of water were added, and the resulting phases were separated. The organic phase was washed with 100 mL of water and was evaporated to yield a two-phase mixture. Hexanes (100 mL) were added and the phases were separated. The aqueous phase was extracted with 50 mL of hexanes and the combined organic phases were washed with 50 mL of water, dried over magnesium sulfate, and evaporated to give 21.24 g (87.4% yield) of the titled compound as a clear yellow oil: ¹H NMR (CDCl₃, 399.77 MHz) δ 0.84-0.88 (m, 6H), 1.07 (d, J=7.1 Hz, 3H), 1.24 (t, J=7.1 Hz, 3H), 1.4-1.6 (m, 2H), 2.24 (m, 1H), 3.08 (d, J=15.8 Hz, 1H), 3.19 (d, J=15.8 Hz, 1H), 3.80 (s, 3H), 4.10-4.2 (m, 3H). Low resolution mass spec: nominal m/e calc'd for C₁₂H₂₃NO₃ (M+H)⁺: 230. Found: m/e 230.

### (4R,5R)-3-Amino-4,5-dimethyl-hept-2-(Z)-enoic acid ethyl ester

A solution of 21.1 g (92 mmol) of (4R,5R)-3-methoxyimino-4,5-dimethyl-heptanoic acid ethyl ester in methanol (200 mL) was treated with Sponge nickel (10 g, Johnson Matthey A7000). The resulting slurry was hydrogenated on a Parr shaker type hydrogenator at 50 psig and room temperature for 20 hours. At this time an additional 10 g of the nickel catalyst was added and hydrogenation was continued for a total of 42.0 hours. The slurry was filtered, the solids were washed with fresh methanol, and the combined filtrate was evaporated to give 17.75 g (96.8% yield) of the titled compound as a colorless oil: ¹H NMR (CDCl₃, 399.77 MHz) δ 0.83-0.89 (m, 6H), 1.1 (d, J=6.8 Hz, 3H), 1.25 (t, J=7.1 Hz, 2H), 1.35-1.6 (m, 4H), 1.85-1.93 (m, 1H), 4.1 (q, J=7.0 Hz, 2H), 4.5 (s, 1H). Low resolution mass spec: nominal m/e calc'd for C₁₁H₂₁NO₂ (M+H)⁺: 200. Found: m/e 200.

### (4R,5R)-3-Acetylamino-4,5-dimethyl-hept-2-(Z)-enoic acid ethyl ester

A solution of 15.84 g (79.84 mmol) of (4R,5R)-3-amino-4,5-dimethyl-hept-2-(Z)-enoic acid ethyl ester and 6.89 g (7.04 mL, 87.82 mL) of pyridine was stirred in 200 mL of methylene chloride and cooled to 0° C. A solution of 6.85 g (6.21 mL, 87.82 mL) of acetyl chloride in 20 mL of methylene chloride was added dropwise over 1 hour. The solution was warmed to room temperature and stirred for two hours. 1M hydrochloric acid (100 mL) was added and the phases were separated. The organic phase was washed with saturated aqueous NaHCO₃ solution and dried briefly over Na₂SO₄. The solvent was evaporated and then the resulting oil was passed through a short column of silica (200 g silica, 230-400 mesh) with 8:1 (v/v) hexane/EtOAc. The product-containing fractions were evaporated to give 13.75 g (71.7% yield) of the titled compound as a clear, nearly colorless oil: ¹H NMR (CDCl₃, 399.77 MHz) δ 0.84 (t, J=7.1 Hz, 3H), 0.95 (d, J=6.8 Hz, 3H), 1.0 (d, J=7.0 Hz, 3H), 1.29 (t, J=7.2 Hz, 3H), 1.30-1.45 (m, 3H), 2.13 (s, 3H), 3.79-3.82 (m, 1H), 4.11-4.18 (m, 2H), 5.01 (s, 1H). Low resolution mass spec: nominal m/e calc'd for C₁₃H₂₃NO₃ (M+H)⁺: 242. Found: m/e 242.

### (3R,4R,5R)-3-Acetylamino-4,5-dimethyl-heptanoic acid ethyl ester

A solution containing 13.75 g (57 mmol) of (4R,5R)-3-acetylamino-4,5-dimethyl-hept-2-(Z)-enoic acid ethyl ester in 200 mL of methanol was treated with 5% Pd/Al₂O₃ (1.5 g, Johnson Matthey #2127, lot 13449). The resulting slurry was hydrogenated on a Parr shaker type hydrogenator at 40 psig to 50 psig and room temperature for a total of 3.8 hours. The slurry was filtered and the solids were washed with fresh methanol. The combined filtrate was evaporated to give 13.63 g (98.6% yield) of the titled compound as a colorless oil: ¹H NMR (CDCl₃, 399.77 MHz) δ 0.82 (d, J=7.0 Hz, 3H), 0.86 (t, J=7.3 Hz, 3H), 0.90 (d, J=6.5 Hz, 3H), 0.98-1.1 (m, 2H), 1.25 (t, J=7.2 Hz, 2H), 1.3-1.6 (m, 2H), 1.96 (s, 3H), 2.48 (dd, J=16, 5.65 Hz, 1H), 2.53 (dd, J=16, 5.2 Hz, 1H), 4.08-4.19 (m, 2H), 4.27-4.34 (m, 1H), 5.86 (br d, J=8.9 Hz, 1H). Low resolution mass spec: nominal m/e calc'd for C₁₃H₂₅NO₃ (M+H)⁺: 244. Found: m/e 244.

### (3R,4R,5R)-3-Amino-4,5-dimethyl-heptanoic acid hydrochloride

(3R,4R,5R)-3-Acetylamino-4,5-dimethyl-heptanoic acid ethyl ester (13.63 g, 56.0 mmol) was heated under reflux with 200 mL of 1M hydrochloric acid for 72 hours. The solution was cooled and extracted 2× with 50 mL aliquots of MTBE. The aqueous phase was evaporated to a semisolid. Acetonitrile (4×100 mL) was added and evaporated to give 10.75 g (89% yield) of the titled compound as a white crystalline solid: ¹H NMR (CD₃OD, δ99.77 MHz) 0.87 (t, J=7.3 Hz, 3H), 0.94 (t, J=6.6 Hz, 6H), 1.02-1.15 (m, 1H), 1.37-1.53 (m, 2H), 1.58-1.68 (m, 1H), 2.64 (dd, J=17.5, 7.4 Hz, 1H), 2.73 (dd, J+17.5, 4.8 Hz, 1H), 3.54-3.61 (m, 1H). Low resolution mass spec: nominal m/e calc'd for C₉H₂₀ClNO₂ (M+H)⁺: 174. Found: m/e 174.

### (3R,4R,5R)-3-Amino-4,5-dimethyl-heptanoic acid

(3R,4R,5R)-3-Amino-4,5-dimethyl-heptanoic acid hydrochloride (10.8 g, 51.5 mmol) was dissolved in 50 mL of methanol. To this solution was added triethylamine (5.2 g, 7.2 mL, 51.5 mmol). The solution was stirred for 10 minutes and then evaporated to a flocculent solid. Dichloromethane (376 mL) was added and the resulting slurry was stirred at room temperature for 45 minutes. Next, 188 mL of acetonitrile was added and the slurry was stirred for 30 minutes and then filtered. The solids were washed with 20 mL of 2:1 (v/v) dichloromethane-acetonitrile and dried on a nitrogen press to give 7.64 g (85.6% yield) of the titled compound as a white solid: ¹H NMR (CD₃OD, δ99.77 MHz) 0.88 (t, J=7.5 Hz, 3H), 0.91 (d, J=7.0 Hz, 3H), 0.94 (d, J=6.6 Hz, 3H), 0.98-1.12 (m, 1H), 1.32-1.43 (m, 1H), 1.43-1.64 (m, 2H), 2.26 (dd, J=16.5, 9.9 Hz, 1H), 2.47 (dd, J=19.5, 3.7 Hz, 1H), 3.28-3.36 (m, 1H). Low resolution mass spec: nominal m/e calc'd for C₉H₁₉NO₂(M+H)⁺: 174. Found: m/e 174.

### (3R,4R,5R)-3-Amino-4,5-dimethyl-heptanoic Acid-1/6-succinic acid complex-1/6-hydrate, i.e., 6-((3R,4R,5R)-3-amino-4,5-dimethyl-heptanoic acid):1-(succinic acid): 1-(H₂O)

(3R,4R,5R)-3-Amino-4,5-dimethyl-heptanoic acid (7.6 g, 44 mmol) and succinic acid (2.6 g, 22 mmol) were suspended in 20.2 mL of water. The slurry was heated to 100° C. to dissolve the solids. Acetonitrile (253 mL) was added to the hot solution. The mixture was stirred at 55° C. for 1 hour, and then cooled gradually to room temperature overnight. The resulting solids were filtered, washed with 10 mL of acetonitrile, and dried on a nitrogen press to give 6.21 g (72% yield) of the titled compound as fluffy white crystals: ¹H NMR (CD₃OD, δ99.77 MHz) ¹H NMR (CD₃OD, δ99.77 MHz) 0.88 (t, J=7.5 Hz, 3H), 0.91 (d, J=7.0 Hz, 3H), 0.94 (d, J=6.6 Hz, 3H), 0.98-1.12 (m, 1H), 1.32-1.43 (m, 1H), 1.43-1.64 (m, 2H), 2.26 (dd, J=16.5, 9.9 Hz, 1H), 2.47 (dd, J=19.5, 3.7 Hz, 1H), 2.50 (s, 0.67H), 3.28-3.36 (m, 1H). Low resolution mass spec: nominal m/e calc'd for C₉H₁₉NO₂ (M+H)⁺: 174. Found: m/e 174. Anal. calc'd for 6-((3S,4R,5R 3-amino-4,5-dimethyl-heptanoic Acid):1-(succinic Acid): 1-(H₂O), C₅₈H₁₂₂N₆O₁₃: C, 59.26; H, 10.46; N, 7.15. Found: C, 59.28; H, 10.58; N, 7.09. KF calc'd for C₅₈H₁₂₂N₆O₁₃:H₂O, 1.43 wt %. Found: H₂O, 1.50 wt %.

### EXAMPLE 8

### (4S,5R)-4,5-Diphenyl-oxazolidin-2-one

To a 5 L round bottom flask equipped with an overhead stirrer, thermocouple and distillation head, was charged 550 g (2.579 mol) of (1R,2S)-diphenyl-2-aminoethanol, 457 g (3.868 mol, 1.5 eq) of diethylcarbonate, 18 g (0.258 mol, 0.1 eq) of NaOEt in 100 mL of EtOH and 3.5 L of toluene. The reaction was heated until an internal temperature of 90° C. was reached and EtOH distillation began. The reaction was refluxed until an internal temperature of 110° C. was reached (7 hours). For every 500 mL of solvent that was removed via the distillation head, 500 mL of toluene was added back to the reaction. A total of about 1.6 L of solvent was removed. The reaction was allowed to cool to room temperature and then filtered on a 3 L coarse fritted funnel with 2 psig N₂. Nitrogen was blown over the cake overnight to give 580 g (94% yield) of the titled compound: ¹H NMR (DMSO) 7.090-6.985 (m, 6H), 6.930-6.877 (m, 4H), 5.900 (d, 1H, J=8.301), 5.206 (d, 1H, J=8.301).

### (4S,5R)-3-((E)-2-Methyl-hex-2-enoyl)-4,5-diphenyl-oxazolidin-2-one (Alternative A)

A 20 L jacketed reactor was fitted with a reflux condenser. To the reactor was charged 1100 g (4.597 mol) of (4S,5R)-4,5-diphenyl-oxazolidin-2-one, 884 g (6.896 mol) (E)-2-methyl-2-pentenoic acid, 1705 g (6.896 mol) of EEDQ, 48 g (1.149 mol) of LiCl and 16 L of EtOAc. The reaction mixture was heated to 65° C. and was held for 200 minutes. The reaction mixture was cooled to room temperature and was extracted 3× with 3.5 L aliquots of 1N HCl. The combined aqueous extracts were filtered to give a white solid. The recovered white solid was added back to the organic layer. The 20 L reactor was fitted with a distillation head and the organic layer was distilled to remove in succession: 13.5 L of EtOAc, after which 5 L of heptane was added to the reactor; 5 L of EtOAc/heptane, after which 5 L of heptane was added to the reactor; and 2.7 L of EtOAc/heptane, after which 2.7 L of heptane was added to the reactor. The contents of the reactor were cooled to 25° C. and the resulting mixture was filtered under 5 psig nitrogen while washing with 4 L of heptane. The wet cake was dried under nitrogen pressure overnight to give 1521 g of the titled compound: ¹H NMR (DMSO) 7.12-6.94 (m, 8H), 6.834 (dd, 2H, J=7.813, 1.709), 6.060 (d, 1H, J=8.057), 6.050 (td, 1H, J=7.447, 1.221), 5.795 (d, 1H, J=8.057), 2.119-2.064 (m, 2H), 1.778 (d, 3H, J=0.997), 1.394 (m, 2H), 0.874 (t, 3H, J=7.324); Anal Calc'd for C₂₂H₂₃N₁O₃: C, 75.62; H, 6.63; N, 4.01. Found: C, 75.26; H, 6.72; N, 3.95.

### (4S,5R)-3-(2-(E)-Methyl-hex-2-enoyl)-4,5-diphenyl-oxazolidin-2-one (Alternative B)

To a solution of (E)-2-methyl-2-hexenoic acid (6.0 g, 47 mmol) in 250 mL of THF at 0° C. was added 16.3 mL (117 mmol) of triethylamine, then 5.8 mL (47 mmol) of pivaloyl chloride resulting in a thick suspension. The mixture was stirred for 1 hour at 0° C. at which time 2.0 g (47 mmol) of lithium chloride was added in one portion, followed by 10.0 g (42 mmol) of (4S,5R)-4,5-diphenyl-2-oxazolidinone in four batches. Stirring was maintained throughout the solid additions. The resulting mixture was stirred for 1 hour at 0° C., then for 1 hour at ambient temperature, and was vacuum filtered through a coarse frit and concentrated. The residue was partitioned between EtOAc/water, and the organics were dried over MgSO₄ and concentrated. To the residue was added 100 mL of MTBE and the mixture warmed cautiously with swirling. The warm slurry was filtered to provide 10.5 g (64% yield) of the titled compound as a colorless solid: ¹H NMR (CDCl₃) δ 7.12 (m, 3H), 7.07 (m, 3H), 6.94 (m, 2H), 6.84 (m, 2H), 6.17 (m, 1H), 5.89 (d, J=7.8 Hz, 1H), 5.68 (d, J=7.8 Hz, 1H), 2.18 (m, 2H), 1.92 (s, 3H), 1.50 (m, 2H), 0.96 (t, J=7.6 Hz, 3H).

### (4S,5R)-3-((2R,3R)-2,3-Dimethyl-hexanoyl)-4,5-diphenyl-oxazolidin-2-one

A 22 L 4-neck round bottom flask was equipped with an addition funnel, mechanical stirrer, and nitrogen inlet. The system was purged with nitrogen for 1 hour. THF (6 L) were charged to the flask followed by 1236 g (6.01 mol) of CuBr.S(CH₃)₂ and 364 g (8.59 mol) of LiCl. The reaction was stirred for 15 minutes at ambient temperature. The solution was cooled to −35° C. and 3.96 L (11.88 mol) of a 3M solution of CH₃MgCl in THF was charged at a rate as to keep the internal temperature of the reaction mixture below −25° C. The reaction was stirred for 1 hour after the addition of CH₃MgCl was complete. (4S,5R)-3-((E)-2-Methyl-hex-2-enoyl)-4,5-diphenyl-oxazolidin-2-one (1.00 Kg, 2.86 mol) was added as a solid in one portion and the reaction was stirred at −30° C. for 4 hours. The reaction mixture was transferred over a 2 hour period into another 22 L flask equipped with a mechanical stirrer, transfer line, vacuum line, and containing 4 L of 1:1 acetic acid:THF solution cooled in an ice-water bath. The quenched solution was stirred for 30 minutes and then diluted with 4 L of 2M NH₄OH in saturated aqueous NH₄Cl and 2 L of water. The biphasic mixture was stirred for 15 minutes and the phases separated. The organic phase was washed 4× with 4 L aliquots of the 2M NH₄OH solution. No more blue color was observed in the washes or the organic phase so the organic phase was diluted with 8 L of water and the THF was distilled off until the internal temperature of the distillation pot reached 95° C. The suspension was cooled to ambient temperature and filtered. The solids were washed with 4 L of water and suction dried to give 868.2 g of an off white solid. This material was recrystallized from 2 L of 95:5 heptane:toluene with a cooling rate of 5° C. per hour to provide 317.25 g of the titled compound as a white solid: ¹H NMR (CDCl₃) 7.12-6.85 (m, 10H), 5.90 (d, 1H, J=8.06 Hz), 5.72 (d, 1H, J=7.81), 3.83-3.76 (m, 1H), 1.95-1.89 (m, 1H), 1.35-1.31 (m, 1H). 1.11 (d, 3H, J=6.84), 1.10-0.95 (m, 3H), 0.92 (d, 3H, J=6.59), 0.76 (t, 3H, J=7.20) MS (APCl) M+1=366.2.

### (2R,3R)-2,3-Dimethyl-hexanoic acid

A 12 L, 4-necked round bottom flask, equipped with a mechanical stirrer, 500 mL addition funnel, nitrogen inlet, and thermometer, was charged with 4515 mL of THF and 330.0 g of (4S,5R)-3-((2R,3R)-2,3-dimethyl-hexanoyl)-4,5-diphenyl-oxazolidin-2-one. The resulting liquid mixture (all solids dissolved) was cooled to −5° C. to 0° C. using an acetone/ice bath. A solution of 60.6 g of LiOH—H₂O in 1800 mL of deionized water was cooled to 0° C. to 5° C. and was combined with 512 g of cold 30% (wt/wt) hydrogen peroxide in a 2 L Erlenmeyer flask. The solution was kept cold using an ice/water bath. After the oxazolidinone/THF solution in the 12 L reaction flask reached −5° C. to 0° C., the addition funnel was charged with approximately one quarter of the cold LiOH/water/H₂O₂ solution. While maintaining a nitrogen sweep to minimize oxygen concentration in the reactor headspace, the LiOH/water/H₂O₂ solution was added dropwise to the vigorously stirred oxazolidinone/THF solution at such a rate as to maintain the reaction temp at 0° C. to 5° C. The addition funnel was recharged with approximately one quarter of the cold LiOH/water/H₂O₂ solution as required until all of the solution had been added to the reaction mixture (about 40 minutes for 0.45 mol scale). After the addition was completed, the mixture was stirred at 0° C. to 5° C. for 5 hours, during which the reaction mixture changed from a homogeneous solution to white slurry. A solution of 341 g of Na₂SO₃ and 188 g of NaHSO₃ in 2998 mL of deionized water (15 wt %) was added dropwise to the reaction mixture over about a 1.5 hour period (reaction was exothermic) via the addition funnel, while maintaining the reaction temperature at 0° C. to 10° C. Following the addition, the reaction mixture was stirred at 0° C. to 10° C. for 1 hour. The reaction mixture was tested with potassium iodide-starch test paper to ensure the absence of peroxides. The reaction mixture was charged with 2000 mL of EtOAc and was stirred 5 minutes. The phases were separated and the aqueous phase was extracted with 2000 mL of EtOAc. The combined organic extract was washed with brine (2×1500 mL). The colorless organic solution was concentrated under vacuum (35° C.-40° C.) to a “wet,” white solid. Heptane (1000 mL) was added and the slurry was concentrated under vacuum (35° C.-40° C.) to a wet, white solid. Heptane (5000 mL) was added and the slurry was maintained at 0° C. to 5° C. for 16 hours and then at −10° C. to −5° C. for 1 hour. The cold slurry was filtered through a thin pad of celite, and the filter cake was washed with 100 mL of −10° C. to −5° C. heptane. The colorless filtrate was concentrated under vacuum (40° C.-45° C.) to give 130 g of the titled compound as a pale yellow oil: ¹H NMR (400 MHz, CHLOROFORM-D) 0.89 (t, J=7.00 Hz, 3H), 0.94 (d, J=6.8 Hz, 3H), 1.13 (d, J=7.0 Hz, 3H), 1.75-1.82 (m, 1H), 2.34-2.41 (m, 1H); GC Chiral purity: 99.18% (with 0.82% diastereomer) (direct acid method). Chemical purity: 100%. Anal. Calc'd for C₈H₁₆O₂: C, 66.63; H, 11.18. Found: C, 66.15; H, 11.41.

### (4R,5R)-4,5-Dimethyl-3-oxo-octanoic acid ethyl ester (Alternative A)

A 5 L 3-neck round bottom flask, equipped with a reflux condenser, mechanical stirrer, nitrogen inlet, and thermometer, was charged with 1390 mL of dry THF and 389.3 g of potassium ethyl malonate. MgCl₂ (217.8 g) was added in three equal portions so that the internal temperature was less than 50° C. The resulting grey slurry was heated to 55° C. to 60° C. using a temperature controlled heating mantle. The mixture was stirred at 55° C. to 60° C. for 5 hours. A 2 L 3-neck round bottom flask, equipped with a 500 mL addition funnel, mechanical stirrer, nitrogen inlet, and thermometer, was charged with 680 mL of dry THF and 286.8 g of 1,1′-carbonyldiimidazole (CDI). The addition funnel was charged portion-wise with a solution of 219.9 g of (2R,3R)-2,3-dimethyl-hexanoic acid in 350 mL of dry THF. The entire dimethyl-hexanoic acid acid/THF solution was added dropwise to the stirred CDI/THF suspension at such a rate so as to control the evolution of CO₂ and to maintain the reaction at a temperature of 20° C. to 25° C. Following the addition, the reaction mixture was stirred at 20° C. to 25° C. for 1 hour, during which the slurry became a pale yellow solution. After the 5-hour reaction time, the malonate/MgCl₂ reaction mixture was cooled to 20° C. to 25° C. and the condenser was replaced with a 1 L addition funnel. The addition funnel was charged portion-wise with the dimethylhexanoic acid/CDl/THF reaction mixture. This entire reaction mixture was added dropwise to the stirred malonate/MgCl₂/THF reaction mixture over about 10 minutes. After the addition was completed, the reaction mixture was heated to 35° C. to 40° C. Some effervescence was noted. The reaction mixture was stirred at 35° C. to 40° C. for 16 hour. The reaction mixture was cooled to 20° C. to 25° C. A 12 L 3-neck round bottom flask, equipped with a mechanical stirrer and thermometer, was charged with 3060 mL of 2N aq. HCl. The reaction mixture (a grey suspension) was added portion-wise to the aq. HCl solution while maintaining an internal temperature of 20° C.-25° C. The reaction temperature was moderated with an ice/water bath; the reaction mixture pH was about 1. Following the addition, the reaction mixture was stirred at 20° C. to 25° C. for 2 hours. The reaction mixture was subsequently charged with 4000 mL of EtOAc and was stirred for 5 minutes. The phases were separated and the aqueous phase was extracted with 2000 mL of EtOAc. The combined organic extract was washed sequentially with: 1N aq. HCl (2×1500 mL); 1000 mL of water (incomplete phase separation); half saturated aq. Na₂CO₃ (2×1500 mL); 1000 mL water; and brine (2×1000 mL). (The aqueous base wash removed unreacted malonate ester-acid.) The straw colored organic solution was concentrated under vacuum (35° C.-40° C.) to give a cloudy, pale yellow oil with some white solid present. The oil was redissolved in 1500 mL of n-heptane and was filtered. The filtrate was concentrated under vacuum (40° C.-45° C.) to give 327 g of the titled compound as a pale yellow oil: ¹H NMR (400 MHz, CHLOROFORM-D) d ppm 0.82 (t, J=7.1 Hz, 3H), 0.85 (d, J=6.8 Hz, 3H), 0.99 (d, J=7.1 Hz, 3H), 1.20 (t, J=7.3 Hz, 3H), 2.42-2.49 (m, 1H), 3.39 (s, 2H) 4.12 (q, J=7.16 Hz, 3H). GC Chemical purity: 96.24%.

### (4R,5R)-4,5-Dimethyl-3-oxo-octanoic acid ethyl ester (Alternative B)

To a solution containing 2.0 g (13.9 mmol) of (2R,3R)-2,3-dimethyl-hexanoic acid in 20 mL of dichloromethane was added 2.1 g (16.6 mmol) of chloromethylene dimethyl-ammonium chloride. After stirring the resulting solution under nitrogen for 1.5 hours, the solvent was evaporated to give (2R,3R)-2,3-dimethyl-hexanoyl chloride. Butyl lithium (32.7 ml, 52.4 mmol) was added to a solution of diisopropylamine (4.9 g, 48.5 mmol) in dry THF (20 mL) under nitrogen at 0° C. and stirred for 20 minutes. The solution was cooled to −78° C. and 4.3 g (48.5 mmol) of ethyl acetate was added. The solution was stirred at that temperature for 45 minutes. (2R,3R)-2,3-Dimethyl-hexanoyl chloride in dry THF (20 mL) was slowly added to the ethyl acetate enolate at −78° C. and the resulting reaction mixture was allowed to warm to room temperature. The reaction mixture was stirred at room temperature for 2.5 hours and was cooled to 0° C. The reaction was quenched with a saturated solution of ammonium chloride and extracted into ethyl acetate. The solution was washed with brine, dried over MgSO₄ and concentrated. The resulting residue was filtered through a silica plug, eluting with 60/40 solution of hexane/ethyl acetate to afford 2.7 g (89.2% yield) of the titled compound as an oil.

### (4R,5R)-4,5-Dimethyl-3-oxo-octanoic acid ethyl ester (Alternative C)

To a solution containing 1.0 g (6.9 mmol) of (2R,3R)-2,3-dimethyl-hexanoic acid in 10 mL of dichloromethane was added 1.1 g of chloromethylene dimethyl-ammonium chloride (8.3 mmol). The resulting solution was stirred under nitrogen for 1.5 hours. The solvent was subsequently evaporated to give (2R,3R)-2,3-dimethyl-hexanoyl chloride. To a solution containing 2.5 g (14.6 mmol) of potassium monoethyl malonate in 50 mL of acetonitrile was added 1.7 g (17.3 mmol) of magnesium chloride and 1.2 g (11.4 mmol) of triethylamine. The resulting mixture was stirred at room temperature for 2.5 hours. The reaction was cooled to 0° C. and a solution of the (2R,3R)-2,3-dimethyl-hexanoyl chloride in acetonitrile (20 mL) was slowly added followed by the addition of triethylamine (0.4 g, 0.4 mmol). The reaction was heated to 40° C. and stirred at that temperature for 6 hours. The reaction mixture was cooled to 25° C., quenched with a saturated solution of ammonium chloride and extracted into ethyl acetate. The solution was washed with brine, dried over MgSO₄ and concentrated. The resulting residue was filtered through a silica plug, eluting with 60/40 solution of hexane/ethyl acetate to afford 1.3 g (87.8% yield) of the titled compound as an oil.

### (4R,5R)-3-Methoxyamino-4,5-dimethyl-(Z)-oct-2-enoic acid ethyl ester

A 2 L 3-necked round bottom flask, equipped with magnetic stirring and nitrogen inlet, was charged with 153 g (0.71 mol) of (4R,5R)-4,5-dimethyl-3-oxo-octanoic acid ethyl ester and 600 mL of anhydrous EtOH. The solution was cooled to 0° C.-5° C. with an ice bath and 65.6 g (0.79 mol) of methoxylamine hydrochloride was added, followed by 58.6 g (0.71 mol) of sodium acetate. This flask contents were slowly warmed to room temperature (about 2 hours) and the reaction mixture was stirred at room temperature for another 24 hours. The solvent (EtOH) was removed under reduced pressure and the mixture was charged with CH₂Cl₂ (2×300 mL), which was subsequently removed. The mixture was cooled to RT, diluted with CH₂Cl₂ (300 mL), stirred at room temperature for 0.5 hours, and filtered under 5 psig of nitrogen. The filter cake was washed with CH₂Cl₂ (150 mL). The filtrate was concentrated under vacuum (50° C.) to give 172 g (99% yield) of the titled compound as a light yellow oil: ¹H NMR (400 MHz, CHLOROFORM-D) 0.87 (t, J=3.5 Hz, 5H), 0.89 (d, J=7.2 Hz, 3H), 1.08 (d, J=7.0 Hz, 3H), 1.24 (t, J=7.2 Hz, 4H), 1.3-1.55 (m, 2H), 2.25 (m, 1H), 3.15 (q, J=19.5 Hz, 2H) 3.81 (s, 3H), 4.14 (q, J=7.0 Hz, 2H).

### (4R,5R)-3-Amino-4,5-dimethyl-(Z)-oct-2-enoic acid ethyl ester

A reactor vessel charged with 171 g of (4R,5R)-3-methoxyamino-4,5-dimethyl-(Z)-oct-2-enoic acid ethyl ester, 1600 mL of MeOH, and 65 g of Raney nickel (Ra—Ni) catalyst. The methoxyamino ester was reacted with hydrogen at 50 psig to 55 psig. During the hydrogenation, additional Ra—Ni was added at reaction times of 8 hours (20 g), 21 hours (20 g), and 37 hours (8 g). After the reaction was completed (51 hours), the Ra—Ni was filtered off and the filtrate was concentrated under reduced pressure to give 150 g (>99% yield) of the titled compound as an oil: ¹H NMR (400 MHz, CHLOROFORM-D): 0.86 (t, J=4.5 Hz, 3H), 0.88 (d, J=4.9 Hz, 3H), 1.05-1.50 (m, 6H), 1.10 (d, J=7.0 Hz, 3H), 1.24 (t, J=7.2 Hz, 3H), 1.87 (m, 1H), 3.45 (s, 2H) 4.08 (q, J=7.0 Hz, 2H).

### (4R,5R)-3-Acetylamino-4,5-dimethyl-(Z)-oct-2-enoic acid ethyl ester

To a 1 L 3-necked round bottom flask equipped with an overhead stirrer, thermocouple, addition funnel, and nitrogen inlet, was charged 150 g (0.70 mol) of (4R,5R)-3-amino-4,5-dimethyl-(Z)-oct-2-enoic acid ethyl ester and 50 mL of dry CH₂Cl₂. The reaction mixture was cooled to −20° C. To the mixture was added, successively, acetyl chloride (60 mL, 0.84 mol) and pyridine (66.8 g, 0.84 mol) over 0.5-hour time intervals. After the additions, the mixture was stirred at −20° C. to 0° C. for 2 hours and then filtered to remove the pyridine.HCl salt. The filtrate was diluted with 200 mL of CH₂Cl₂ and washed 2× with aliquots of aq NH₄Cl. The organic solution was treated with silica gel (50 g), MgSO₄ (20 g) and charcoal (20 g), and stirred at room temperature for 0.5 hours. The solids were filtered off and the filtrate was concentrated under reduced pressure to give 166.5 g (93% yield) of the titled compound as an oil: ¹H NMR (400 MHz, CHLOROFORM-D) 0.85 (t, J=7.4 Hz, 3H), 0.95 (d, J=6.8 Hz, 3H), 1.00 (d, J=7.0 Hz, 3H), 1.11 (m, 1H) 1.29 (t, J=5.8 Hz, 3H), 1.40-1.25 (m, 2H), 1.65 (m, 1H) 2.13 (s, 3H), 3.80 (m, 1H) 4.2-4.14 (m, 3H), 5.01 (s, 1H), 11.28 (s, 1H).

### (3R,4R,5R)-3-Acetylamino-4,5-dimethyl-octanoic acid ethyl ester

A reactor was charged with 166 g of (4R,5R)-3-acetylamino-4,5-dimethyl-(Z)-oct-2-enoic acid ethyl ester (substrate), 2650 mL of MeOH, and 36 g of Pd/SrCO₃ (lot#D25N17) catalyst. The substrate was reacted with H₂ at a pressure of 50 psig to 51 psig of. During hydrogenation, additional catalyst was added at a reaction time of 67 hours (10 g). After the reaction was completed (90 hours), Pd/SrCO₃ was filtered off and the filtrate was concentrated under reduced pressure to give 167 g (>99% yield) of the titled compound as an oil: ¹H NMR (400 MHz, CHLOROFORM-D): 0.82 (d, J=6.8 Hz, 3H), 0.88 (t, J=7.2 Hz, 3H), 0.90 (d, J=6.6 Hz, 3H), 1.25 (t, J=7.3 Hz, 3H), 1.00-1.58 (m, 6H), 1.96 (s, 3H), 2.52 (q, J=5.2 Hz, 2H), 3.47 (s, 1H), 4.10-4.30 (m, 2H), 4.12 (t, J=7.1 Hz, 1H), 5.9 (d, 1H).

### (3R,4R,5R)-3-Amino-4,5-dimethyl-octanoic acid hydrochloride

Under nitrogen, 167 g of crude (3R,4R,5R)-3-acetylamino-4,5-dimethyl-octanoic acid ethyl ester was diluted 1100 mL of 6N HCl, stirred at room temperature for 16 hours, and then heated to reflux for another 24 hours. The reaction mixture was concentrated and recharged with 500 mL of isopropyl alcohol (IPA), which was subsequently removed. Acetonitrile (500 mL) was added to the crude white HCl salt and the mixture stirred at 20° C. to 25° C. for 1 hour. The resulting slurry was filtered, and the solids isolated to give 97 g of the titled compound (67% yield, 89.7% chemical purity; 90.7% chiral purity with two major diastereomers, 6.8% and 1.5%): ¹H NMR (CD₃OD): δ 0.89t J=7.0 Hz, 3H), 0.94t, J=6.9 Hz, 6H), 1.65-1.0 (m, 4H), 2.61 (dd, J=7.6 Hz, 1H), 2.73 (dd, J=4.6 HZ, 1H), 3.27 (m, J=1.6 Hz, 2H), 3.56 (m, 1H), 4.82 (s, 3H).

### (3R,4R,5R)-3-Amino-4,5-dimethyl-octanoic acid

(3R,4R,5R)-3-Amino-4,5-dimethyl-octanoic acid hydrochloride (92 g, 0.41 mol) was dissolved in 250 mL to 260 mL of dry MeOH in a 2 L 3-necked round bottom flask. To this solution was added Et₃N (0.45 mol, 45.8 g) dropwise, which formed a white precipitate. The resulting slurry was stirred at room temperature for 15 minutes. The solvent was removed to dryness. The white solid was dispersed in 1 L of CH₂Cl₂ (1 L) and stirred for 1 hour. CH₃CN (0.6 L) was added, and the slurry was stirred for another 0.5 hours. The slurry was filtered and the solids were washed 2× with 50 mL aliquots of CH₃CN, giving 71 g of the titled compound as a white solid (92% yield; 98.8% chiral purity; 99.7% chemical purity): ¹H NMR (400 MHz, CD₃OD): 0.89 (t, J=7.2 Hz, 3H), 0.91 (d, J=5.1 Hz, 3H), 0.93 (d, J=6.6 Hz, 3H), 1.02-1.65 (m, 4H), 2.26 (dd, J=10.2 Hz, 1H), 2.50 (dd, J=3.7 Hz, 1H), 3.27 (m, J=1.6 Hz, 2H) 3.33-3.28 (m, 1H), 4.82 (s, 3H).

The combination of the invention can be administered alone but one or both elements will generally be administered in an admixture with suitable pharmaceutical excipient(s), diluent(s) or carrier(s) selected with regard to the intended route of administration and standard pharmaceutical practice. If appropriate, auxiliaries can be added. Auxiliaries are preservatives, anti-oxidants, flavours or colourants. The compounds of the invention may be of immediate-, delayed-, modified-, sustained-, pulsed- or controlled-release type.

Thus, as a further aspect of the present invention, there is provided a pharmaceutical composition comprising an alpha-2-delta ligand, an EP4-receptor antagonist, or pharmaceutically acceptable salts thereof, and a suitable excipient, diluent or carrier. The composition is suitable for use in the treatment of pain, particularly inflammatory, neuropathic, visceral or nociceptive pain.

As an alternative aspect of the present invention, there is provided a pharmaceutical composition comprising a synergistic combination comprising an alpha-2-delta ligand, EP4-receptor antagonist, or pharmaceutically acceptable salts thereof, and a suitable excipient, diluent or carrier. The composition is suitable for use in the treatment of pain, particularly inflammatory, neuropathic, visceral or nociceptive pain.

For the avoidance of doubt, references herein to “treatment” include references to curative, palliative and prophylactic treatment.

For non-human animal administration, the term ‘pharmaceutical’ as used herein may be replaced by ‘veterinary.’

When the components are formulated separately, each element of the combination of the present invention is preferably in unit dosage form, each unit dose containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The quantity of active component in a unit dose preparation may be varied or adjusted from 0.1 mg to 1 g according to the particular application and the potency of the active components. In medical use the drug may be administered three times daily as, for example, capsules of 100 or 300 mg. In therapeutic use, the compounds utilized in the pharmaceutical method of this invention are administered at the initial dosage of about 0.01 mg to about 100 mg/kg daily. A daily dose range of about 0.01 mg to about 100 mg/kg is preferred. The dosages, however, may be varied depending upon the requirements of the patient, the severity of the condition being treated, and the compounds being employed. Determination of the proper dosage for a particular situation is within the skill of the art. Generally, treatment is initiated with smaller dosages which are less than the optimum dose of the compounds. Thereafter, the dosage is increased by small increments until the optimum effect under the circumstances is reached. For convenience, the total daily dosage may be divided and administered in portions during the day, if desired.

The combination of the present invention in a single dosage form is suitable for administration to any mammalian subject, preferably human. Administration may be once (o.d.), twice (b.i.d.) or three times (t.i.d.) daily, suitably b.i.d. or t.i.d., more suitably b.i.d, most suitably o.d.

For veterinary use, a combination according to the present invention or veterinarily acceptable salts or solvates thereof, is administered as a suitably acceptable formulation in accordance with normal veterinary practice and the veterinary surgeon will determine the dosing regimen and route of administration which will be most appropriate for a particular animal.

The elements of the combination of the present invention can be administered, for example but not limited to, the following route: orally, buccally or sublingually in the form of tablets, capsules, multi- and nano-particulates, gels, films (incl. muco-adhesive), powder, ovules, elixirs, lozenges (inc. liquid-filled), chews, solutions, suspensions and sprays. The compounds of the invention may also be administered as osmotic dosage form, or in the form of a high energy dispersion or as coated particles or fast-dissolving, fast-disintegrating dosage form as described in Ashley Publications, 2001 by Liang and Chen. The compounds of the invention may be administered as crystalline or amorphous products, freeze dried or spray dried. Suitable formulations of the compounds of the invention may be in hydrophilic or hydrophobic matrix, ion-exchange resin complex, coated or uncoated form and other types as described in U.S. Pat. No. 6,106,864 as desired. Such pharmaceutical compositions of the individual components of the combination, or the combination itself, for example, tablets, may contain excipients such as microcrystalline cellulose, lactose, sodium citrate, calcium carbonate, dibasic calcium phosphate, glycine and starch (preferably corn, potato or tapioca starch), mannitol, disintegrants such as sodium starch glycolate, crosscarmellose sodium and certain complex silicates, and granulation binders such as polyvinylpyrrolidone, hydroxypropylmethylcellulose (HPMC), triglycerides, hydroxypropylcellulose (HPC), bentonite sucrose, sorbitol, gelatin and acacia. Additionally, lubricating agents may be added to solid compositions such as magnesium stearate, stearic acid, glyceryl behenate, PEG and talc or wetting agents, such as sodium lauryl sulphate. Additionally, polymers such as carbohydrates, phosphoholipids and proteins may be included.

Fast dispersing or dissolving dosage formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol or xylitol. The terms dispersing or dissolving as used herein to describe FDDFs are dependent upon the solubility of the drug substance used, i.e. where the drug substance is insoluble a fast dispersing dosage form can be prepared and where the drug substance is soluble a fast dissolving dosage form can be prepared.

The solid dosage form, such as tablets are manufactured by a standard process, for example, direct compression or a wet, dry or melt granulation, melt congealing and extrusion process. The tablet cores which may be mono or multi-layer may be coated with appropriate overcoats known in the art.

Solid compositions of a similar type may also be employed as fillers in capsules such as gelatin, starch or HPMC capsules. Preferred excipients in this regard include lactose, starch, a cellulose, milk sugar or high molecular weight polyethylene glycols. Liquid compositions may be employed as fillers in soft or hard capsules such as gelatin capsule. For aqueous and oily suspensions, solutions, syrups and/or elixirs, the compounds of the invention may be combined with various sweetening or flavouring agents, colouring matter or dyes, with emulsifying and/or suspending agents and with diluents such as water, ethanol, propylene glycol, methylcellulose, alginic acid or sodium alginate, glycerin, oils, hydrocolloid agents and combinations thereof. Moreover, formulations containing these compounds and excipients may be presented as a dry product for constitution with water or other suitable vehicles before use.

Liquid form preparations include solutions, suspensions, and emulsions, for example, water or water propylene glycol solutions. For parenteral injection, liquid preparations can be formulated in solution in aqueous polyethylene glycol solution. Aqueous solutions suitable for oral use can be prepared by dissolving the active component in water and adding suitable colorants, flavors, stabilizing and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

The elements of the combination of the present invention can also be administered by injection, that is, intravenously, intramuscularly, intracutaneously, intraduodenally, or intraperitoneally, intraarterially, intrathecally, intraventricularly, intraurethrally, intrasternally, intracranially, intraspinally or subcutaneously, or they may be administered by infusion, needle-free injectors or implant injection techniques. For such parenteral administration they are best used in the form of a sterile aqueous solution, suspension or emulsion (or system so that can include micelles) which may contain other substances known in the art, for example, enough salts or carbohydrates such as glucose to make the solution isotonic with blood. The aqueous solutions should be suitably buffered (preferably to a pH of from 3 to 9), if necessary. For some forms of parenteral administration they may be used in the form of a sterile non-aqueous system such as fixed oils, including mono- or diglycerides, and fatty acids, including oleic acid. The preparation of suitable parenteral formulations under sterile conditions for example lyophilisation is readily accomplished by standard pharmaceutical techniques well-known to those skilled in the art. Alternatively, the active ingredient may be in powder form for constitution with a suitable vehicle (e.g. sterile, pyrogen-free water) before use.

Also, the elements of the combination of the present invention can be administered intranasally or by inhalation. They are conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomiser (preferably an atomiser using electrohydrodynamics to produce a fine mist) or nebuliser, with or without the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A [trade mark]) or 1,1,1,2,3,3,3-heptafluoropropane (HFA 227EA [trade mark]), carbon dioxide, a further perfluorinated hydrocarbon such as Perflubron (trade mark) or other suitable gas. In the case of a pressurised aerosol, the dosage unit may be determined by providing a valve to deliver a metered amount. The pressurised container, pump, spray, atomiser or nebuliser may contain a solution or suspension of the active compound, e.g. using a mixture of ethanol (optionally, aqueous ethanol) or a suitable agent for dispersing, solubilising or extending release and the propellant as the solvent, which may additionally contain a lubricant, e.g. sorbitan trioleate. Capsules, blisters and cartridges (made, for example, from gelatin or HPMC) for use in an inhaler or insufflator may be formulated to contain a powder mix of the compound of the invention, a suitable powder base such as lactose or starch and a performance modifier such as l-leucine, mannitol or magnesium stearate.

Prior to use in a dry powder formulation or suspension formulation for inhalation the elements of the combination of the invention will be micronised to a size suitable for delivery by inhalation (typically considered as less than 5 microns). Micronisation could be achieved by a range of methods, for example spiral jet milling, fluid bed jet milling, use of supercritical fluid crystallisation or by spray drying.

A suitable solution formulation for use in an atomiser using electrohydrodynamics to produce a fine mist may contain from 1 μg to 10 mg of the compound of the invention per actuation and the actuation volume may vary from 1 to 100 μl. A typical formulation may comprise the elements of the combination of the invention, propylene glycol, sterile water, ethanol and sodium chloride. Alternative solvents may be used in place of propylene glycol, for example glycerol or polyethylene glycol.

Alternatively, the elements of the combination of the invention may be administered topically to the skin, mucosa, dermally or transdermally, for example, in the form of a gel, hydrogel, lotion, solution, cream, ointment, dusting powder, dressing, foam, film, skin patch, wafers, implant, sponges, fibres, bandage, microemulsions and combinations thereof. For such applications, the compounds of the invention can be suspended or dissolved in, for example, a mixture with one or more of the following: mineral oil, liquid petrolatum, white petrolatum, propylene glycol, polyoxyethylene polyoxypropylene compound, emulsifying wax, fixed oils, including synthetic mono- or diglycerides, and fatty acids, including oleic acid, water, sorbitan monostearate, a polyethylene glycol, liquid paraffin, polysorbate 60, cetyl esters wax, cetearyl alcohol, 2-octyldodecanol, benzyl alcohol, alcohols such as ethanol. Alternatively, penetration enhancers may be used. The following may also be used; polymers, carbohydrates, proteins, phospholipids in the form of nanoparticles (such as niosomes or liposomes) or suspended or dissolved. In addition, they may be delivered using iontophoresis, electroporation, phonophoresis and sonophoresis.

Alternatively, the elements of the combination of the invention can be administered rectally, for example in the form of a suppository or pessary. They may also be administered by vaginal route. For example, these compositions may be prepared by mixing the drug with suitable non-irritant excipients, such as cocoa butter, synthetic glyceride esters or polyethylene glycols, which are solid at ordinary temperatures, but liquefy and/or dissolve in the cavity to release the drug.

The elements of the combination of the invention may also be administered by the ocular route. For ophthalmic use, the compounds can be formulated as micronised suspensions in isotonic, pH adjusted, sterile saline, or, preferably, as solutions in isotonic, pH adjusted, sterile saline. A polymer may be added such as crossed-linked polyacrylic acid, polyvinylalcohol, hyaluronic acid, a cellulosic polymer (e.g. hydroxypropylmethylcellulose, hydroxyethylcellulose, methyl cellulose), or a heteropolysaccharide polymer (e.g. gelan gum). Alternatively, they may be formulated in an ointment such as petrolatum or mineral oil, incorporated into bio-degradable (e.g. absorbable gel sponges, collagen) or non-biodegradable (e.g. silicone) implants, wafers, drops, lenses or delivered via particulate or vesicular systems such as niosomes or liposomes. Formulations may be optionally combined with a preservative, such as benzalkonium chloride. In addition, they may be delivered using iontophoresis. They may also be administered in the ear, using for example but not limited to the drops.

The elements of the combination of the invention may also be used in combination with a cyclodextrin. Cyclodextrins are known to form inclusion and non-inclusion complexes with drug molecules. Formation of a drug-cyclodextrin complex may modify the solubility, dissolution rate, taste-masking, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

The term ‘administered’ includes delivery by viral or non-viral techniques. Viral delivery mechanisms include but are not limited to adenoviral vectors, adeno-associated viral (AAV) vectors, herpes viral vectors, retroviral vectors, lentiviral vectors, and baculoviral vectors. Non-viral delivery mechanisms include lipid mediated transfection, lipsomes, immunoliposomes, lipofectin, cationic facial amphiphiles (CFAs) and combinations thereof. The routes for such delivery mechanisms include but are not limited to mucosal, nasal, oral, parenteral, gastrointestinal, topical or sublingual routes.

The elements of the combination of the instant invention may be administered separately, simultaneously or sequentially for the treatment of pain. The combination may also optionally be administered with one or more other pharmacologically active agents. Suitable optional agents include:
i) opioid analgesics, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine and pentazocine;
ii) nonsteroidal antiinflammatory drugs (NSAIDs), e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, nabumetone, naproxen, oxaprozin, phenylbutazone, piroxicam, sulindac, tolmetin, zomepirac, and their pharmaceutically acceptable salts;
iii) barbiturate sedatives, e.g. amobarbital, aprobarbital, butabarbital, butabital, mephobarbital, metharbital, methohexital, pentobarbital, phenobartital, secobarbital, talbutal, theamylal, thiopental and their pharmaceutically acceptable salts;
iv) benzodiazepines having a sedative action, e.g. chlordiazepoxide, clorazepate, diazepam, flurazepam, lorazepam, oxazepam, temazepam, triazolam and their pharmaceutically acceptable salts,
v) H₁ antagonists having a sedative action, e.g. diphenhydramine, pyrilamine, promethazine, chlorpheniramine, chlorcyclizine and their pharmaceutically acceptable salts;
vi) miscellaneous sedatives such as glutethimide, meprobamate, methaqualone, dichloralphenazone and their pharmaceutically acceptable salts;
vii) skeletal muscle relaxants, e.g. baclofen, carisoprodol, chlorzoxazone, cyclobenzaprine, methocarbamol, orphrenadine and their pharmaceutically acceptable salts,
viii) NMDA receptor antagonists, e.g. dextromethorphan ((+)-3-hydroxy-N-methylmorphinan) and its metabolite dextrorphan ((+)-3-hydroxy-N-methylmorphinan), ketamine, memantine, pyrroloquinoline quinone and cis-4-(phosphonomethyl)-2-piperidinecarboxylic acid and their pharmaceutically acceptable salts;
ix) alpha-adrenergic active compounds, e.g. doxazosin, tamsulosin, clonidine and 4-amino-6,7-dimethoxy-2-(5-methanesulfonamido-1,2,3,4-tetrahydroisoquinol-2-yl)-5-(2-pyridyl)quinazoline;
x) tricyclic antidepressants, e.g. desipramine, imipramine, amytriptiline and nortriptiline;
xi) anticonvulsants, e.g. carbamazepine and valproate;
xii) Tachykinin (NK) antagonists, particularly Nk-3, NK-2 and NK-1 e.g.
xiii) antagonists, (αR,9R)-7-[3,5-bis(trifluoromethyl)benzyl]-8,9,10,11-tetrahydro-9-methyl-5-(4-methyl phenyl)-7H-[1,4]diazocino[2,1-g][1,7]naphthridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]methyl]-1,2-dihydro-3H-1,2,4-triazol-3-one (MK-869), lanepitant, dapitant and 3-[[2-methoxy-5-(trifluoromethoxy)phenyl]methylamino]-2-phenyl-piperidine (2S,3S)
xiv) Muscarinic antagonists, e.g. oxybutin, tolterodine, propiverine, tropsium chloride and darifenacin;
xv) COX-2 inhibitors, e.g. celecoxib, rofecoxib and valdecoxib;
xvi) Non-selective COX inhibitors (preferably with GI protection), e.g. nitroflurbiprofen (HCT-1026);
xvii) coal-tar analgesics, in particular, paracetamol;
xviii) neuroleptics, such as droperidol;
xix) Vanilloid receptor agonists, e.g. resinferatoxin;
xx) Beta-adrenergic compounds such as propranolol;
xxi) Local anaesthetics, such as mexiletine;
xxii) Corticosteriods, such as dexamethasone
xxiii) serotonin receptor agonists and antagonists;
xxiv) cholinergic (nicotinic) analgesics;
xxv) miscellaneous agents such as Tramadol®;
xxvi) PDEV inhibitors, such as sildenafil, vardenafil or taladafil;
xxvii) serotonin reuptake inhibitors, e.g. fluoxetine, paroxetine, citalopram and sertraline;
xxviii) mixed serotonin-noradrenaline reuptake inhibitors, e.g. milnacipran, venlafaxine and duloxetine; and
xxix) noradrenaline reuptake inhibitors, e.g. reboxetine and S,S-reboxetine.

The present invention extends to a product comprising an alpha-2-delta ligand, an EP4-receptor antagonist and one or more other therapeutic agents, such as those listed above, for simultaneous, separate or sequential use in the curative, prophylactic treatment of pain, particularly inflammatory, neuropathic, visceral or nociceptive pain.

### BIOLOGY EXAMPLES

### Methods

### Carrageenan-Induced Mechanical Hyperalgesia

Carrageenan-induced mechanical hyperalgesia (CIMH)[1] is utilized to evaluate analgesic activity of drugs against acute inflammatory pain.

1% carrageenan is prepared at least two days before use. An appropriate amount of λ-carrageenain is weighed into a 10 ml screw vial. Sterile saline is added to make 1% (w/v) suspension solution and the suspension is stirred for 8 hours with gentle heating to be dissolved obtain homogeneous suspension.

### Animals

Male Sprague-Dawley rats, 4 W (Japan SLC or Charles River Japan), 100-130 g are used. Environment conditions are controlled at a 12-h light/dark cycle with (lights on 076:00 a.m.). and an ambient temperature of 23+/−2 deg. C. Prior to start the experiment, animals are housed under this condition for 4-5 days. Each group is used a group of 6-8 rats. The rats are fasted for 16-18 hours before use and the rats are trained for measurement of mechanical threshold using the apparatus for two days before use.

The paw withdrawal response of the rat to increased pressure on a right hindpaw is recorded as mechanical threshold. The threshold is defined as “pre value”. Hyperalgesia is induced by intraplantar injection of 0.1 ml of 1% carrageenain in the right hindpaw. The paw withdrawal threshold is measured at 3.5 and 4.5 hours after the carrageenan injection. Rats are randomly grouped by the paw withdrawal threshold at 4.5 hours and pre value after the carrageenan injection. Vehicle or test compounds (10 ml of 0.1% methylcellulose/1 kg body weight) are given per orally at 5.5 hours after the carrageenain injection. The paw withdrawal Mechanical threshold is measured by an analgesy meter at 4, 5, 6.5 and 7.5 hours after the carrageenin carrageenan injection. The threshold at 6.5 or 7.5 hours is determined as ‘post value’.

### Data Analysis

% change in threshold=(preost value−postre value)/(pre value)×100% inhibition=(B−A)/(C−A)×100

A=average of % change in threshold post values in disease control (vehicle treated) group
B=average of % change in threshold post values in compound treated group
C=average of pre values of all rats used

% inhibition=(A−B)/|A|×100

The data is analyzed with unpaired t-test or one-way ANOVA (plus multiple comparison test) for % change in thresholdpost values. Results are considered significant when p<0.05 versus disease control group.

| | | 2 hours post dosing |
|---|---|---|
| | Dose (mg/kg) | Inhibition (%) |
| 4-[(1S)-1-({[5-chloro-2-(3- | 0.3 | 19.2 |
| fluorophenoxy)pyridin-3- | 1 | 41.9 |
| yl]carbonyl}amino)ethyl]benzoic | 3 | 50 |
| acid | | |
| Pregabalin | 3 | 1.6 |
| | 10 | 8.4 |
| | 30 | 16.4 |
| | 100 | 31 |
| Pregabalin/4-[(1S)-1-({[5- | 3/0.06 | 5.9 |
| chloro-2-(3- | 10/0.2 | 12.8 |
| fluorophenoxy)pyridin-3- | 30/0.6 | 44.7 |
| yl]carbonyl}amino)ethyl]benzoic | | |
| acid | | |

The biological activity of the alpha-2-delta ligands of the invention may be measured in a radioligand binding assay using [³H]gabapentin and the α₂δ subunit derived from porcine brain tissue (Gee N. S., Brown J. P., Dissanayake V. U. K., Offord J., Thurlow R., Woodruff G. N., J. Biol. Chem., 1996; 271:5879-5776). Results may be expressed in terms of μM or nM α2δ binding affinity.

The biological activity of EP4-receptor antagonists may be measured using a Rat EP receptor cell membrane binding assay and described below:

### Stable Expression of rat EP1, 2, 3 and 4 Receptors in the Human Embryonic Kidney (HEK293) Cell Line

The cDNA clones of rat EP1, 2, 3 and 4 receptors are obtained by polymerase chain reaction (PCR) from rat kidney or heart cDNA libraries (Clontech). Human embryonic kidney cells (HEK 293) are stably transfected with expression vectors for rat EP1, 2, 3 and 4 receptors in according to the method described in the article; the journal of biological chemistry vol. 271 No. 39, pp 23642-23645.

### Preparation of Membrane Fraction:

The EP1, 2, 3 and 4 transfectant are grown in Dulbecco's modified Eagle's medium containing 10% fetal calf serum, 100 U/ml penicillin, 100 μg/ml streptomycin and 600 μg/ml G418 (selection medium) at 37° C. in a humidified atmosphere of 5% CO₂ in air. For the membrane preparation, cells are harvested with phosphate buffered saline (PBS) and centrifuged at 400×g for 5 min. The pellet is suspended with child (4° C.) PBS containing 1 mM Pefabloc (4-(2-aminoethyl)-benzenesulfonyl fluoride (AEBSF)), 10 μM Phosphoramidon, 1 μM Pepstatin A, 10 μM Elastatinal, 100 μM Antipain. Cells are lysed with ultrasonic cell disrupter for 20-sec sonication. Then cell mixtures are centrifuged at 45,000×g for 30 minutes. The pellet is resuspended in assay buffer (10 mM 2-morpholinoeth-anesulfonic acid (MES)-KOH, 1 mM etylenediamine tetra-acetic acid (EDTA), 10 mM MgCl₂, pH 6.0), and protein concentration is determined by Bradford method (Bio-Rad assay). This membrane preparation is stored at −80° C. freezer until use for binding assay.

### Binding Assay:

### Membrane Binding Assay

[³H]-PGE₂ membrane binding assays are performed in the reaction mixture of 10 mM MES/KOH (pH6.0), 10 mM MgCl₂, 1 mM EDTA, 1 nM [³H]-PGE₂ (Amersham TRK431, 164 Ci/mmol), 2˜10 μg of protein from membrane fraction (rat EP1, 2, 3 and 4/HEK293 transfectant) and test compound (total volume is 0.1 ml in 96 well polypropylene plate). Incubation is conducted for 60 min at room temperature prior to separation of the bound and free radioligand by rapid filtration through glass fiber filters (Printed Filtermat B, 1205-404, glass fiber, double thickness, size 102×258 mm, Wallac inc., presoaked in 0.2% polyethylenimine). Filters are washed with assay buffer and the residual [³H]-PGE₂ bound to the filter is determined by liquid scintillation counter (1205 Betaplate™). Specific binding is defined as the difference between total binding and nonspecific binding which is determined in the presence of 10 μM PGE₂.

cAMP Assay in Rat EP₄ Transfectant

HEK293 cells expressing rat EP₄ receptors (rEP₄ cells) are maintained in DMEM containing 10% FCS and 600 μg/ml geneticin. For harvesting rEP₄ cells, culture medium is aspirated and cells in 75 cm² flask are washed with 10 ml of phosphate buffered saline (PBS). Another 10 ml of PBS is added to the cells and incubated for 20 min at room temperature. Rat EP₄ cells are harvested by pipetting and centrifuged at 300 g for 4 min. Cells are resuspended in DMEM without neutral red at a density of 5×10⁵ cells/ml. The cells (70 μl) are mixed with 70 μl of DMEM (without neutral red) containing 2 mM IBMX (PDE inhibitor), 1 nM PGE₂ and test compounds in PCR-tubes, and incubated at 37° C. for 10 min. The reaction is stopped by heating at 100° C. for 10 min with thermal cycler. Concentration of cAMP in reaction mixtures is determined with SPA cAMP Kit (Amersham) according to the manufacture's instruction.

Reference: Eur. J. Pharmacol. 340 (1997) 227-241

The ability of compounds to act as EP4-receptor antagonists can be measured according to established procedures, particularly those described in the documents mentioned hereinabove.

Suitable EP4-receptor antagonist compounds of the present invention may be prepared as described herein below or in the aforementioned patent literature references, which are illustrated by the following non-limiting examples and intermediates.

The following examples illustrate the preparation of EP4-receptor antagonists described in WO-A-02/32900:

### Example 1

### 2-ETHYL-5,7-DIMETHYL-3-(4-{2-[({[(4-METHYLPHENYL)SULFONYL]AMINO}CARBONYL)AMINO]ETHYL}PHENYL)-3H-IMIDAZO[4,5-b]PYRIDINE

### STEP 1. 4,6-Dimethyl-3-nitro-2(1H)-pyridinone

A mixture of ethyl nitroacetate (80.0 g, 601 mmol) in ammonium hydroxide (25% NH₃ in water, 400 mL) was stirred at room temperature for 3 days, and then the solution was concentrated by air-drying. The residue was dissolved in water (450 mL). To the solution was added 2,4-pentanedione (73.1 g, 730 mmol), pyridine (16.2 mL, 200 mmol) and acetic acid (11.4 mL, 200 mmol), and the mixture was stirred for an additional 7 days. The resulting precipitates were collected by filtration and dried under reduced pressure to give 35.0 g (35%) of the title compound as yellow solids: ¹H-NMR (DMSO-d₆) δ 12.44 (1H, br.s), 6.06 (1H, s), 2.19 (3H, s), 2.13 (3H, s).

### STEP 2. 2-Chloro-4,6-dimethyl-3-nitropyridine

A mixture of 4,6-dimethyl-3-nitro-2(1H)-pyridinone (step 1, 10.0 g, 29.7 mmol) in phosphorus oxychloride (35 mL, 187.3 mmol) was stirred at 95° C. for 3 h, then cooled to 45° C. The excess amount of phosphorus oxychloride was removed by distillation under reduced pressure at 45° C. The residue was cooled to room temperature, and diluted with dichloromethane (75 mL). The resulting solution was cooled to 0° C., and 2N hydrochloric acid (50 mL) was added dropwise into the solution. The organic layer was separated, and washed with 2N hydrochloric acid (4×25 mL), 2N aqueous NaOH (2×50 mL) and brine (50 mL). The organic phase was dried (MgSO₄) and concentrated under reduced pressure to give 10.0 g (90%) of the title compound as white solids: ¹H-NMR (CDCl₃) δ 7.07 (1H, s), 2.56 (3H, s), 2.35 (3H, s).

### STEP 3. 2-{4-[(4,6-Dimethyl-3-nitro-2-pyridinyl)amino]phenyl}ethanol

A mixture of 2-chloro-4,6-dimethyl-3-nitropyridine (step 2, 1.3 g, 7.0 mmol) and 4-aminophenylethyl alcohol (1.4 g, 10.2 mmol) was placed in a sealed tube and heated at 150° C. for 3 h. The reaction mixture was cooled and purified by flash column chromatography on silica gel eluting with hexane/ethyl acetate (2:1) to afford 1.6 g (80%) of the title compound as orange solids: ¹H-NMR (CDCl₃) δ 9.55 (1H, br.s), 7.57 (2H, d, J=8.4 Hz), 7.20 (2H, d, J=8.4 Hz), 6.52 (1H, s), 3.84 (2H, t, J=6.4 Hz), 2.85 (2H, t, J=6.4 Hz), 2.54 (3H, s), 2.42 (3H, s).

### STEP 4. 2-{4-[(3-Amino-4,6-dimethyl-2-pyridinyl)amino]phenyl}ethanol

To a stirred solution of 2-{4-[(4,6-dimethyl-3-nitro-2-pyridinyl)amino]phenyl}ethanol (step 3, 1.6 g, 5.6 mmol) in ethyl acetate (15 mL) was added 10% Pd—C (160 mg). The mixture was stirred at room temperature for 6 h under hydrogen atmosphere. The palladium catalyst was removed by filtration and washed with ethanol (100 mL). The filtrate was concentrated under reduced pressure to afford 1.3 g (92%) of the title compound as pale yellow solids: ¹H-NMR (CDCl₃) δ 7.10 (4H, s), 6.61 (1H, s), 3.81 (2H, t, J=6.4 Hz), 2.80 (2H, t, J=6.4 Hz), 2.36 (3H, s), 2.19 (3H, s).

### STEP 5. 2-[4-(2-Ethyl-5,7-dimethyl-3H-imidazo[4,5-b]pyridin-3-yl)phenyl]ethyl propionate

To a stirred suspension of 2-{4-[(3-amino-4,6-dimethyl-2-pyridinyl)amino]phenyl}ethanol (step 4, 1.3 g, 5.1 mmol) in toluene (30 mL) was added dropwise propionyl chloride (990 mg, 10.7 mmol) at 0° C., and the reaction mixture was heated at reflux temperature for 2 h. After cooling, the mixture was poured into water (50 mL) and extracted with ethyl acetate (100 mL). The organic layer was washed with 2N aqueous NaOH (50 mL) and brine (50 mL), then dried (MgSO₄). Removal of solvent gave 1.8 g (quant.) of the title compound as brown solids: ¹H-NMR (CDCl₃) δ 7.41 (2H, d, J=8.4 Hz), 7.33 (2H, d, J=8.4 Hz), 6.90 (1H, s), 4.37 (2H, t, J=6.9 Hz), 3.04 (2H, t, J=6.9 Hz), 2.82 (2H, q, J=7.6 Hz), 2.65 (3H, s), 2.52 (3H, s), 2.35 (2H, q, J=7.6 Hz), 1.27 (3H, t, J=7.6 Hz), 1.14 (3H, t, J=7.6 Hz).

### STEP 6. 2-[4-(2-Ethyl-5,7-dimethyl-3H-imidazo[4,5-b]pyridin-3-yl)phenyl]ethanol

To a solution of 2-[4-(2-ethyl-5,7-dimethyl-3H-imidazo[4,5-b]pyridin-3-yl)phenyl]ethyl propionate (step 5, 1.75 g, 5.1 mmol) in methanol/THF (v/v, 1:1, 28 mL) was added 4N aqueous LiOH (4.6 mL, 18.4 mmol) and the resulting mixture was stirred at room temperature. After 3 h, the mixture was concentrated. The residue was dissolved in water (30 mL) and extracted with ethyl acetate (100 mL). The organic layer was washed with brine (50 mL), dried (MgSO₄), and concentrated. Purification by flash column chromatography on silica gel eluting with hexane/ethyl acetate (gradient elution from 2:1 to 0:1) to afford 1.3 g (86%) of the title compound as pale brown solids: ¹H-NMR (CDCl₃) δ 7.40 (2H, d, J=8.4 Hz), 7.31 (2H, d, J=8.4 Hz), 6.91 (1H, s), 3.81-3.75 (2H, m), 3.47 (1H, br.s), 2.92 (2H, t, J=6.9 Hz), 2.81 (2H, q, J=7.6 Hz), 2.66 (3H, s), 2.51 (3H, s), 1.27 (3H, t, J=7.6 Hz).

### STEP 7. 3-[4-(2-Chloroethyl)phenyl]-2-ethyl-5,7-dimethyl-3H-imidazo[4,5-b]pyridine

To a solution of 2-[4-(2-ethyl-5,7-dimethyl-3H-imidazo[4,5-b]pyridin-3-yl)phenyl]ethanol (step 6, 2.2 g, 7.4 mmol) in toluene (40 mL) was added thionyl chloride (2.0 mL, 23.6 mmol), and the resulting mixture was stirred at 80° C. for 3 h. The volatile components were removed under reduced pressure, and the residue was purified by flash column chromatography on silica gel eluting with hexane/ethyl acetate (gradient elution from 2:1 to 1:1) to afford 2.1 g (90%) of the title compound as white solids: ¹H-NMR (CDCl₃) δ 7.41 (2H, d, J=8.4 Hz), 7.35 (2H, d, J=8.4 Hz), 6.90 (1H, s), 3.78 (2H, t, J=7.4 Hz), 3.15 (2H, t, J=7.4 Hz), 2.83 (2H, q, J=7.6 Hz), 2.71 (3H, s), 2.54 (3H, s), 1.28 (3H, t, J=7.6 Hz).

### STEP 8. 2-[4-(2-Ethyl-5,7-dimethyl-3H-imidazo[4,5-b]pyridin-3-yl)phenyl]ethyl azide

To a stirred solution of 3-[4-(2-chloroethyl)phenyl]-2-ethyl-5,7-dimethyl-3H-imidazo[4,5-b]pyridine (step 7, 2.8 g, 9.0 mmol) and KI (1.5 g, 9.0 mmol) in DMF (50 mL) was added sodium azide (1.2 g, 18.0 mmol), and then the resulting mixture was stirred overnight at 100° C. The reaction mixture was poured into water (100 mL), and extracted with ethyl acetate (100 mL). The organic layer was washed with water (50 mL) and brine (50 mL), then dried (Na₂SO₄). After removal of solvent, the crude product was purified by flash column chromatography on silica gel eluting with hexane/ethyl acetate (1:1) to afford 2.35 g (85%) of the title compound as white solids: ¹H-NMR (CDCl₃) δ 7.41 (2H, d, J=8.4 Hz), 7.35 (2H, d, J=8.4 Hz), 6.90 (1H, s), 3.59 (2H, t, J=7.1 Hz), 2.99 (2H, t, J=7.1 Hz), 2.83 (2H, q, J=7.6 Hz), 2.65 (3H, s), 2.52 (3H, s), 1.27 (3H, t, J=7.6 Hz).

### STEP 9. 2-[4-(2-Ethyl-5,7-dimethyl-3H-imidazo[4,5-b]pyridin-3-yl)phenyl]ethylamine

To a solution of 2-[4-(2-ethyl-5,7-dimethyl-3H-imidazo[4,5-b]pyridin-3-yl)phenyl]ethyl azide (step 8, 2.35 g, 7.3 mmol) in methanol (50 mL) was added 10% Pd—C (200 mg). The resulting mixture was stirred for 4 h under hydrogen atmosphere. The mixture was filtered through a pad of Celite and the filtrate was concentrated. The residue was purified by flash column chromatography on silica gel eluting with dichloromethane/methanol/triethylamine (100:5:1) to afford 2.01 g (94%) of the title compound as white solids: ¹H-NMR (CDCl₃) δ 7.39 (2H, d, J=8.4 Hz), 7.32 (2H, d, J=8.4 Hz), 6.90 (1H, s), 3.05 (2H, t, J=7.3 Hz), 2.88-2.78 (4H, m), 2.65 (3H, s), 2.51 (3H, s), 1.28 (3H, t, J=7.6 Hz).

### STEP 10. 2-Ethyl-5,7-dimethyl-3-(4-{2-[({[(4-methylphenyl)sulfonyl]amino}carbonyl)amino]ethyl}phenyl)-3H-imidazo[4,5-b]pyridine

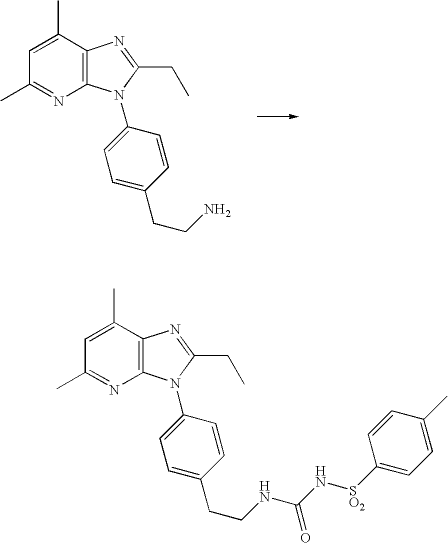

To a solution of 2-[4-(2-ethyl-5,7-dimethyl-3H-imidazo[4,5-b]pyridin-3-yl)phenyl]ethylamine (step 9, 1.2 g, 4.0 mmol) in dichloromethane (15 mL) was added p-toluenesulfonyl isocyanate (805 mg, 4.0 mmol). The resulting mixture was stirred at room temperature for 3 h. After removal of solvent, the residue was purified by flash column chromatography on silica gel eluting with dichloromethane/methanol (20:1) to afford 1.10 g (56%) of the title compound as white solids: ¹H-NMR (CDCl₃) δ 7.85 (2H, d, J=8.2 Hz), 7.32 (2H, d, J=8.2 Hz), 7.23 (2H, d, J=8.4 Hz), 7.16 (2H, d, J=8.4 Hz), 6.91 (1H, s), 6.12 (1H, br.s), 3.55-3.46 (2H, m), 2.85 (2H, t, J=6.3 Hz), 2.74-2.64 (5H, m), 2.42 (3H, s), 2.41 (3H, s), 1.21 (3H, t, J=7.6 Hz).

### Example 2

### Eg 42, CJ-23,423

### 2-ETHYL-4,6-DIMETHYL-1-(4-{2-[({[(4-METHYLPHENYL)SULFONYL]AMINO}CARBONYL)AMINO]ETHYL}PHENYL)-1H-IMIDAZO[4,5-c]PYRIDINE

### STEP 1. 2-{4-[(2,6-Dimethyl-3-nitro-4-pyridinyl)amino]phenyl}ethanol

The title compound was prepared according to the procedure described in step 3 of Example 1 from 4-chloro-2,6-dimethyl-3-nitropyridine (Tanaka, A.; et al. J. Med. Chem., 1999, 41, 4408) and 4-aminophenylethyl alcohol.

¹H-NMR (CDCl₃) δ 8.74 (1H, br.s), 7.31 (2H, d, J=8.2 Hz), 7.18 (2H, d, J=8.2 Hz), 6.68 (1H, s), 3.95-3.89 (2H, m), 2.91 (2H, t, J=6.6 Hz), 2.72 (3H, s), 2.36 (3H, s).

### STEP 2. 2-{4-[(3-Amino-2,6-dimethyl-4-pyridinyl)amino]phenyl}ethanol

The title compound was prepared according to the procedure described in step 4 of Example 1 from 2-{4-[(2,6-dimethyl-3-nitro-4-pyridinyl)amino]phenyl}ethanol (step 1).

¹H-NMR (CDCl₃) δ 7.19 (2H, d, J=8.4 Hz), 7.01 (2H, d, J=8.6 Hz), 6.76 (1H, s), 5.82 (1H, br.s), 3.87 (2H, t, J=6.4 Hz), 3.18 (2H, br.s), 2.85 (2H, t, J=6.4 Hz), 2.44 (3H, s), 2.35 (3H, s).

### STEP 3. 2-[4-(2-Ethyl-4,6-dimethyl-1H-imidazo[4,5-c]pyridin-1-yl)phenyl]ethyl propionate

A mixture of 2-{4-[(3-amino-2,6-dimethyl-4-pyridinyl)amino]phenyl}ethanol (step 2, 2.4 g, 9.3 mmol), propionic anhydride (13 mL, 101 mmol) and propionic acid (13 mL, 174 mmol) was stirred at 120° C. for 16 h. After cooling, the mixture was diluted with 2N aqueous NaOH (150 mL) and extracted with dichloromethane (3×150 mL). The combined organic extracts were washed with brine (50 mL), dried (MgSO₄), and concentrated. Purification by flash column chromatography on silica gel eluting with dichloromethane/methanol (gradient elution from 20:1 to 10:1) afforded 2.3 g (69%) of the title compound as a brown oil: ¹H-NMR (CDCl₃) δ 7.44 (2H, d, J=8.1 Hz), 7.27 (2H, d, J=8.2 Hz), 6.72 (1H, s), 4.38 (2H, t, J=6.9 Hz), 3.07 (2H, t, J=7.1 Hz), 2.88 (3H, s), 2.82 (2H, q, J=7.6 Hz), 2.56 (3H, s), 2.36 (2H, q, J=7.6 Hz), 1.29 (3H, t, J=7.6 Hz), 1.15 (3H, t, J=7.7 Hz).

### STEP 4. 2-[4-(2-Ethyl-4,6-dimethyl-1H-imidazo[4,5-c]pyridin-1-yl)phenyl]ethanol

The title compound was prepared according to the procedure described in step 6 of Example 1 from 2-[4-(2-ethyl-4,6-dimethyl-1H-imidazo[4,5-c]pyridin-1-yl)phenyl]ethyl propionate (step 3).

¹H-NMR (CDCl₃) δ 7.46 (2H, d, J=8.1 Hz), 7.26 (2H, d, J=8.1 Hz), 6.73 (1H, s), 4.00 (2H, t, J=6.6 Hz), 3.01 (2H, t, J=6.4 Hz), 2.88 (3H, s), 2.81 (2H, q, J=7.5 Hz), 2.54 (3H, s), 1.29 (3H, t, J=7.5 Hz).

### STEP 5. 1-[4-(2-Chloroethyl)phenyl]-2-ethyl-4,6-dimethyl-1H-imidazo[4,5-c]pyridine

The title compound was prepared according to the procedure described in step 7 of Example 1 from 2-[4-(2-ethyl-4,6-dimethyl-1H-imidazo[4,5-c]pyridin-1-yl)phenyl]ethanol (step 4).

TLC Rf=0.1 (ethyl acetate).

### STEP 6. 1-[4-(2-Azidoethyl)phenyl]-2-ethyl-4,6-dimethyl-1H-imidazo[4,5-c]pyridine

The title compound was prepared according to the procedure described in step 8 of Example 1 from 1-[4-(2-chloroethyl)phenyl]-2-ethyl-4,6-dimethyl-1H-imidazo[4,5-c]pyridine (step 5).

¹H-NMR (CDCl₃) δ 7.46 (2H, d, J=8.0 Hz), 7.29 (2H, d, J=7.7 Hz), 6.72 (1H, s), 3.62 (2H, t, J=6.9 Hz), 3.02 (2H, t, J=6.9 Hz), 2.88 (3H, s), 2.81 (2H, q, J=7.4 Hz), 2.56 (3H, s), 1.29 (3H, t, J=7.6 Hz).

### STEP 7. 2-[4-(2-Ethyl-4,6-dimethyl-1H-imidazo[4,5-c]pyridin-1-yl)phenyl]ethylamine

The title compound was prepared according to the procedure described in step 9 of Example 1 from 1-[4-(2-azidoethyl)phenyl]-2-ethyl-4,6-dimethyl-1H-imidazo[4,5-c]pyridine (step 6).

¹H-NMR (CDCl₃) δ 7.42 (2H, d, J=8.2 Hz), 7.26 (2H, d, J=8.4 Hz), 6.73 (1H, s), 3.08 (2H, t, J=6.9 Hz), 2.90-2.78 (4H, m), 2.88 (3H, s), 2.56 (3H, s), 1.30 (3H, t, J=7.3 Hz).

### STEP 8. 2-Ethyl-4,6-dimethyl-1-(4-{2-[({[(4-methylphenyl)sulfonyl]amino}carbonyl)amino]ethyl}phenyl)-1H-imidazo[4,5-c]pyridine

The title compound was prepared according to the procedure described in step 10 of Example 1 from 2-[4-(2-ethyl-4,6-dimethyl-1H-imidazo[4,5-c]pyridin-1-yl)phenyl]ethylamine (step 7).

mp 143° C.; MS (ESI) m/z 492.12 (M+H)⁺; ¹H-NMR (CDCl₃) δ 7.77 (2H, d, J=8.3 Hz), 7.38 (2H, d, J=8.4 Hz), 7.25 (2H, d, J=8.4 Hz), 7.20 (2H, d, J=8.4 Hz), 6.77 (1H, s), 3.58-3.51 (2H, m), 2.92 (2H, t, J=7.0 Hz), 2.89 (3H, s), 2.79 (2H, q, J=7.5 Hz), 2.53 (3H, s), 2.38 (3H, s), 1.28 (3H, t, J=7.5 Hz).

The following examples illustrate the preparation of the EP4-receptor antagonists disclosed in U.S. Application Number US60/500,131:

### Example 1

### 4-[({[5-FLUORO-2-(4-FLUOROPHENOXY)PYRIDIN-3-YL]CARBONYL}AMINO)METHYL]BENZOIC ACID

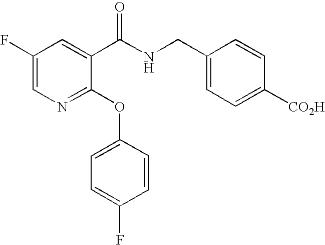

### STEP 1. Methyl 5-fluoro-2-(4-fluorophenoxy)nicotinate

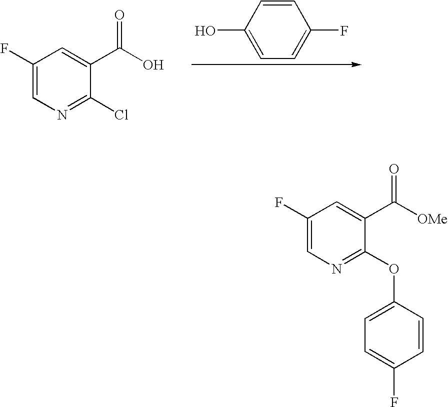

A mixture of 2-chloro-5-fluoronicotinic acid (2.61 g, 14.9 mmol), 4-fluorophenol (2.02 g, 18 mmol), potassium carbonate (4.56 g, 33 mmol), copper bronze (211 mg, 3.3 mmol), and cuprous iodide (230 mg, 1.2 mmol) in N,N-dimethylforamide (40 mL) was heated under reflux in an oil bath for 6 h. The reaction mixture was filtered through a pad of celite. The filtrate was partitioned between ethyl acetate (200 mL) and 2 N hydrochloric acid (200 mL). The organic phase was separated and the aqueous phase was extracted with ethyl acetate (200 mL). The combined organic extracts were washed with brine (50 mL) and dried (sodium sulfate). After removal of solvent, the residual oil was dissolved in methanol (50 mL). To the solution were added conc. hydrochloric acid (1 mL) and the mixture was heated under reflux for 4 h. The volatile components were removed under reduced pressure, and the residue was purified by flash column chromatography on silica gel (150 g) eluting with hexane/ethyl acetate (3/1) to afford 2.63 g (67%) of the title compound: ¹H-NMR (CDCl₃) δ 8.11 (1H, d, J=3.1 Hz), 8.02 (1H, dd, J=7.7, 3.1 Hz), 7.11-7.07 (4H, m), 3.96 (3H, s); MS (ESI) m/z 266 (M+H)⁺.

### STEP 2. 5-Fluoro-2-(4-fluorophenoxy)nicotinic acid

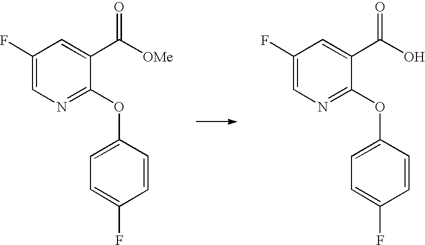

To a stirred solution of methyl 5-fluoro-2-(4-fluorophenoxy)nicotinate (step 1, 2.63 g, 9.9 mmol) in methanol (50 ml) was added 2 N sodium hydroxide aqueous solution (10 ml). The reaction mixture was stirred at 40° C. for 3 h. After cooling, the pH value was adjusted to 4.0 by the addition of 2 N hydrochloric acid. The mixture was diluted with water (100 ml), and extracted with dichloromethane (100 ml×3). The combined organic layer was washed with brine (100 ml), dried (sodium sulfate), and concentrated to afford 2.26 g (91%) of the title compound as off white solids: ¹H-NMR (CDCl₃) [symbol] 8.25 (1H, dd, J=7.5, 3.1 Hz), 8.16 (1H, d, J=3.1 Hz), 7.16-7.13 (4H, m); MS (ESI) m/z 252 (M+H)⁺.

### STEP 3. Methyl 4-[({[5-fluoro-2-(4-fluorophenoxy)pyridin-3-yl]carbonyl}amino)methyl]benzoate

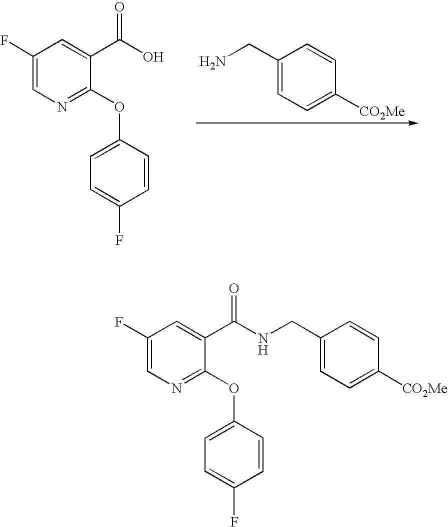

To a stirred solution of 5-fluoro-2-(4-fluorophenoxy)nicotinic acid (step 2, 300 mg, 1.2 mmol) and methyl 4-(aminomethyl)benzoate hydrochloride (284 mg, 1.4 mmol) in dichloromethane (10 mL) were successively added 1-(3-dimethylaminopropyl)-3-ethylcarbodiimide hydrochloride (EDCl) (670 mg, 3.5 mmol), 1-hydroxybenzotriazole hydrate (HOBT) (368 mg, 2.4 mmol), and triethylamine (3 mL). After being stirred overnight, the reaction was quenched by the addition of saturated sodium bicarbonate aqueous solution (50 mL). The aqueous layer was extracted with dichloromethane (50 mL×2) and the combined organic layers were washed with brine (50 mL), dried (sodium sulfate), and evaporated. The remaining residue was purified by flash column chromatography on silica gel (50 g) eluting with hexane/ethyl acetate (3/1) to afford 407 mg (85%) of the title compounds as white solids: ¹H-NMR (CDCl₃) [symbol] 8.39 (1H, dd, J=8.3, 3.1 Hz), 8.28 (1H, br.s), 8.05 (1H, d, J=3.1 Hz), 8.01 (2H, d, J=8.1 Hz), 7.42 (2H, d, J=8.1 Hz), 7.17-7.05 (4H, m), 4.76 (2H, d, J=5.9 Hz), 3.91 (3H, s); MS (ESI) m/z 399 (M+H)⁺, 397 (M−H)⁻.

### STEP 4. 4-[({[5-Fluoro-2-(4-fluorophenoxy)pyridin-3-yl]carbonyl}amino)methyl]benzoic acid

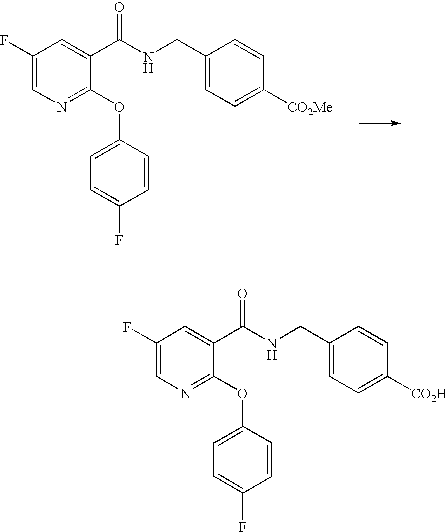

To a stirred solution of methyl 4-[({[5-fluoro-2-(4-fluorophenoxy)pyridin-3-yl]carbonyl}amino)methyl]benzoate (step 3, 407 mg, 1.02 mmol) in methanol (10 ml) was added 2 N sodium hydroxide aqueous solution (2 ml). The reaction mixture was stirred at room temperature for 3 h and then evaporated. The residue was partitioned between ethyl acetate (100 mL) and 2 N hydrochloric acid (100 mL). The organic phase was separated and the aqueous phase was extracted with ethyl acetate (100 mL). The combined organic extracts were washed with brine (50 mL), dried (sodium sulfate), and concentrated. The residual solids were recrystallized from ethyl acetate to afford 248 mg (64%) of the title compound as white solids: ¹H-NMR (CDCl₃) [symbol] 8.40 (1H, dd, J=8.3, 3.1 Hz), 8.30 (1H, br.s), 8.09-8.04 (3H, m), 7.45 (2H, d, J=8.1 Hz), 7.17-7.06 (4H, m), 4.79 (2H, d, J=5.9 Hz); MS (EI) m/z 384 (M⁺), (ESI) m/z 385 (M+H)⁺, 383 (M−H)⁻.

### Example 2

### 4-[1-({[5-FLUORO-2-(4-FLUOROPHENOXY)PYRIDIN-3-YL]CARBONYL}AMINO)ETHYL]BENZOIC ACID

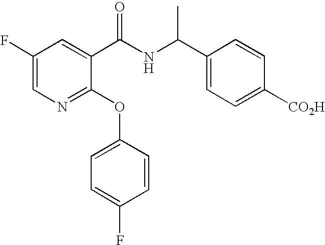

### STEP 1. N-[1-(4-Bromophenyl)ethyl]-5-fluoro-2-(4-fluorophenoxy)nicotinamide

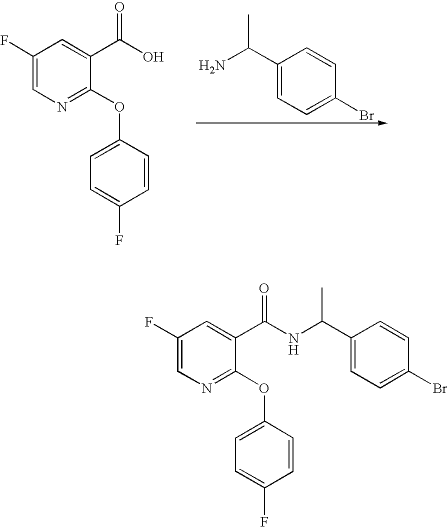

The title compound was prepared according to the procedure described in step 3 of Example 1 from 5-fluoro-2-(4-fluorophenoxy)nicotinic acid (step 2 of Example 1) and [1-(4-bromophenyl)ethyl]amine: ¹H-NMR (CDCl₃) [symbol] 8.31 (1H, ddd, J=8.2, 3.1, 0.9 Hz), 8.14 (1H, d, J=7.2 Hz), 8.03 (1H, dd, J=3.1, 1.1 Hz), 7.45 (2H, dd, J=7.0, 0.9 Hz), 7.25-7.09 (6H, m), 5.28 (1H, dq, J=7.2, 7.0 Hz), 1.57 (3H, d, J=7.0 Hz); MS (ESI) m/z 433 (M+H)⁺, 431 (M−H)⁻.

### STEP 2. Methyl 4-[1-({[5-fluoro-2-(4-fluorophenoxy)pyridin-3-yl]carbonyl}amino)ethyl]benzoate

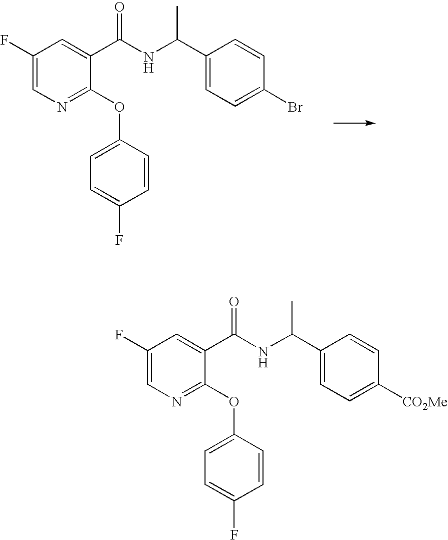

A mixture of N-[1-(4-bromophenyl)ethyl]-5-fluoro-2-(4-fluorophenoxy)nicotinamide (step 1, 398 mg, 0.92 mmol), 1,3-bis(diphenylphosphino)-propane (38 mg, 0.09 mmol), palladium (II) acetate (21 mg, 0.09 mmol), triethylamine (0.38 mL, 2.76 mmol), N,N-dimethylforamide (6 mL) and methanol (4 mL) was stirred at 80° C. for 16 h under carbon monoxide atmosphere. After cooling to room temperature, the mixture was diluted with ether (100 mL) and washed with water (60 mL×3). The organic layer was dried over magnesium sulfate and evaporated. The residue was purified by flash column chromatography on silica gel eluting with hexane/ethyl acetate (2:1) to afford 296 mg (78%) of the title compounds as white solids: ¹H-NMR (CDCl₃) [symbol] 8.32 (1H, dd, J=8.1, 3.1 Hz), 8.21 (1H, d, J=7.3 Hz), 8.04-7.99 (3H, m), 7.43 (2H, d, J=8.2 Hz), 7.27-7.13 (4H, m), 5.38 (1H, dq, J=7.3, 6.9 Hz), 3.90 (3H, s), 1.60 (3H, d, J=6.9 Hz); MS (ESI) m/z 413 (M+H)⁺, 411 (M−H)⁻.

### STEP 3. 4-[1-({[5-Fluoro-2-(4-fluorophenoxy)pyridin-3-yl]carbonyl}amino)ethyl]benzoic acid

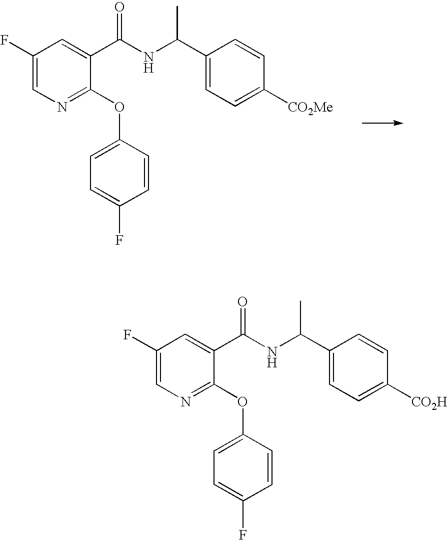

The title compound was prepared according to the procedure described in step 4 of Example 1 from methyl 4-[1-({[5-fluoro-2-(4-fluorophenoxy)pyridin-3-yl]carbonyl}amino)ethyl]benzoate (step 2): ¹H-NMR (DMSO-d₆) [symbol] 9.01 (1H, d, J=7.9 Hz), 8.23 (1H, dd, J=3.1, 1.3 Hz), 8.02 (1H, ddd, J=7.9, 3.1, 1.3 Hz), 7.86 (2H, d, J=8.3 Hz), 7.52 (2H, d, J=7.5 Hz), 7.30-7.24 (4H, m), 5.18 (1H, dq, J=7.9, 7.0 Hz), 1.46 (3H, d, J=7.0 Hz); MS (ESI) m/z 399 (M+H)⁺, 397 (M−H)⁻.

### Example 3

### 4-[1-({[5-FLUORO-2-(4-FLUOROPHENOXY)PYRIDIN-3-YL]CARBONYL}AMINO)PROPYL]BENZOIC ACID

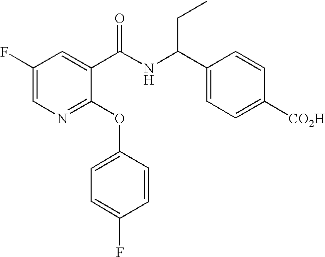

### STEP 1. Methyl 4-[1-({[5-fluoro-2-(4-fluorophenoxy)pyridin-3-yl]carbonyl}amino)propyl]benzoate

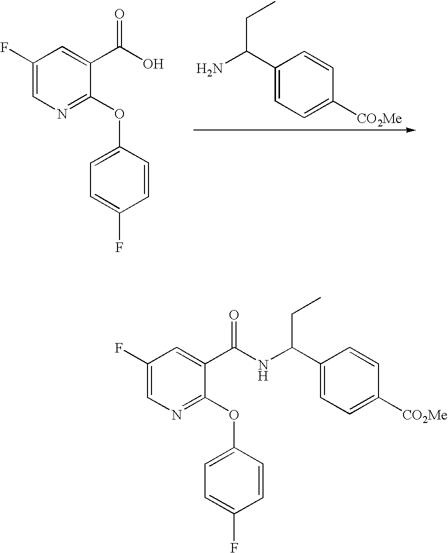

The title compound was prepared according to the procedure described in step 3 of Example 1 from 5-fluoro-2-(4-fluorophenoxy)nicotinic acid (step 3 of Example 1) and methyl 4-(1-aminopropyl)benzoate: ¹H-NMR (CDCl₃) δ 8.33-8.26 (2H, m), 8.05-7.99 (3H, m), 7.39 (2H, d, J=8.4 Hz), 7.20-7.15 (4H, m), 5.15 (1H, q, J=7.3 Hz), 3.90 (3H, s), 1.92 (2H, dq, J=7.3, 7.3 Hz), 0.95 (3H, t, J=7.3 Hz); MS (ESI) m/z 427 (M+H)⁺, 425 (M−H)⁻.

### STEP 2. 4-[1-({[5-Fluoro-2-(4-fluorophenoxy)pyridin-3-yl]carbonyl}amino)propyl]benzoic acid

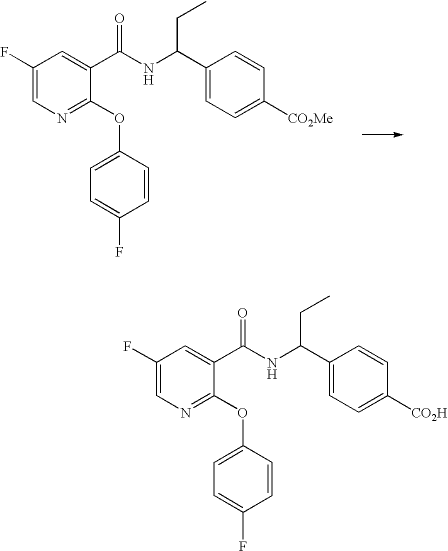

The title compound was prepared according to the procedure described in step 4 of Example 1 from methyl 4-[1-({[5-fluoro-2-(4-fluorophenoxy)pyridin-3-yl]carbonyl}amino)propyl]benzoate (step 1): ¹H-NMR (DMSO-d₆) δ 8.97 (1H, d, J=8.0 Hz), 8.23 (1H, d, J=2.9 Hz), 7.99 (1H, dd, J=7.9, 3.1 Hz), 7.86 (2H, d, J=8.3 Hz), 7.50 (2H, d, J=8.3 Hz), 7.30-7.21 (4H, m), 4.96 (1H, q, J=7.7 Hz), 1.77 (2H, dq, J=7.7, 7.2 Hz), 0.92 (3H, t, J=7.2 Hz); MS (ESI) m/z 413 (M+H)⁺, 411 (M−H)⁻.

### Example 4

### 4-[1-({[5-FLUORO-2-(4-FLUOROPHENOXY)PYRIDIN-3-YL]CARBONYL}AMINO)-1-METHYLETHYL]BENZOIC ACID

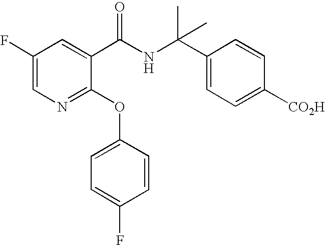

### STEP 1. Methyl 4-[1-({[5-Fluoro-2-(4-fluorophenoxy)pyridin-3-yl]carbonyl}amino)-1-methylethyl]benzoate

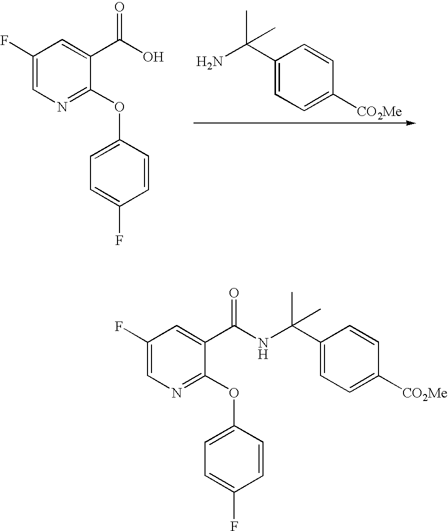

The title compound was prepared according to the procedure described in step 3 of Example 1 from 5-fluoro-2-(4-fluorophenoxy)nicotinic acid (step 3 of Example 1) and methyl 4-(1-amino-1-methylethyl)benzoate (EP 29320): ¹H-NMR (CDCl₃) [symbol] 8.33 (1H, br.s), 8.24 (1H, dd, J=8.2, 3.1 Hz), 8.04-7.99 (3H, m), 7.51 (2H, dd, J=6.7, 1.9 Hz), 7.18-7.16 (4H, m), 3.90 (3H, s), 1.80 (6H, s); MS (ESI) m/z 427 (M+H)⁺, 425 (M−H)⁻.

### STEP 2. 4-[1-({[5-Fluoro-2-(4-fluorophenoxy)pyridin-3-yl]carbonyl}amino)-1-methylethyl]benzoic acid

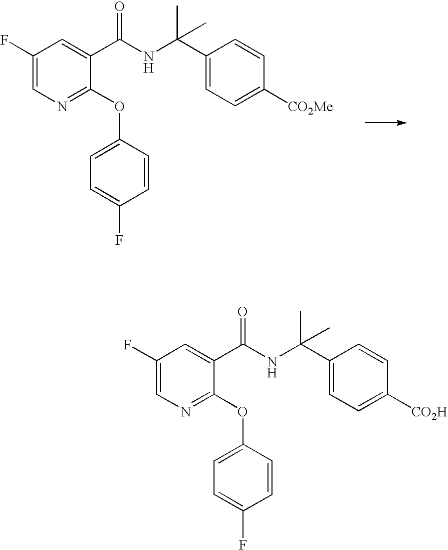

The title compound was prepared according to the procedure described in step 4 of Example 1 from methyl 4-[1-({[5-Fluoro-2-(4-fluorophenoxy)pyridin-3-yl]carbonyl}amino)-1-methylethyl]benzoate (step 1): ¹H-NMR (DMSO-d₆) [symbol] 8.82 (1H, s), 8.23 (1H, d, J=3.1 Hz), 8.00 (1H, dd, J=7.9, 3.1 Hz), 7.83 (2H, d, J=8.4 Hz), 7.57 (2H, d, J=8.4 Hz), 7.29-7.27 (4H, m), 1.65 (6H, s); MS (ESI) m/z 413 (M+H)⁺, 411 (M−H)⁻.

### Example 5

### 4-[(1S)-1-({[5-FLUORO-2-(4-FLUOROPHENOXY)PYRIDIN-3-YL]CARBONYL}AMINO)ETHYL]BENZOIC ACID

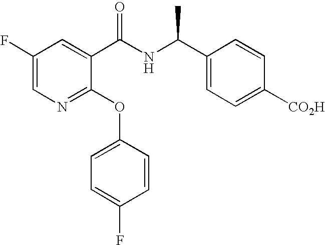

### STEP 1. tert-Butyl [(1S)-1-(4-bromophenyl)ethyl]carbamate

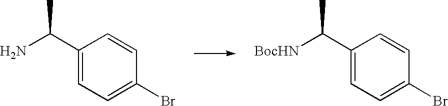

A mixture of [(1S)-1-(4-bromophenyl)ethyl]amine (10.00 g, 50.0 mmol) and di-tert-butyl dicarbonate (11.45 g, 52.5 mmol), triethylamine (7.66 mL, 55.0 mmol) in dichloromethane (200 mL) was stirred at room temperature for 1 h. The mixture was diluted with dichloromethane (500 mL) and washed with 1 M hydrochloric acid (300 mL), saturated sodium hydrogen carbonate aqueous (300 mL), and brine (300 mL). The organic layer was dried over magnesium sulfate, and concentrated under reduced pressure. The residue was washed with cold hexane to afford 14.73 g (98%) of the title compounds as white solids: ¹H-NMR (CDCl₃) [symbol] 7.47-7.42 (2H, m), 7.18 (2H, d, J=8.4 Hz), 5.30 (2H, br.s), 1.41 (12H, br.s{tilde over ())}.

### STEP 2. Methyl 4-{(1S)-1-[(tert-butoxycarbonyl)amino]ethyl}benzoate

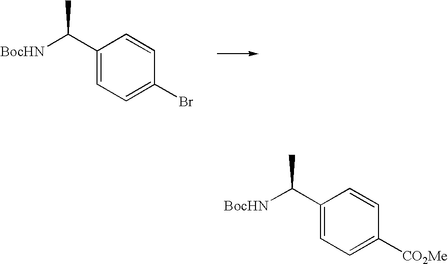

A mixture of tert-butyl [(1S)-1-(4-bromophenyl)ethyl]carbamate (step 1, 14.73 g, 49.1 mmol), 1,3-bis(diphenylphosphino)-propane (2.03 g, 4.91 mmol), palladium (II) acetate (1.10 g, 4.91 mmol), triethylamine (20.5 mL, 147 mmol), N,N-dimethylforamide (120 mL) and methanol (180 mL) was stirred at 80° C. for 16 h under carbon monoxide atmosphere. After cooling to room temperature, the mixture was diluted with ether (800 mL) and washed with water (500 mL×3). The organic layer was dried over magnesium sulfate and evaporated. The residue was purified by flash column chromatography on silica gel eluting with hexane/ethyl acetate (5:1) to afford 12.83 g (94%) of the title compounds as white solids: ¹H-NMR (CDCl₃) [symbol] 8.02-7.99 (2H, m), 7.37 (2H, d, J=8.4 Hz), 4.83 (2H, br.s), 3.91 (3H, s), 1.46-1.42 (12H, m{tilde over ())}.

### STEP 3. Methyl 4-[(1S)-1-aminoethyl]benzoate hydrochloride

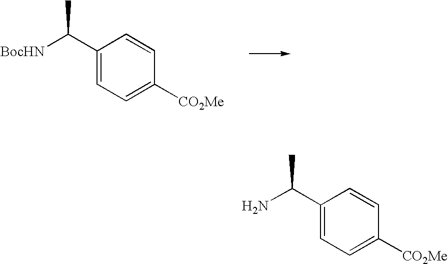

Methyl 4-{(1S)-1-[(tert-butoxycarbonyl)amino]ethyl}benzoate (12.83 g, 45.9 mmol) was treated with trifluoroacetic acid (100 mL) and dichloromethane (100 mL) at room temperature for 16 h. After removal of the solvent, the residue was diluted with 10% hydrogen chloride solution in methanol (100 mL). The mixture was concentrated under reduced pressure and the residue was washed with ethylacetate to give 9.40 g (95%) of the title compounds as white solids: ¹H-NMR (DMSO-d₆) [symbol] 8.67 (2H, br.s), 8.01 (2H, d, J=8.4 Hz), 7.68 (2H, d, J=8.4 Hz), 4.49 (1H, q, J=6.9 Hz), 3.87 (3H, s), 1.53 (3H, d, J=6.9 Hz{tilde over ())}.

### STEP 4. Methyl 4-[(1S)-1-({[5-fluoro-2-(4-fluorophenoxy)pyridin-3-yl]carbonyl}amino)ethyl]benzoate

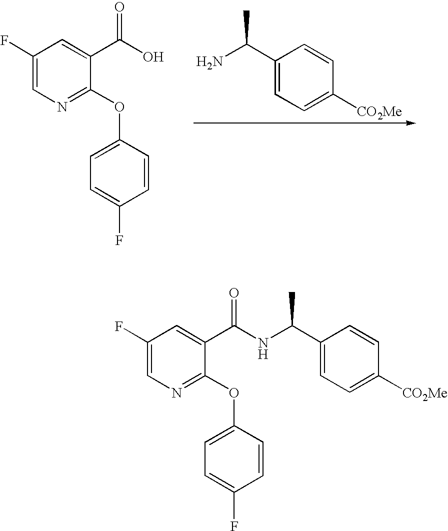

The title compound was prepared according to the procedure described in step 3 of Example 1 from 5-fluoro-2-(4-fluorophenoxy)nicotinic acid (step 3 of Example 1) and methyl 4-[(1S)-1-aminoethyl]benzoate hydrochloride (step 3): ¹H-NMR (CDCl₃) the data of the title compound were identical with that of the racemate (step 2 of Example 2); MS (ESI) m/z 413 (M+H)⁺, 411 (M−H)⁻.

### STEP 5. 4-[(1S)-1-({[5-Fluoro-2-(4-fluorophenoxy)pyridin-3-yl]carbonyl}amino)ethyl]benzoic acid

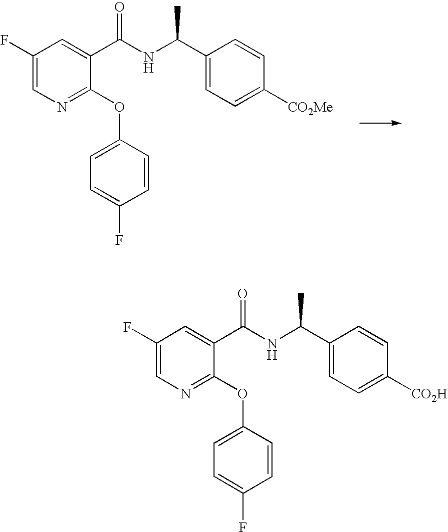

The title compound was prepared according to the procedure described in step 4 of Example 1 from methyl 4-[(1S)-1-({[5-fluoro-2-(4-fluorophenoxy)pyridin-3-yl]carbonyl}amino)ethyl]benzoate (step 4): ¹H-NMR (DMSO-d₆) the data of the title compound were identical with that of the racemate (step 3 of Example 2); MS (ESI) m/z 399 (M+H)⁺, 397 (M−H)⁻.

### Example 6

### 4-[(1S)-1-({[5-FLUORO-2-(3-FLUOROPHENOXY)PYRIDIN-3-YL]CARBONYL}AMINO)ETHYL]BENZOIC ACID

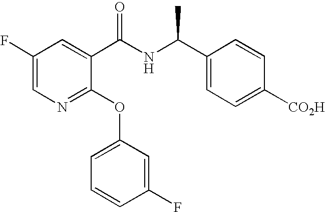

### STEP 1. 5-Fluoro-2-(3-fluorophenoxy)nicotinic acid

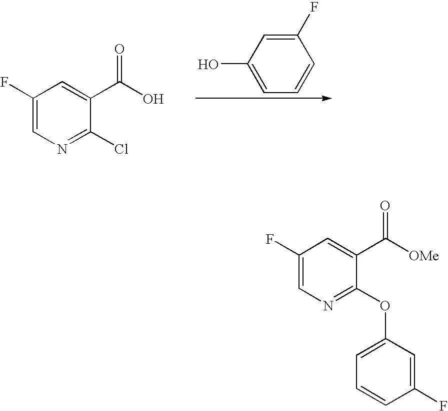

The title compound was prepared according to the procedure described in step 1 & 2 of Example 1 from 2-hydroxy-5-fluoronicotinic acid and 3-fluorophenol; ¹H-NMR (DMSO-d₆) δ 8.37 (1H, m), 8.23-8.15 (1H, m), 7.49-7.35 (1H, m), 7.10-6.90 (3H, m).

### STEP 2. Methyl 4-[(1S)-1-({[5-fluoro-2-(3-fluorophenoxy)pyridin-3-yl]carbonyl}amino)ethyl]benzoate

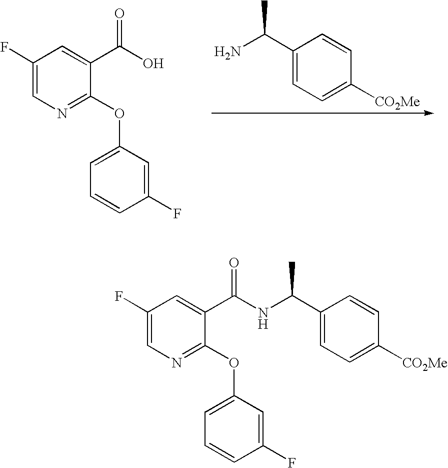

The title compound was prepared according to the procedure described in step 3 of Example 1 from 5-fluoro-2-(3-fluorophenoxy)nicotinic acid (step 1) and methyl 4-[(1S)-1-aminoethyl]benzoate hydrochloride (step 3 of Example 5): ¹H-NMR (CDCl₃) [symbol] 8.33 (1H, dd, J=8.2, 3.1 Hz), 8.12-7.98 (4H, m), 7.47-7.38 (3H, m), 7.05-6.89 (3H, m), 5.36 (1H, m), 3.90 (3H, s), 1.60 (3H, d, J=6.9 Hz{tilde over ())}.

### STEP 3. 4-[(1S)-1-({[5-Fluoro-2-(3-fluorophenoxy)pyridin-3-yl]carbonyl}amino)ethyl]benzoic acid

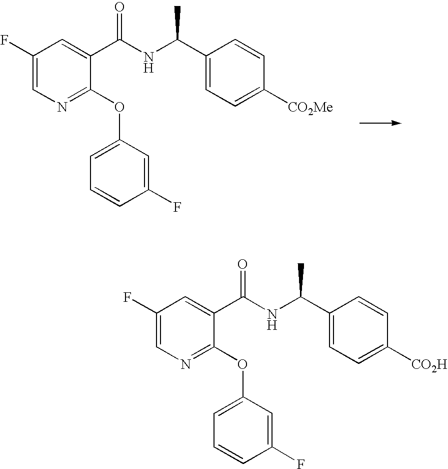

The title compound was prepared according to the procedure described in step 4 of Example 1 from methyl 4-[(1S)-1-({[5-fluoro-2-(3-fluorophenoxy)pyridin-3-yl]carbonyl}amino)ethyl]benzoate (step 2): ¹H-NMR (CDCl₃) [symbol] 8.34 (1H, dd, J=8.2, 3.1 Hz), 8.14-8.02 (4H, m), 7.47-7.38 (3H, m), 7.27-6.89 (3H, m), 5.36 (1H, m), 1.59 (3H, d, J=6.9 Hz); MS (ESI) m/z 399 (M+H)⁺, 397 (M−H)⁻.

### Example 7

### 4-[({([5-FLUORO-2-(4-FLUOROPHENOXY)PYRIDIN-3-YL]CARBONYL}AMINO)METHYL]-3-METHYLBENZOIC ACID

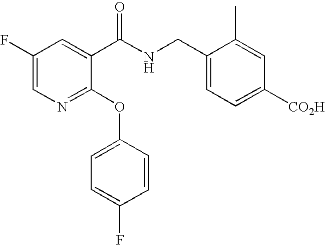

### STEP 1. Methyl 4-[({[5-fluoro-2-(4-fluorophenoxy)pyridin-3-yl]carbonyl}amino)methyl]-3-methylbenzoate

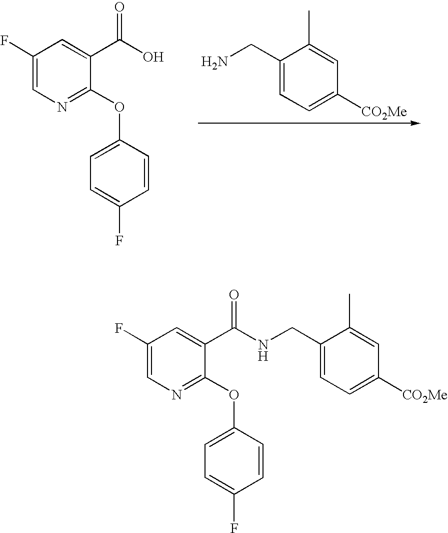

The title compound was prepared according to the procedure described in step 3 of Example 1 from 5-fluoro-2-(4-fluorophenoxy)nicotinic acid (step 3 of Example 1) and methyl 4-(aminomethyl)-3-methylbenzoate (WO 0324955): ¹H-NMR (CDCl₃) δ 8.36 (1H, dd, J=8.2, 3.1 Hz), 8.25-8.18 (1H, m), 8.04 (1H, d, J=3.1 Hz), 7.85-7.81 (2H, m), 7.37 (1H, d, J=7.7 Hz), 7.15-7.07 (4H, m), 4.73 (2H, d, J=5.8 Hz), 3.89 (3H, s), 2.40 (3H, s); MS (ESI) m/z 413 (M+H)⁺, 411 (M−H)⁻.

### STEP 2. 4-[({[5-Fluoro-2-(4-fluorophenoxy)pyridin-3-yl]carbonyl}amino)methyl]-3-methylbenzoic acid

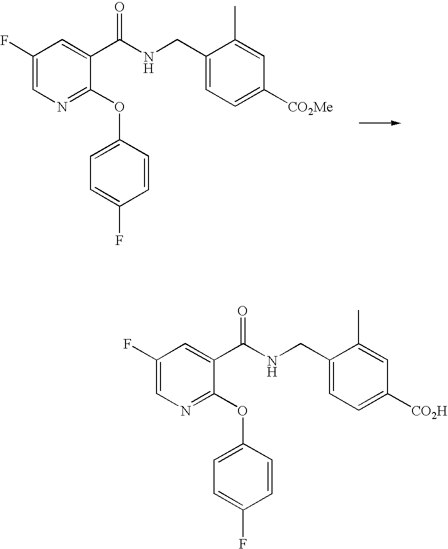

The title compound was prepared according to the procedure described in step 4 of Example 1 from methyl 4-[({[5-fluoro-2-(4-fluorophenoxy)pyridin-3-yl]carbonyl}amino)methyl]-3-methylbenzoate (step 1): ¹H-NMR (DMSO-d₆) δ 9.05 (1H, t, J=6.1 Hz), 8.25 (1H, d, J=2.9 Hz), 8.09 (1H, dd, J=8.1, 3.1 Hz), 7.74 (1H, s), 7.68 (1H, d, J=7.9 Hz), 7.44 (1H, d, J=8.1 Hz), 7.31-7.25 (4H, m), 4.54 (2H, d, J=5.9 Hz), 2.36 (3H, s); MS (ESI) m/z 399 (M+H)⁺, 397 (M−H)⁻.

### Example 8

### 3-FLUORO-4-[({[5-FLUORO-2-(4-FLUOROPHENOXY)PYRIDIN-3-YL]CARBONYL}AMINO)METHYL]BENZOIC ACID

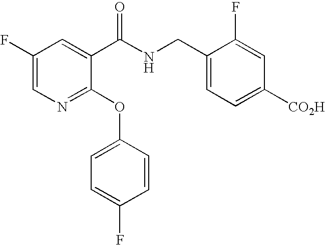

### STEP 1. Methyl 3-fluoro-4-[({[5-fluoro-2-(4-fluorophenoxy)pyridin-3-yl]carbonyl}amino)methyl]benzoate

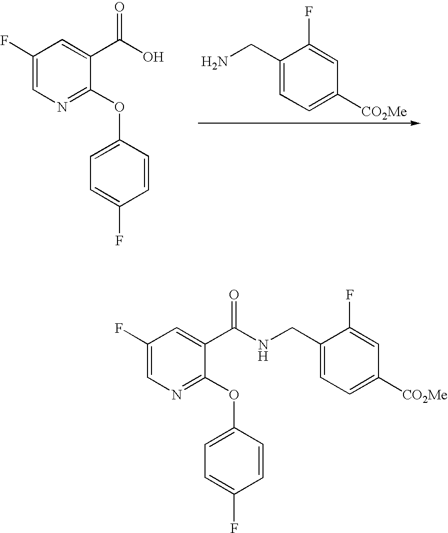

The title compound was prepared according to the procedure described in step 3 of Example 1 from 5-fluoro-2-(4-fluorophenoxy)nicotinic acid (step 3 of Example 1) and methyl 4-(aminomethyl)-3-fluorobenzoate (WO 9926923): ¹H-NMR (CDCl₃) δ 8.45-8.33 (2H, m), 8.04 (1H, d, J=3.1 Hz), 7.80 (1H, dd, J=7.9, 1.5 Hz), 7.71 (1H, dd, J=10.5, 1.5 Hz), 7.49 (1H, t, J=7.6 Hz), 7.17-7.12 (4H, m), 4.78 (2H, d, J=6.1 Hz), 3.91 (3H, s); MS (ESI) m/z 417 (M+H)⁺, 415 (M−H)⁻.

### STEP 2. 3-Fluoro-4-[({[5-fluoro-2-(4-fluorophenoxy)pyridin-3-yl]carbonyl}amino)methyl]benzoic acid

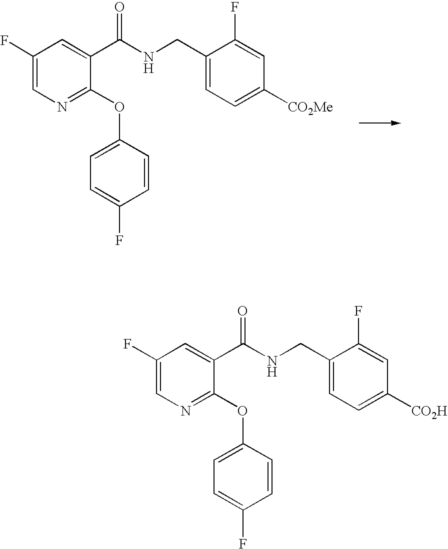

The title compound was prepared according to the procedure described in step 4 of Example 1 from methyl 3-fluoro-4-[({[5-fluoro-2-(4-fluorophenoxy)pyridin-3-yl]carbonyl}amino)methyl]benzoate (step 1): ¹H-NMR (DMSO-d₆) [symbol] 9.14 (1H, t, J=5.9 Hz), 8.26 (1H, d, J=3.1 Hz), 8.11 (1H, dd, J=8.3, 3.1 Hz), 7.72-7.53 (3H, m), 7.31-7.25 (4H, m), 4.62 (2H, d, J=5.9 Hz); MS (ESI) m/z 403 (M+H)⁺, 401 (M−H)⁻.

### Example 9

### 4-[({[5-FLUORO-2-(4-FLUOROPHENOXY)PYRIDIN-3-YL]CARBONYL}AMINO)METHYL]-2-METHYLBENZOIC ACID

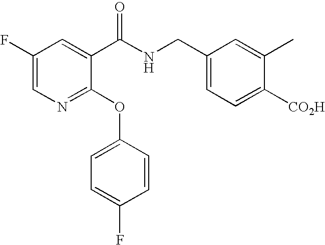

### STEP 1. Methyl 4-[({[5-fluoro-2-(4-fluorophenoxy)pyridin-3-yl]carbonyl}amino)methyl]-2-methylbenzoate

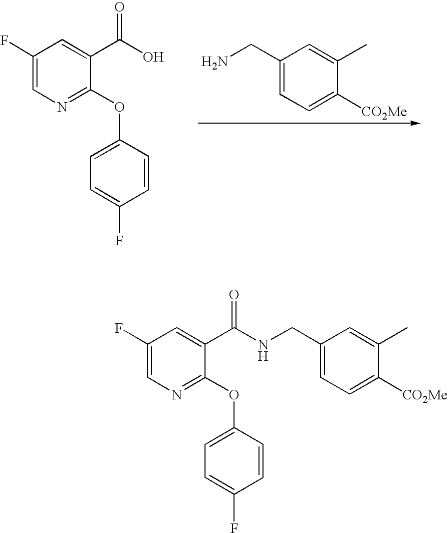

The title compound was prepared according to the procedure described in step 3 of Example 1 from 5-fluoro-2-(4-fluorophenoxy)nicotinic acid (step 3 of Example 1) and methyl 4-(aminomethyl)-2-methylbenzoate (WO 0324955): ¹H-NMR (CDCl₃) [symbol] 8.38 (1H, dd, J=8.1, 3.1 Hz), 8.30-8.24 (1H, m), 8.05 (1H, d, J=3.1 Hz), 7.88 (1H, d, J=8.1 Hz), 7.26-7.08 (6H, m), 4.71 (2H, d, J=5.9 Hz), 3.90 (3H, s), 3.87 (3H, s); MS (ESI) m/z 413 (M+H)⁺, 411 (M−H)⁻.

### STEP 2. 4-[({[5-Fluoro-2-(4-fluorophenoxy)pyridin-3-yl]carbonyl}amino)methyl]-2-methylbenzoic acid

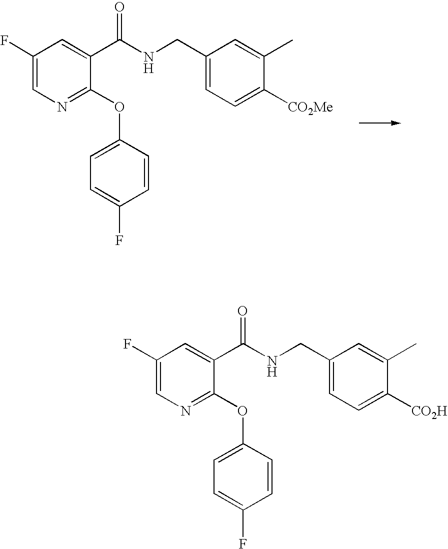

The title compound was prepared according to the procedure described in step 4 of Example 1 from methyl 4-[({[5-fluoro-2-(4-fluorophenoxy)pyridin-3-yl]carbonyl}amino)methyl]-2-methylbenzoate (step 1): ¹H-NMR (DMSO-d₆) [symbol] 9.10 (1H, t, J=5.9 Hz), 8.24 (1H, d, J=3.1 Hz), 8.09 (1H, dd, J=8.3, 3.1 Hz), 7.76 (1H, d, J=8.3 Hz), 7.27-7.25 (6H, m), 4.54 (2H, d, J=5.9 Hz), 2.42 (3H, s); MS (ESI) m/z 399 (M+H)⁺, 397 (M−H)⁻.

### The Synthetic Procedure of Examples 10-42

The compounds disclosed hereinafter were prepared according to the following procedure:

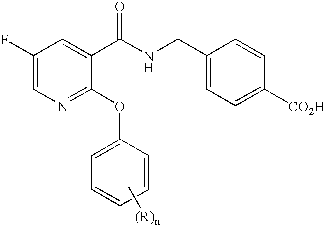

### STEP 1. tert-Butyl 4-({[(2-chloro-5-fluoropyridin-3-yl)carbonyl]amino}methyl)benzoate

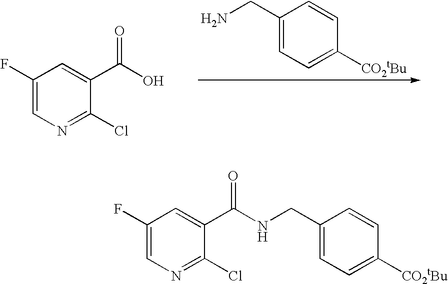

To a stirred solution of 2-chloro-5-fluoronicotinic acid (EP 634413, 2.0 g, 10 mmol) and tert-butyl 4-(aminomethyl)benzoate (WO 9950249, 1.65 g, 8 mmol) in dichloromethane (25 mL) were successively added 1-(3-dimethylaminopropyl)-3-ethylcarbodiimide hydrochloride (EDCl) (2.88 g, 15 mmol), 1-hydroxybenzotriazole hydrate (HOBT) (1.53 g, 10 mmol), and triethylamine (5 mL). After being stirred overnight, the reaction mixture was poured into water (100 mL). The organic layer was separated and the aqueous layer was extracted with dichloromethane (50 mL×2). The combined organic layers were washed with brine (100 mL), dried (sodium sulfate), and evaporated. The residue was purified by flash column chromatography on silica gel (200 g) eluting with dichloromethane/ethyl acetate (20/1) to afford 2.39 g (82%) of the title compounds as white solids: ¹H-NMR (CDCl₃) δ 8.33 (1H, d, J=3.1 Hz), 7.97 (2H, d, J=8.4 Hz), 7.91 (1H, dd, J=7.9, 3.1 Hz), 7.40 (2H, d, J=8.4 Hz), 7.04 (1H, br.s), 4.70 (2H, d, J=5.9 Hz), 1.58 (9H, s).

### STEP 2. 4-[({[5-Fluoro-2-(substituted-phenoxy)pyridin-3-yl]carbonyl}amino)methyl]benzoic acid

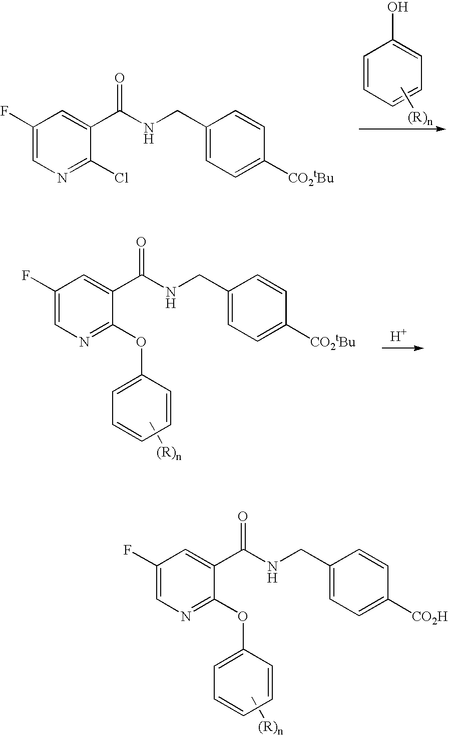

To a solution of substituted-phenol (0.15 mmol) were added a solution of tert-butyl 4-({[(2-chloro-5-fluoropyridin-3-yl)carbonyl]amino}methyl)benzoate (step 1, 0.05 mmol) in toluene (0.6 mL) and 2-tert-Butylimino-2-diethylamino-1,3-dimethyl-perhydro-1,3,2-diazaphosphorine on polystyrene (PS-BEMP, 0.15 mmol). Then the mixture was agitated at 110° C. overnight. To the resultant mixture were added AcOEt (0.5 mL) and 0.5 N aq.HCl (0.5 mL). The organic layer was extracted and concentrated in vacuo. The crude product was purified by preparative LCMS (XTerra® C18, 20×50 mm) eluting with H₂O/MeOH/1% aqueous HCO₂H (90/5/5 to 10/85/5). After a TFA-DCE solution (1-1, 0.6 mL) was added to the purified material, the mixture was left at room temperature for 1.5 h. Then the mixture was concentrate in vacuo to afford the desired product.

### Example 10

### 4-[({[5-FLUORO-2-(3-METHOXY-5-METHYLPHENOXY)PYRIDIN-3-YL]CARBONYL}AMINO)METHYL]BENZOIC ACID

Observed MS (ESI) m/z 411.01 (M+H)⁺

Exact Mass calcd for C22H19FN2O5: m/z 410.13

### Example 11

### 4-[({[2-(2-CHLOROPHENOXY)-5-FLUOROPYRIDIN-3-YL]CARBONYL}AMINO)METHYL]BENZOIC ACID

Observed MS (ESI) m/z 400.96 (M+H)⁺

Exact Mass calcd for C20H14ClFN2O4: m/z 400.06

### Example 12

### 4-[({[2-(3-CHLOROPHENOXY)-5-FLUOROPYRIDIN-3-YL]CARBONYL}AMINO)METHYL]BENZOIC ACID

Observed MS (ESI) m/z 400.96 (M+H)⁺

Exact Mass calcd for C20H14ClFN2O4: m/z 400.06

### Example 13

### 4-[({[2-(2,3-DIHYDRO-1H-INDEN-5-YLOXY)-5-FLUOROPYRIDIN-3-YL]CARBONYL}AMINO)METHYL]BENZOIC ACID

Observed MS (ESI) m/z 407.05 (M+H)⁺

Exact Mass calcd for C23H19FN2O4: m/z 406.13

### Example 14

### 4-[({[2-(BIPHENYL-4-YLOXY)-5-FLUOROPYRIDIN-3-YL]CARBONYL}AMINO)METHYL]BENZOIC ACID

Observed MS (ESI) m/z 443.03 (M+H)⁺

Exact Mass calcd for C26H19F N2O4: m/z 442.13

### Example 15

### 4-[({[2-(3-CHLORO-4-METHYLPHENOXY)-5-FLUOROPYRIDIN-3-YL]CARBONYL}AMINO)METHYL]BENZOIC ACID

Observed MS (ESI) m/z 414.96 (M+H)⁺

Exact Mass calcd for C21H16ClFN2O4: m/z 414.08

### Example 16

### 4-[({[2-(3,5-DIFLUOROPHENOXY)-5-FLUOROPYRIDIN-3-YL]CARBONYL}AMINO)METHYL]BENZOIC ACID

Observed MS (ESI) m/z 402.97 (M+H)⁺

Exact Mass calcd for C20H13F3N2O4: m/z 402.08

### Example 17

### 4-[({[2-(4-CYCLOPENTYLPHENOXY)-5-FLUOROPYRIDIN-3-YL]CARBONYL}AMINO)METHYL]BENZOIC ACID

Observed MS (ESI) m/z 435.05 (M+H)⁺

Exact Mass calcd for C25H23FN2O4: m/z 434.16

### Example 18

### 4-[({[5-FLUORO-2-(3-METHOXYPHENOXY)PYRIDIN-3-YL]CARBONYL}AMINO)METHYL]BENZOIC ACID

Observed MS (ESI) m/z 397.01 (M+H)⁺

Exact Mass calcd for C21H17FN2O5: m/z 396.11

### Example 19

### 4-({[(5-FLUORO-2-PHENOXYPYRIDIN-3-YL)CARBONYL]AMINO}METHYL)BENZOIC ACID

Observed MS (ESI) m/z 367.01 (M+H)+

Exact Mass calcd for C20H15F N2O4: m/z 366.1

### Example 20

### 4-[({[5-FLUORO-2-(2-FLUOROPHENOXY)PYRIDIN-3-YL]CARBONYL}AMINO)METHYL]BENZOIC ACID

Observed MS (ESI) m/z 385.02 (M+H)⁺

Exact Mass calcd for C20H14F2N2O4: m/z 384.09

### Example 21

### 4-{[({2-[4-(BENZYLOXY)PHENOXY]-5-FLUOROPYRIDIN-3-YL}CARBONYL)AMINO]METHYL}BENZOIC ACID

Observed MS (ESI) m/z 473.04 (M+H)⁺

Exact Mass calcd for C27H21FN2O5: m/z 472.14

### Example 22

### 4-[({[5-FLUORO-2-(3-FLUOROPHENOXY)PYRIDIN-3-YL]CARBONYL}AMINO)METHYL]BENZOIC ACID

Observed MS (ESI) m/z 385.02 (M+H)⁺

Exact Mass calcd for C20H14F2N2O4: m/z 384.09

### Example 23

### 4-[({[2-(3-ETHYNYLPHENOXY)-5-FLUOROPYRIDIN-3-YL]CARBONYL}AMINO)METHYL]BENZOIC ACID

Observed MS (ESI) m/z 390.98 (M+H)⁺

Exact Mass calcd for C22H15FN2O4: m/z 390.1

### Example 24

### 4-[({[2-(2-CHLORO-5-METHYLPHENOXY)-5-FLUOROPYRIDIN-3-YL]CARBONYL}AMINO)METHYL]BENZOIC ACID

Observed MS (ESI) m/z 414.96 (M+H)⁺

Exact Mass calcd for C₂₁H₁₆ClFN2O4: m/z 414.08

### Example 25

### 4-[({[2-(3-CHLORO-4-FLUOROPHENOXY)-5-FLUOROPYRIDIN-3-YL]CARBONYL}AMINO)METHYL]BENZOIC ACID

Observed MS (ESI) m/z 418.95 (M+H)⁺

Exact Mass calcd for C20H13ClF2N2O4: m/z 418.05

### Example 26

### 4-[({[2-(2,6-DIFLUOROPHENOXY)-5-FLUOROPYRIDIN-3-YL]CARBONYL}AMINO)METHYL]BENZOIC ACID

Observed MS (ESI) m/z 402.97 (M+H)⁺

Exact Mass calcd for C20H13F3N2O4: m/z 402.08

### Example 27

### 4-[({[2-(3-ETHYLPHENOXY)-5-FLUOROPYRIDIN-3-YL]CARBONYL}AMINO)METHYL]BENZOIC ACID

Observed MS (ESI) m/z 395.06 (M+H)⁺

Exact Mass calcd for C22H19FN2O4: m/z 394.13

### Example 28

### 4-[({[2-(3,4-DIFLUOROPHENOXY)-5-FLUOROPYRIDIN-3-YL]CARBONYL}AMINO)METHYL]BENZOIC ACID

Observed MS (ESI) m/z 402.97 (M+H)⁺

Exact Mass calcd for C20H13F3N2O4: m/z 402.08

### Example 29

### 4-{[({5-FLUORO-2-[3-(TRIFLUOROMETHOXY)PHENOXY]PYRIDIN-3-YL}CARBONYL)AMINO]METHYL}BENZOIC ACID

Observed MS (ESI) m/z 451.00 (M+H)⁺

Exact Mass calcd for C21H14F4N2O5: m/z 450.08

### Example 30

### 4-[({[5-FLUORO-2-(4-FLUORO-3-METHYLPHENOXY)PYRIDIN-3-YL]CARBONYL}AMINO)METHYL]BENZOIC ACID

Observed MS (ESI) m/z 399.02 (M+H)⁺

Exact Mass calcd for C21H16F2N2O4: m/z 398.11

### Example 31

### 4-[({[2-(BIPHENYL-3-YLOXY)-5-FLUOROPYRIDIN-3-YL]CARBONYL}AMINO)METHYL]BENZOIC ACID

Observed MS (ESI) m/z 443.03 (M+H)⁺

Exact Mass calcd for C26H19FN2O4: m/z 442.13

### Example 32

### 4-[({[5-FLUORO-2-(3-METHYLPHENOXY)PYRIDIN-3-YL]CARBONYL}AMINO)METHYL]BENZOIC ACID

Observed MS (ESI) m/z 381.00 (M+H)⁺

Exact Mass calcd for C21H17FN2O4: m/z 380.12

### Example 33

### 4-[({2-(3-ACETYLPHENOXY)-5-FLUOROPYRIDIN-3-YL]CARBONYL}AMINO)METHYL]BENZOIC ACID

Observed MS (ESI) m/z 409.00 (M+H)⁺

Exact Mass calcd for C22H17 FN2O5: m/z 408.11

### Example 34

### 4-[({[5-FLUORO-2-(2-NAPHTHYLOXY)PYRIDIN-3-YL]CARBONYL}AMINO)METHYL]BENZOIC ACID

Observed MS (ESI) m/z 417.03 (M+H)⁺

Exact Mass calcd for C₂₄H₁₇F N2O4: m/z 416.12

### Example 35

### 4-[({[5-FLUORO-2-(1-NAPHTHYLOXY)PYRIDIN-3-YL]CARBONYL}AMINO)METHYL]BENZOIC ACID

Observed MS (ESI) m/z 417.02 (M+H)⁺

Exact Mass calcd for C24H17FN2O4: m/z 416.12

### Example 36

### 4-{[({2-[(4-CHLORO-1-NAPHTHYL)OXY]-5-FLUOROPYRIDIN-3-YL}CARBONYL)AMINO]METHYL}BENZOIC ACID

Observed MS (ESI) m/z 450.98 (M+H)⁺

Exact Mass calcd for C24H16ClFN2O4: m/z 450.08

### Example 37

### 4-[({[2-(3-BENZOYLPHENOXY)-5-FLUOROPYRIDIN-3-YL]CARBONYL}AMINO)METHYL]BENZOIC ACID

Observed MS (ESI) m/z 471.03 (M+H)⁺

Exact Mass calcd for C27H19FN2O5: m/z 470.13

### Example 38

### 4-[({[5-FLUORO-2-(2-METHYLPHENOXY)PYRIDIN-3-YL]CARBONYL}AMINO)METHYL]BENZOIC ACID

Observed MS (ESI) m/z 381.00 (M+H)⁺

Exact Mass calcd for C21H17FN2O4: m/z 380.12

### Example 39

### 4-[({[5-FLUORO-2-(QUINOLIN-8-YLOXY)PYRIDIN-3-YL]CARBONYL}AMINO)METHYL]BENZOIC ACID

Observed MS (ESI) m/z 418.01 (M+H)⁺

Exact Mass calcd for C23H16F N3O4: m/z 417.11

### Example 40

### 4-{[({5-FLUORO-2-[4-(2-METHYL-13-THIAZOL-4-YL)PHENOXY]PYRIDIN-3-YL}CARBONYL)AMINO]METHYL}BENZOIC ACID

Observed MS (ESI) m/z 463.96 (M+H)⁺

Exact Mass calcd for C24H18FN3O4 S: m/z 463.10

### Example 41

### 4-{[({5-FLUORO-2-[(5-FLUOROQUINOLIN-8-YL)OXY]PYRIDIN-3-YL}CARBONYL)AMINO]METHYL}BENZOIC ACID

Observed MS (ESI) m/z 435.97 (M+H)⁺

Exact Mass calcd for C23H15F2N3O4: m/z 435.1

### Example 42

### 4-[({[5-FLUORO-2-(4-PYRIDIN-2-YLPHENOXY)PYRIDIN-3-YL]CARBONYL}AMINO)METHYL]BENZOIC ACID

Observed MS (ESI) m/z 444.00 (M+H)⁺

Exact Mass calcd for C25H18FN3O4: m/z 443.13

### Example 43

### 4-[({[5-CHLORO-2-(4-FLUOROPHENOXY)PYRIDIN-3-YL]CARBONYL}AMINO)METHYL]BENZOIC ACID

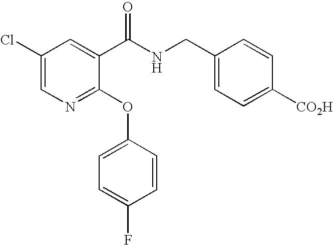

### STEP 1. Methyl 4-[({[5-chloro-2-(4-fluorophenoxy)pyridin-3-yl]carbonyl}amino)methyl]benzoate

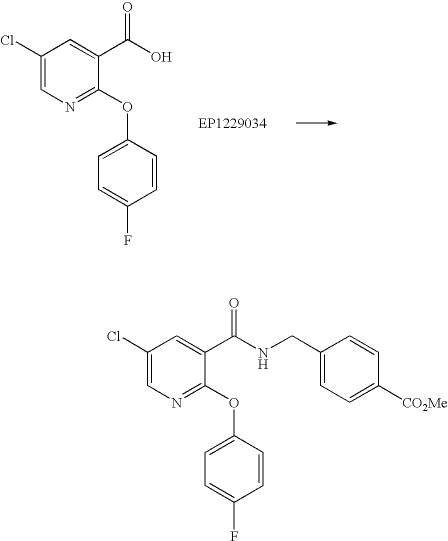

To a stirred solution of 5-chloro-2-(4-fluorophenoxy)nicotinic acid (EP 1229034, 150 mg, 0.56 mmol), methyl 4-(aminomethyl)benzoate hydrochloride (136 mg, 0.67 mmol), and triethylamine (0.31 mL, 2.24 mmol) in dichloromethane (8 mL) was added 2-bromo-1-ethylpyridinium tetrafluoroborate (230 mg, 0.84 mmol) at 0° C. The resulting mixture was warmed to room temperature and stirred for 16 h. The mixture was diluted with dichloromethane (50 mL) and washed with 1 M hydrochloric acid (30 mL), saturated aqueous sodium hydrogen carbonate solution (30 mL), and brine (30 mL). The organic layer was dried over magnesium sulfate, and concentrated under reduced pressure. The residue was purified by flash column chromatography on silica gel eluting with hexane/ethyl acetate (2:1) to afford 155 mg (67%) of the title compounds as white solids: H-NMR (CDCl₃) δ 8.59 (1H, dd, J=2.6, 0.9 Hz), 8.26-8.17 (1H, m), 8.13 (1H, dd, J=2.8, 0.9 Hz), 8.00 (2H, d, J=8.1 Hz), 7.41 (2H, d, J=8.1 Hz), 7.15-7.07 (4H, m), 4.76 (2H, d, J=5.9 Hz), 3.90 (3H, s); MS (ESI) m/z 415 (M+H)⁺, 413 (M−H)⁻.

### STEP 2. 4-[({[5-Chloro-2-(4-fluorophenoxy)pyridin-3-yl]carbonyl}amino)methyl]benzoic acid

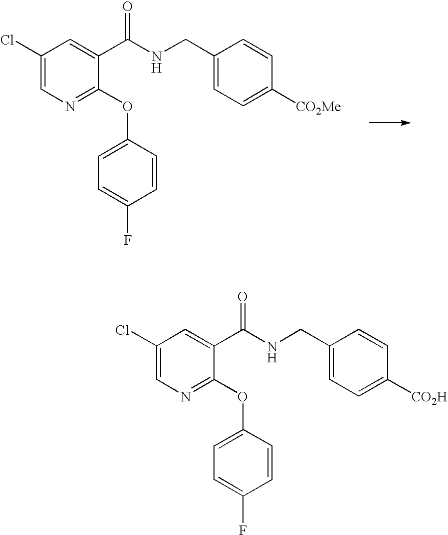

A mixture of methyl 4-[({[5-chloro-2-(4-fluorophenoxy)pyridin-3-yl]carbonyl}amino)methyl]benzoate (step 1, 154 mg, 0.37 mmol), tetrahydrofuran (2 mL), methanol (2 mL), and 2 M sodium hydroxide (2 mL) was stirred at room temperature for 4 h. The mixture was poured into 1 M hydrochloric acid (30 mL), and extracted with ethyl acetate (50 mL). The organic layer was dried over magnesium sulfate and evaporated. The residue was purified by TLC [dichloromethane/ethyl acetate (1:2)] to give 98 mg (66%) of the title compound as white solids: ¹H-NMR (DMSO-d₆) [symbol] 9.13 (1H, t, J=6.0 Hz), 8.29 (1H, dd, J=2.6, 1.5 Hz), 8.22 (1H, dd, J=2.6, 1.5 Hz), 7.88 (2H, d, J=8.1 Hz), 7.47 (2H, d, J=8.1 Hz), 7.29-7.26 (4H, m), 4.60 (2H, d, J=6.0 Hz); MS (ESI) m/z 401 (M+H)⁺, 399 (M−H)⁻.

### Example 44

### 4-[(1S)-1-({[5-CHLORO-2-(4-FLUOROPHENOXY)PYRIDIN-3-YL]CARBONYL}AMINO)ETHYL]BENZOIC ACID

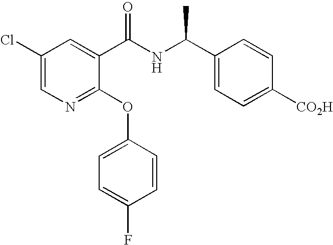

### STEP 1. 4-((1S)-1-{[(Benzyloxy)carbonyl]amino}ethyl)benzoic acid

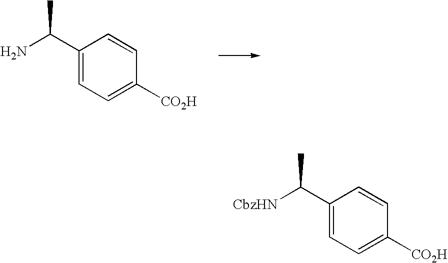

To a cooled (0° C.) mixture of 4-[(1S)-1-aminoethyl]benzoic acid (Chem. Eur. J. 1999, 5, 1095-1105, 16.2 g, 98 mmol) and 2 N sodium hydroxide aqueous solution (100 ml) was added benzyl chloroformate (20.5 g, 120 mmol) dropwise over 30 min period followed by the addition of additional 2 N sodium hydroxide aqueous solution (70 ml). The reaction mixture was stirred overnight at room temperature and then acidified to pH 1 with concentrated hydrochloric acid. The resulting precipitate was filtered, washed with water (100 mL), and then vacuum dried to yield 26 g (88%) of the title compounds as white solids: ¹H-NMR (CDCl₃) [symbol] 7.95 (2H, d, J=8.1 Hz), 7.40-7.15 (7H, m), 5.12-4.94 (3H, m), 4.82 (1H, br.s), 1.40 (3H, d, J=7.0 Hz); MS (ESI) m/z 300 (M+H)⁺, 298 (M−H{tilde over ())}⁻

### STEP 2. tert-Butyl 4-((1S)-1-{[(benzyloxy)carbonyl]amino}ethyl)benzoate

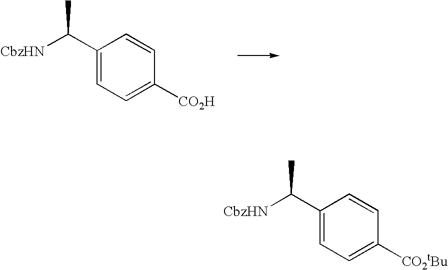

To a solution of 4-((1S)-1-{[(benzyloxy)carbonyl]amino}ethyl)benzoic acid (step 1, 3.7 g, 12.4 mmol) and benzyltriethylammonium chloride (3.0 g, 13 mmol) in N,N-dimethylacetamide (100 mL) was added anhydrous potassium carbonate (47 g, 340 mmol) followed by 2-bromo-2-methylpropane (89 g, 650 mmol). The resulting mixture stirred for 24 h at 55° C. After cooling to room temperature, the reaction mixture was poured into cold water (500 mL) under stirring. The resulting solution was extracted with ethyl acetate (500 ml). The organic phase was washed with water (300 ml) and brine (200 mL), dried (sodium sulfate), and evaporated. The residue was purified by flash column chromatography on silica gel (150 g) eluting with hexane/ethyl acetate (3/1) to afford 3.48 g (79%) of the title compounds as white solids: ¹H-NMR (CDCl₃) [symbol] 7.95 (2H, d, J=8.3 Hz), 7.44-7.24 (7H, m), 5.15-4.99 (3H, m), 4.88 (1H, br.s), 1.58 (9H, s), 1.47 (3H, d, J=7.0 H\tilde{z}) .

### STEP 3. tert-Butyl 4-[(1S)-1-aminoethyl]benzoate

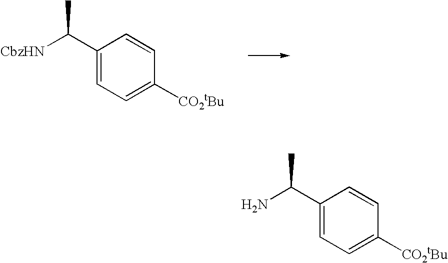

To a stirred solution of tert-butyl 4-((1S)-1-{[(benzyloxy)carbonyl]amino}ethyl)benzoate (step 2, 3.48 g, 9.8 mmol) in a mixture of ethanol (25 mL) and acetic acid (25 mL) was added 10% palladium-carbon (400 mg). The mixture was stirred at room temperature for 2 h under hydrogen atmosphere. The palladium catalyst was removed by filtration and washed with ethanol (100 mL). The filtrate was concentrated under reduced pressure and the residue was partitioned between ethyl acetate (200 mL) and saturated sodium bicarbonate aqueous solution (200 mL). The organic phase was separated and the aqueous phase was extracted with ethyl acetate (200 mL). The combined organic extracts were washed with brine (200 mL) and dried (sodium sulfate), and concentrated to give 2.02 g (93%) of the title compounds as white solids: ¹H-NMR (CDCl₃) [symbol] 7.95 (2H, d, J=8.3 Hz), 7.39 (2H, d, J=8.3 Hz), 4.22-4.12 (1H, dq, J=7.3, 6.6 Hz), 1.80 (2H, br.s), 1.58 (9H, s), 1.38 (3H, d, J=6.6 H\tilde{z}).

### STEP 4. tert-Butyl 4-[(1S)-1-({[5-Chloro-2-(4-fluorophenoxy)pyridin-3-yl]carbonyl}amino)ethyl]benzoate

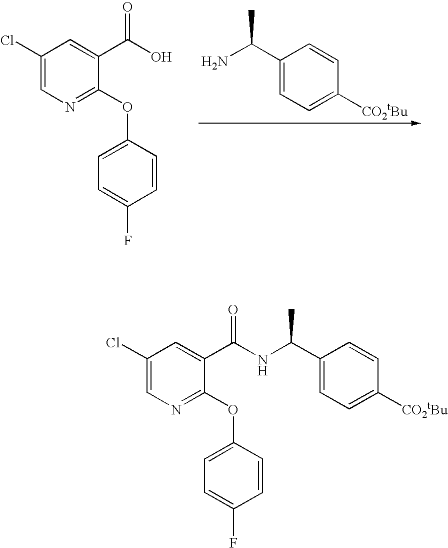

The title compound was prepared according to the procedure described in step 3 of Example 1 from 5-chloro-2-(4-fluorophenoxy)nicotinic acid (EP 1229034) and tert-butyl 4-[(1S)-1-aminoethyl]benzoate (step 3): ¹H-NMR (CDCl₃) [symbol] 8.54 (1H, d, J=2.7 Hz), 8.14 (1H, br.s), 8.13 (1H, d, J=2.7 Hz), 7.95 (2H, d, J=8.3 Hz), 7.39 (2H, d, J=8.3 Hz), 7.17-7.11 (4H, m), 5.36 (1H, dq, J=7.2, 7.0 Hz), 1.59 (3H, d, J=7.0 Hz), 1.58 (9H, s); MS (ESI) m/z 415 (M+H)⁺.

### STEP 5. 4-[(1S)-1-({[5-Chloro-2-(4-fluorophenoxy)pyridin-3-yl]carbonyl}amino)ethyl]benzoic acid

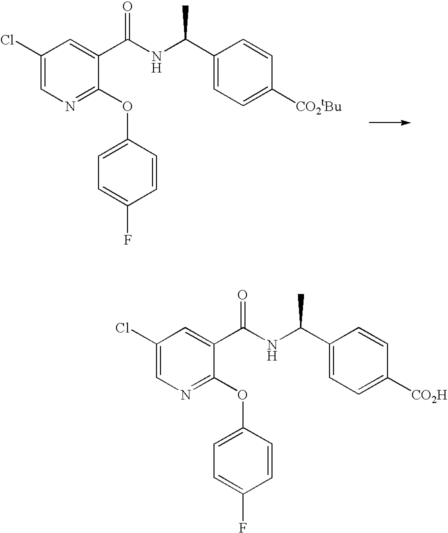

Trifluoroacetic acid (10 mL) was added to a solution of tert-butyl 4-[(1S)-1-({[5-Chloro-2-(4-fluorophenoxy)pyridin-3-yl]carbonyl}amino)ethyl]benzoate (step 4, 2.1 g; 4.3 mmol) in dichloromethane (30 mL). The reaction mixture was stirred at room temperature until the starting material was fully consumed (4 h). The solvent and most of the trifluoroacetic acid were removed under reduced pressure. The residue was purified by flash silica gel column chromatography on silica gel (50 g) eluting with dichloromethane/methanol (20/1) and recrystallization (ethyl acetate-diisopropyl ether) to give 1.24 g, (86%) of the title compounds as white crystals: mp 198.2° C.; ¹H-NMR (CDCl₃) δ 8.56 (1H, d, J=2.6 Hz), 8.16 (1H, br.s), 8.14 (1H, d, J=2.6 Hz), 8.07 (2H, d, J=8.3 Hz), 7.46 (2H, d, J=8.3 Hz), 7.19-7.12 (4H, m), 5.46-5.30 (1H, m), 1.61 (3H, d, J=7.1 Hz); MS (ESI) m/z 415 (M+H)⁺, 413 (M−H)⁻.

### Example 45

### 4-{(1S)-1-[({5-CHLORO-2-[3-(1,3-THIAZOL-2-YL)PHENOXY]PYRIDIN-3-YL}CARBONYL)AMINO]ETHYL}BENZOIC ACID

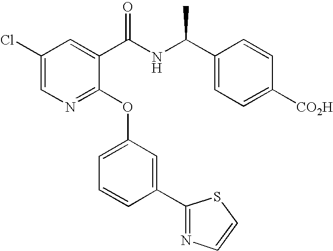

### STEP 1. tert-butyl 4-((1S)-1-{[(2,5-dichloropyridin-3-yl)carbonyl]amino}ethyl)benzoate

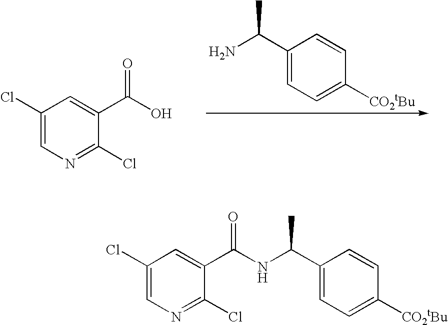

To a stirred solution of 2,5-dichloronicotinic acid (Syn. Commun. 1989, 19, 553-9, 1.92 g, 10 mmol) and tert-butyl 4-[(1S)-1-aminoethyl]benzoate (example 44 step 3, 2.02 g, 9.1 mmol) in dichloromethane (20 mL) were successively added 1-(3-dimethylaminopropyl)-3-ethylcarbodiimide hydrochloride (EDCl) (2.59 g, 13.5 mmol), 1-hydroxybenzotriazole hydrate (HOBT) (2.07 g, 13.5 mmol), and triethylamine (4 mL). After being stirred overnight, the reaction was quenched by the addition of saturated sodium bicarbonate aqueous solution (100 mL). The aqueous layer was extracted with dichloromethane (50 mL×3) and the combined organic layers were washed with brine (100 mL), dried (sodium sulfate), and evaporated. The remaining residue was purified by flash column chromatography on silica gel (100 g) eluting with dichloromethane/ethyl acetate (20/1) to afford 2.51 g (70%) of the title compounds as white solids: ¹H-NMR (CDCl₃) [symbol] 8.41 (1H, d, J=2.6 Hz), 8.09 (1H, d, J=2.6 Hz), 7.99 (2H, d, J=8.3 Hz), 7.43 (2H, d, J=8.3 Hz), 6.81 (1H, d, J=7.2 Hz), 5.33 (1H, dq, J=7.2, 7.0 Hz), 1.62 (3H, d, J=7.0 Hz), 1.58 (9H, s).

### STEP 2. tert-butyl 4-{(1S)-1-[({5-chloro-2-[3-(1,3-thiazol-2-yl)phenoxy]pyridin-3-yl}carbonyl)amino]ethyl}benzoate

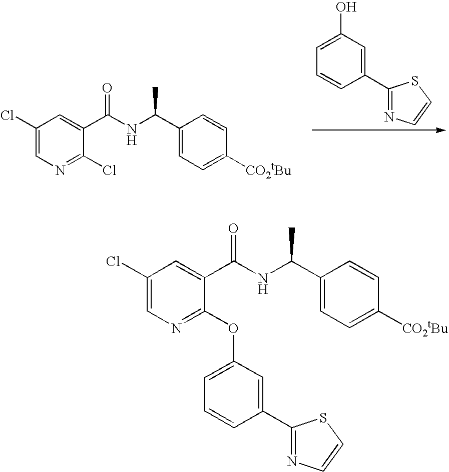

A mixture of tert-butyl 4-((1S)-1-{[(2,5-dichloropyridin-3-yl)carbonyl]amino}ethyl)benzoate (step 1, 178 mg, 0.45 mmol), 3-(1,3-thiazol-2-yl)phenol (DE 2130709, 162 mg, 0.91 mmol) and 2-tert-butylimino-2-diethylamino-1,3-dimethylperhydro-1,3,2-diazaphosphorine (BEMP, 217 μL, 0.75 mmol) in toluene (2 mL) was stirred at 110° C. for 5 h. After removal of solvent, the residue was eluted on silica gel short column (hexane/ethyl acetate (4/1)) to afford 259 mg (quant.) of the title compound as a white amorphous: ¹H-NMR (CDCl₃) [symbol] 8.57 (1H, d, J=2.7 Hz), 8.15-8.11 (2H, m), 7.97-7.94 (2H, m), 7.90-7.83 (3H, m), 7.58-7.38 (3H, m), 7.34-7.20 (2H, m), 5.42-5.32 (1H, m), 1.61-1.57 (12H, m); MS (ESI) m/z 536 (M+H)⁺, 534 (M−H)⁻.

### STEP 3. 4-{(1S)-1-[({5-Chloro-2-[3-(1,3-thiazol-2-yl)phenoxy]pyridin-3-yl}carbonyl)amino]ethyl}benzoic acid

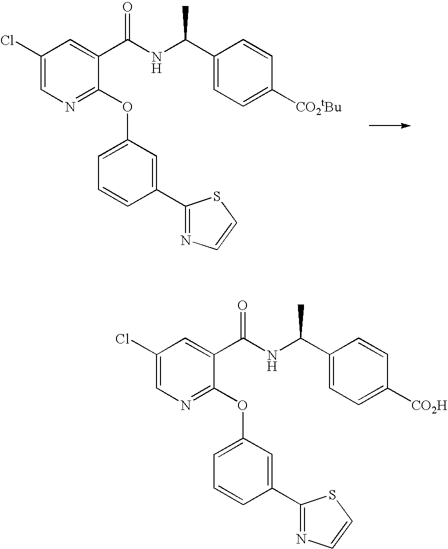

The title compound was prepared according to the procedure described in step 5 of Example 44 from tert-butyl 4-{(1S)-1-[({5-chloro-2-[3-(1,3-thiazol-2-yl)phenoxy]pyridin-3-yl}carbonyl)amino]ethyl}benzoate (step 2): ¹H-NMR (CDCl₃) δ 8.56 (1H, d, J=2.7 Hz), 8.16-8.14 (2H, m), 8.07-8.04 (2H, m), 7.90-7.85 (3H, m), 7.58-7.52 (1H, m), 7.48-7.45 (2H, m), 7.39 (1H, d, J=3.2 Hz), 7.29-7.21 (1H, m), 5.44-5.34 (1H, m), 1.61 (3H, d, J=6.8 Hz); MS (ESI) m/z 480 (M+H)⁺, 478 (M−H)⁻.

### Example 46

### 4-{(1S)-1-[({5-CHLORO-2-[(5-CHLOROPYRIDIN-3-YL)OXY]PYRIDIN-3YL}CARBONYL)AMINO]ETHYL}BENZOIC ACID

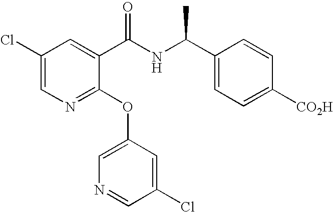

### STEP 1. 5-Chloro-2-[(5-chloropyridin-3-yl)oxy]nicotinic acid

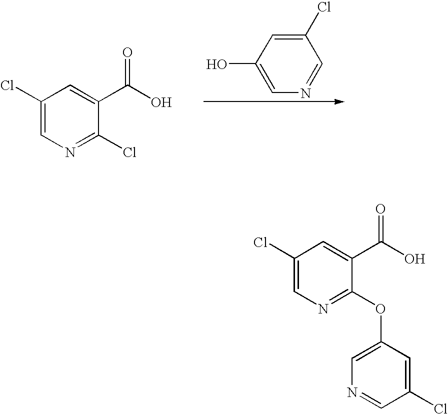

A mixture of 2,5-dichloronicotinic acid (Syn. Commun. 1989, 19, 553-9, 500 mg, 2.6 mmol), 3-chloro-5-hydroxypyridine (404 mg, 3.1 mmol), copper bronze (36 mg, 0.57 mmol), cuprous iodide (40 mg, 0.21 mmol) and potassium carbonate (792 mg, 5.7 mmol) in N,N-dimethylforamide (7 mL) was heated under reflux for 3 h. The reaction mixture was filtered through a pad of celite and the filtrate was concentrated. The residue was diluted with water (10 mL) and the mixture was acidified with 2 N hydrochloric acid (2 mL). Precipitated solids were collected by filtration and dried under reduced pressure at 40° C. to afford 349 mg of the title compound: ¹H-NMR (CDCl₃) [symbol] 8.37 (3H, br.s), 7.96 (2H, br.s), a peak of COOH was not observed; MS (ESI) m/z 285 (M+H)⁺.

### STEP 2. tert-Butyl 4-{(1S)-1-[({5-chloro-2-[(5-chloropyridin-3-yl)oxy]pyridin-3-yl}carbonyl)amino]ethyl}benzoate

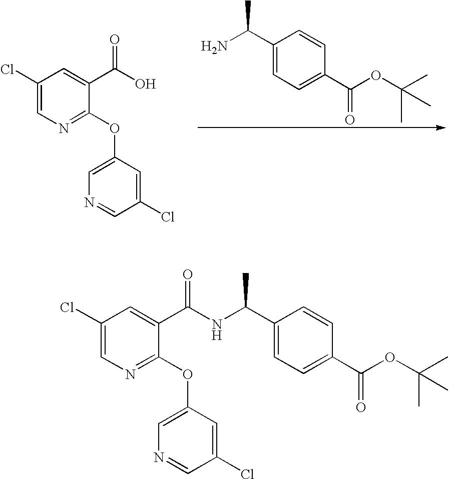

The title compound was prepared according to the procedure described in step 3 of Example 1 from 5-chloro-2-[(5-chloropyridin-3-yl)oxy]nicotinic acid (step 1) and tert-butyl 4-[(1S)-1-aminoethyl]benzoate (step 3 of Example 44): ¹H-NMR (CDCl₃) [symbol] 8.56 (1H, d, J=2.7 Hz), 8.55-8.54 (1H, m), 8.42-8.41 (1H, m), 8.13 (1H, d, J=2.7 Hz), 7.99-7.96 (2H, m), 7.80-7.82 (1H, m), 7.58 (1H, t, J=2.3 Hz), 7.42-7.39 (2H, m), 5.42-5.31 (1H, m), 1.62-1.58 (12H, m); MS (ESI) m/z 488 (M+H)⁺, 486 (M−H)⁻.

### STEP 3. 4-{(1S)-1-[({5-chloro-2-[(5-chloropyridin-3-yl)oxy]pyridin-3-yl}carbonyl)amino]ethyl}benzoic acid

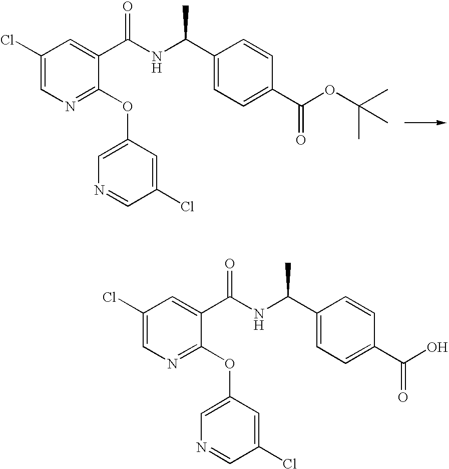

The title compound was prepared according to the procedure described in step 5 of Example 44 from tert-butyl 4-{(1S)-1-[({5-chloro-2-[(5-chloropyridin-3-yl)oxy]pyridin-3-yl}carbonyl)amino]ethyl}benzoate (step 2): ¹H-NMR (DMSO-d₆) [symbol] 9.04 (1H, d, J=7.8 Hz), 8.55 (1H, J=2.3 Hz), 8.52 (1H, d, J=2.3 Hz), 8.34 (1H, d, J=2.6 Hz), 8.19 (1H, d, J=2.6 Hz), 7.98 (1H, t, J=2.3 Hz), 7.89-7.86 (2H, m), 7.53-7.50 (2H, m), 5.21-5.16 (1H, m), 1.46 (3H, d, J=6.8 Hz), a peak of COOH was not observed; MS (ESI) m/z 432 (M+H)⁺, 430 (M−H)⁻.

### Example 47

### 4-[(1S)-1-({[5-CHLORO-2-(3-CYANOPHENOXY)PYRIDIN-3-YL]CARBONYL}AMINO)ETHYL]BENZOIC ACID

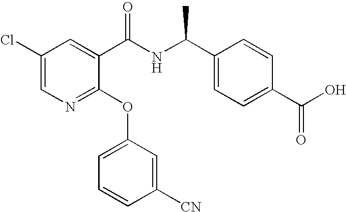

### STEP 1. 5-Chloro-2-(3-cyanophenoxy)nicotinic acid

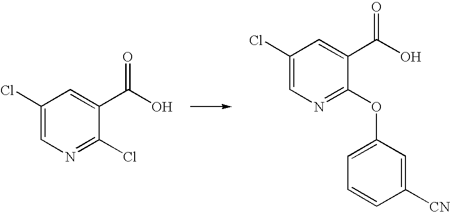

The title compound was prepared according to the procedure described in step 1 of Example 46 from 2,5-dichloronicotinic acid (Syn. Commun. 1989, 19, 553-9) and 3-hydroxybenzonitrile: ¹H-NMR (CDCl₃) δ 8.40-8.33 (2H, m), 7.74-7.52 (4H, m), a peak of COOH was not observed; MS (ESI) m/z 229 (M−COOH)⁻.

### STEP 2. tert-Butyl 4-[(1S)-1-({[5-chloro-2-(3-cyanophenoxy)pyridin-3-yl]carbonyl}amino)ethyl]benzoate

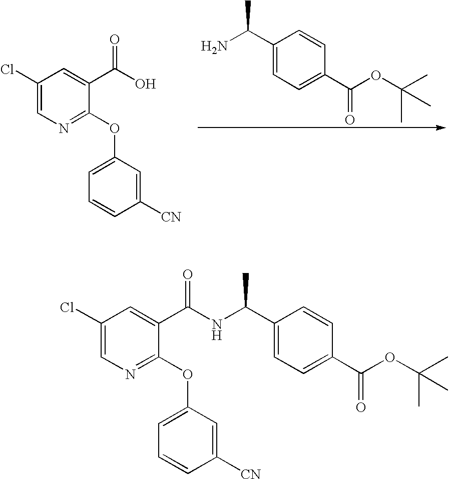

The title compound was prepared according to the procedure described in step 3 of Example 1 from 5-chloro-2-(3-cyanophenoxy)nicotinic acid (step 1) and tert-butyl 4-[(1S)-1-aminoethyl]benzoate (step 3 of Example 44): ¹H-NMR (CDCl₃) [symbol] 8.56 (1H, d, J=2.7 Hz), 8.13 (1H, d, J=2.7 Hz), 7.98-7.95 (2H, m), 7.89-7.86 (1H, m), 7.64-7.38 (6H, m), 5.42-5.31 (1H, m), 1.61-1.58 (12H, m); MS (ESI) m/z 476 (M−H)⁻.

### STEP 3. 4-[(1S)-1-({[5-chloro-2-(3-cyanophenoxy)pyridin-3-yl]carbonyl}amino)ethyl]benzoic acid

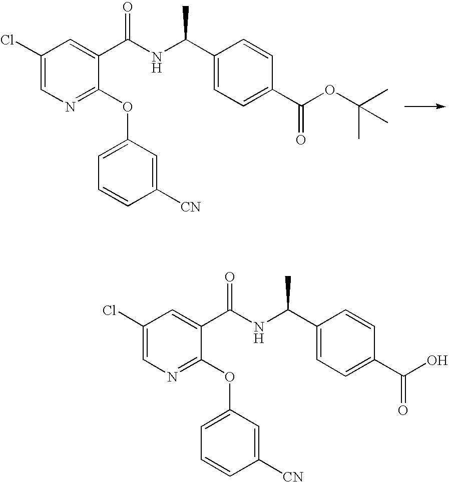

The title compound was prepared according to the procedure described in step 5 of Example 44 from tert-butyl 4-[(1S)-1-({[5-chloro-2-(3-cyanophenoxy)pyridin-3-yl]carbonyl}amino)ethyl]benzoate (step 2): ¹H-NMR (CDCl₃) [symbol] 8.56 (1H, d, J=2.7 Hz), 8.15 (1H, d, J=2.7 Hz), 8.09-8.06 (2H, m), 7.91 (1H, d, J=7.0 Hz), 7.64-7.39 (6H, m), 5.39 (1H, quint, J=7.0 Hz), 1.62 (3H, d, J=7.0 Hz), a peak of COOH was not observed; MS (ESI) m/z 422 (M+H)⁺, 420 (M−H)⁻.

### Example 48

### 4-[(1S)-1-({[5-CHLORO-2-(3-FLUOROPHENOXY)PYRIDIN-3-YL]CARBONYL}AMINO)ETHYL]BENZOIC ACID

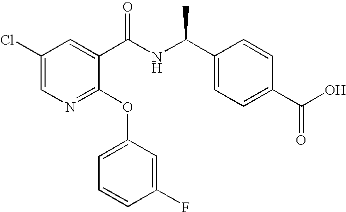

### STEP 1. tert-Butyl 4-[(1S)-1-({[5-chloro-2-(3-fluorophenoxy)pyridin-3-yl]carbonyl}amino)ethyl]benzoate

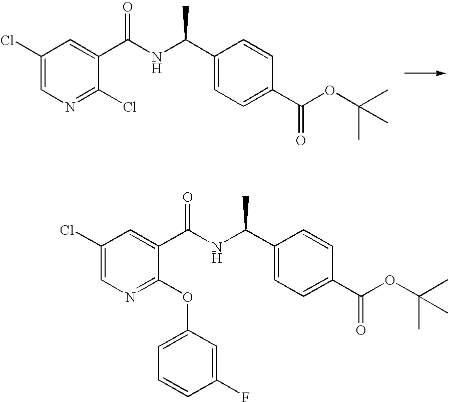

The title compound was prepared according to the procedure described in step 2 of Example 45 from tert-butyl 4-((1S)-1-{[(2,5-dichloropyridin-3-yl)carbonyl]amino}ethyl)benzoate (step 1 of Example 45) and 3-fluorophenol: H-NMR (CDCl₃) [symbol] 8.55 (1H, d, J=2.7 Hz), 8.15 (1H, d, J=2.7 Hz), 8.03-8.01 (1H, m), 7.97-7.94 (2H, m), 7.47-7.38 (3H, m), 7.07-7.00 (1H, m), 6.97-6.89 (2H, m), 5.41-5.31 (1H, m), 1.60-1.58 (12H, m); MS (ESI) m/z 471 (M+H)⁺, 469 (M−H)⁻.

### STEP 2. 4-[(1S)-1-({[5-chloro-2-(3-fluorophenoxy)pyridin-3-yl]carbonyl}amino)ethyl]benzoic acid

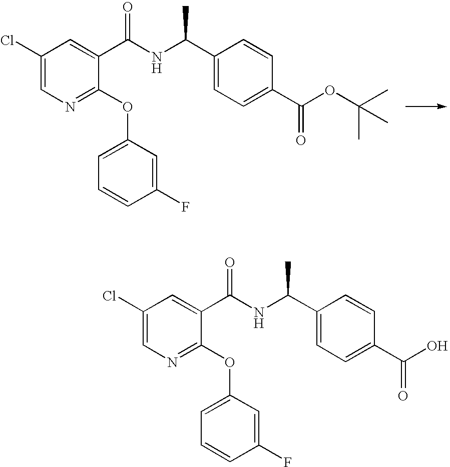

The title compound was prepared according to the procedure described in step 5 of Example 44 from tert-butyl 4-[(1S)-1-({[5-chloro-2-(3-fluorophenoxy)pyridin-3-yl]carbonyl}amino)ethyl]benzoate (step 1): ¹H-NMR (CDCl₃) [symbol] 8.56 (1H, d, J=3.0 Hz), 8.16 (1H, d, J=3.0 Hz), 8.08-8.05 (3H, m), 7.47-7.40 (3H, m), 7.07-6.91 (3H, m), 5.43-5.33 (1H, m), 1.60 (3H, d, J=7.1 Hz), a peak of COOH was not observed; MS (ESI) m/z 415 (M+H)⁺, 413 (M−H)⁻.

### Example 49

### 4-[(1S)-1-({[5-CHLORO-2-(3-CHLOROPHENOXY)PYRIDIN-3-YL]CARBONYL}AMINO)ETHYL]BENZOIC ACID

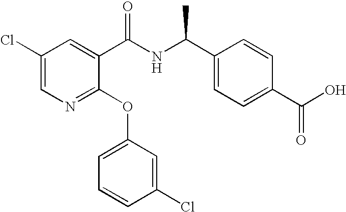

### STEP 1. tert-Butyl 4-[(1S)-1-({[5-chloro-2-(3-chlorophenoxy)pyridin-3-yl]carbonyl}amino)ethyl]benzoate

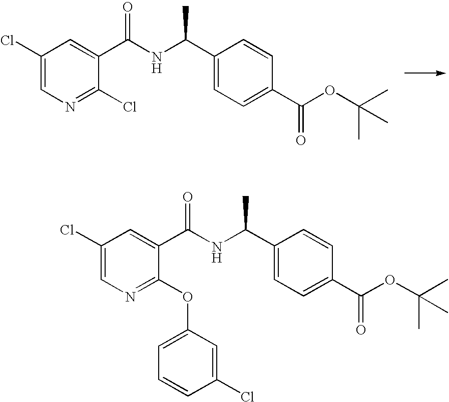

The title compound was prepared according to the procedure described in step 1 of Example 48 substituting 3-chlorophenol for 3-fluorophenol: ¹H-NMR (CDCl₃) δ 8.55 (1H, d, J=2.7 Hz), 8.15 (1H, d, J=2.7 Hz), 8.02-7.94 (3H, m), 7.43-7.28 (4H, m), 7.19-7.18 (1H, m), 7.07-7.04 (1H, m), 5.40-5.30 (1H, m), 1.60-1.58 (12H, m); MS (ESI) m/z 487 (M+H)⁺, 485 (M−H)⁻.

### STEP 2. 4-[(1S)-1-({[5-chloro-2-(3-chlorophenoxy)pyridin-3-yl]carbonyl}amino)ethyl]benzoic acid

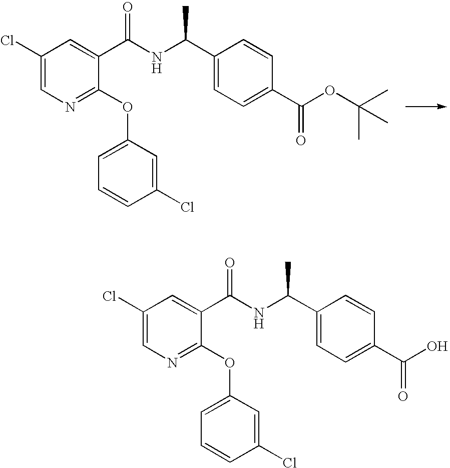

The title compound was prepared according to the procedure described in step 5 of Example 44 from tert-butyl 4-[(1S)-1-({[5-chloro-2-(3-chlorophenoxy)pyridin-3-yl]carbonyl}amino)ethyl]benzoate (step 1): ¹H-NMR (CDCl₃) [symbol] 8.56 (1H, d, J=2.7 Hz), 8.16 (1H, d, J=2.7 Hz), 8.08-8.03 (3H, m), 7.47-7.38 (3H, m), 7.33-7.30 (1H, m), 7.21-7.20 (1H, m), 7.08-7.05 (1H, m), 5.38 (1H, quint, J=7.0 Hz), 1.60 (3H, d, J=7.0 Hz), a peak of COOH was not observed; MS (ESI) m/z 431 (M+H)⁺, 429 (M−H)⁻.

### Example 50

### 4-[(1S)-1-({5-CHLORO-2-(3-METHOXYPHENOXY)PYRIDIN-3-YL]CARBONYL}AMINO)ETHYL]BENZOIC ACID

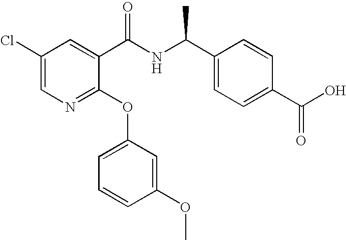

### STEP 1. tert-Butyl 4-[(1S)-1-({[5-chloro-2-(3-methoxyphenoxy)pyridin-3-yl]carbonyl}amino)ethyl]benzoate

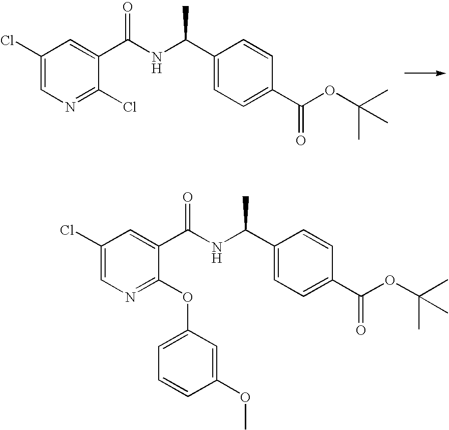

The title compound was prepared according to the procedure described in step 1 of Example 48 substituting 3-methoxyphenol for 3-fluorophenol: ¹H-NMR (CDCl₃) δ 8.55 (1H, d, J=2.7 Hz), 8.18-8.15 (2H, m), 7.96-7.94 (2H, m), 7.41-7.34 (3H, m), 6.88-6.84 (1H, m), 6.76-6.70 (2H, m), 5.40-5.31 (1H, m), 3.83 (3H, s), 1.69-1.57 (12H, m); MS (ESI) m/z 483 (M+H)⁺, 481 (M−H)⁻.

### STEP 2. 4-[(1S)-1-({[5-chloro-2-(3-methoxyphenoxy)pyridin-3-yl]carbonyl}amino)ethyl]benzoic acid

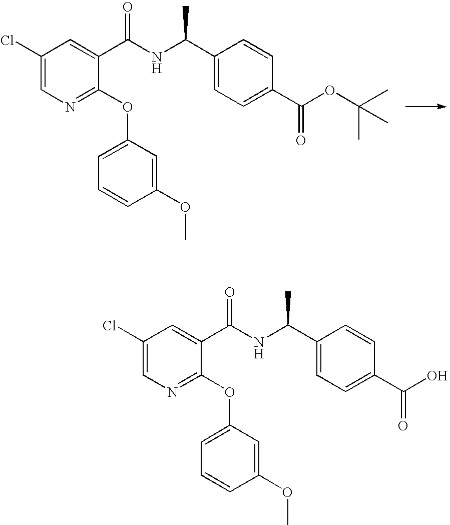

The title compound was prepared according to the procedure described in step 5 of Example 44 from tert-butyl 4-[(1S)-1-({[5-chloro-2-(3-methoxyphenoxy)pyridin-3-yl]carbonyl}amino)ethyl]benzoate (step 1): ¹H-NMR (CDCl₃) [symbol] 8.55 (1H, d, J=2.7 Hz), 8.20-8.16 (2H, m), 8.07-8.05 (2H, m), 7.47-7.44 (2H, m), 7.37 (1H, t, J=8.1 Hz), 6.88-6.85 (1H, m), 6.76-6.70 (2H, m), 5.43-5.33 (1H, m), 3.83 (3H, s), 1.59 (3H, d, J=7.0 Hz), a peak of COOH was not observed; MS (ESI) m/z 427 (M+H)⁺, 425 (M−H)⁻.

### Example 51

### 4-[(1S)-1-({[5-CHLORO-2-(2,4-DIFLUOROPHENOXY)PYRIDIN-3-YL]CARBONYL}AMINO)ETHYL]BENZOIC ACID

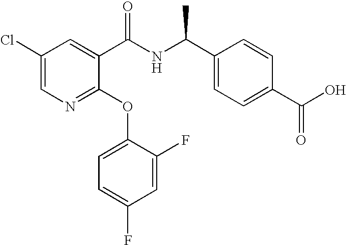

### STEP 1. tert-Butyl 4-[(1S)-1-({[5-chloro-2-(2,4-difluorophenoxy)pyridin-3-yl]carbonyl}amino)ethyl]benzoate

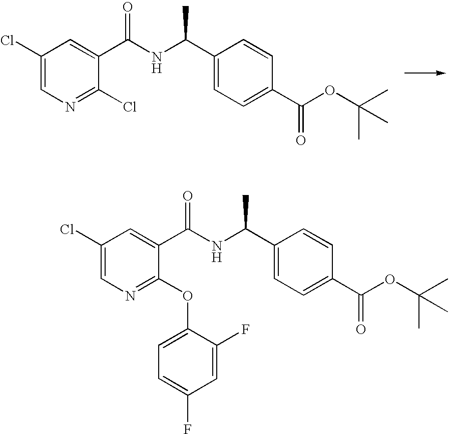

The title compound was prepared according to the procedure described in step 1 of Example 48 substituting 2,4-difluorophenol for 3-fluorophenol: ¹H-NMR (CDCl₃) [symbol] 8.54 (1H, d, J=2.7 Hz), 8.10 (1H, d, J=2.7 Hz), 8.01-7.94 (3H, m), 7.43-7.39 (2H, m), 7.34-7.25 (1H, m), 7.05-6.94 (2H, m), 5.42-5.32 (1H, m), 1.61-1.58 (12H, m); MS (ESI) m/z 489 (M+H)⁺, 487 (M−H)⁻.

### STEP 2. 4-[(1S)-1-({[5-chloro-2-(2,4-difluorophenoxy)pyridin-3-yl]carbonyl}amino)ethyl]benzoic acid

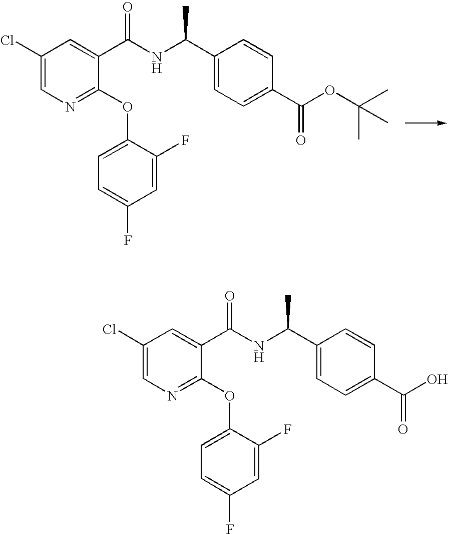

The title compound was prepared according to the procedure described in step 5 of Example 44 from tert-butyl 4-[(1S)-1-({[5-chloro-2-(2,4-difluorophenoxy)pyridin-3-yl]carbonyl}amino)ethyl]benzoate (step 1): ¹H-NMR (CDCl₃) δ 8.54 (1H, d, J=2.6 Hz), 8.11 (1H, d, J=2.6 Hz), 8.09-8.02 (3H, m), 7.49-7.46 (2H, m), 7.36-7.28 (1H, m), 7.06-6.94 (2H, m), 5.45-5.34 (1H, m), 1.62 (3H, d, J=7.0 Hz), a peak of COOH was not observed; MS (ESI) m/z 433 (M+H)⁺, 431 (M−H)⁻.

### Example 52

### 4-[(1S)-1-({[5-CHLORO-2-(4-CHLORO-3-FLUOROPHENOXY)PYRIDIN-3-YL]CARBONYL}AMINO)ETHYL]BENZOIC ACID

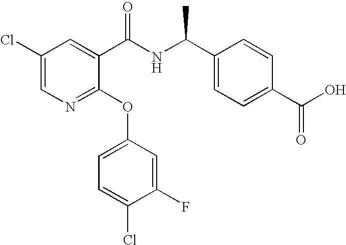

### STEP 1. tert-Butyl 4-[(1S)-1-({[5-chloro-2-(4-chloro-3-fluorophenoxy)pyridin-3-yl]carbonyl}amino)ethyl]benzoate

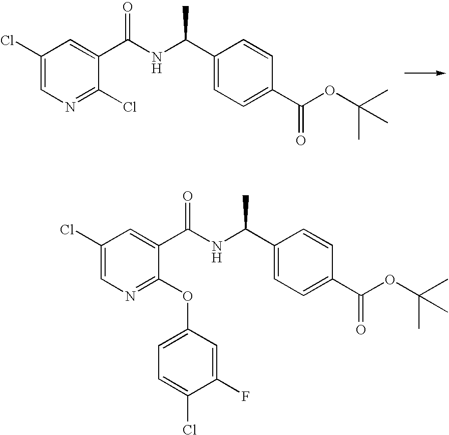

The title compound was prepared according to the procedure described in step 1 of Example 48 substituting 4-chloro-3-fluorophenol for 3-fluorophenol: ¹H-NMR (CDCl₃) [symbol] 8.55 (1H, d, J=3.0 Hz), 8.14 (1H, d, J=3.0 Hz), 7.98-7.94 (2H, m), 7.91-7.89 (1H, m), 7.51-7.45 (1H, m), 7.41-7.38 (2H, m), 7.04-7.00 (1H, m), 6.94-6.89 (1H, m), 5.41-5.30 (1H, m), 1.60-1.58 (12H, m); MS (ESI) m/z 505 (M+H)⁺, 503 (M−H)⁻.

### STEP 2. 4-[(1S)-1-({[5-chloro-2-(4-chloro-3-fluorophenoxy)pyridin-3-yl]carbonyl}amino)ethyl]benzoic acid

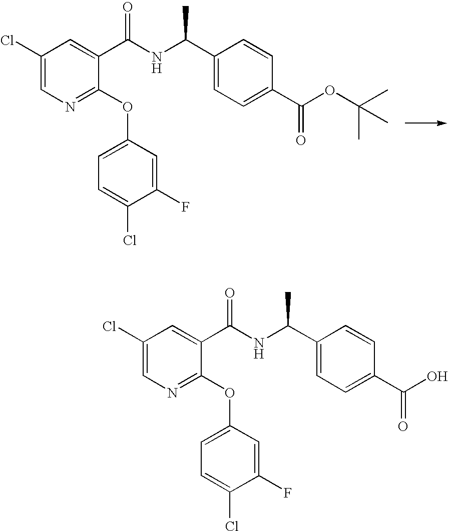

The title compound was prepared according to the procedure described in step 5 of Example 44 from tert-butyl 4-[(1S)-1-({[5-chloro-2-(4-chloro-3-fluorophenoxy)pyridin-3-yl]carbonyl}amino)ethyl]benzoate (step 1): ¹H-NMR (CDCl₃) [symbol] 8.56 (1H, d, J=2.7 Hz), 8.15 (1H, d, J=2.7 Hz), 8.09-8.06 (2H, m), 7.94-7.92 (1H, m), 7.52-7.44 (3H, m), 7.04 (1H, dd, J=9.2, 2.7 Hz), 6.93 (1H, ddd, J=8.6, 2.7, 1.4 Hz), 5.43-5.35 (1H, m), 1.61 (3H, d, J=7.0 Hz), a peak of COOH was not observed; MS (ESI) m/z 449 (M+H)⁺, 447 (M−H)⁻.

### Example 53

### 4-[(1S)-1-({[5-CHLORO-2-(2-CHLORO-4-FLUOROPHENOXY)PYRIDIN-3-YL]CARBONYL}AMINO)ETHYL]BENZOIC ACID

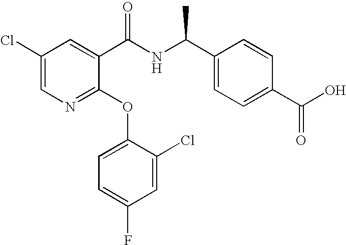

### STEP 1. tert-Butyl 4-[(1S)-1-({[5-chloro-2-(2-chloro-4-fluorophenoxy)pyridin-3-yl]carbonyl}amino)ethyl]benzoate

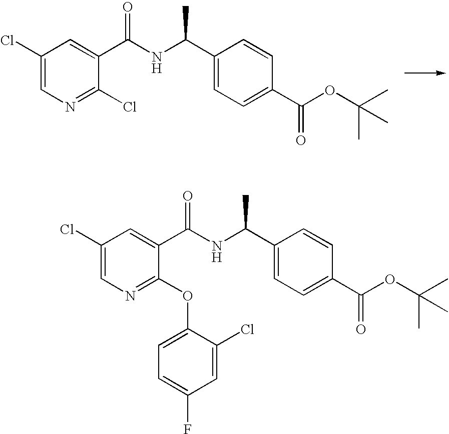

The title compound was prepared according to the procedure described in step 1 of Example 48 substituting 2-chloro-4-fluorophenol for 3-fluorophenol: ¹H-NMR (CDCl₃) δ 8.54 (1H, d, J=3.0 Hz), 8.11-8.08 (2H, m), 7.98-7.93 (2H, m), 7.43-7.40 (2H, m), 7.31-7.25 (2H, m), 7.15-7.07 (1H, m), 5.44-5.32 (1H, m), 1.61-1.58 (12H, m); MS (ESI) m/z 505 (M+H)⁺, 503 (M−H)⁻.

### STEP 2. 4-[(1S)-1-({[5-chloro-2-(2-chloro-4-fluorophenoxy)pyridin-3-yl]carbonyl}amino)ethyl]benzoic acid

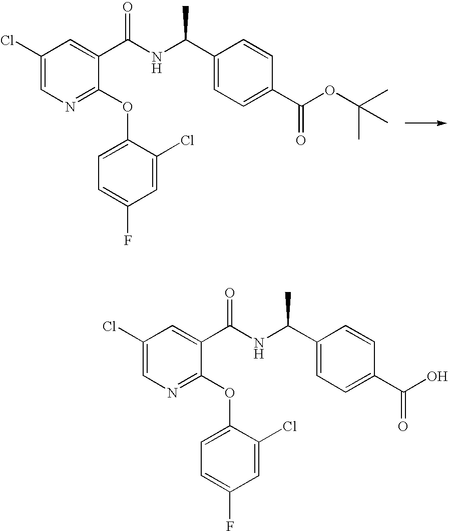

The title compound was prepared according to the procedure described in step 5 of Example 44 from tert-butyl 4-[(1S)-1-({[5-chloro-2-(2-chloro-4-fluorophenoxy)pyridin-3-yl]carbonyl}amino)ethyl]benzoate (step 1): ¹H-NMR (CDCl₃) [symbol] 8.55 (1H, d, J=2.6 Hz), 8.13-8.05 (4H, m), 7.49-7.46 (2H, m), 7.34-7.26 (2H, m), 7.15-7.08 (1H, m), 5.45-5.35 (1H, m), 1.62 (3H, d, J=7.0 Hz), a peak of COOH was not observed; MS (ESI) m/z 449 (M+H)⁺, 447 (M−H)⁻.

### Example 54

### 4-[(1S)-1-({[5-CHLORO-2-(2,6-DIFLUOROPHENOXY)PYRIDIN-3-YL]CARBONYL}AMINO)ETHYL]BENZOIC ACID

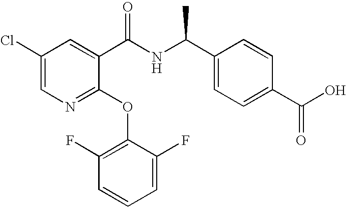

### STEP 1. tert-Butyl 4-[(1S)-1-({[5-chloro-2-(2,6-difluorophenoxy)pyridin-3-yl]carbonyl}amino)ethyl]benzoate

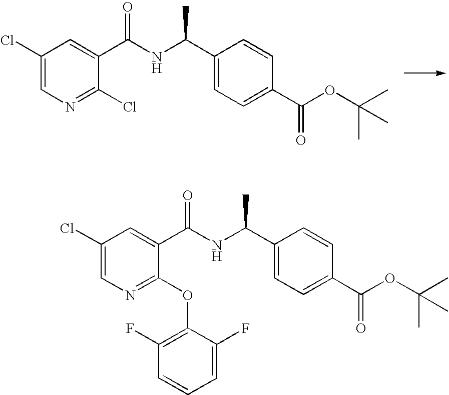

The title compound was prepared according to the procedure described in step 1 of Example 48 substituting 2,6-difluorophenol for 3-fluorophenol: ¹H-NMR (CDCl₃) [symbol] 8.54 (1H, d, J=2.7 Hz), 8.10 (1H, d, J=2.7 Hz), 7.98-7.95 (3H, m), 7.44-7.41 (2H, m), 7.33-7.23 (1H, m), 7.12-7.05 (2H, m), 5.43-5.32 (1H, m), 1.61-1.58 (12H, m); MS (ESI) m/z 489 (M+H)⁺, 487 (M−H)⁻.

### STEP 2. 4-[(1S)-1-({[5-chloro-2-(2,6-difluorophenoxy)pyridin-3-yl]carbonyl}amino)ethyl]benzoic acid

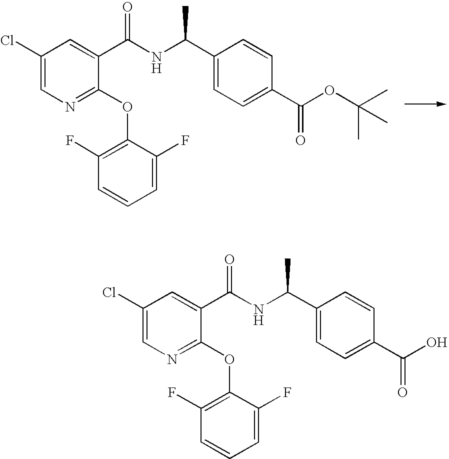

The title compound was prepared according to the procedure described in step 5 of Example 44 from tert-butyl 4-[(1S)-1-({[5-chloro-2-(2,6-difluorophenoxy)pyridin-3-yl]carbonyl}amino)ethyl]benzoate (step 1): ¹H-NMR (CDCl₃) δ 8.54 (1H, d, J=2.6 Hz), 8.13-8.06 (3H, m), 8.00-7.91 (1H, m), 7.50-7.47 (2H, m), 7.34-7.23 (1H, m), 7.12-7.05 (2H, m), 5.45-5.35 (1H, m), 1.62 (3H, d, J=7.0 Hz), a peak of COOH was not observed; MS (ESI) m/z 433 (M+H)⁺, 431 (M−H)⁻.

### Example 55

### 4-[(1S)-1-({[5-CHLORO-2-(3,4-DIFLUOROPHENOXY)PYRIDIN-3-YL]CARBONYL}AMINO)ETHYL]BENZOIC ACID

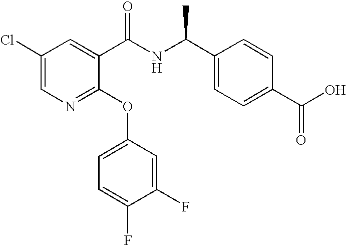

### STEP 1. tert-Butyl 4-[(1S)-1-({[5-chloro-2-(3,4-difluorophenoxy)pyridin-3-yl]carbonyl}amino)ethyl]benzoate

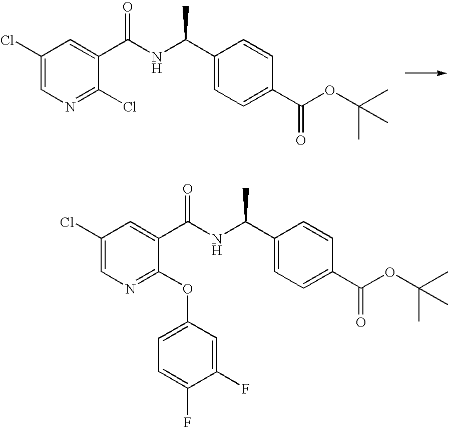

The title compound was prepared according to the procedure described in step 1 of Example 48 substituting 3,4-difluorophenol for 3-fluorophenol: ¹H-NMR (CDCl₃) δ 8.54 (1H, d, J=2.6 Hz), 8.13 (1H, d, J=2.6 Hz), 8.00-7.91 (3H, m), 7.41-7.38 (2H, m), 7.30-7.20 (1H, m), 7.08-7.00 (1H, m), 6.93-6.86 (1H, m), 5.36 (1H, quint, J=7.0 Hz), 1.61-1.58 (12H, m); MS (ESI) m/z 489 (M+H)⁺, 487 (M−H)⁻.

### STEP 2. 4-[(1S)-1-({[5-chloro-2-(3,4-difluorophenoxy)pyridin-3-yl]carbonyl}amino)ethyl]benzoic acid

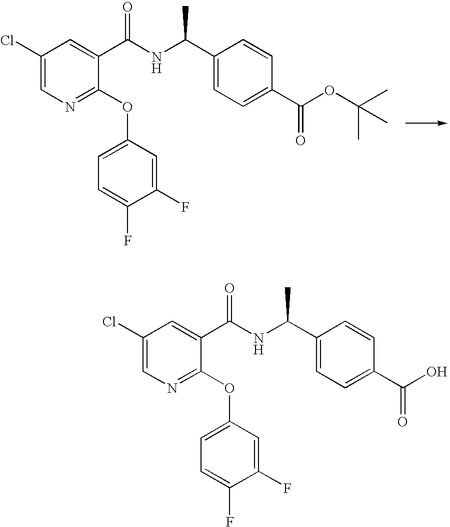

The title compound was prepared according to the procedure described in step 5 of Example 44 from tert-butyl 4-[(1S)-1-({[5-chloro-2-(3,4-difluorophenoxy)pyridin-3-yl]carbonyl}amino)ethyl]benzoate (step 1): ¹H-NMR (CDCl₃) δ 8.55 (1H, d, J=2.7 Hz), 8.14 (1H, d, J=2.7 Hz), 8.09-8.06 (2H, m), 7.99-7.96 (1H, m), 7.47-7.44 (2H, m), 7.31-7.21 (1H, m), 7.09-7.02 (1H, m), 6.94-6.88 (1H, m), 5.44-5.34 (1H, m), 1.61 (3H, d, J=7.0 Hz), a peak of COOH was not observed; MS (ESI) m/z 433 (M+H)⁺, 431 (M−H)⁻.

### Example 56

### 5. 4-[({[2-(4-FLUOROPHENOXY)-5-(TRIFLUOROMETHYL)PYRIDIN-3-YL]CARBONYL}AMINO)METHYL]BENZOIC ACID

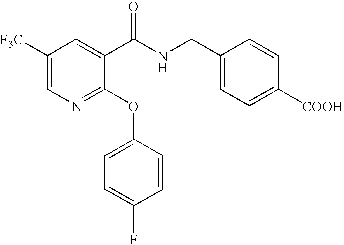

### STEP 1. methyl 2-(4-fluorophenoxy)-5-iodonicotinate

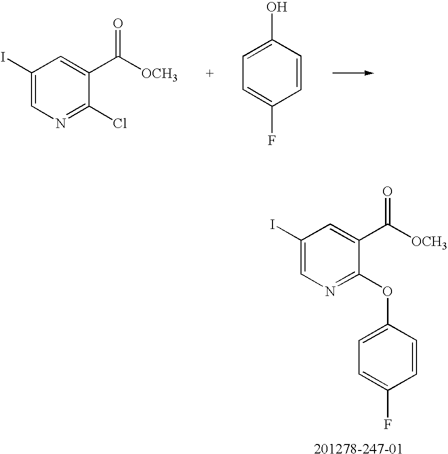

To a solution of 4-fluorophenol (224 mg, 2.0 mmol) in DMF (5.0 mL) was added sodium hydride (48 mg, 2.0 mmol) at room temperature. After stirring for 10 min, methyl 2-chloro-5-iodonicotinate (J. Org. Chem. 1989, 54, 3618-3624, 594 mg, 2.0 mmol) was added to the reaction mixture. The reaction mixture was stirred under reflux for 16 h. Then the reaction mixture was poured into water (50 mL) and extracted with ether (50 mL×3). The combined organic extracts were washed with brine (50 mL) and dried (sodium sulfate). After removal of the solvent, the residue was purified by flash column chromatography on silica gel (50 g) eluting with hexane/ethyl acetate (2/1) to afford 644 mg (86%) of the title compound: ¹H-NMR (CDCl₃) [symbol] 8.51 (1H, d, J=2.3 Hz), 8.41 (1H, s), 7.09 (4H, d, J=6.2 Hz), 3.95 (3H, s); MS (ESI) m/z 374 (M+H)⁺.

### STEP 2. methyl 2-(4-fluorophenoxy)-5-(trifluoromethyl)nicotinate

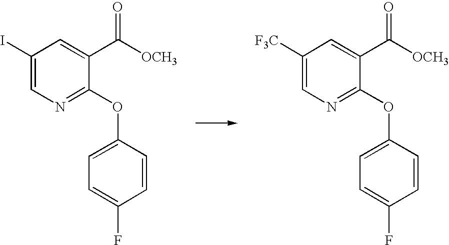

A mixture of methyl 2-(4-fluorophenoxy)-5-iodonicotinate (step 1, 373 mg, 1.0 mmol), sodium trifluoroacetate (1.36 g, 10 mmol), and copper(I) iodide (960 mg, 5.0 mmol) in 1-methyl-pyrrolidine (8.0 mL) was stirred at 160° C. for 16 h under nitrogen atmosphere. The reaction mixture was poured into water (20 mL) and extracted with dichloromethane (50 mL×3). The combined organic extracts were washed with brine (50 mL) and dried (sodium sulfate). After removal of the solvent, the residue was purified by TLC plate developing with hexane/ethyl acetate (1/1) to afford 32 mg (10%) of the title compound: ¹H-NMR (CDCl₃) δ 8.5 (2H, s), 7.13 (1H, d, J=6.3 Hz), 3.99 (3H, s); MS (ESI) m/z 316 (M+H)⁺.

### STEP 3. 2-(4-fluorophenoxy)-5-(trifluoromethyl)nicotinic acid

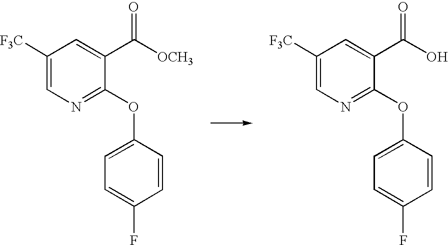

A mixture of methyl 2-(4-fluorophenoxy)-5-(trifluoromethyl)nicotinate (step 2, 32 mg, 0.10 mmol) and 4.0 M lithium hydroxide aqueous solution (1.0 mL, 4.0 mmol) in a mixture of tetrahydrofuran (2 mL) and dioxane (10 mL) was stirred for 3 h at room temperature. The pH value was adjusted to 4.0 by the addition of 2 M hydrochloric acid. The mixture was diluted with water (100 mL) and extracted with dichloromethane (20 mL×3). The combined organic extracts were washed with brine (50 mL), dried (sodium sulfate), and concentrated to afford 29 mg (99%) of the title compound: MS (ESI) m/z 256 (M−45)⁻.

### STEP 4. methyl 4-[({[2-(4-fluorophenoxy)-5-(trifluoromethyl)pyridin-3-yl]carbonyl}amino)methyl]benzoate

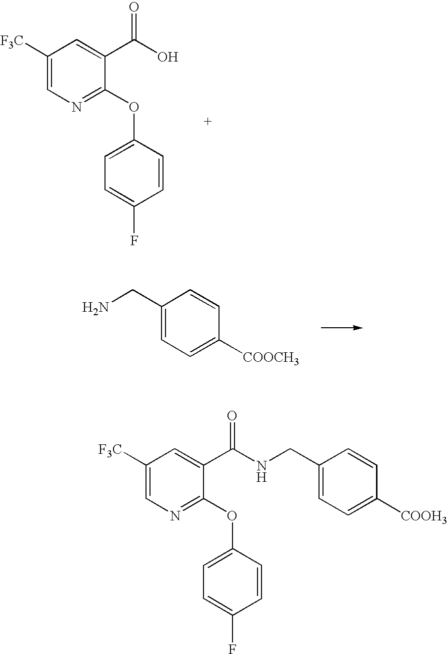

The title compound was prepared according to the procedure described in step 3 of Example 1 from 2-(4-fluorophenoxy)-5-(trifluoromethyl)nicotinic acid (step 3) and methyl 4-(aminomethyl)benzoate hydrochloride: ¹H-NMR (CDCl₃) [symbol] 8.90 (1H, d, J=2.7 Hz), 8.45 (1H, s), 8.01 (2H, d, J=8.2 Hz), 7.43 (2H, d, J=8.2 Hz), 4.78 (2H, d, J=5.9 Hz), 3.90 (3H, s); MS (ESI) m/z 449 (M+H)⁺.

### STEP 5. 4-[({[2-(4-fluorophenoxy)-5-(trifluoromethyl)pyridin-3-yl]carbonyl}amino)methyl]benzoic acid

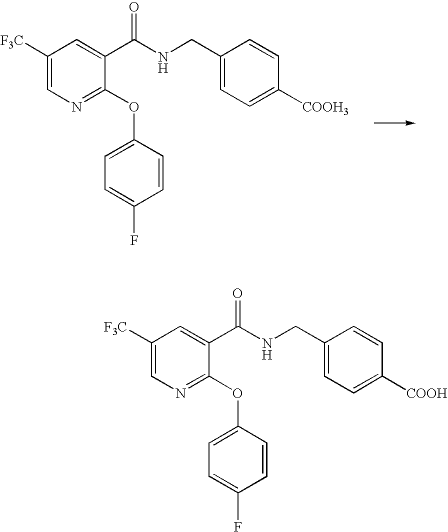

The title compound was prepared according to the procedure described in step 2 of Example 56 from methyl 4-[({[2-(4-fluorophenoxy)-5-(trifluoromethyl)pyridin-3-yl]carbonyl}amino)methyl]benzoate: ¹H-NMR (CDCl₃) [symbol] 8.90 (1H, d, J=2.1 Hz), 8.46 (1H, s), 8.22 (2H, br.s), 7.45 (2H, br.s), 7.23-7.10 (4H, m), 4.80 (2H, d, J=6.8 Hz); MS (ESI) m/z 435 (M+H)⁺, 433 (M−H)⁺.

### Example 57

### 4-[({[5-CYANO-2-(4-FLUOROPHENOXY)PYRIDIN-3-YL]CARBONYL}AMINO)METHYL]BENZOIC ACID

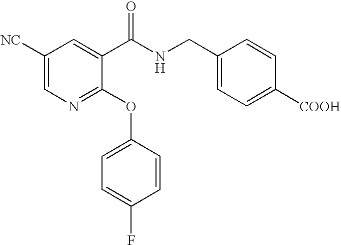

### STEP 1. methyl 5-bromo-2-(4-fluorophenoxy)nicotinate

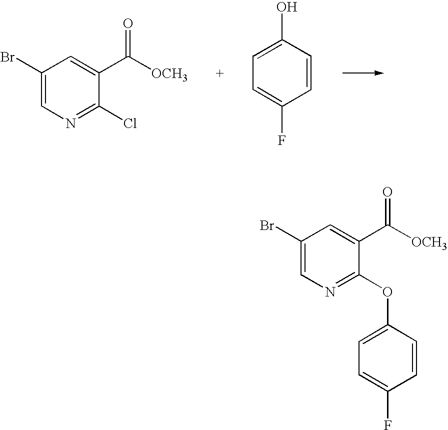

The title compound was prepared according to the procedure described in step 1 of Example 56 from methyl 5-bromo-2-chloronicotinate (J. Org. Chem. 1989, 54, 3618-3624) and 4-fluorophenol: ¹H-NMR (CDCl₃) δ 8.37 (1H, d, J=2.5 Hz), 8.27 (1H, d, J=2.5 Hz), 7.10 (2H, d, J=6.2 Hz), 3.95 (3H, s); MS (ESI) m/z 326 (M+H)⁺.

### STEP 2. methyl 5-cyano-2-(4-fluorophenoxy)nicotinate

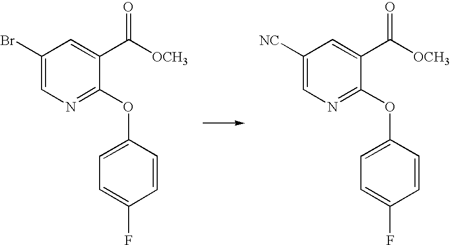

A mixture of methyl 5-bromo-2-(4-fluorophenoxy)nicotinate (step 1, 163 mg, 0.50 mmol), sodium cyanide (49 mg, 1.0 mmol), tetrakis(triphenylphohphine) palladium(0) (29 mg, 0.025 mmol), and copper iodide (9.5 mg, 0.05 mmol) in propionitrile (4.0 mL) was heated under reflux for 4.5 h with stirring. The reaction mixture was filtered through a pad of celite. The filtrate was partitioned between water (10 mL) and dichloromethane (30 mL). The organic phase was separated, dried (sodium sulfate), and concentrated. The residue was purified by TLC plate developing with hexane/ethyl acetate (3/1) to afford 97 mg (71%) of the title compound: ¹H-NMR (CDCl₃) δ 8.5 (2H, m), 7.14 (2H, d, J=1.2 Hz), 7.12 (2H, s), 3.99 (3H, s); MS (ESI) m/z 272 (M⁺).

### STEP 3. 5-cyano-2-(4-fluorophenoxy)nicotinic acid

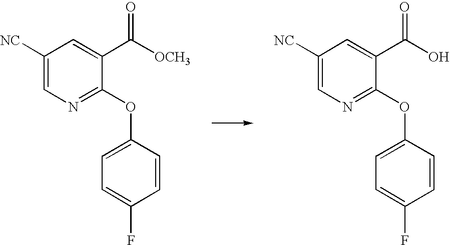

The title compound was prepared according to the procedure described in step 2 of Example 56 from methyl 5-cyano-2-(4-fluorophenoxy)nicotinate (step 2): H-NMR (CDCl₃) [symbol] 8.66 (1H, d, J=1.8 Hz), 8.54 (1H, d, J=2.3 Hz), 7.15 (4H, d, J=6.3 Hz); MS (ESI) m/z 259 (M+H)⁺, 257 (M−H)⁺.

### STEP 4. methyl 4-[({[5-cyano-2-(4-fluorophenoxy)pyridin-3-yl]carbonyl}amino)methyl]benzoate

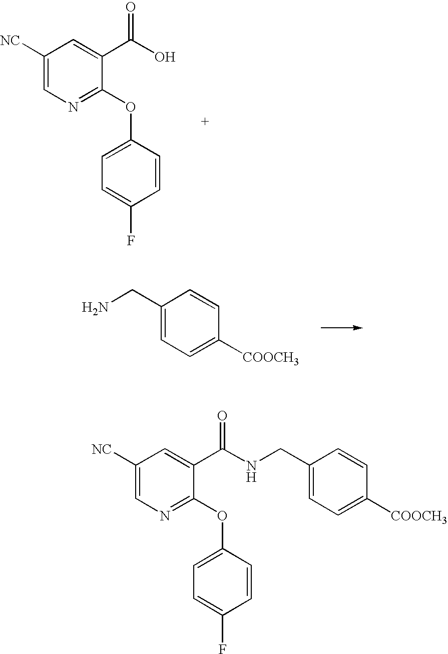

The title compound was prepared according to the procedure described in step 3 of Example 1 from 5-cyano-2-(4-fluorophenoxy)nicotinic acid (step 3) and methyl 4-(aminomethyl)benzoate hydrochloride: ¹H-NMR (CDCl₃) [symbol] 8.91 (1H, d, J=1.8 Hz), 8.48 (1H, d, J=1.8 Hz), 8.03 (2H, d, J=8.1 Hz), 7.43 (2H, d, J=8.1 Hz), 7.17-7.12 (4H, m), 4.78 (2H, d, J=5.4 Hz), 3.91 (3H, s); MS (ESI) m/z 406 (M+H) 404 (M+H)⁺.

### STEP 5. 4-[({[5-cyano-2-(4-fluorophenoxy)pyridin-3-yl]carbonyl}amino)methyl]benzoic acid

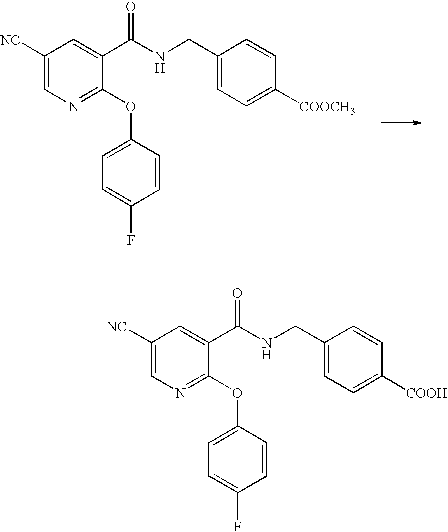

The title compound was prepared according to the procedure described in step 2 of Example 56 from methyl 4-[({[5-cyano-2-(4-fluorophenoxy)pyridin-3-yl]carbonyl}amino)methyl]benzoate: ¹H-NMR (DMSO-d₆) □δ8.87 (1H, d, J=2.3 Hz), 8.46 (1H, d, J=2.3 Hz), 8.02 (2H, d, J=8.2 Hz), 7.41 (2H, d, J=8.2 Hz), 7.15-7.10 (4H, m), 4.76 (2H, d, J=5.7 Hz); MS (ESI) m/z 392 (M+H)⁺, 390 (M−H)⁺.

### Example 58

### 4-[({[5-FLUORO-2-(4-FLUOROBENZYL)PYRIDIN-3-YL]CARBONYL}AMINO)METHYL]BENZOIC ACID

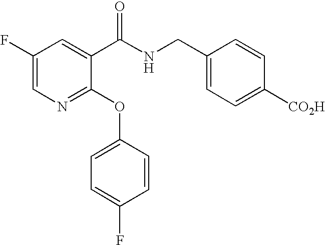

### STEP 1. Methyl 5-fluoro-2-(4-fluorobenzyl)nicotinate

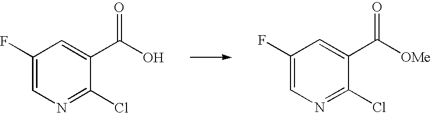

2-Chloro-5-fluoronicotinic acid (EP 634413, 1.00 g, 5.70 mmol) was treated with 2 M solution of (Trimethylsilyl)diazomethane in hexane (5.70 mL, 11.4 mmol), methanol (4 mL), and dichloromethane (14 mL) at 0° C. for 1 h. The mixture was quenched with acetic acid and concentrated under reduced pressure. The residue was purified by flash column chromatography on silica gel eluting with hexane/ethyl acetate (10/1) to afford 0.78 g (72%) of the title compounds as colorless oil: ¹H-NMR (CDCl₃) δ 8.41 (1H, d, J=3.1 Hz), 7.93 (1H, dd, J=7.6, 3.1 Hz), 3.98 (3H, s).

### STEP 2. Methyl 5-fluoro-2-(4-fluorobenzyl)nicotinate

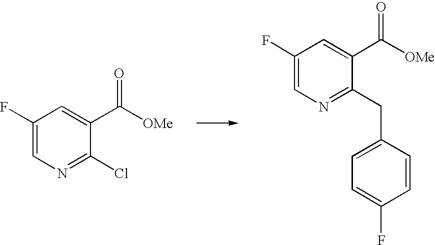

To a stirred solution of methyl 2-chloro-5-fluoronicotinate (step 1, 350 mg, 1.85 mmol) and dichlorobis[triphenylphosphine]nickel (II) (362 mg, 0.55 mmol) in tetrahydrofuran (15 mL) was added a 0.5 M solution of 4-fluorobenzylzinc chloride in tetrahydrofuran (5.54 mL, 2.77 mmol) at 0° C. under nitrogen. The resulting mixture was warmed to room temperature and stirred for 16 h. The mixture was poured into saturated aqueous ammonium chloride solution (50 mL) and extracted with ethyl acetate (100 mL). The organic layer was dried over magnesium sulfate and concentrated under reduced pressure. The residue was purified by flash column chromatography on silica gel eluting with hexane/ethyl acetate (10/1) to afford 439 mg (90%) of the title compounds as colorless oil: H-NMR (CDCl₃) δ 8.56 (1H, d, J=2.8 Hz), 7.91 (1H, dd, J=8.6, 2.9 Hz), 7.26-7.19 (2H, m), 6.98-6.92 (2H, m), 4.52 (2H, s), 3.89 (3H, s); MS (ESI) m/z 264 (M+H)⁺.

### STEP 3. 5-Fluoro-2-(4-fluorobenzyl)nicotinic acid

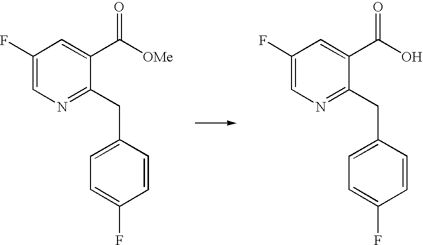

The title compound was prepared according to the procedure described in step 2 of Example 1 from methyl 5-fluoro-2-(4-fluorobenzyl)nicotinate (step 2): MS (ESI) m/z 250 (M+H)⁺.

### STEP 4. Methyl 4-[({[5-fluoro-2-(4-fluorobenzyl)pyridin-3-yl]carbonyl}amino)methyl]benzoate

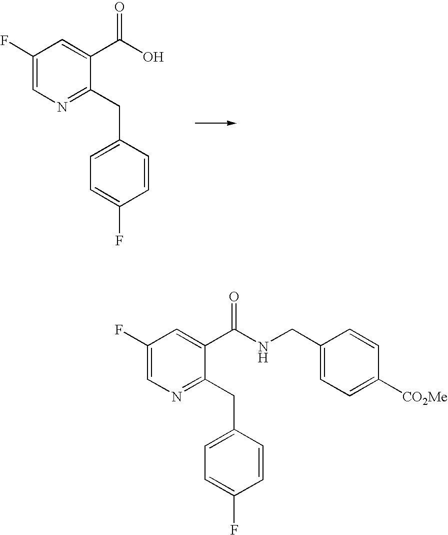

The title compound was prepared according to the procedure described in step 3 of Example 1 from 5-fluoro-2-(4-fluorobenzyl)nicotinic acid (step 3) and methyl 4-(aminomethyl)benzoate hydrochloride: ¹H-NMR (CDCl₃) δ 8.49 (1H, d, J=2.8 Hz), 8.01-7.97 (2H, m), 7.44 (1H, dd, J=7.9, 2.8 Hz), 7.27 (2H, dd, J=4.6, 4.0 Hz), 7.14-7.09 (2H, m), 6.93-6.86 (2H, m), 6.08 (1H, br.s), 4.56 (2H, d, J=5.9 Hz), 4.29 (2H, s), 3.92 (3H, s); MS (ESI) m/z 397 (M+H)⁺, 395 (M−H)⁻.

### STEP 5. 4-[({[5-Fluoro-2-(4-fluorobenzyl)pyridin-3-yl]carbonyl}amino)methyl]benzoic acid

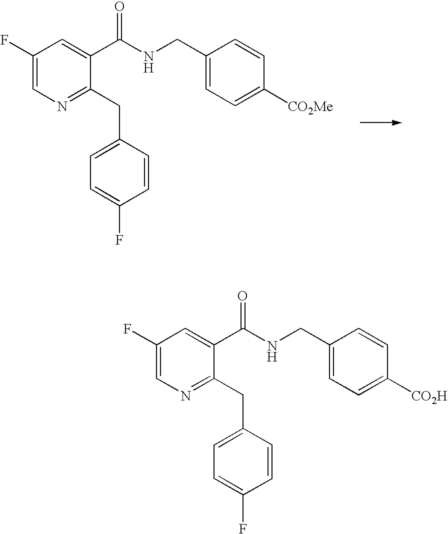

The title compound was prepared according to the procedure described in step 4 of Example 1 from methyl 4-[({[5-fluoro-2-(4-fluorobenzyl)pyridin-3-yl]carbonyl}amino)methyl]benzoate (step 4): ¹H-NMR (DMSO-d₆) δ 9.20 (1H, t, J=5.7 Hz), 8.60 (1H, d, J=2.8 Hz), 7.90 (2H, d, J=2.8 Hz), 7.82 (1H, dd, J=8.9, 2.8 Hz), 7.38 (2H, d, J=8.1 Hz), 7.18-7.14 (2H, m), 7.02 (2H, dd, J=8.8, 8.6 Hz), 4.50 (2H, d, J=5.7 Hz), 4.20 (2H, s); MS (ESI) m/z 383 (M+H)⁺, 381 (M−H)⁻.

### Example 59

### 4-[(1S)-1-({[5-FLUORO-2-(4-FLUOROBENZYL)PYRIDIN-3-YL]CARBONYL}AMINO)ETHYL]BENZOIC ACID

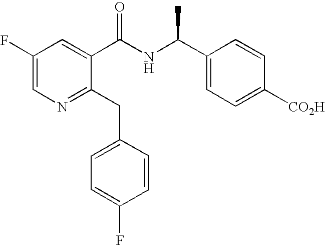

### STEP 1. Methyl 4-[(1S)-1-({[5-fluoro-2-(4-fluorobenzyl)pyridin-3-yl]carbonyl}amino)ethyl]benzoate

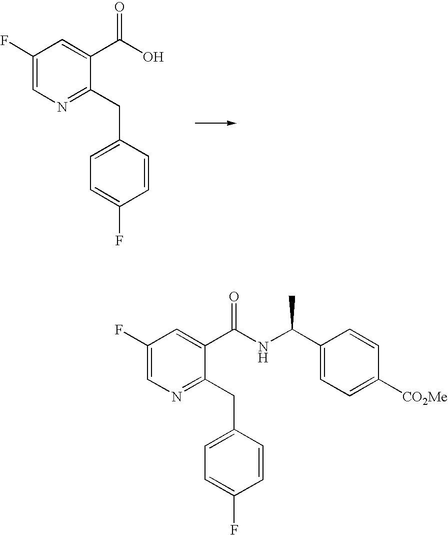

The title compound was prepared according to the procedure described in step 3 of Example 1 from 5-fluoro-2-(4-fluorobenzyl)nicotinic acid (step 3 of Example 58) and methyl 4-[(1S)-1-aminoethyl]benzoate hydrochloride: ¹H-NMR (CDCl₃) δ 9.13 (1H, d, J=7.7 Hz), 8.59 (1H, d, J=3.0 Hz), 7.93 (2H, d, J=8.4 Hz), 7.79 (1H, dd, J=8.7, 2.8 Hz), 7.51 (2H, d, J=8.2 Hz), 7.15-7.09 (2H, m), 7.03-6.96 (2H, m), 5.19-5.09 (1H, m), 4.17 (1H, d, J=13.7 Hz), 4.08 (1H, d, J=13.7 Hz), 3.85 (3H, s), 1.42 (3H, d, J=7.1 Hz); MS (ESI) m/z 411 (M+H)⁺, 409 (M−H)⁻.

### STEP 2. 4-[(1S)-1-({[5-Fluoro-2-(4-fluorobenzyl)pyridin-3-yl]carbonyl}amino)ethyl]benzoic acid

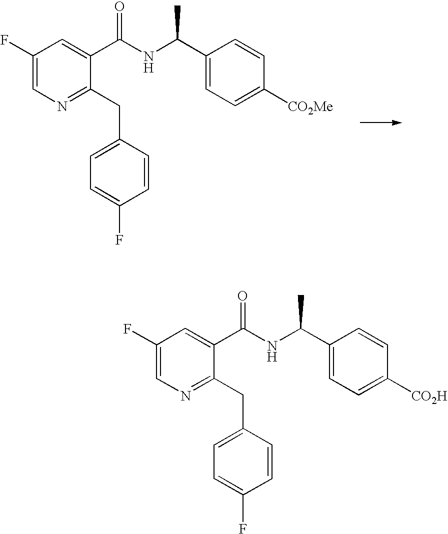

The title compound was prepared according to the procedure described in step 4 of Example 1 from methyl 4-[(1S)-1-({[5-fluoro-2-(4-fluorobenzyl)pyridin-3-yl]carbonyl}amino)ethyl]benzoate (step 1): ¹H-NMR (DMSO-d₆) [symbol] 9.11 (1H, d, J=7.9 Hz), 8.59 (1H, d, J=3.0 Hz), 7.91 (2H, d, J=8.2 Hz), 7.79 (1H, dd, J=8.8, 2.9 Hz), 7.48 (2H, d, J=8.4 Hz), 7.15-6.96 (4H, m), 5.20-5.10 (1H, m), 4.18 (1H, d, J=13.9 Hz), 4.09 (1H, d, J=13.9 Hz), 1.42 (3H, d, J=7.1 Hz); MS (ESI) m/z 397 (M+H)⁺, 395 (M−H)⁻.

### Example 60

### 4-[(1S)-1-({[5-CHLORO-2-(4-FLUOROBENZYL)PYRIDIN-3-YL]CARBONYL}AMINO)ETHYL]BENZOIC ACID

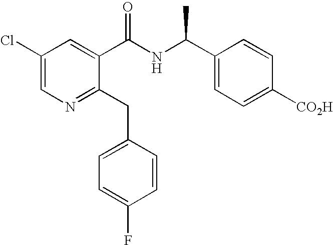

### STEP 1. Methyl 5-chloro-2-(4-fluorobenzyl)nicotinate

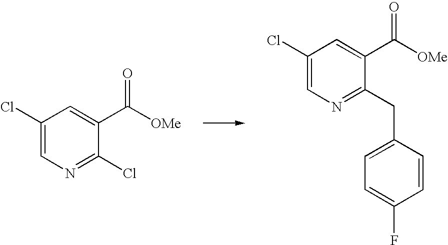

To a stirred solution of methyl 2,5-dichloronicotinate (Journal of Chemical and Engineering Data 1981, 26, 332, 350 mg, 1.70 mmol) and tetrakis(triphenylphosphine)palladium (0) (196 mg, 0.17 mmol) in tetrahydrofuran (6 mL) was added a 0.5 M solution of 4-fluorobenzylzinc chloride in tetrahydrofuran (4.08 mL, 2.04 mmol) at 0° C. under nitrogen. The resulting mixture was heated at 60° C. for 16 h. The mixture was poured into water (50 mL) and extracted with ethyl acetate (100 mL). The organic layer was dried over magnesium sulfate and concentrated under reduced pressure. The residue was purified by flash column chromatography on silica gel eluting with hexane/ethyl acetate (6/1) to afford 416 mg (88%) of the title compounds as colorless oil: H-NMR (CDCl₃) [symbol] 8.64 (1H, d, J=2.6 Hz), 8.17 (1H, d, J=2.6 Hz), 7.24-7.20 (2H, m), 6.96-6.90 (2H, m), 4.50 (2H, s), 3.89 (3H, s).

### STEP 2. 5-Chloro-2-(4-fluorobenzyl)nicotinic acid

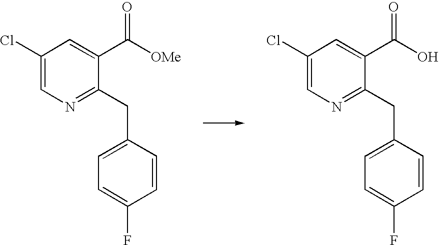

The title compound was prepared according to the procedure described in step 2 of Example 1 from methyl 5-chloro-2-(4-fluorobenzyl)nicotinate (step 1): ¹H-NMR (DMSO-d₆) [symbol] 8.73 (1H, d, J=2.5 Hz), 8.22 (1H, d, J=2.5 Hz), 7.24-7.04 (4H, m), 3.35 (2H, s{tilde over ())}

### STEP 3. Methyl 4-[(1S)-1-({[5-chloro-2-(4-fluorobenzyl)pyridin-3-yl]carbonyl}amino)ethyl]benzoate

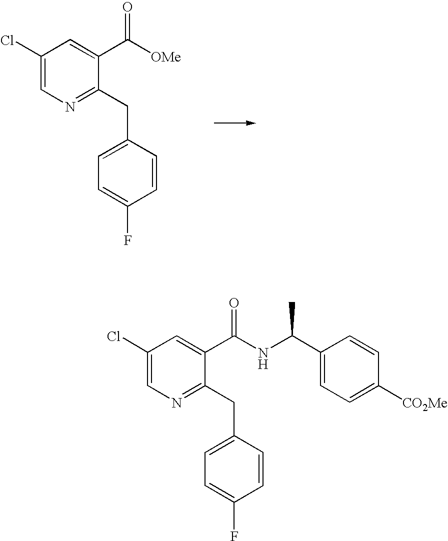

The title compound was prepared according to the procedure described in step 3 of Example 1 from 5-chloro-2-(4-fluorobenzyl)nicotinic acid (step 2) and methyl 4-[(1S)-1-aminoethyl]benzoate hydrochloride: ¹H-NMR (CDCl₃) δ 9.15 (1H, d, J=7.7 Hz), 8.63 (1H, d, J=2.4 Hz), 7.96-7.92 (3H, m), 7.50 (2H, d, J=8.4 Hz), 7.15-6.96 (4H, m), 5.19-5.09 (1H, m), 4.17 (1H, d, J=14.0 Hz), 4.08 (1H, d, J=14.0 Hz), 3.85 (3H, s), 1.42 (2H, d, J=7.0 Hz); MS (ESI) m/z 427 (M+H)⁺, 425 (M−H)⁻.

### STEP 4. 4-[(1S)-1-({[5-Chloro-2-(4-fluorobenzyl)pyridin-3-yl]carbonyl}amino)ethyl]benzoic acid

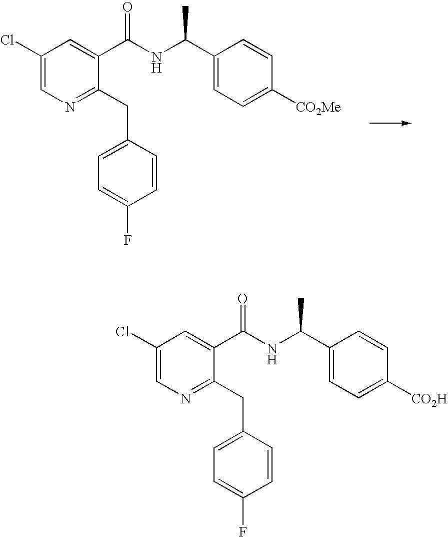

The title compound was prepared according to the procedure described in step 4 of Example 1 from methyl 4-[(1S)-1-({[5-chloro-2-(4-fluorobenzyl)pyridin-3-yl]carbonyl}amino)ethyl]benzoate (step 3): ¹H-NMR (DMSO-d₆) δ 9.13 (1H, d, J=7.6 Hz), 8.63 (1H, d, J=2.5 Hz), 7.95-7.90 (3H, m), 7.47 (2H, d, J=8.2 Hz), 7.15-6.96 (4H, m), 5.19-5.09 (1H, m), 4.18 (1H, d, J=14.0 Hz), 4.09 (1H, d, J=14.0 Hz), 1.42 (3H, d, J=6.9 Hz); MS (ESI) m/z 413 (M+H)⁺, 411 (M−H)⁻.

### Example 61

### 4-[(1S)-1-({[5-CHLORO-2-(3-FLUOROBENZYL)PYRIDIN-3-YL]CARBONYL}AMINO)ETHYL]BENZOIC ACID

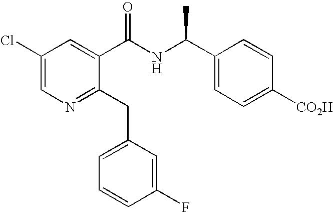

### STEP 1. Methyl 5-chloro-2-(3-fluorobenzyl)nicotinate

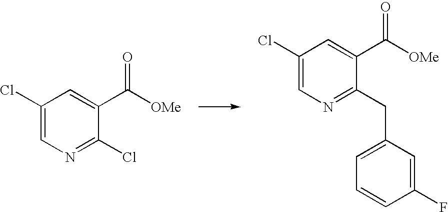

The title compound was prepared according to the procedure described in step 2 of Example 58 from methyl 2,5-dichloronicotinate and 3-fluorobenzylzinc chloride: ¹H-NMR (CDCl₃) δ 8.65 (1H, d, J=2.4 Hz), 8.19 (1H, d, J=2.6 Hz), 7.26-6.84 (4H, m), 4.54 (2H, s), 3.89 (3H, s{tilde over ())}

### STEP 2. 5-Chloro-2-(3-fluorobenzyl)nicotinic acid

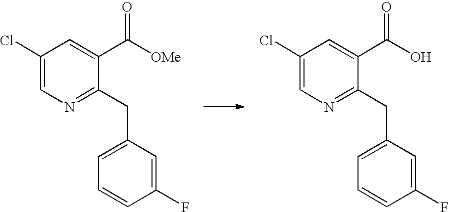

The title compound was prepared according to the procedure described in step 2 of Example 1 from methyl 5-chloro-2-(3-fluorobenzyl)nicotinate (step 1): ¹H-NMR (DMSO-d₆) δ 8.74 (1H, d, J=2.6 Hz), 8.24 (1H, d, J=2.6 Hz), 7.34-7.26 (1H, m), 7.03-6.98 (3H, m), 4.48 (2H, s)

### STEP 3. Methyl 4-[(1S)-1-({[5-chloro-2-(3-fluorobenzyl)pyridin-3-yl]carbonyl}amino)ethyl]benzoate

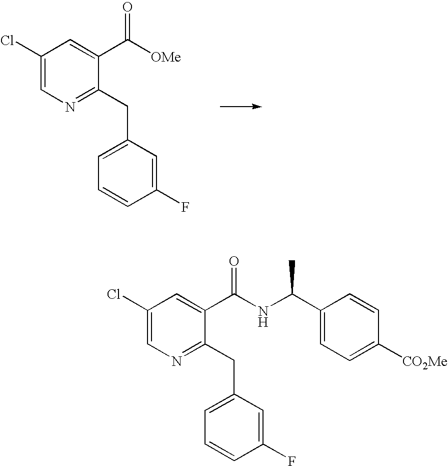

The title compound was prepared according to the procedure described in step 3 of Example 1 from 5-chloro-2-(3-fluorobenzyl)nicotinic acid (step 2) and methyl 4-[(1S)-1-aminoethyl]benzoate hydrochloride: ¹H-NMR (CDCl₃) [symbol] 9.18 (1H, d, J=7.7 Hz), 8.65 (1H, d, J=2.6 Hz), 7.96 (1H, d, J=2.6 Hz), 7.93 (2H, d, J=8.3 Hz), 7.50 (2H, d, J=8.4 Hz), 7.27-7.20 (1H, m), 7.02-6.89 (3H, m), 5.20-5.10 (1H, m), 4.20 (1H, d, J=14.1 Hz), 4.13 (1H, d, J=14.1 Hz), 3.85 (3H, s), 1.42 (3H, d, J=7.2 Hz); MS (ESI) m/z 427 (M+H)⁺, 425 (M−H)⁻.

### STEP 4. 4-[(1S)-1-({[5-Chloro-2-(3-fluorobenzyl)pyridin-3-yl]carbonyl}amino)ethyl]benzoic acid

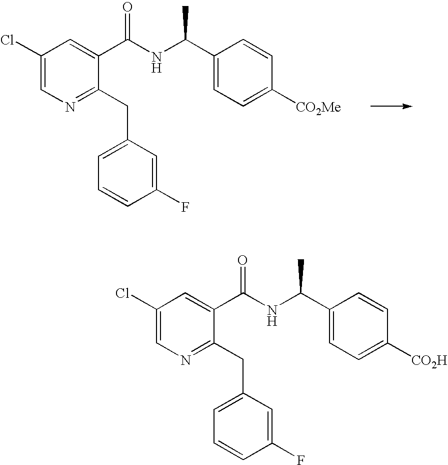

The title compound was prepared according to the procedure described in step 4 of Example 1 from methyl 4-[(1S)-1-({[5-chloro-2-(3-fluorobenzyl)pyridin-3-yl]carbonyl}amino)ethyl]benzoate (step 3): ¹H-NMR (DMSO-d₆) [symbol] 9.16 (1H, d, J=7.7 Hz), 8.65 (1H, d, J=2.5 Hz), 7.97 (1H, d, J=2.3 Hz), 7.90 (2H, d, J=8.2 Hz), 7.47 (2H, d, J=8.4 Hz), 7.28-7.20 (1H, m), 7.01-6.91 (3H, m), 5.18-8.08 (1H, m), 4.21 (1H, d, J=14.2 Hz), 4.13 (1H, d, J=14.2 Hz), 1.42 (3H, d, J=7.1 Hz); MS (ESI) m/z 413 (M+H)⁺, 411 (M−H)⁻.

### Example 62

### 4-[(1S)-1-({[5-CHLORO-2-(3-CHLOROBENZYL)PYRIDIN-3-YL]CARBONYL}AMINO)ETHYL]BENZOIC ACID

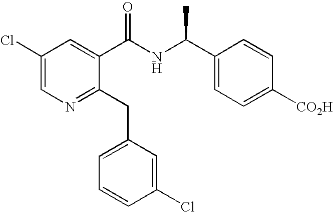

### STEP 1. Methyl 5-chloro-2-(3-chlorobenzyl)nicotinate

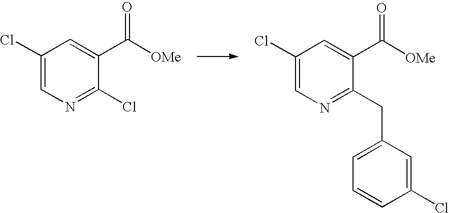

The title compound was prepared according to the procedure described in step 2 of Example 58 from methyl 2,5-dichloronicotinate and 3-chlorobenzylzinc chloride: ¹H-NMR (CDCl₃) [symbol] 8.65 (1H, d, J=2.6 Hz), 8.17 (1H, d, J=2.6 Hz), 7.26-7.12 (4H, m), 4.52 (2H, s), 3.89 (3H, s)

### STEP 2. 5-Chloro-2-(3-chlorobenzyl)nicotinic acid

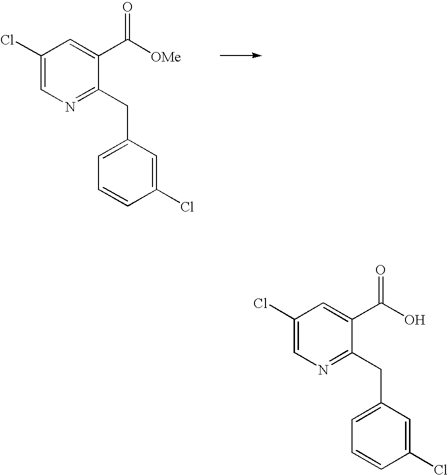

The title compound was prepared according to the procedure described in step 2 of Example 1 from methyl 5-chloro-2-(3-chlorobenzyl)nicotinate (step 1): ¹H-NMR (DMSO-d₆) δ 8.74 (1H, d, J=2.6 Hz), 8.24 (1H, d, J=2.6 Hz), 7.32-7.13 (4H, m), 4.47 (2H, s{tilde over ())}.

### STEP 3. Methyl 4-[(1S)-1-({[5-chloro-2-(3-chlorobenzyl)pyridin-3-yl]carbonyl}amino)ethyl]benzoate

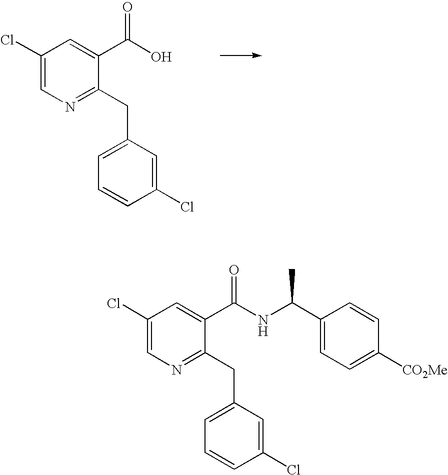

The title compound was prepared according to the procedure described in step 3 of Example 1 from 5-chloro-2-(3-chlorobenzyl)nicotinic acid (step 2) and methyl 4-[(1S)-1-aminoethyl]benzoate hydrochloride: ¹H-NMR (CDCl₃) [symbol] 9.19 (1H, d, J=7.7 Hz), 8.65 (1H, d, J=2.4 Hz), 7.98-7.92 (3H, m), 7.51 (2H, d, J=8.4 Hz), 7.23-7.18 (3H, m), 7.09-7.06 (1H, m), 5.20-5.10 (1H, m), 4.18 (1H, d, J=14.2 Hz), 4.12 (1H, d, J=14.2 Hz), 3.85 (3H, s), 1.42 (3H, d, J=7.0 Hz); MS (ESI) m/z 443 (M+H)⁺, 441 (M−H)⁻.

### STEP 4. 4-[(1S)-1-({[5-Chloro-2-(3-chlorobenzyl)pyridin-3-yl]carbonyl}amino)ethyl]benzoic acid

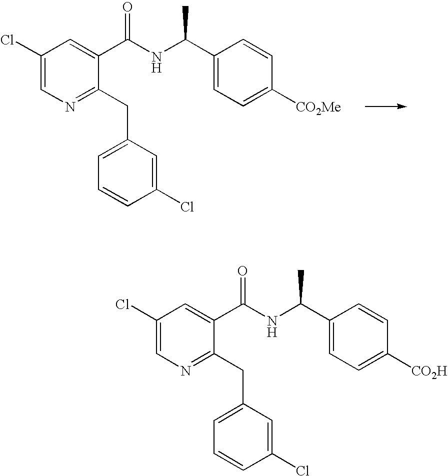

The title compound was prepared according to the procedure described in step 4 of Example 1 from methyl 4-[(1S)-1-({[5-chloro-2-(3-chlorobenzyl)pyridin-3-yl]carbonyl}amino)ethyl]benzoate (step 3): ¹H-NMR (DMSO-d₆) [symbol] 9.17 (1H, d, J=7.7 Hz), 8.65 (1H, d, J=2.3 Hz), 7.98-7.90 (3H, m), 7.48 (2H, d, J=8.2 Hz), 7.24-7.19 (3H, m), 7.10-7.06 (1H, m), 5.20-5.10 (1H, m), 4.19 (1H, d, J=13.9 Hz), 4.12 (1H, d, J=13.9 Hz), 1.43 (3H, d, J=7.1 Hz); MS (ESI) m/z 429 (M+H)⁺, 427 (M−H)⁻.

### Example 63

### 4-[(1S)-1-({[5-CHLORO-2-(3-METHOXYBENZYL)PYRIDIN-3-YL]CARBONYL}AMINO)ETHYL]BENZOIC ACID

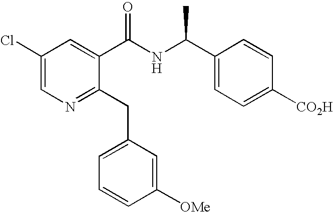

### STEP 1. Methyl 5-chloro-2-(3-methoxybenzyl)nicotinate

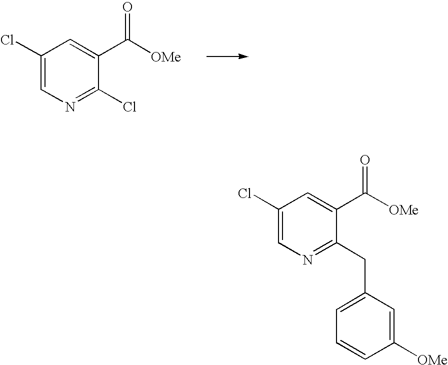

The title compound was prepared according to the procedure described in step 2 of Example 58 from methyl 2,5-dichloronicotinate and 3-methoxybenzylzinc chloride: ¹H-NMR (CDCl₃) [symbol] 8.64 (1H, d, J=2.5 Hz), 8.16 (1H, d, J=2.5 Hz), 7.17 (1H, t, J=7.9 Hz), 6.83-6.70 (3H, m), 4.53 (2H, s), 3.88 (3H, s), 3.75 (3H, s).

### STEP 2. 5-Chloro-2-(3-methoxybenzyl)nicotinic acid

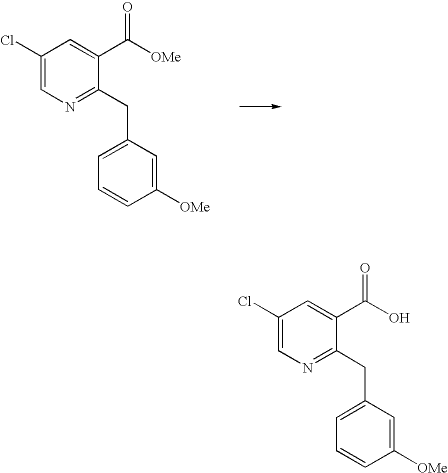

The title compound was prepared according to the procedure described in step 2 of Example 1 from methyl 5-chloro-2-(3-methoxybenzyl)nicotinate (step 1): H-NMR (DMSO-d₆) [symbol] 8.73 (1H, d, J=2.6 Hz), 8.21 (1H, d, J=2.6 Hz), 7.18-7.13 (1H, m), 6.75-6.71 (3H, m), 4.34 (2H, s), 3.69 (3H, s{tilde over ())}.

### STEP 3. Methyl 4-[(1S)-1-({[5-chloro-2-(3-methoxybenzyl)pyridin-3-yl]carbonyl}amino)ethyl]benzoate

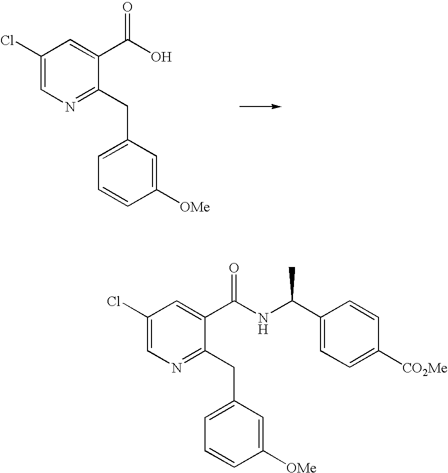

The title compound was prepared according to the procedure described in step 3 of Example 1 from 5-chloro-2-(3-methoxybenzyl)nicotinic acid (step 2) and methyl 4-[(1S)-1-aminoethyl]benzoate hydrochloride: ¹H-NMR (CDCl₃) [symbol] 9.15 (1H, d, J=7.5 Hz), 8.63 (1H, d, J=2.4 Hz), 7.94-7.90 (3H, m), 7.49 (2H, d, J=8.4 Hz), 7.09 (1H, t, J=7.8 Hz), 6.74-6.63 (3H, m), 5.18-5.08 (1H, m), 4.17 (1H, d, J=13.8 Hz), 4.09 (1H, d, J=13.8 Hz), 3.85 (3H, s), 3.66 (3H, s), 1.41 (3H, d, J=7.0 Hz); MS (ESI) m/z 439 (M+H)⁺, 437 (M−H)⁻.

### STEP 4. 4-[(1S)-1-({[5-Chloro-2-(3-methoxybenzyl)pyridin-3-yl]carbonyl}amino)ethyl]benzoic acid

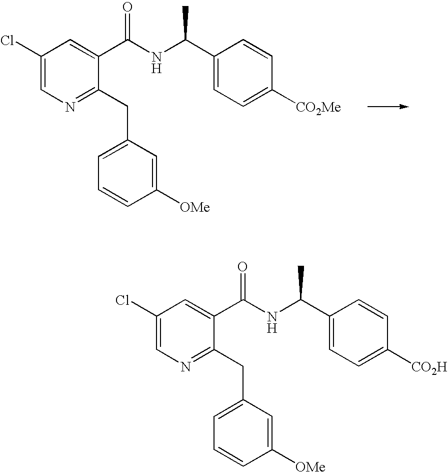

The title compound was prepared according to the procedure described in step 4 of Example 1 from methyl 4-[(1S)-1-({[5-chloro-2-(3-methoxybenzyl)pyridin-3-yl]carbonyl}amino)ethyl]benzoate (step 3): ¹H-NMR (DMSO-d₆) [symbol] 9.13 (1H, d, J=7.7 Hz), 8.63 (1H, d, J=2.5 Hz), 7.94-7.88 (3H, m), 7.46 (2H, d, J=8.2 Hz), 7.10 (1H, t, J=7.7 Hz), 6.74-6.64 (3H, m), 5.19-5.09 (1H, m), 4.17 (1H, d, J=13.9 Hz), 4.10 (1H, d, J=13.9 Hz), 3.66 (3H, s), 1.41 (3H, d, J=7.1 Hz); MS (ESI) m/z 425 (M+H)⁺, 423 (M−H)⁻.

### Example 64

### 4-[(1S)-1-({[5-CHLORO-2-(3-CYANOBENZYL)PYRIDIN-3-YL]CARBONYL}AMINO)ETHYL]BENZOIC ACID

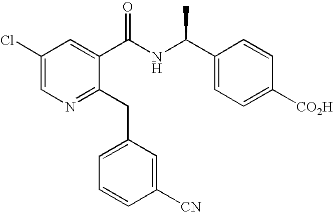

### STEP 1. Methyl 5-chloro-2-(3-cyanobenzyl)nicotinate

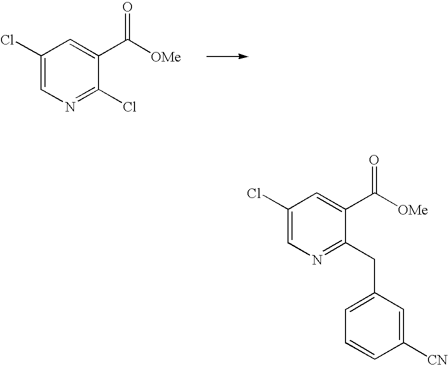

The title compound was prepared according to the procedure described in step 2 of Example 58 from methyl 2,5-dichloronicotinate and 3-cyanobenzylzinc bromide: ¹H-NMR (CDCl₃) [symbol] 8.66 (1H, d, J=2.5 Hz), 8.23 (1H, d, J=2.5 Hz), 7.58-7.34 (4H, m), 4.57 (2H, s), 3.91 (3H, s).

### STEP 2. 5-Chloro-2-(3-cyanobenzyl)nicotinic acid

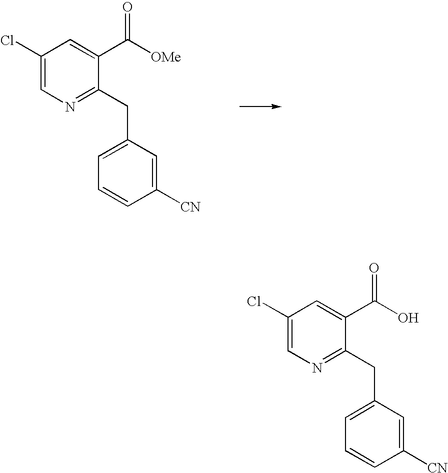

The title compound was prepared according to the procedure described in step 2 of Example 1 from methyl 5-chloro-2-(3-cyanobenzyl)nicotinate (step 1): ¹H-NMR (DMSO-d₆) [symbol] 8.74 (1H, d, J=2.6 Hz), 8.26 (1H, d, J=2.6 Hz), 7.68-7.65 (2H, m), 7.55-7.46 (2H, m), 4.52 (2H, s).

### STEP 3. Methyl 4-[(1S)-1-({[5-chloro-2-(3-cyanobenzyl)pyridin-3-yl]carbonyl}amino)ethyl]benzoate

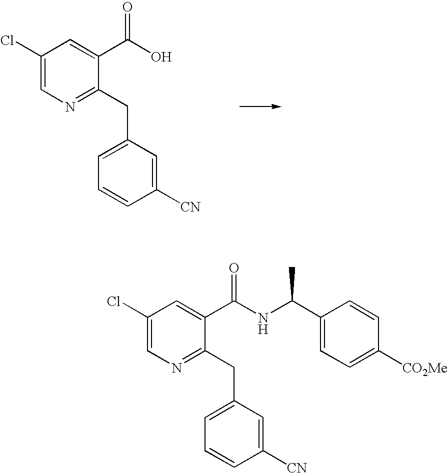

The title compound was prepared according to the procedure described in step 3 of Example 1 from 5-chloro-2-(3-cyanobenzyl)nicotinic acid (step 2) and methyl 4-[(1S)-1-aminoethyl]benzoate hydrochloride: ¹H-NMR (CDCl₃) [symbol] 8.58 (1H, d, J=2.4 Hz), 8.05-8.02 (2H, m), 7.66 (1H, d, J=2.4 Hz), 7.50-7.27 (6H, m), 6.01 (1H, d, J=8.1 Hz), 5.32-5.23 (1H, m), 4.30 (2H, s). 3.93 (3H, s), 1.54 (3H, d, J=7.0 Hz); MS (ESI) m/z 432 (M−H)⁻.

### STEP 4. 4-[(1S)-1-({[5-Chloro-2-(3-cyanobenzyl)pyridin-3-yl]carbonyl}amino)ethyl]benzoic acid

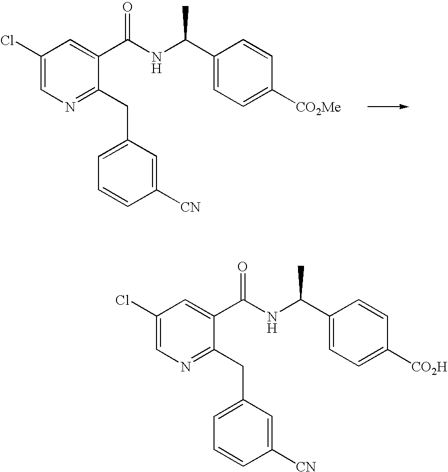

The title compound was prepared according to the procedure described in step 4 of Example 1 from methyl 4-[(1S)-1-({[5-chloro-2-(3-cyanobenzyl)pyridin-3-yl]carbonyl}amino)ethyl]benzoate (step 3): ¹H-NMR (DMSO-d₆) [symbol] 9.20 (1H, d, J=7.7 Hz), 8.65 (1H, d, J=2.4 Hz), 8.01 (1H, d, J=2.6 Hz), 7.91 (2H, d, J=8.3 Hz), 7.66-7.40 (6H, m), 5.18-5.08 (1H, m), 4.23 (1H, d, J=14.4 Hz), 4.17 (1H, d, J=14.4 Hz), 1.42 (3H, d, J=7.2 Hz); MS (ESI) m/z 420 (M+H)⁺, 418 (M−H)⁻.

### Example 65

### 4-({[5-FLUORO-2-(4-FLUOROPHENOXY)BENZOYL]AMINO}METHYL)BENZOIC ACID

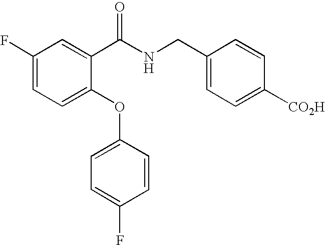

### STEP 1. Methyl 4-({[5-fluoro-2-(4-fluorophenyoxy)benzoyl]amino}methyl)benzoate

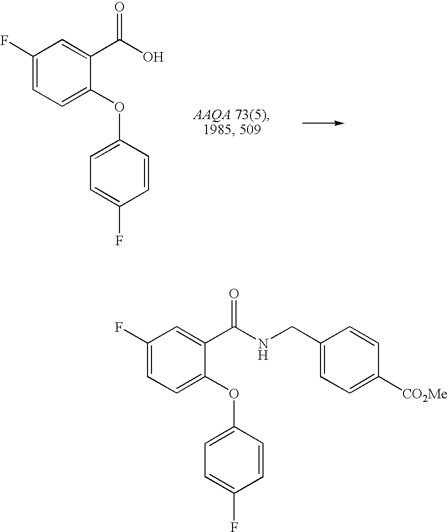

The title compound was prepared according to the procedure described in step 3 of Example 1 from 5-fluoro-2-(4-fluorophenoxy)benzoic acid (Anales de la Asociacion Quimica Argentina 1985, 73, 509) and methyl 4-(aminomethyl)benzoate hydrochloride: ¹H-NMR (CDCl₃) [symbol] 7.99 (4H, m), 7.30 (2H, d, J=7.6 Hz), 7.14-7.03 (3H, m), 6.96-6.92 (2H, m), 6.80 (1H, dd, J=9.2, 4.4 Hz), 4.70 (2H, d, J=5.9 Hz), 3.91 (3H, s); MS (ESI) m/z 398 (M+H)⁺, 396 (M−H)⁻.

### STEP 2. 4-({[5-Fluoro-2-(4-fluorophenoxy)benzoyl]amino}methyl)benzoic acid

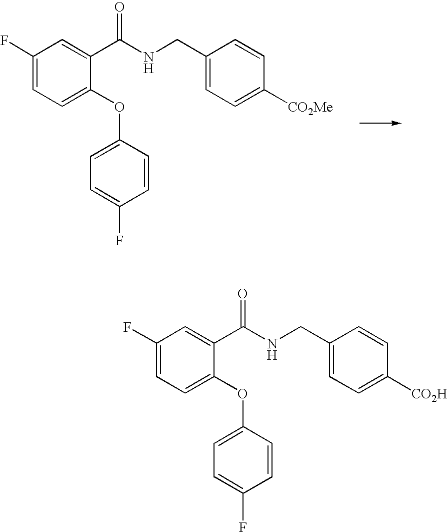

The title compound was prepared according to the procedure described in step 4 of Example 1 from methyl 4-({[5-fluoro-2-(4-fluorophenoxy)benzoyl]amino}methyl)benzoate (step 1): ¹H-NMR (DMSO-d₆) [symbol] 8.94 (1H, t, J=5.9 Hz), 7.81 (2H, d, J=8.1 Hz), 7.48 (1H, dd, J=8.8, 3.1 Hz), 7.37-7.19 (5H, m), 7.08-6.99 (3H, m), 4.47 (2H, d, J=5.9 Hz); MS (ESI) m/z 384 (M+H)⁺, 382 (M−H)⁻.

### Example 66

### 4-({[4-FLUORO-2-(4-FLUOROPHENOXY)BENZOYL]AMINO}METHYL)BENZOIC ACID

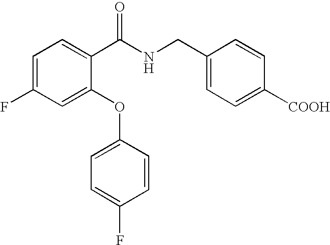

### STEP 1. 4-fluoro-2-(4-fluorophenoxy)benzoic acid

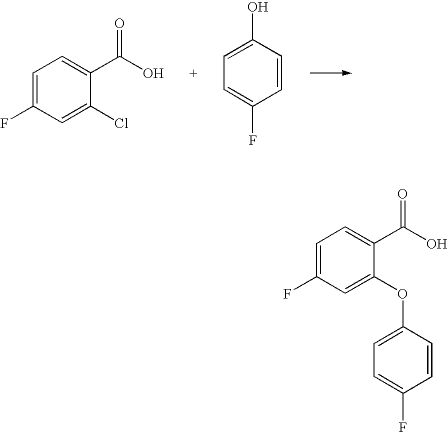

A mixture of 2-chloro-4-fluorobenzoic acid (1.74 g, 10 mmol), 4-fluorophenol (2.24 g, 20 mmol), copper (50 mg, 0.78 mmol), copper(I) iodide (50 mg, 0.28 mmol), potassium carbonate (2.76 g, 20 mmol), and pyridine (0.40 mL, 5.0 mmol) in water (6.0 mL) was heated under reflux for 2 h with stirring. The reaction mixture was diluted with water and filtered through a pad of celite. The pH value of the filtrate was adjusted to 9.0 by the addition of 2 M sodium carbonate. The aqueous mixture was extracted with dichloromethane (50 mL×3). The combined organic extracts were washed with brine (50 mL), dried (sodium sulfate), and concentrated to afford 29 mg (99%) of the title compound: MS (ESI) m/z 250 (M⁺).

### STEP 2. methyl 4-({[4-fluoro-2-(4-fluorophenoxy)benzoyl]amino}methyl)benzoate

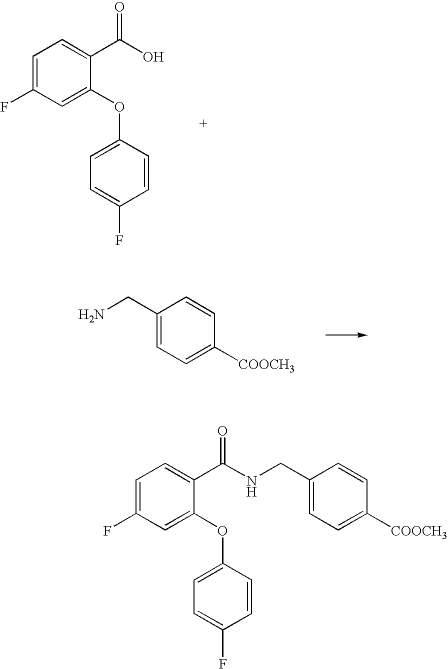

The title compound was prepared according to the procedure described in step 3 of Example 1 from 4-fluoro-2-(4-fluorophenoxy)benzoic acid (step 1) and methyl 4-(aminomethyl)benzoate hydrochloride: ¹H-NMR (CDCl₃) [symbol] 7.99-7.89 (2H, m), 7.38-7.28 (4H, m), 7.12-7.00 (4H, m), 6.41 (1H, dd, J=2.7, 8.1 Hz), 4.63 (2H, br.s), 3.86 (3H, s); MS (ESI) m/z 398 (M+H)⁺, 396 (M+H)⁺.

### STEP 3. 4-({[4-fluoro-2-(4-fluorophenoxy)benzoyl]amino}methyl)benzoic acid

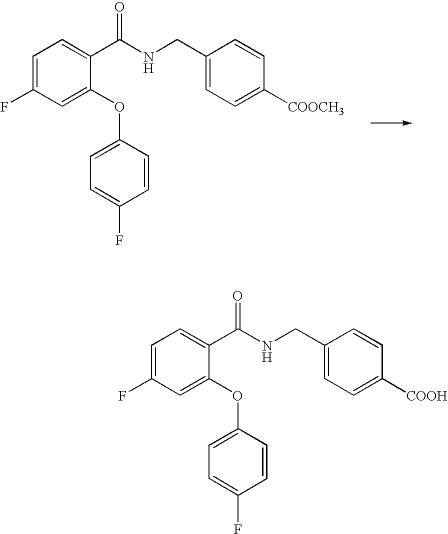

The title compound was prepared according to the procedure described in step 4 of Example 1 from methyl 4-({[4-fluoro-2-(4-fluorophenoxy)benzoyl]amino}methyl)benzoate (step 2): ¹H-NMR (CDCl₃) [symbol] 8.32 (1H, t, J=8.7 Hz), 8.04 (2H, d, J=7.9 Hz), 7.40 (1H, d, J=7.9 Hz), 7.17-7.03 (4H, m), 6.92 (1H, dt, J=2.5, 8.9 Hz), 6.45 (1H, d, J=8.9 Hz), 4.75 (2H, d, J=5.7 Hz); MS (ESI) m/z 384 (M+H)⁺, 382 (M−H)⁻.

### Example 67

### 4-({[5-CHLORO-2-(4-FLUOROPHENOXY)BENZOYL]AMINO})METHYL)BENZOIC ACID

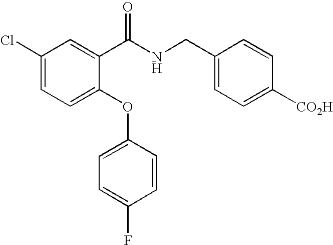

### STEP 1. Methyl 5-chloro-2-(4-fluorophenoxy)benzoate

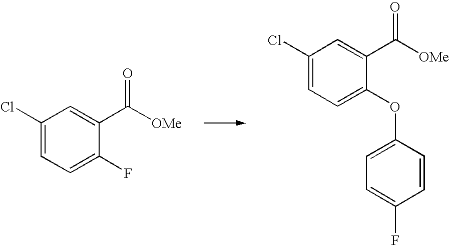

To a stirred solution of 4-fluorophenol (1.60 g, 14.3 mmol) and sodium hydride (0.34 g, 14.3 mmol) in N,N-dimethylforamide (30 mL) was added a solution of methyl 5-chloro-2-fluorobenzoate (2.70 g, 14.3 mmol) in N,N-dimethylforamide (30 mL) at 0° C. The resulting mixture was heated at 120° C. for 16 h. After cooling to room temperature, the mixture was diluted with ether (300 mL) and washed with water (150 mL×3). The organic layer was dried over magnesium sulfate and concentrated under reduced pressure. The residue was purified by flash column chromatography on silica gel eluting with hexane/ethyl acetate (20:1) to afford 2.60 g (65%) of the title compounds as slight yellow oil: ¹H-NMR (CDCl₃) [symbol] 7.88 (1H, d, J=2.8 Hz), 7.40 (1H, dd, J=8.8, 2.8 Hz), 7.06-6.85 (5H, m), 3.84 (3H, s{tilde over ())}

### STEP 2. 5-Chloro-2-(4-fluorophenoxy)benzoic acid

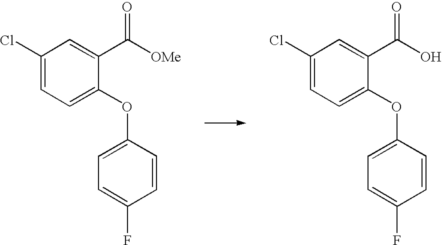

A mixture of methyl 5-chloro-2-(4-fluorophenoxy)benzoate (step 1, 2.60 g, 9.26 mmol), tetrahydrofuran (20 mL), methanol (20 mL), and 2 M sodium hydroxide (20 mL) was stirred at room temperature for 3 h. The mixture was poured into 2 M hydrochloric acid (50 mL), and extracted with ethyl acetate (200 mL). The organic layer was dried over magnesium sulfate and evaporated to give 2.41 g (98%) of the title compound as white solids: ¹H-NMR (CDCl₃) [symbol] 8.11 (1H, d, J=2.8 Hz), 7.43 (1H, dd, J=9.0, 2.8 Hz), 7.14-7.02 (4H, m), 6.80 (1H, d, J=8.8 Hz); MS (ESI) m/z 265 (M−H)⁻.

### STEP 3. Methyl 4-({[5-chloro-2-(4-fluorophenoxy)benzoyl]amino}methyl)benzoate

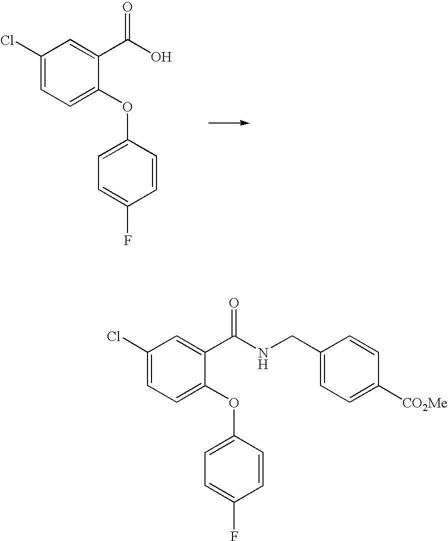

The title compound was prepared according to the procedure described in step 3 of Example 1 from 5-chloro-2-(4-fluorophenoxy)benzoic acid (step 2) and methyl 4-(aminomethyl)benzoate hydrochloride: ¹H-NMR (CDCl₃) [symbol] 8.24 (1H, d, J=2.8 Hz), 7.97-7.94 (3H, m), 7.36-7.32 (3H, m), 7.11-6.96 (4H, m), 6.74 (1H, d, J=8.8 Hz), 4.71 (2H, d, J=5.9 Hz), 3.90 (3H, s); MS (ESI) m/z 414 (M+H)⁺, 412 (M−H)⁻

### STEP 4. 4-({[5-Chloro-2-(4-fluorophenoxy)benzoyl]amino}methyl)benzoic acid

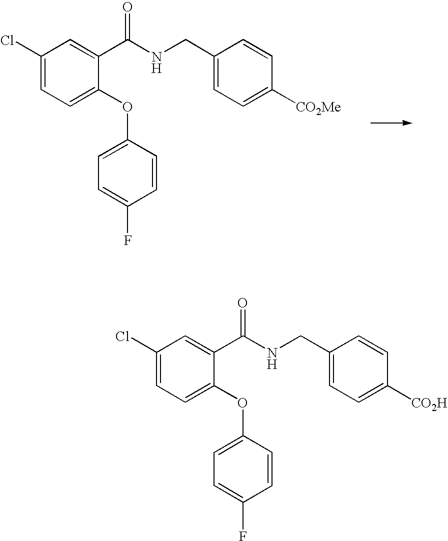

The title compound was prepared according to the procedure described in step 4 of Example 1 from methyl 4-({[5-chloro-2-(4-fluorophenoxy)benzoyl]amino}methyl)benzoate (step 3): ¹H-NMR (DMSO-d₆) [symbol] 8.98 (1H, t, J=5.9 Hz), 7.82 (2H, d, J=8.2 Hz), 7.68 (1H, d, J=2.6 Hz), 7.51 (1H, dd, J=8.7, 2.6 Hz), 7.36-7.08 (6H, m), 6.94 (1H, d, J=8.7 Hz), 4.50 (2H, d, J=5.9 Hz); MS (ESI) m/z 400 (M+H)⁺, 398 (M−H)⁻.

### Example 68

### 4-((1S)-1-{[5-CHLORO-2-(4-FLUOROPHENOXY)BENZOYL]AMINO}ETHYL)BENZOIC ACID

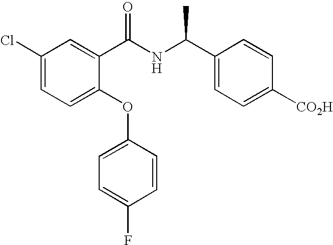

### STEP 1. Methyl 4-((1S)-1-{[5-chloro-2-(4-fluorophenoxy)benzoyl]amino}ethyl)benzoate

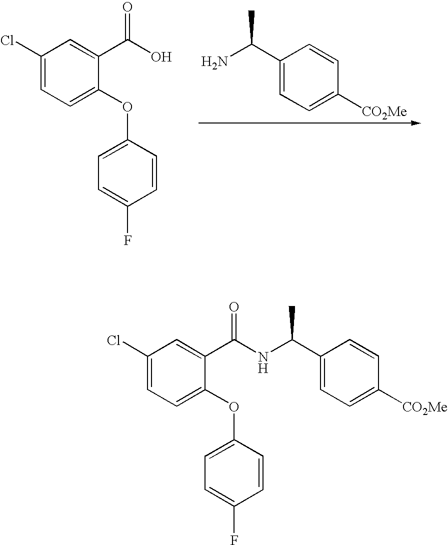

The title compound was prepared according to the procedure described in step 3 of Example 1 from 5-chloro-2-(4-fluorophenoxy)benzoic acid (step 2 of Example 67) and methyl 4-[(1S)-1-aminoethyl]benzoate hydrochloride (step 3 of Example 5): ¹H-NMR (CDCl₃) [symbol] 8.16 (1H, d, J=2.6 Hz), 7.95 (2H, dd, J=6.6, 1.8 Hz), 7.88 (1H, d, J=7.4 Hz), 7.36-7.29 (3H, m), 7.23-6.96 (4H, m), 6.78 (1H, d, J=8.7 Hz), 5.32 (1H, dq, J=7.4, 6.9 Hz), 3.90 (3H, s), 1.51 (3H, d, J=6.9 Hz); MS (ESI) m/z 428 (M+H)⁺, 426 (M−H)⁻.

### STEP 2. 4-((1S)-1-{[5-Chloro-2-(4-fluorophenoxy)benzoyl]amino}ethyl)benzoic acid

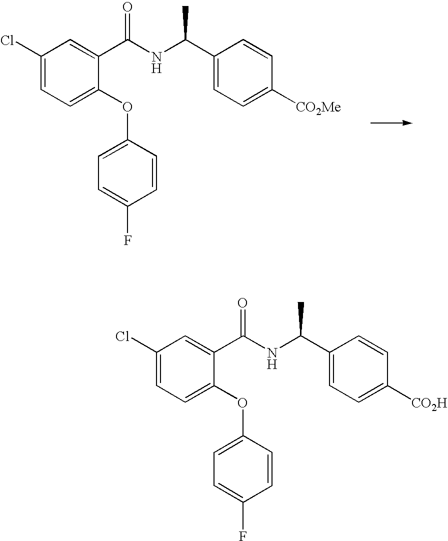

The title compound was prepared according to the procedure described in step 4 of Example 1 from methyl 4-((1S)-1-{[5-chloro-2-(4-fluorophenoxy)benzoyl]amino}ethyl)benzoate (step 1): ¹H-NMR (DMSO-d₆) [symbol] 8.17 (1H, d, J=2.8 Hz), 8.01 (2H, d, J=8.4 Hz), 7.94 (1H, d, J=7.3 Hz), 7.38-7.32 (3H, m), 7.14-6.98 (4H, m), 6.78 (1H, d, J=8.8 Hz), 5.34 (1H, dq, J=7.3, 7.0 Hz), 1.53 (3H, d, J=7.0 Hz); MS (ESI) m/z 414 (M+H)⁺, 412 (M−H)⁻.

### Example 69

### 4-({[5-CHLORO-2-(4-FLUOROPHENOXY)BENZOYL]AMINO}METHYL)-2-FLUOROBENZOIC ACID

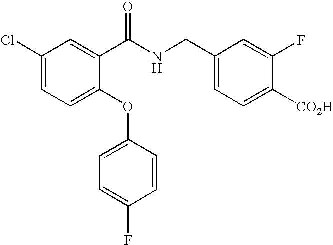

### STEP 1. Methyl 4-({[5-chloro-2-(4-fluorophenoxy)benzoyl]amino}methyl)-2-fluorobenzoate

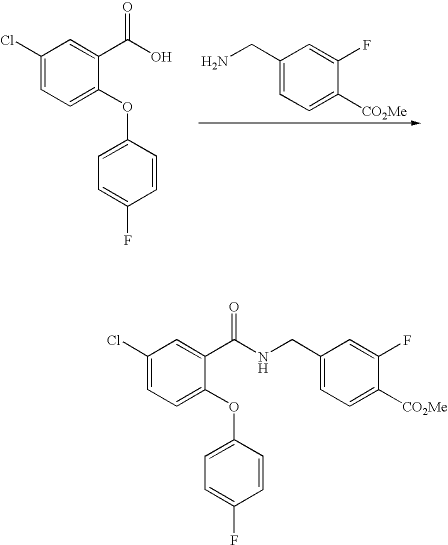

The title compound was prepared according to the procedure described in step 3 of Example 1 from 5-chloro-2-(4-fluorophenoxy)benzoic acid (step 2 of Example 67) and methyl 4-(aminomethyl)-2-fluorobenzoate: ¹H-NMR (CDCl₃) [symbol] 8.22 (1H, d, J=2.6 Hz), 8.06-7.98 (1H, m), 7.86 (1H, t, J=7.7 Hz), 7.35 (1H, dd, J=8.9, 2.6 Hz), 7.13-6.98 (6H, m), 6.75 (1H, d, J=8.9 Hz), 4.69 (2H, d, J=5.9 Hz), 3.91 (3H, s); MS (ESI) m/z 432 (M+H)⁺, 430 (M−H)⁻.

### STEP 2. 4-({[5-Chloro-2-(4-fluorophenoxy)benzoyl]amino}methyl)-2-fluorobenzoic acid

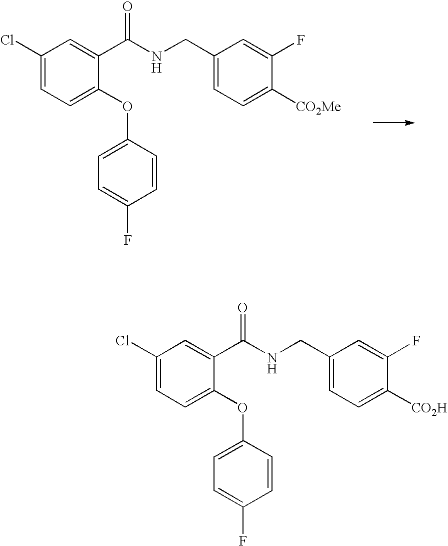

The title compound was prepared according to the procedure described in step 4 of Example 1 from methyl 4-({[5-chloro-2-(4-fluorophenoxy)benzoyl]amino}methyl)-2-fluorobenzoate (step 1): ¹H-NMR (DMSO-d₆) [symbol] 8.19 (1H, d, J=2.8 Hz), 8.09 (1H, t, J=6.0 Hz), 7.86 (1H, t, J=7.8 Hz), 7.35-6.97 (8H, m), 6.73 (1H, d, J=8.8 Hz), 4.66 (2H, d, J=6.1 Hz); MS (ESI) m/z 418 (M+H)⁺, 416 (M−H)⁻.

### Example 70

### 4-((1S)-1-{[5-CHLORO-2-(3-CHLOROPHENOXY)BENZOYL]AMINO}ETHYL)BENZOIC ACID

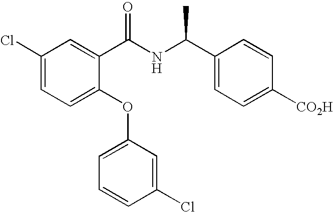

### STEP 1. Methyl 5-chloro-2-(3-chlorophenoxy)benzoate

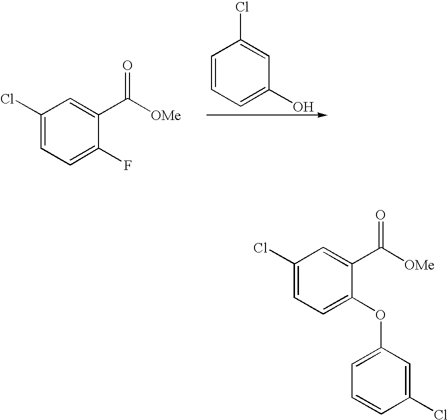

The title compound was prepared according to the procedure described in step 1 of Example 67 from methyl 5-chloro-2-fluorobenzoate and 3-chlorophenol: H-NMR (CDCl₃) [symbol] 7.92 (1H, d, J=2.8 Hz), 7.46 (1H, dd, J=8.8, 2.8 Hz), 7.25-6.80 (5H, m), 3.81 (3H, s).

### STEP 2. 5-Chloro-2-(3-chlorophenoxy)benzoic acid

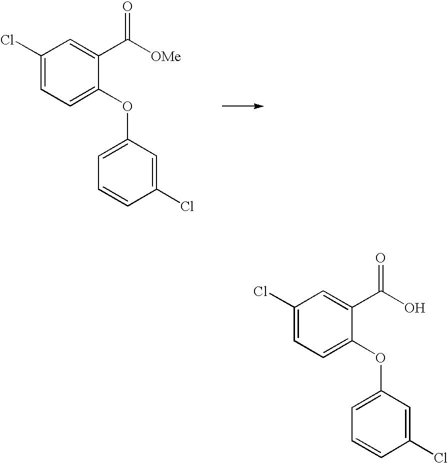

The title compound was prepared according to the procedure described in step 2 of Example 67 from methyl 5-chloro-2-(3-chlorophenoxy)benzoate (step 1): ¹H-NMR (CDCl₃) δ 8.13 (1H, d, J=2.8 Hz), 7.45 (1H, dd, J=9.0, 2.8 Hz), 7.14-6.80 (5H, m).

### STEP 3. Methyl 4-((1S)-1-{[5-chloro-2-(3-chlorophenoxy)benzoyl]amino}ethyl)benzoate

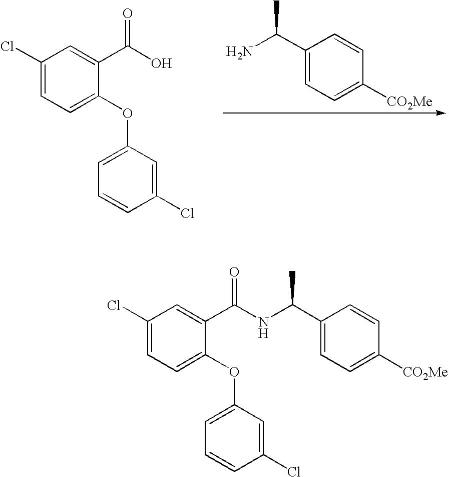

The title compound was prepared according to the procedure described in step 3 of Example 1 from 5-chloro-2-(3-chlorophenoxy)benzoic acid (step 2) and methyl 4-[(1S)-1-aminoethyl]benzoate hydrochloride (step 3 of Example 5): ¹H-NMR (CDCl₃) [symbol] 8.17 (1H, d, J=2.8 Hz), 7.93 (2H, m), 7.65 (1H, d, J=7.4 Hz), 7.42-7.19 (5H, m), 6.97-6.81 (3H, m), 5.32 (1H, dq, J=7.4, 6.9 Hz), 3.90 (3H, s), 1.49 (3H, d, J=6.9 Hz).

### STEP 4. 4-((1S)-1-{[5-Chloro-2-(3-chlorophenoxy)benzoyl]amino}ethyl)benzoic acid

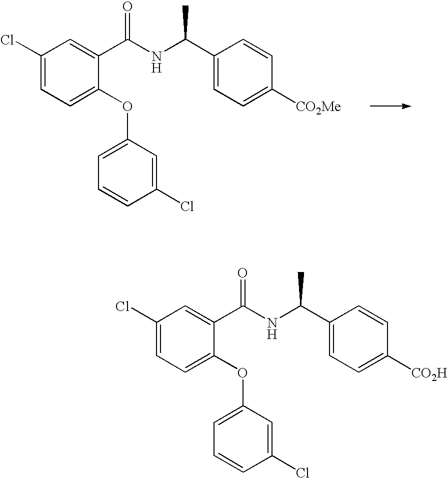

The title compound was prepared according to the procedure described in step 4 of Example 1 from methyl 4-((1S)-1-{[5-chloro-2-(3-chlorophenoxy)benzoyl]amino}ethyl)benzoate (step 3): ¹H-NMR (DMSO-d₆) [symbol] 8.91 (1H, d, J=7.9 Hz), 7.81 (2H, d, J=8.2 Hz), 7.61-7.54 (2H, m), 7.40-7.34 (3H, m), 7.19-6.91 (4H, m), 5.03 (1H, dq, J=7.9, 7.0 Hz), 1.35 (3H, d, J=7.0 Hz); MS (ESI) m/z 430 (M+H)⁺, 428 (M−H)⁻.

### Example 71

### 4-((1S)-1-{[5-CHLORO-2-(3-FLUOROPHENOXY)BENZOYL]AMINO}ETHYL)BENZOIC ACID

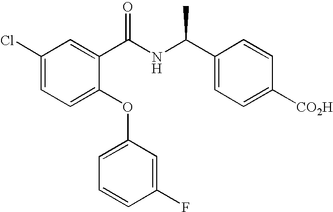

### STEP 1. Methyl 5-chloro-2-(3-fluorophenoxy)benzoate

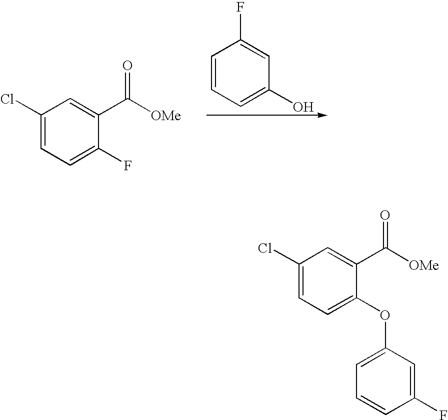

The title compound was prepared according to the procedure described in step 1 of Example 67 from methyl 5-chloro-2-fluorobenzoate and 3-fluorophenol: H-NMR (CDCl₃) δ 7.91 (1H, d, J=2.8 Hz), 7.46 (1H, dd, J=8.7, 2.8 Hz), 7.31-7.06 (1H, m), 6.99 (1H, d, J=8.7 Hz), 6.83-6.61 (3H, m), 3.81 (3H, s).

### STEP 2. 5-Chloro-2-(3-fluorophenoxy)benzoic acid

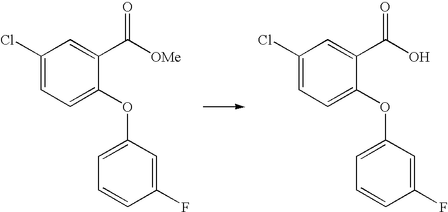

The title compound was prepared according to the procedure described in step 2 of Example 67 from methyl 5-chloro-2-(3-fluorophenoxy)benzoate (step 1): H-NMR (DMSO-d₆) δ 7.85 (1H, d, J=2.8 Hz), 7.66 (1H, dd, J=8.9, 2.8 Hz), 7.42-7.33 (1H, m), 7.15 (1H, d, J=8.9 Hz); 6.98-6.90 (1H, m), 6.84-6.71 (2H, m).

### STEP 3. Methyl 4-((1S)-1-{[5-chloro-2-(3-fluorophenoxy)benzoyl]amino}ethyl)benzoate

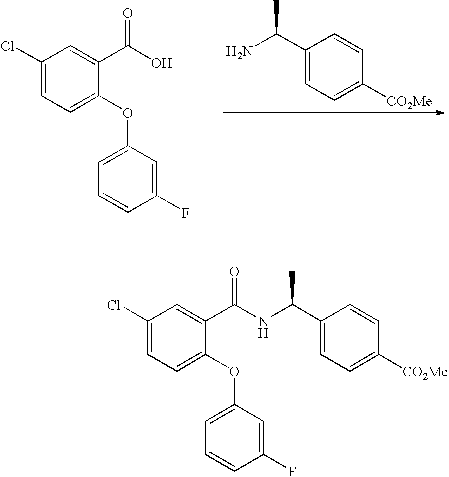

The title compound was prepared according to the procedure described in step 3 of Example 1 from 5-chloro-2-(3-fluorophenoxy)benzoic acid (step 2) and methyl 4-[(1S)-1-aminoethyl]benzoate hydrochloride (step 3 of Example 5): ¹H-NMR (CDCl₃) δ 8.17 (1H, d, J=2.8 Hz), 7.93 (2H, d, J=8.4 Hz), 7.65 (1H, d, J=7.4 Hz), 7.42-7.26 (4H, m), 6.94-6.88 (2H, m), 6.74-6.65 (2H, m), 5.28 (1H, dq, J=7.4, 7.3 Hz), 3.90 (3H, s), 1.48 (3H, d, J=7.3 Hz).

### STEP 4. 4-((1S)-1-{[5-Chloro-2-(3-fluorophenoxy)benzoyl]amino}ethyl)benzoic acid

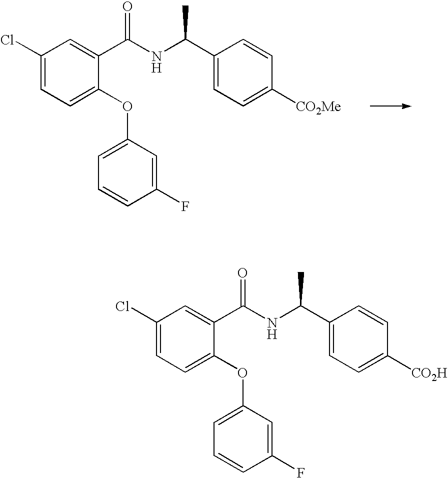

The title compound was prepared according to the procedure described in step 4 of Example 1 from methyl 4-((1S)-1-{[5-chloro-2-(3-fluorophenoxy)benzoyl]amino}ethyl)benzoate (step 3): ¹H-NMR (CDCl3) [symbol] 8.17 (1H, d, J=2.6 Hz), 7.98 (2H, d, J=8.4 Hz), 7.68 (1H, d, J=7.0 Hz), 7.43-7.29 (4H, m), 6.95-6.89 (2H, m), 6.75-6.67 (2H, m), 5.29 (1H, dq, J=8.4, 7.0 Hz), 1.50 (3H, d, J=7.0 Hz); MS (ESI) m/z 414 (M+H)⁺, 412 (M−H)⁻.

### Example 72

### 4-((1S)-1-{[5-CHLORO-2-(3-METHOXYPHENOXY)BENZOYL]AMINO}ETHYL)BENZOIC ACID

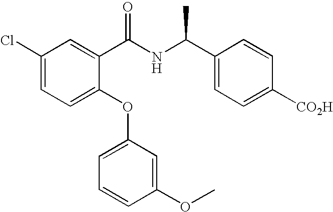

### STEP 1. Methyl 5-chloro-2-(3-methoxyphenoxy)benzoate

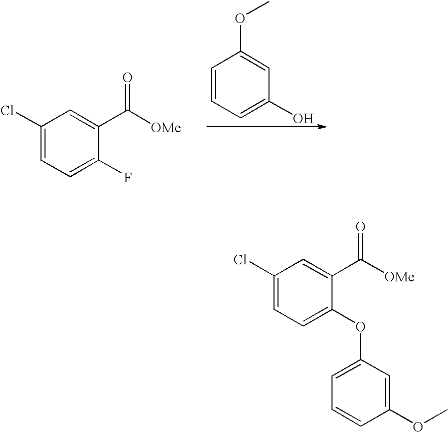

The title compound was prepared according to the procedure described in step 1 of Example 67 from methyl 5-chloro-2-fluorobenzoate and 3-methoxyphenol: ¹H-NMR (CDCl₃) [symbol] 7.89 (1H, d, J=2.8 Hz), 7.41 (1H, dd, J=8.4, 2.8 Hz), 7.24-7.19 (1H, m), 6.95 (1H, d, J=8.4 Hz), 6.67-6.64 (1H, m), 6.53-6.49 (2H, m), 3.83 (3H, s), 3.78 (3H, s).

### STEP 2. Methyl 4-((1S)-1-{[5-chloro-2-(3-methoxyphenoxy)benzoyl]amino}ethyl)benzoate

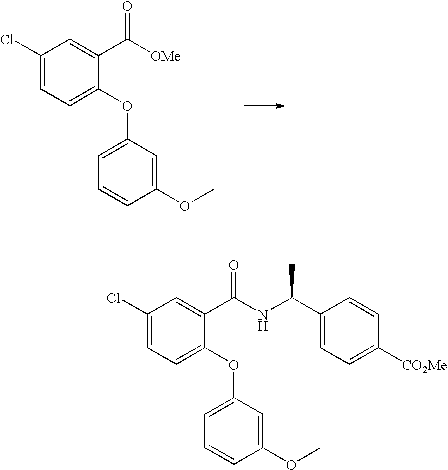

A mixture of methyl 5-chloro-2-(3-methoxyphenoxy)benzoate (step 1, 220 mg, 0.75 mmol) and 2 M sodium hydroxide (2 mL) in methanol (10 mL) was stirred for 7 h at room temperature. The reaction mixture was poured into 2 M hydrochloric acid (50 mL) and the aqueous mixture was extracted with ethyl acetate (200 mL). The organic layer was dried (sodium sulfate) and evaporated to give 168 mg (80%) of the corresponding carboxylic acid. This acid was used for next reaction without further purification. To a stirred solution of this acid (168 mg, 0.60 mmol) and methyl 4-[(1S)-1-aminoethyl]benzoate hydrochloride (step 3 of Example 5, 143 mg, 0.66 mmol) in dichloromethane (20 mL) were successively added 1-(3-dimethylaminopropyl)-3-ethylcarbodiimide hydrochloride (EDCl) (172 mg, 0.90 mmol), 1-hydroxybenzotriazole hydrate (HOBT) (137 mg, 0.90 mmol), and triethylamine (91 uL). After being stirred overnight, the reaction was quenched by the addition of water (50 mL). The organic layer was separated and the aqueous layer was extracted with dichloromethane (50 mL×2). The combined organic layers were washed with brine (50 mL), dried (sodium sulfate), and evaporated. The residue was purified by flash column chromatography on silica gel (50 g) eluting with hexane/ethyl acetate (4/1) to afford 245 mg (93%) of the title compounds as a colorless oil: ¹H-NMR (CDCl₃) [symbol] 8.17 (1H, d, J=2.8 Hz), 7.94-7.86 (3H, m), 7.38-7.25 (4H, m), 6.87 (1H, d, J=8.7 Hz), 6.77-6.73 (1H, m), 6.57-6.52 (2H, m), 5.29 (1H, m), 3.90 (3H, s), 3.78 (3H, s), 1.49 (3H, d, J=6.9 Hz).

### STEP 3. 4-((1S)-1-{[5-Chloro-2-(3-methoxyphenoxy)benzoyl]amino}ethyl)benzoic acid

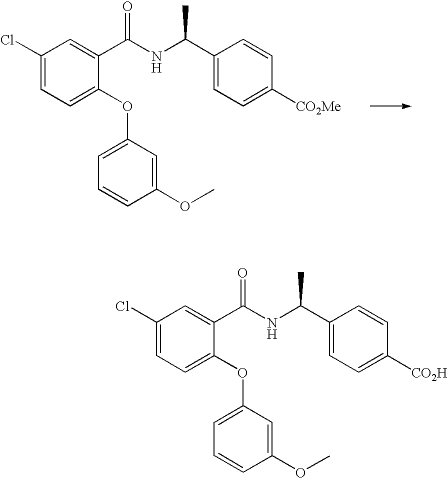

The title compound was prepared according to the procedure described in step 4 of Example 1 from methyl 4-((1S)-1-{[5-chloro-2-(3-methoxyphenoxy)benzoyl]amino}ethyl)benzoate (step 3): ¹H-NMR (DMSO-d₆) [symbol] 8.84 (1H, d, J=7.9 Hz), 7.80 (2H, d, J=8.3 Hz), 7.58 (1H, d, J=2.8 Hz), 7.51 (1H, dd, J=7.9, 7.1 Hz), 7.39 (2H, d, J=8.3 Hz), 7.27 (1H, m), 7.03 (1H, d, J=8.8 Hz), 6.74-6.71 (1H, m), 6.58-6.52 (2H, m), 5.06 (1H, dq, J=7.0 Hz), 3.72 (3H, s), 1.37 (3H, d, J=7.1 Hz); MS (ESI) m/z 426 (M+H)⁺, 424 (M−H)⁻.

### Example 73

### 5-FLUORO-2-(4-FLUOROPHENOXY)-N-[4-(2H-TETRAZOL-5-YL)BENZYL]NICOTINAMIDE

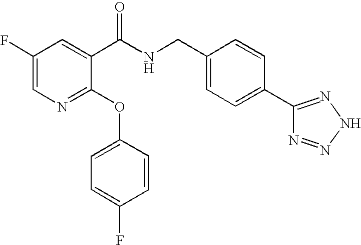

### STEP 1. N-(4-Cyanobenzyl)-5-fluoro-2-(4-fluorophenoxy)nicotinamide

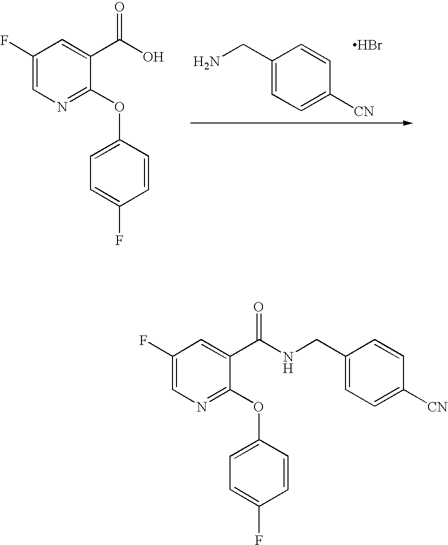

The title compound was prepared according to the procedure described in step 3 of Example 1 from 5-fluoro-2-(4-fluorophenoxy)nicotinic acid (step 2 of Example 1) and 4-cyanobenzylhexamine hydrobromide (Synthesis 1979, 161): ¹H-NMR (CDCl₃) [symbol] 8.38 (1H, dd, J=8.3, 3.1 Hz), 8.33 (1H, br.s), 8.06 (1H, d, J=3.1 Hz), 7.64 (2H, d, J=8.1 Hz), 7.46 (2H, d, J=8.1 Hz), 7.20-7.06 (4H, m), 4.76 (2H, d, J=6.1 Hz); MS (ESI) m/z 366 (M+H)⁺, 364 (M−H)⁻.

### STEP 2. 5-Fluoro-2-(4-fluorophenoxy)-N-[4-(2H-tetrazol-5-yl)benzyl]nicotinamide

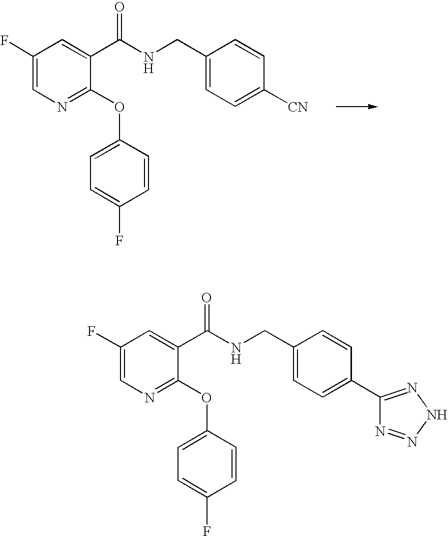

To a solution of N-(4-cyanobenzyl)-5-fluoro-2-(4-fluorophenoxy)nicotinamide (step 1, 220 mg, 0.60 mmol) in 1-methyl-pyrrolidin-2-one (5 mL) were added sodium azide (117 mg, 1.8 mmol) and triethylamine hydrochloride (248 mg, 1.8 mmol) at room temperature. This mixture was heated at 150° C. for 18 h. The reaction mixture was diluted with dichloromethane (100 mL), and the solution was washed with saturated sodium dihydrogenphosphate solution (50 mL). The organic phase was dried (sodium sulfate) and concentrated. The residue was purified by flash column chromatography on silica gel (50 g) eluting with dichloromethane/methanol/acetic acid (100/5/0.5) to give off white solids. The solids were triturated with ethyl acetate to afford 125 mg (50%) of the title compound as white solids: ¹H-NMR (DMSO-d₆) [symbol] 9.18 (1H, t, J=5.8 Hz), 8.14 (1H, d, J=2.8 Hz), 7.99 (1H, dd, J=8.2, 2.8 Hz), 7.90 (2H, d, J=8.2 Hz), 7.51 (2H, d, J=8.2 Hz), 7.22-7.15 (4H, m), 4.56 (2H, d, J=5.8 Hz); MS (ESI) m/z 409 (M+H)⁺, 407 (M−H)⁻.

### Example 74

### 5-CHLORO-2-(4-FLUOROPHENOXY)-N-[4-(2H-TETRAZOL-5-YL)BENZYL]NICOTINAMIDE

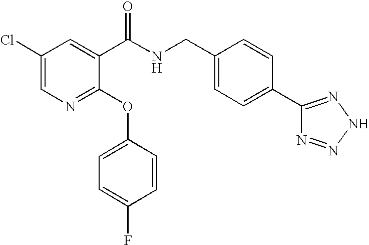

### STEP 1. 5-Chloro-N-(4-cyanobenzyl)-2-(4-fluorophenoxy)nicotinamide

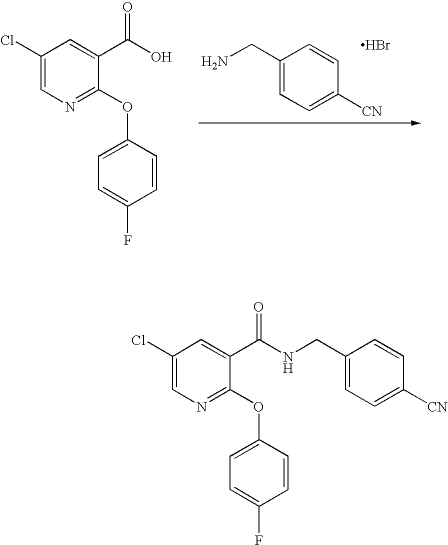

The title compound was prepared according to the procedure described in step 3 of Example 1 from 5-chloro-2-(4-fluorophenoxy)nicotinic acid (EP 1229034) and 4-cyanobenzylhexamine hydrobromide (Synthesis 1979, 161): MS (ESI) m/z 382 (M+H)⁺, 380 (M−H)⁻.

### STEP 2. 5-Chloro-2-(4-fluorophenoxy)-N-[4-(2H-tetrazol-5-yl)benzyl]nicotinamide

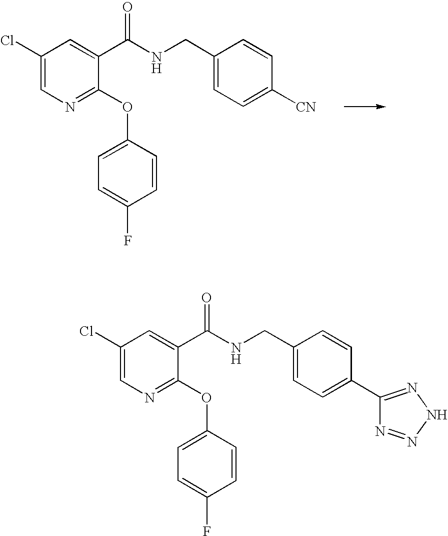

The title compound was prepared according to the procedure described in step 2 of Example 73 from 5-chloro-N-(4-cyanobenzyl)-2-(4-fluorophenoxy)nicotinamide (step 1): MS (ESI) m/z 425 (M+H)⁺, 423 (M−H)⁻.

### Example 75

### 5-FLUORO-2-(4-FLUOROPHENOXY)-N-[4-(2H-TETRAZOL-5-YL)BENZYL]BENZAMIDE

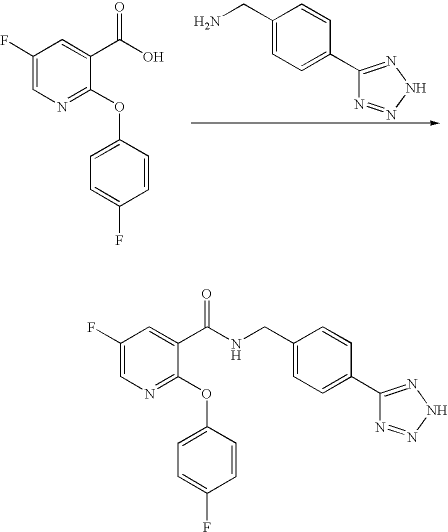

A mixture of 5-fluoro-2-(4-fluorophenoxy)benzoic acid (120 mg, 0.48 mmol), 1-[4-(2H-tetrazol-5-yl)phenyl]methanamine hydrochloride (WO 9604267, WO 9604246,122 mg, 0.58 mmol), 1-hydroxy-1H-benztriazole monohydrate (110 mg, 0.72 mmol), 1-ethyl-3-(3-dimethylaminopropyl)carbodiimide hydrochloride (138 mg, 0.72 mmol), and triethylamine (0.27 mL, 1.92 mmol) in dichloromethane (8 mL) and N,N-dimethylforamide (2 mL) was stirred at room temperature for 16 h. The mixture was diluted with dichloromethane (50 mL) and washed with 5% aqueous sodium dihydrogenphosphate solution (30 mL). The organic fraction was dried over magnesium sulfate, and concentrated under reduced pressure. The residue was purified by flash column chromatography on silica gel eluting with hexane/ethyl acetate/acetic acid (30:60:1) to afford 143 mg (73%) of the title compounds as white solids: ¹H-NMR (DMSO-d₆) [symbol] 8.99 (1H, t, J=5.9 Hz), 7.91 (2H, d, J=8.2 Hz), 7.53-6.99 (8H, m), 4.50 (2H, d, J=5.9 Hz); MS (ESI) m/z 408 (M+H)⁺, 406 (M−H)⁻.

### Example 76

### 5-CHLORO-2-(4-FLUOROPHENOXY)-N-[4-(2H-TETRAZOL-5-YL)BENZYL]BENZAMIDE

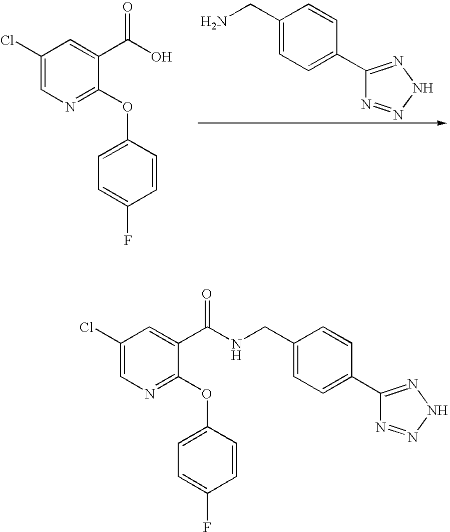

The title compound was prepared according to the procedure described in Example 75 from 5-chloro-2-(4-fluorophenoxy)benzoic acid (step 2 of Example 67) and 1-[4-(2H-tetrazol-5-yl)phenyl]methanamine hydrochloride (WO 9604267, WO 9604246): ¹H-NMR (DMSO-d₆) [symbol] 9.01 (1H, t, J=6.0 Hz), 7.93 (2H, d, J=8.2 Hz), 7.70 (1H, d, J=2.6 Hz), 7.53-7.46 (3H, m), 7.29-7.10 (4H, m), 6.93 (1H, d, J=8.9 Hz), 4.52 (2H, d, J=6.1 Hz); MS (ESI) m/z 424 (M+H)⁺, 422 (M−H)⁻.

### Example 77

### 5-CHLORO-2-(4-FLUOROPHENOXY)-N-{(1S)-1-[4-(2H-TETRAZOL-5-YL)PHENYL]ETHYL}BENZAMIDE

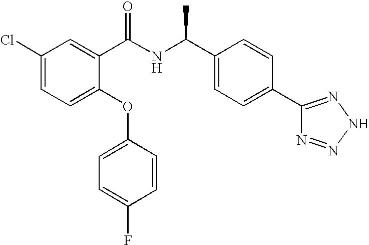

### STEP 1. tert-Butyl [(1S)-1-(4-cyanophenyl)ethyl]carbamate

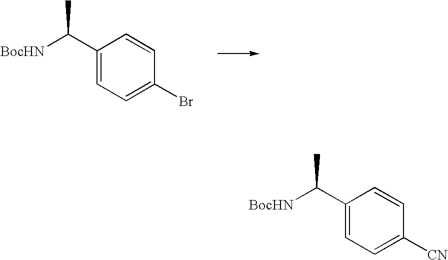

A mixture of tert-butyl [(1S)-1-(4-bromophenyl)ethyl]carbamate (step 1 of Example 5, 1.50 g, 5.00 mmol), tetrakis(triphenylphosphine)palladium (0) (0.58 g, 0.50 mmol), zinc cyanide (0.59 g, 5.00 mmol) and N,N-dimethylforamide (30 mL) was stirred at 80° C. for 16 h under nitrogen atmosphere. After cooling to room temperature, the mixture was diluted with ether (200 mL) and washed with water (100 mL×3). The organic layer was dried over magnesium sulfate and evaporated. The residue was purified by flash column chromatography on silica gel eluting with hexane/ethyl acetate (4:1) to afford 1.11 g (90%) of the title compounds as colorless syrup: ¹H-NMR (CDCl₃) [symbol] 7.64-7.61 (2H, m), 7.41 (2H, d, J=8.3 Hz), 4.83 (2H, br.s), 1.44-1.42 (12H, m).

### STEP 2. tert-Butyl {(1S)-1-[4-(2H-tetrazol-5-yl)phenyl]ethyl}carbamate

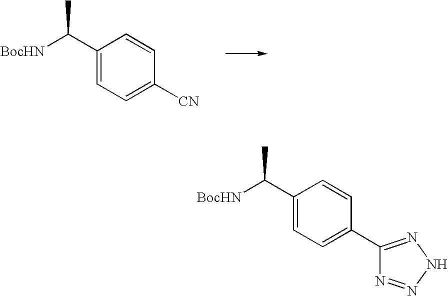

A mixture of tert-butyl [(1S)-1-(4-cyanophenyl)ethyl]carbamate (step 1, 1.11 g, 4.51 mmol), sodium azide (1.75 g, 27.1 mmol) and ammonium chloride (1.15 g, 27.1 mmol) in N,N-dimethylforamide (25 mL) was heated at 110° C. for 24 h. After cooling to room temperature, the mixture was diluted with ether (200 mL) and washed with 1 M hydrochloric acid (100 mL). The organic layer was dried over magnesium sulfate and evaporated. The residue was crystallized from dichloromethane and hexane to give 1.19 g (91%) of the title compounds as white solids: ¹H-NMR (DMSO-d₆) [symbol] 7.98 (2H, d, J=8.3 Hz), 7.51 (2H, d, J=8.3 Hz), 4.74-4.63 (1H, m), 1.37-1.32 (12H, m); MS (ESI) m/z 290 (M+H)⁺, 288 (M−H)⁻.

### STEP 3. {(1S)-1-[4-(2H-Tetrazol-5-yl)phenyl]ethyl}amine hydrochloride

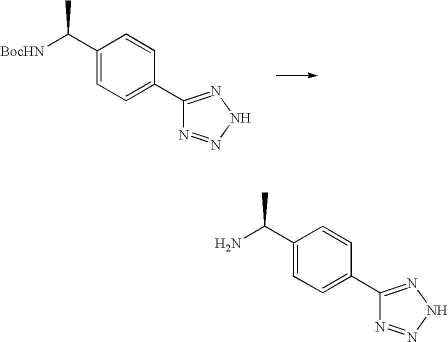

tert-Butyl {(1S)-1-[4-(2H-tetrazol-5-yl)phenyl]ethyl}carbamate (step 2, 1.19 g, 4.10 mmol) was treated with trifluoroacetic acid (10 mL) and dichloromethane (10 mL) at room temperature for 1 h. After removal of the solvent, the residue was diluted with 4 M solution of hydrogen chloride in ethyl acetate (20 mL). The mixture was concentrated under reduced pressure and the residue was washed with ether to give 0.77 g (83%) of the title compounds as white solids: ¹H-NMR (DMSO-d₆) [symbol] 8.60 (3H, br.s), 8.14 (2H, d, J=8.4 Hz), 7.75 (2H, d, J=8.4 Hz), 4.58-4.45 (1H, m), 1.55 (3H, d, J=6.8 Hz); MS (ESI) m/z 188 (M−H)⁻.

### STEP 4. 5-Chloro-2-(4-fluorophenoxy)-n-{(1s)-1-[4-(2h-tetrazol-5-yl)phenyl]ethyl}benzamide

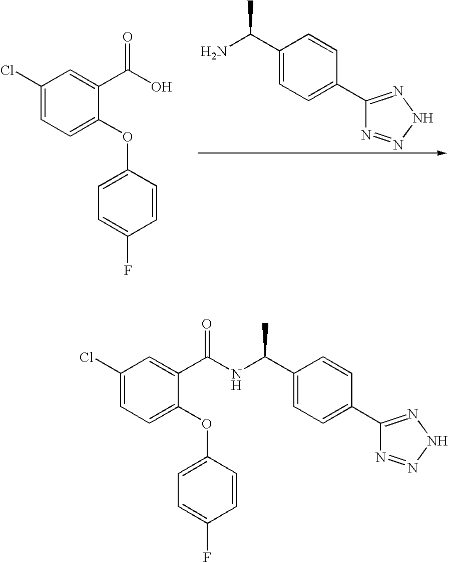

The title compound was prepared according to the procedure described in Example 75 from 5-chloro-2-(4-fluorophenoxy)benzoic acid (step 2 of Example 67) and {(1S)-1-[4-(2H-tetrazol-5-yl)phenyl]ethyl}amine hydrochloride (step 3): H-NMR (DMSO-d₆) [symbol] 8.90 (1H, d, J=7.3 Hz), 7.92 (2H, d, J=8.3 Hz), 7.60-7.49 (4H, m), 7.26-7.20 (2H, m), 7.10-7.06 (2H, m), 6.96 (1H, d, J=8.8 Hz), 5.15-5.05 (1H, m), 1.41 (3H, d, J=6.8 Hz); MS (ESI) m/z 438 (M+H)⁺, 436 (M−H)⁻.

### Example 78

### 5-FLUORO-2-(4-FLUOROBENZYL)-N-[4-(2H-TETRAZOL-5-YL)BENZYL]NICOTINAMIDE

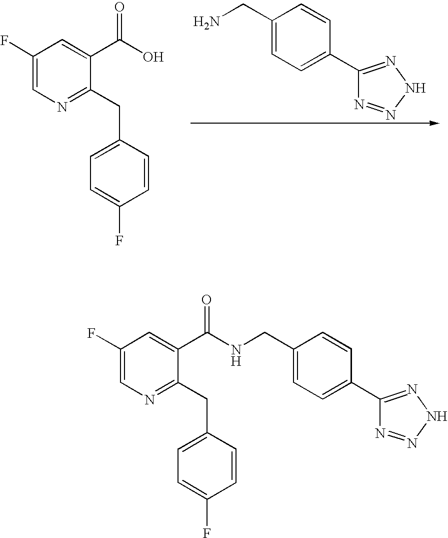

The title compound was prepared according to the procedure described in Example 75 from 5-fluoro-2-(4-fluorobenzyl)nicotinic acid (step 3 of Example 58) and 1-[4-(2H-tetrazol-5-yl)phenyl]methanamine hydrochloride: ¹H-NMR (DMSO-d₆) [symbol] 9.20 (1H, t, J=5.7 Hz), 8.59 (1H, d, J=2.8 Hz), 7.98 (2H, d, J=8.1 Hz), 7.83 (1H, dd, J=8.8, 2.9 Hz), 7.44 (2H, d, J=8.1 Hz), 7.20-7.16 (2H, m), 7.03 (2H, t, J=8.9 Hz), 4.50 (2H, d, J=5.7 Hz), 4.21 (2H, s); MS (ESI) m/z 407 (M+H)⁺, 405 (M−H)⁻.

### Example 79

### 5-CHLORO-N-{(1S)-1-[4-({[(3-CHLOROPHENYL)SULFONYL]AMINO}CARBONYL)PHENYL]ETHYL}-2-(3-FLUOROPHENOXY)NICOTINAMIDE

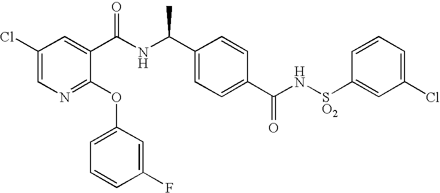

### STEP 1. 5-chloro-N-{(1S)-1-[4-({[(3-chlorophenyl)sulfonyl]amino}carbonyl)phenyl]ethyl}-2-(3-fluorophenoxy)nicotinamide

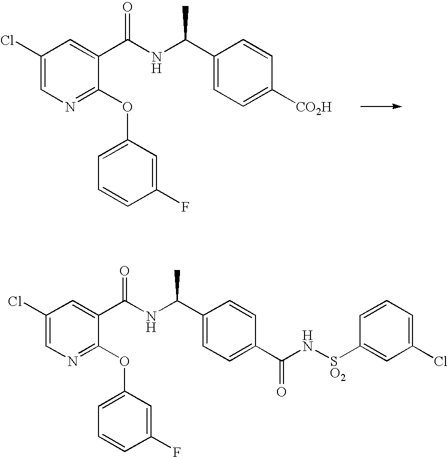

To a stirred solution of 4-[(1S)-1-({[5-Chloro-2-(4-fluorophenoxy)pyridin-3-yl]carbonyl}amino)ethyl]benzoic acid (200 mg, 0.48 mmol) in dry dichloromethane (5 mL) under argon was added 3-chlorobenzenesulfonamide (105 mg, 0.55 mmol), 4-(dimethylamino)pyridine (67 mg, 0.55 mmol), and finally 1-(3-dimethylaminopropyl)-3-ethylcarbodiimide hydrochloride (105 mg, 0.55 mmol). The resulting mixture was stirred at room temperature for 48 h. The reaction mixture was partitioned between dichloromethane (50 mL) and water (50 mL). The organic layer was separated and washed with brine (50 mL), dried (sodium sulfate), and concentrated. The residue was purified by flash column chromatography on silica gel (30 g) eluting with dichloromethane/ethyl acetate (20/1) to give a desired product. Recrystallization of the product from ethyl acetate afforded 68 mg (24%) of the title compounds as a colorless needle: H-NMR (DMSO-d₆) [symbol] 8.99 (1H, d, J=7.6 Hz), 8.26 (1H, d, J=2.5 Hz), 8.09 (1H, d, J=2.5 Hz), 7.97-7.89 (2H, m), 7.80 (4H, d, J=8.3 Hz), 7.67 (1H, dd, J=7.9, 7.9 Hz), 7.51 (2H, d, J=8.3 Hz), 7.29-7.19 (4H, m), 5.15 (1H, dq, J=7.6, 7.0 Hz), 1.42 (3H, d, J=7.0 Hz).

The following examples illustrate the preparation of EP4-receptor antagonists described in U.S. 60/568,7088:

### Example 1

### 4-[(1S)-1-({5-CHLORO-2-[(2-CHLOROPHENOXY)METHYL]BENZOYL}AMINO)ETHYL]BENZOIC ACID

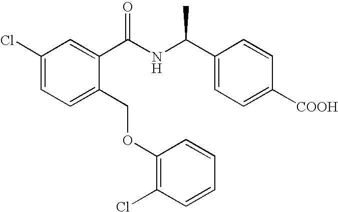

### STEP 1. Methyl 5-chloro-2-[(2-chlorophenoxy)methyl]benzoate

A mixture of methyl 2-(bromoethyl)-5-chlorobenzoate (100 mg, 0.38 mmol), 2-chlorophenol (43 μL, 0.42 mmol) and potassium carbonate (105 mg, 0.76 mmol) in N,N-dimethylforamide (2 mL) was stirred at room temperature for 3 h and at 50° C. for 4 hours. Water (5 mL) was added and the mixture was extracted with diethyl ether (15 mL×2). The combined organic extracts were washed with brine (15 mL) and dried (sodium sulfate). After removal of solvent, the residue was purified by pTLC eluting with hexane/ethyl acetate (9/1) to afford 103 mg (87%) of the title compound:

¹H-NMR (CDCl₃) δ 8.04-8.03 (1H, m), 7.91-7.87 (1H, m), 7.59-7.55 (1H, m), 7.42-7.39 (1H, m), 7.24-7.18 (1H, m), 7.02-6.90 (2H, m), 5.53 (2H, s), 3.93 (3H, s).

### STEP 2. 5-Chloro-2-[(2-chlorophenoxy)methyl]benzoic acid

To a solution of methyl 5-chloro-2-[(2-chlorophenoxy)methyl]benzoate (step 1, 103 mg, 0.33 mmol) in methanol (4 mL) and tetrahydrofuran (4 mL) was 2 N sodium hydroxide (1 mL) and the mixture was stirred at room temperature for 16 hours. After removal of solvent, the residue was diluted with water (5 mL) and the solution was acidified with 2 N hydrochloric acid. Precipitate was collected by filtration, washed with water and dried in vacuo to afford 85 mg (86%) of the title compound:

¹H-NMR (DMSO-d₆) δ7.92 (1H, br.s), 7.33 (2H, br.s), 7.48-7.45 (1H, m), 7.35-7.28 (1H, m), 7.15-7.12 (1H, m), 7.02-6.96 (1H, m), 5.52 (2H, s), a peak of COOH was not observed;

MS (ESI) m/z 295 (M−H)⁻.

### STEP 3. tert-Butyl [(1S)-1-(4-bromophenyl)ethyl]carbamate

A mixture of [(1S)-1-(4-bromophenyl)ethyl]amine (10.00 g, 50.0 mmol) and di-tert-butyl dicarbonate (11.45 g, 52.5 mmol), triethylamine (7.66 mL, 55.0 mmol) in dichloromethane (200 mL) was stirred at room temperature for 1 hour. The mixture was diluted with dichloromethane (500 mL) and washed with 1 M hydrochloric acid (300 mL), saturated sodium hydrogen carbonate aqueous (300 mL), and brine (300 mL). The organic layer was dried over magnesium sulfate, and concentrated under reduced pressure. The residue was washed with cold hexane to afford 14.73 g (98%) of the title compound as white solids:

¹H-NMR (CDCl₃) δ7.47-7.42 (2H, m), 7.18 (2H, d, J=8.4 Hz), 5.30 (2H, br.s), 1.41 (12H, br.s).

### STEP 4. Methyl 4-{(1S)-1-[(tert-butoxycarbonyl)amino]ethyl}benzoate

A mixture of tert-butyl [(1S)-1-(4-bromophenyl)ethyl]carbamate (step 3, 14.73 g, 49.1 mmol), 1,3-bis(diphenylphosphino)-propane (2.03 g, 4.91 mmol), palladium (II) acetate (1.10 g, 4.91 mmol), triethylamine (20.5 mL, 147 mmol), N,N-dimethylforamide (120 mL) and methanol (180 mL) was stirred at 80° C. for 16 h under carbon monoxide atmosphere. After cooling to room temperature, the mixture was diluted with ether (800 mL) and washed with water (500 mL×3). The organic layer was dried over magnesium sulfate and evaporated. The residue was purified by flush column chromatography on silica gel eluting with hexane/ethyl acetate (5:1) to afford 12.83 g (94%) of the title compound as white solids:

¹H-NMR (CDCl₃) δ 8.02-7.99 (2H, m), 7.37 (2H, d, J=8.4 Hz), 4.83 (2H, br.s), 3.91 (3H, s), 1.46-1.42 (12H, m).

### STEP 5. Methyl 4-[(1S)-1-aminoethyl]benzoate hydrochloride

Methyl 4-{(1S)-1-[(tert-butoxycarbonyl)amino]ethyl}benzoate (step 4, 12.83 g, 45.9 mmol) was treated with trifluoroacetic acid (100 mL) and dichloromethane (100 mL) at room temperature for 16 hours. After removal of the solvent, the residue was diluted with 10% hydrogen chloride solution in methanol (100 mL). The mixture was concentrated under reduced pressure and the residue was washed with ethylacetate to give 9.40 g (95%) of the title compound as white solids:

¹H-NMR (DMSO-d₆) δ 8.67 (2H, br.s), 8.01 (2H, d, J=8.4 Hz), 7.68 (2H, d, J=8.4 Hz), 4.49 (1H, q, J=6.9 Hz), 3.87 (3H, s), 1.53 (3H, d, J=6.9 Hz).

### STEP 6. Methyl 4-[(1S)-1-({5-chloro-2-[(2-chlorophenoxy)methyl]benzoyl}amino)ethyl]benzoate

A mixture of 5-chloro-2-[(2-chlorophenoxy)methyl]benzoic acid (step 2, 85 mg, 0.28 mmol), methyl 4-[(1S)-1-aminoethyl]benzoate hydrochloride (step 5, 73 mg, 0.34 mmol), 1-(3-dimethylaminopropyl)-3-ethylcarbodiimide hydrochloride (EDCl) (107 mg, 0.56 mmol), 1-hydroxybenzotriazole hydrate (HOBT) (76 mg, 0.56 mmol) and triethylamine (117 μL, 0.84 mmol) in dichloromethane (3 mL) was stirred at room temperature for 19 hours. Water (5 mL) was added and the organic phase was separated. The aqueous phase was extracted with ethyl acetate (10 mL×2) and the combined organic extracts were dried (sodium sulfate). After removal of solvent, the residue was purified by pTLC eluting with hexane/ethyl acetate (2/1) to afford 105 mg (82%) of the title compound:

¹H-NMR (CDCl₃) δ 7.90-7.87 (2H, m), 7.64 (1H, d, J=2.2 Hz), 7.50-7.31 (5H, m), 7.24-7.18 (1H, m), 6.97-6.87 (3H, m). 5.36-5.25 (1H, m), 5.06 (2H, dd, J=19.6, 11.2 Hz), 3.91 (3H, s), 1.27 (3H, d, J=7.3 Hz);

MS (ESI) m/z 458 (M+H)⁺, 456 (M−H)⁻.

### STEP 7. 4-[(1S)-1-({5-Chloro-2-[(2-chlorophenoxy)methyl]benzoyl}amino)ethyl]benzoic acid

To a stirred solution of methyl 4-[(1S)-1-({5-chloro-2-[(2-chlorophenoxy)methyl]benzoyl}amino)ethyl]benzoate (step 6, 407 mg, 1.02 mmol) in methanol (10 ml) was added 2 N sodium hydroxide aqueous solution (2 ml). The reaction mixture was stirred at room temperature for 3 h and then evaporated. The residue was partitioned between ethyl acetate (100 mL) and 2 N hydrochloric acid (100 mL). The organic phase was separated and the aqueous phase was extracted with ethyl acetate (100 mL). The combined organic extracts were washed with brine (50 mL), dried (sodium sulfate), and concentrated. The residual solids were recrystallized from ethyl acetate to afford 248 mg (64%) of the title compound as white solids:

¹H-NMR (DMSO-d₆) δ9.10-9.07 (1H, m), 7.87-7.84 (2H, m), 7.67-7.59 (3H, m), 7.48-7.42 (3H, m), 7.29-7.23 (1H, m), 7.03-6.94 (2H, m), 5.23 (1H, s), 5.17-5.06 (1H, m), 1.44 (3H, d, J=7.0 Hz), a peak of COOH was not observed;

MS (ESI) m/z 444 (M+H)⁺, 442 (M−H)⁻.

### Example 2

### 4-[(1S)-1-({5-CHLORO-2-[(3-CHLOROPHENOXY)METHYL]BENZOYL}AMINO)ETHYL]BENZOIC ACID

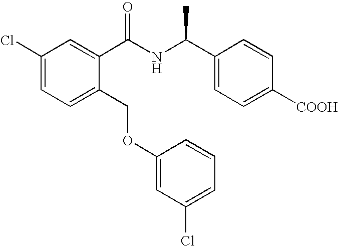

### STEP 1. Methyl 5-chloro-2-[(3-chlorophenoxy)methyl]benzoate

The title compound was prepared according to the procedure described in step 1 of Example 1 from methyl 2-(bromoethyl)-5-chlorobenzoate and 3-chlorophenol:

¹H-NMR (CDCl₃) δ 8.02 (1H, d, J=2.4 Hz), 7.67 (1H, d, J=8.4 Hz), 7.53 (1H, dd, J=8.4, 2.4 Hz), 7.21 (1H, t, J=8.1 Hz), 7.00-6.85 (3H, m), 5.44 (2H, s), 3.92 (3H, s).

### STEP 2. 5-Chloro-2-[(3-chlorophenoxy)methyl]benzoic acid

The title compound was prepared according to the procedure described in step 2 of Example 1 from methyl 5-chloro-2-[(3-chlorophenoxy)methyl]benzoate (step 1):

¹H-NMR (DMSO-d₆) δ 7.90-7.89 (1H, m), 7.70-7.62 (2H, m), 7.36-7.30 (1H, m), 7.08-6.74 (3H, m), 5.44 (2H, s), a peak of COOH was not observed;

MS (ESI) m/z 295 (M−H)⁻.

### STEP 3. Methyl 4-[(1S)-1-({5-chloro-2-[(3-chlorophenoxy)methyl]benzoyl}amino)ethyl]benzoate

The title compound was prepared according to the procedure described in step 6 of Example 1 from 5-chloro-2-[(3-chlorophenoxy)methyl]benzoic acid (step 2) and methyl 4-[(1S)-1-aminoethyl]benzoate hydrochloride (step 5 of Example 1):

¹H-NMR (CDCl₃) δ 7.93-7.90 (2H, m), 7.61 (1H, br.s), 7.45-7.44 (2H, m), 7.33-7.30 (2H, m), 7.22-7.16 (1H, m). 6.99-6.96 (1H, m), 6.85-6.84 (1H, m), 6.77-6.73 (1H, m), 6.66-6.63 (1H, m), 5.34-5.23 (1H, m), 5.02 (2H, s), 3.92 (3H, s), 1.49 (3H, d, J=7.0 Hz);

MS (ESI) m/z 458 (M+H)⁺, 456 (M−H)⁻.

### STEP 4. 4-[(1S)-1-({5-Chloro-2-[(3-chlorophenoxy)methyl]benzoyl}amino)ethyl]benzoic acid

The title compound was prepared according to the procedure described in step 7 of Example 1 from methyl 4-[(1S)-1-({5-chloro-2-[(3-chlorophenoxy)methyl]benzoyl}amino)ethyl]benzoate (step 3):

¹H-NMR (DMSO-d₆) δ 9.05-9.02 (1H, m), 7.82-7.85 (2H, m), 7.61-7.55 (3H, m), 7.42-7.39 (2H, m), 7.30-7.24 (1H, m), 7.01-6.94 (2H, m), 6.83-6.79 (1H, m), 5.17 (2H, s), 5.15-5.05 (1H, m), 1.42 (3H, d, J=7.3 Hz), a peak of COOH was not observed;

MS (ESI) m/z 444 (M+H)⁺, 442 (M−H)⁻.

### Example 3

### 4-[(1S)-1-({5-CHLORO-2-[(4-CHLOROPHENOXY)METHYL]BENZOYL}AMINO)ETHYL]BENZOIC ACID

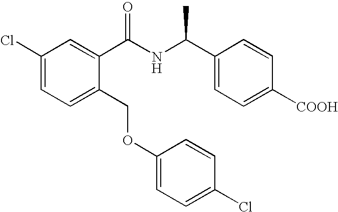

### STEP 1. Methyl 5-chloro-2-[(4-chlorophenoxy)methyl]benzoate

The title compound was prepared according to the procedure described in step 1 of Example 1 from methyl 2-(bromoethyl)-5-chlorobenzoate and 4-chlorophenol:

¹H-NMR (CDCl₃) δ 8.02 (1H, d, J=2.3 Hz), 7.68 (1H, d, J=8.5 Hz), 7.52 (1H, dd, J=8.5, 2.3 Hz), 7.28-7.22 (2H, m), 6.94-6.88 (2H, m), 5.43 (2H, s), 3.91 (3H, s).

### STEP 2. 5-Chloro-2-[(4-chlorophenoxy)methyl]benzoic acid

The title compound was prepared according to the procedure described in step 2 of Example 1 from methyl 5-chloro-2-[(4-chlorophenoxy)methyl]benzoate (step 1):

¹H-NMR (DMSO-d₆) δ 7.89-7.88 (1H, m), 7.69-7.61 (2H, m), 7.38-7.32 (2H, m), 7.03-6.97 (2H, m), 5.42 (2H, s), a peak of COOH was not observed;

MS (ESI) m/z 295 (M−H)⁻.

### STEP 3. Methyl 4-[(1S)-1-({5-chloro-2-[(4-chlorophenoxy)methyl]benzoyl}amino)ethyl]benzoate

The title compound was prepared according to the procedure described in step 6 of Example 1 from 5-chloro-2-[(4-chlorophenoxy)methyl]benzoic acid (step 2) and methyl 4-[(1S)-1-aminoethyl]benzoate hydrochloride (step 5 of Example 1):

¹H-NMR (CDCl₃) δ7.91-7.88 (2H, m), 7.62 (1H, br.s), 7.44 (2H, br.s), 7.37-7.19 (4H, m), 6.82-6.74 (2H, m), 6.67-6.39 (1H, m), 5.34-5.23 (1H, m), 5.00 (2H, s), 3.92 (3H, s), 1.48 (3H, d, J=6.8 Hz);

MS (ESI) m/z 458 (M+H)⁺, 456 (M−H)⁻.

### STEP 4. 4-[(1S)-1-({5-Chloro-2-[(4-chlorophenoxy)methyl]benzoyl}amino)ethyl]benzoic acid

The title compound was prepared according to the procedure described in step 7 of Example 1 from methyl 4-[(1S)-1-({5-chloro-2-[(4-chlorophenoxy)methyl]benzoyl}amino)ethyl]benzoate (step 3):

¹H-NMR (DMSO-d₆) δ 9.07-9.04 (1H, m), 7.87-7.84 (2H, m), 7.60-7.54 (3H, m), 7.48-7.45 (2H, m), 7.29-7.26 (2H, m), 6.87-6.84 (2H, m), 5.17-5.05 (3H, m), 1.43 (3H, d, J=7.0 Hz), a peak of COOH was not observed;

MS (ESI) m/z 444 (M+H)⁺, 442 (M−H)⁻.

### Example 4

### 4-[(1S)-1-({5-CHLORO-2-[(4-FLUOROPHENOXY)METHYL]BENZOYL}AMINO)ETHYL]BENZOIC ACID

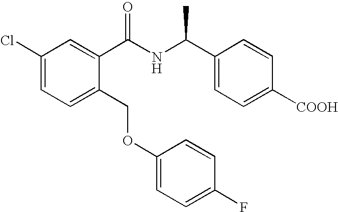

### STEP 1. Methyl 5-chloro-2-[(4-fluorophenoxy)methyl]benzoate

The title compound was prepared according to the procedure described in step 1 of Example 1 from methyl 2-(bromoethyl)-5-chlorobenzoate and 4-fluorophenol:

¹H-NMR (CDCl₃) δ 8.01 (1H, d, J=2.2 Hz), 7.69 (1H, d, J=8.5 Hz), 7.52 (1H, dd, J=8.5, 2.2 Hz), 7.02-6.89 (4H, m), 5.42 (2H, s), 3.91 (3H, s).

### STEP 2. 5-Chloro-2-[(4-fluorophenoxy)methyl]benzoic acid

The title compound was prepared according to the procedure described in step 2 of Example 1 from methyl 5-chloro-2-[(4-fluorophenoxy)methyl]benzoate (step 1):

¹H-NMR (DMSO-d₆) δ7.90-7.89 (1H, m), 7.71-7.63 (2H, m), 7.17-7.10 (2H, m), 7.01-6.96 (2H, m), 5.40 (2H, s), a peak of COOH was not observed;

MS (ESI) m/z 279 (M−H)⁻.

### STEP 3. Methyl 4-[(1S)-1-({5-chloro-2-[(4-fluorophenoxy)methyl]benzoyl}amino)ethyl]benzoate

The title compound was prepared according to the procedure described in step 6 of Example 1 from 5-chloro-2-[(4-fluorophenoxy)methyl]benzoic acid (step 2) and methyl 4-[(1S)-1-aminoethyl]benzoate hydrochloride (step 5 of Example 1):

¹H-NMR (CDCl₃) δ7.91-7.88 (2H, m), 7.63 (1H, br.s), 7.47-7.40 (2H, m), 7.32-7.29 (2H, m), 6.99-6.92 (2H, m), 6.81-6.75 (3H, m), 5.33-5.23 (1H, m), 4.98 (2H, s), 3.92 (3H, s), 1.47 (3H, d, J=7.0 Hz);

MS (ESI) m/z 442 (M+H)⁺, 440 (M−H)⁻.

### STEP 4. 4-[(1S)-1-({5-Chloro-2-[(4-fluorophenoxy)methyl]benzoyl}amino)ethyl]benzoic acid

The title compound was prepared according to the procedure described in step 7 of Example 1 from methyl 4-[(1S)-1-({5-chloro-2-[(4-fluorophenoxy)methyl]benzoyl}amino)ethyl]benzoate (step 3):

¹H-NMR (DMSO-d₆) δ 9.07-9.04 (1H, m), 7.86-7.83 (2H, m), 7.61-7.54 (3H, m), 7.49-7.46 (2H, m), 7.10-7.03 (2H, m), 6.88-6.82 (2H, m), 5.13-5.05 (3H, m), 1.42 (3H, d, J=6.8 Hz), a peak of COOH was not observed;

MS (ESI) m/z 428 (M+H)⁺, 426 (M−H)⁻.

### Example 5

### 4-[(1S)-1-({5-CHLORO-2-[(3-FLUOROPHENOXY)METHYL]BENZOYL}AMINO)ETHYL]BENZOIC ACID

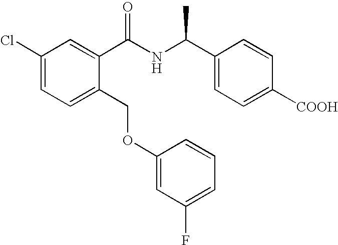

### STEP 1. Methyl 5-chloro-2-[(3-fluorophenoxy)methyl]benzoate

The title compound was prepared according to the procedure described in step 1 of Example 1 from methyl 2-(bromoethyl)-5-chlorobenzoate and 3-fluorophenol:

¹H-NMR (CDCl₃) δ 8.02 (1H, d, J=2.3 Hz), 7.68 (1H, d, J=8.6 Hz), 7.53 (1H, dd, J=8.6, 2.3 Hz), 7.28-7.19 (1H, m), 6.78-6.65 (3H, m), 5.45 (2H, s), 3.92 (3H, s).

### STEP 2. 5-Chloro-2-[(3-fluorophenoxy)methyl]benzoic acid

The title compound was prepared according to the procedure described in step 2 of Example 1 from methyl 5-chloro-2-[(3-fluorophenoxy)methyl]benzoate (step 1):

¹H-NMR (DMSO-d₆) δ 7.91-7.89 (1H, m), 7.71-7.63 (2H, m), 7.38-7.29 (1H, m), 6.89-6.75 (3H, m), 5.44 (2H, s), a peak of COOH was not observed.

### STEP 3. Methyl 4-[(1S)-1-({5-chloro-2-[(3-fluorophenoxy)methyl]benzoyl}amino)ethyl]benzoate

The title compound was prepared according to the procedure described in step 6 of Example 1 from 5-chloro-2-[(3-fluorophenoxy)methyl]benzoic acid (step 2) and methyl 4-[(1S)-1-aminoethyl]benzoate hydrochloride (step 5 of Example 1)

¹H-NMR (CDCl₃) δ7.93-7.90 (2H, m), 7.61 (1H, br.s), 7.45 (2H, br.s), 7.33-7.18 (3H, m), 6.75-6.54 (4H, m), 5.31-5.26 (1H, m), 5.03 (2H, s), 3.91 (3H, s), 1.48 (3H, d, J=7.1 Hz);

MS (ESI) m/z 442 (M+H)⁺.

### STEP 4. 4-[(1S)-1-({5-Chloro-2-[(3-fluorophenoxy)methyl]benzoyl}amino)ethyl]benzoic acid

The title compound was prepared according to the procedure described in step 7 of Example 1 from methyl 4-[(1S)-1-({5-chloro-2-[(3-fluorophenoxy)methyl]benzoyl}amino)ethyl]benzoate (step 3):

¹H-NMR (DMSO-d₆) δ9.08-9.05 (1H, m), 7.87-7.84 (2H, m), 7.61-7.55 (3H, m), 7.49-7.46 (2H, m), 7.31-7.23 (1H, m), 6.79-6.67 (3H, m), 5.20-5.06 (3H, m), 1.43 (3H, d, J=7.0 Hz), a peak of COOH was not observed;

MS (ESI) m/z 428 (M+H)⁺, 426 (M−H)⁻.

### Example 6

### 4-[(1S)-1-({5-CHLORO-2-[(2-FLUOROPHENOXY)METHYL]BENZOYL}AMINO)ETHYL]BENZOIC ACID

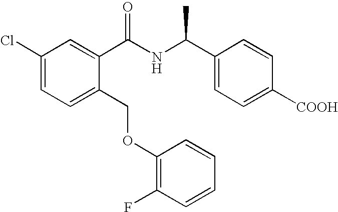

### STEP 1. Methyl 5-chloro-2-[(2-fluorophenoxy)methyl]benzoate

The title compound was prepared according to the procedure described in step 1 of Example 1 from methyl 2-(bromoethyl)-5-chlorobenzoate and 2-fluorophenol: ¹H-NMR (CDCl₃) δ 8.02 (1H, d, J=2.4 Hz), 7.80-7.77 (1H, m), 7.57-7.53 (1H, m), 7.15-6.89 (4H, m), 5.52 (2H, s), 3.92 (3H, s).

### STEP 2. 5-Chloro-2-[(3-fluorophenoxy)methyl]benzoic acid

The title compound was prepared according to the procedure described in step 2 of Example 1 from methyl 5-chloro-2-[(2-fluorophenoxy)methyl]benzoate (step 1):

¹H-NMR (DMSO-d₆) δ 7.91-7.90 (1H, m), 7.74-7.66 (2H, m), 7.28-7.09 (3H, m), 7.01-6.93 (1H, m), 5.49 (2H, s), a peak of COOH was not observed.

### STEP 3. Methyl 4-[(1S)-1-({5-chloro-2-[(2-fluorophenoxy)methyl]benzoyl}amino)ethyl]benzoate

The title compound was prepared according to the procedure described in step 6 of Example 1 from 5-chloro-2-[(2-fluorophenoxy)methyl]benzoic acid (step 2) and methyl 4-[(1S)-1-aminoethyl]benzoate hydrochloride (step 5 of Example 1):

¹H-NMR (CDCl₃) δ 7.91 (2H, d, J=8.2 Hz), 7.63 (1H, br.s), 7.42 (2H, br.s), 7.35 (2H, d, J=8.2 Hz), 7.11-6.89 (5H, m), 5.35-5.24 (1H, m), 5.15-5.05 (2H, m), 3.91 (3H, s), 1.50 (3H, d, J=6.9 Hz);

MS (ESI) m/z 442 (M+H)⁺.

### STEP 4. 4-[(1S)-1-({5-Chloro-2-[(2-fluorophenoxy)methyl]benzoyl}amino)ethyl]benzoic acid

The title compound was prepared according to the procedure described in step 7 of Example 1 from methyl 4-[(1S)-1-({5-chloro-2-[(2-fluorophenoxy)methyl]benzoyl}amino)ethyl]benzoate (step 3):

¹H-NMR (DMSO-d₆) δ 9.11-9.08 (1H, m), 7.86-7.83 (2H, m), 7.64-7.57 (3H, m), 7.49-7.46 (2H, m), 7.25-7.17 (1H, m), 7.11-7.03 (2H, m), 6.98-6.90 (1H, m), 5.21-5.05 (3H, m), 1.43 (3H, d, J=7.0 Hz), a peak of COOH was not observed;

MS (ESI) m/z 428 (M+H)⁺, 426 (M−H)⁻.

### Example 7

### 4-[(1S)-1-({5-CHLORO-2-[(2,3-DIFLUOROPHENOXY)METHYL]BENZOYL}AMINO)ETHYL]BENZOIC ACID

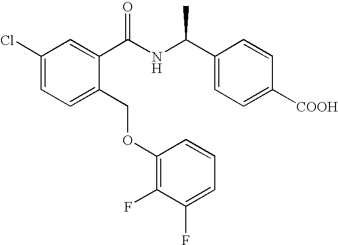

### STEP 1. Methyl 5-chloro-2-[(2,3-difluorophenoxy)methyl]benzoate

The title compound was prepared according to the procedure described in step 1 of Example 1 from methyl 2-(bromoethyl)-5-chlorobenzoate and 2,3-difluorophenol:

¹H-NMR (CDCl₃) δ 8.03 (1H, d, J=2.3 Hz), 7.76 (1H, d, J=8.5 Hz), 7.55 (1H, dd, J=8.5, 2.3 Hz), 7.02-6.92 (1H, m), 6.84-6.75 (2H, m), 5.53 (2H, s), 3.93 (3H, s).

### STEP 2. 5-Chloro-2-[(2,3-difluorophenoxy)methyl]benzoic acid

The title compound was prepared according to the procedure described in step 2 of Example 1 from methyl 5-chloro-2-[(2,3-difluorophenoxy)methyl]benzoate (step 1):

¹H-NMR (DMSO-d₆) δ7.92-7.91 (1H, m), 7.74-7.65 (2H, m), 7.19-7.10 (1H, m), 7.06-6.97 (2H, m), 5.53 (2H, s), a peak of COOH was not observed;

MS (ESI) m/z 297 (M−H)⁻.

### STEP 3. Methyl 4-[(1S)-1-({5-chloro-2-[(2,3-difluorophenoxy)methyl]benzoyl}amino)ethyl]benzoate

The title compound was prepared according to the procedure described in step 6 of Example 1 from 5-chloro-2-[(2,3-difluorophenoxy)methyl]benzoic acid (step 2) and methyl 4-[(1S)-1-aminoethyl]benzoate hydrochloride (step 5 of Example 1)

¹H-NMR (CDCl₃) δ 7.94-7.91 (2H, m), 7.59 (1H, br.s), 7.45 (2H, br.s), 7.39-7.35 (2H, m), 7.00-6.64 (4H, m), 5.35-5.24 (1H, m), 5.18-5.08 (2H, m), 3.91 (3H, s), 1.53 (3H, d, J=7.1 Hz);

MS (ESI) m/z 460 (M+H)⁺.

### STEP 4. 4-[(1S)-1-({5-Chloro-2-[(2,3-difluorophenoxy)methyl]benzoyl}amino)ethyl]benzoic acid

The title compound was prepared according to the procedure described in step 7 of Example 1 from methyl 4-[(1S)-1-({5-chloro-2-[(2,3-difluorophenoxy)methyl]benzoyl}amino)ethyl]benzoate (step 3):

¹H-NMR (DMSO-d₆) δ 9.10-9.07 (1H, m), 7.85-7.82 (2H, m), 7.63 (3H, br.s), 7.47-7.44 (2H, m), 7.09-6.90 (3H, m), 5.30-5.05 (3H, m), 1.43 (3H, d, J=7.0 Hz), a peak of COOH was not observed;

MS (ESI) m/z 446 (M+H)⁺, 444 (M−H)⁻.

### Example 8

### 4-[(1S)-1-({5-CHLORO-2-[(2,4-DIFLUOROPHENOXY)METHYL]BENZOYL}AMINO)ETHYL]BENZOIC ACID

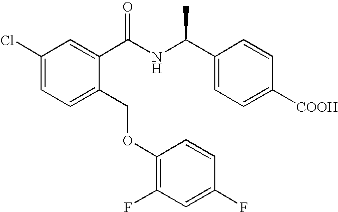

### STEP 1. Methyl 5-chloro-2-[(2,4-difluorophenoxy)methyl]benzoate

The title compound was prepared according to the procedure described in step 1 of Example 1 from methyl 2-(bromoethyl)-5-chlorobenzoate and 2,4-difluorophenol:

¹H-NMR (CDCl₃) δ 8.02 (1H, d, J=2.2 Hz), 7.78-7.75 (1H, m), 7.57-7.53 (1H, m), 7.01-6.73 (3H, m), 5.48 (2H, s), 3.92 (3H, s).

### STEP 2. 5-Chloro-2-[(2,4-difluorophenoxy)methyl]benzoic acid

The title compound was prepared according to the procedure described in step 2 of Example 1 from methyl 5-chloro-2-[(2,4-difluorophenoxy)methyl]benzoate (step 1):

¹H-NMR (DMSO-d₆) δ 7.91-7.90 (1H, m), 7.74-7.65 (2H, m), 7.36-7.27 (1H, m), 7.24-7.15 (1H, m), 7.06-6.97 (1H, m), 5.47 (2H, s), a peak of COOH was not observed;

MS (ESI) m/z 297 (M−H)⁻.

### STEP 3. Methyl 4-[(1S)-1-({5-chloro-2-[(2,4-difluorophenoxy)methyl]benzoyl}amino)ethyl]benzoate

The title compound was prepared according to the procedure described in step 6 of Example 1 from 5-chloro-2-[(2,4-difluorophenoxy)methyl]benzoic acid (step 2) and methyl 4-[(1S)-1-aminoethyl]benzoate hydrochloride (step 5 of Example 1)

¹H-NMR (CDCl₃) δ 7.94-7.91 (2H, m), 7.61 (1H, br.s), 7.42 (2H, br.s), 7.39-7.36 (2H, m), 6.93-6.73 (4H, m), 5.35-5.25 (1H, m), 5.07 (2H, s), 3.91 (3H, s), 1.53 (3H, d, J=6.9 Hz);

MS (ESI) m/z 460 (M+H)⁺.

### STEP 4. 4-[(1S)-1-({5-Chloro-2-[(2,4-difluorophenoxy)methyl]benzoyl}amino)ethyl]benzoic acid

The title compound was prepared according to the procedure described in step 7 of Example 1 from methyl 4-[(1S)-1-({5-chloro-2-[(2,4-difluorophenoxy)methyl]benzoyl}amino)ethyl]benzoate (step 3):

¹H-NMR (DMSO-d₆) δ 9.10-9.07 (1H, m), 7.85-7.82 (2H, m), 7.59 (3H, br.s), 7.48-7.45 (2H, m), 7.30-7.21 (1H, m), 7.12-7.03 (1H, m), 6.98-6.90 (1H, m), 5.26-5.05 (3H, m), 1.43 (3H, d, J=7.0 Hz), a peak of COOH was not observed;

MS (ESI) m/z 446 (M+H)⁺, 444 (M−H)⁻.

### Example 9

### 4-[(1S)-1-({5-CHLORO-2-[(2,5-DIFLUOROPHENOXY)METHYL]BENZOYL}AMINO)ETHYL]BENZOIC ACID

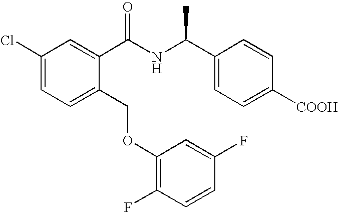

### STEP 1. Methyl 5-chloro-2-[(2,5-difluorophenoxy)methyl]benzoate

The title compound was prepared according to the procedure described in step 1 of Example 1 from methyl 2-(bromoethyl)-5-chlorobenzoate and 2,5-difluorophenol:

¹H-NMR (CDCl₃) δ 8.03 (1H, d, J=2.2 Hz), 7.74 (1H, d, J=8.4 Hz), 7.55 (1H, dd, J=8.4, 2.2 Hz), 7.10-7.01 (1H, m), 6.80-6.73 (1H, m), 6.65-6.57 (1H, m), 5.50 (2H, s), 3.93 (3H, s).

### STEP 2. 5-Chloro-2-[(2,5-difluorophenoxy)methyl]benzoic acid

The title compound was prepared according to the procedure described in step 2 of Example 1 from methyl 5-chloro-2-[(2,5-difluorophenoxy)methyl]benzoate (step 1):

¹H-NMR (DMSO-d₆) δ 7.89-7.88 (1H, m), 7.71-7.62 (2H, m), 7.31-7.23 (1H, m), 7.15-7.09 (1H, m), 6.82-6.75 (1H, m), 5.48 (2H, s), a peak of COOH was not observed.

### STEP 3. Methyl 4-[(1S)-1-({5-chloro-2-[(2,5-difluorophenoxy)methyl]benzoyl}amino)ethyl]benzoate

The title compound was prepared according to the procedure described in step 6 of Example 1 from 5-chloro-2-[(2,5-difluorophenoxy)methyl]benzoic acid (step 2) and methyl 4-[(1S)-1-aminoethyl]benzoate hydrochloride (step 5 of Example 1)

¹H-NMR (CDCl₃) δ 7.95-7.92 (2H, m), 7.59 (1H, br.s), 7.48-7.42 (2H, m), 7.39-7.36 (2H, m), 7.05-6.96 (1H, m), 6.72-6.58 (3H, m), 5.36-5.25 (1H, m), 5.14-5.04 (2H, m), 3.91 (3H, s), 1.53 (3H, d, J=7.1 Hz);

MS (ESI) m/z 460 (M+H)⁺.

### STEP 4. 4-[(1S)-1-({5-Chloro-2-[(2,5-difluorophenoxy)methyl]benzoyl}amino)ethyl]benzoic acid

The title compound was prepared according to the procedure described in step 7 of Example 1 from methyl 4-[(1S)-1-({5-chloro-2-[(2,5-difluorophenoxy)methyl]benzoyl}amino)ethyl]benzoate (step 3):

¹H-NMR (DMSO-d₆) δ 9.11-9.09 (1H, m), 7.84-7.81 (2H, m), 7.60 (3H, br.s), 7.48-7.45 (2H, m), 7.30-7.20 (1H, m), 7.09-7.01 (1H, m), 6.80-6.72 (1H, m), 5.23 (2H, s), 5.15-5.05 (1H, m), 1.43 (3H, d, J=7.0 Hz), a peak of COOH was not observed;

MS (ESI) m/z 446 (M+H)⁺, 444 (M−H)⁻.

### Example 10

### 4-[(1S)-1-({5-CHLORO-2-[(2,6-DIFLUOROPHENOXY)METHYL]BENZOYL}AMINO)ETHYL]BENZOIC ACID

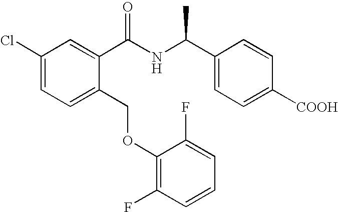

### STEP 1. Methyl 5-chloro-2-[(2,6-difluorophenoxy)methyl]benzoate

The title compound was prepared according to the procedure described in step 1 of Example 1 from methyl 2-(bromoethyl)-5-chlorobenzoate and 2,6-difluorophenol:

¹H-NMR (CDCl₃) δ 7.99 (1H, d, J=2.3 Hz), 7.84 (1H, d, J=8.4 Hz), 7.56 (1H, dd, J=8.4, 2.3 Hz), 7.03-6.84 (3H, m), 5.55 (2H, s), 3.90 (3H, s).

### STEP 2. 5-Chloro-2-[(2,6-difluorophenoxy)methyl]benzoic acid

The title compound was prepared according to the procedure described in step 2 of Example 1 from methyl 5-chloro-2-[(2,6-difluorophenoxy)methyl]benzoate (step 1):

¹H-NMR (DMSO-d₆) δ7.88-7.87 (1H, m), 7.77-7.69 (2H, m), 7.16-7.12 (3H, m), 5.53 (2H, s), a peak of COOH was not observed.

### STEP 3. Methyl 4-[(1S)-1-({5-chloro-2-[(2,6-difluorophenoxy)methyl]benzoyl}amino)ethyl]benzoate

The title compound was prepared according to the procedure described in step 6 of Example 1 from 5-chloro-2-[(2,6-difluorophenoxy)methyl]benzoic acid (step 2) and methyl 4-[(1S)-1-aminoethyl]benzoate hydrochloride (step 5 of Example 1)

¹H-NMR (CDCl₃) δ 8.03-8.00 (2H, m), 7.65-7.64 (1H, m), 7.49-7.46 (2H, m), 7.34-7.21 (4H, m), 7.03-6.85 (2H, m), 5.42-5.18 (3H, m), 3.91 (3H, s), 1.61 (3H, d, J=6.9 Hz);

MS (ESI) m/z 460 (M+H)⁺.

### STEP 4. 4-[(1S)-1-({5-Chloro-2-[(2,6-difluorophenoxy)methyl]benzoyl}amino)ethyl]benzoic acid

The title compound was prepared according to the procedure described in step 7 of Example 1 from methyl 4-[(1S)-1-({5-chloro-2-[(2,6-difluorophenoxy)methyl]benzoyl}amino)ethyl]benzoate (step 3):

¹H-NMR (DMSO-d₆) δ 9.06-9.04 (1H, m), 7.89-7.86 (2H, m), 7.69-7.59 (3H, m), 7.49-7.46 (2H, m), 7.13-7.09 (3H, m), 5.33-5.23 (2H, m), 5.15-5.05 (1H, m), 1.43 (3H, d, J=6.8 Hz), a peak of COOH was not observed;

MS (ESI) m/z 446 (M+H)⁺, 444 (M−H)⁻.

### Example 11

### 4-[(1S)-1-({5-CHLORO-2-[(3,4-DIFLUOROPHENOXY)METHYL]BENZOYL}AMINO)ETHYL]BENZOIC ACID

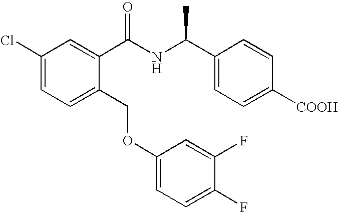

### STEP 1. Methyl 5-chloro-2-[(3,4-difluorophenoxy)methyl]benzoate

The title compound was prepared according to the procedure described in step 1 of Example 1 from methyl 2-(bromoethyl)-5-chlorobenzoate and 3,4-difluorophenol:

¹H-NMR (CDCl₃) δ 8.02 (1H, d, J=2.3 Hz), 7.66 (1H, d, J=8.4 Hz), 7.53 (1H, dd, J=8.4, 2.3 Hz), 7.13-7.02 (1H, m), 6.85-6.77 (1H, m), 6.71-6.65 (1H, m), 5.41 (2H, s), 3.92 (3H, s).

### STEP 2. 5-Chloro-2-[(3,4-difluorophenoxy)methyl]benzoic acid

The title compound was prepared according to the procedure described in step 2 of Example 1 from methyl 5-chloro-2-[(3,4-difluorophenoxy)methyl]benzoate (step 1):

¹H-NMR (DMSO-d₆) δ 7.88-7.87 (1H, m), 7.69-7.61 (2H, m), 7.40-7.30 (1H, m), 7.16-7.08 (1H, m), 6.82-6.77 (1H, m), 5.59 (2H, s), a peak of COOH was not observed.

### STEP 3. Methyl 4-[(1S)-1-({5-chloro-2-[(3,4-difluorophenoxy)methyl]benzoyl}amino)ethyl]benzoate

The title compound was prepared according to the procedure described in step 6 of Example 1 from 5-chloro-2-[(3,4-difluorophenoxy)methyl]benzoic acid (step 2) and methyl 4-[(1S)-1-aminoethyl]benzoate hydrochloride (step 5 of Example 1)

¹H-NMR (CDCl₃) δ 7.93-7.90 (2H, m), 7.59 (1H, br.s), 7.45-7.44 (2H, m), 7.36-7.32 (2H, m), 7.09-6.99 (1H, m), 6.68-6.60 (1H, m), 6.58-6.50 (2H, m), 5.34-5.24 (1H, m), 5.04-4.95 (2H, m), 3.92 (3H, s), 1.52 (3H, d, J=6.9 Hz);

MS (ESI) m/z 460 (M+H)⁺.

### STEP 4. 4-[(1S)-1-({5-Chloro-2-[(3,4-difluorophenoxy)methyl]benzoyl}amino)ethyl]benzoic acid

The title compound was prepared according to the procedure described in step 7 of Example 1 from methyl 4-[(1S)-1-({5-chloro-2-[(3,4-difluorophenoxy)methyl]benzoyl}amino)ethyl]benzoate (step 3):

¹H-NMR (DMSO-d₆) δ 9.08-9.05 (1H, m), 7.87-7.82 (2H, m), 7.57 (3H, br.s), 7.48-7.45 (2H, m), 7.35-7.23 (1H, m), 6.98-6.90 (1H, m), 6.68-6.64 (1H, m), 5.16-5.06 (3H, m), 1.43 (3H, d, J=6.8 Hz), a peak of COOH was not observed;

MS (ESI) m/z 446 (M+H)⁺, 444 (M−H)⁻.

### Example 12

### 4-[(1S)-1-({5-CHLORO-2-[(3,5-DIFLUOROPHENOXY)METHYL]BENZOYL}AMINO)ETHYL]BENZOIC ACID

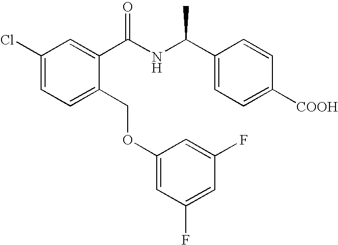

### STEP 1. Methyl 5-chloro-2-[(3,5-difluorophenoxy)methyl]benzoate

The title compound was prepared according to the procedure described in step 1 of Example 1 from methyl 2-(bromoethyl)-5-chlorobenzoate and 3,5-difluorophenol:

¹H-NMR (CDCl₃) δ 8.04-8.03 (1H, m), 7.66-7.63 (1H, m), 7.56-7.52 (1H, m), 6.59-6.40 (3H, m), 5.43 (2H, s), 3.92 (3H, s).

### STEP 2. 5-Chloro-2-[(3,5-difluorophenoxy)methyl]benzoic acid

The title compound was prepared according to the procedure described in step 2 of Example 1 from methyl 5-chloro-2-[(3,5-difluorophenoxy)methyl]benzoate (step 1):

¹H-NMR (DMSO-d₆) δ 7.88 (1H, d, J=2.2 Hz), 7.69-7.60 (2H, m), 6.84-6.74 (3H, m), 5.42 (2H, s), a peak of COOH was not observed.

### STEP 3. Methyl 4-[(1S)-1-({5-chloro-2-[(3,5-difluorophenoxy)methyl]benzoyl}amino)ethyl]benzoate

The title compound was prepared according to the procedure described in step 6 of Example 1 from 5-chloro-2-[(3,5-difluorophenoxy)methyl]benzoic acid (step 2) and methyl 4-[(1S)-1-aminoethyl]benzoate hydrochloride (step 5 of Example 1)

¹H-NMR (CDCl₃) δ 7.96-7.93 (2H, m), 7.57 (1H, br.s), 7.45-7.44 (2H, m), 7.37-7.34 (2H, m), 6.49-6.33 (4H, m), 5.35-5.24 (1H, m), 5.04 (2H, s), 3.92 (3H, s), 1.53 (3H, d, J=6.9 Hz);

MS (ESI) m/z 460 (M+H)⁺.

### STEP 4. 4-[(1S)-1-({5-Chloro-2-[(3,5-difluorophenoxy)methyl]benzoyl}amino)ethyl]benzoic acid

The title compound was prepared according to the procedure described in step 7 of Example 1 from methyl 4-[(1S)-1-({5-chloro-2-[(3,5-difluorophenoxy)methyl]benzoyl}amino)ethyl]benzoate (step 3):

¹H-NMR (DMSO-d₆) δ 9.09-9.06 (1H, m), 7.86-7.83 (2H, m), 7.58 (3H, br.s), 7.49-7.46 (2H, m), 6.80-6.71 (1H, m), 6.64-6.57 (2H, m), 5.22-5.05 (3H, m), 1.43 (3H, d, J=7.0 Hz), a peak of COOH was not observed;

MS (ESI) m/z 446 (M+H)⁺, 444 (M−H)⁻.

### Example 13

### 4-[(1S)-1-({5-CHLORO-2-[(4-METHYLPHENOXY)METHYL]BENZOYL}AMINO)ETHYL]BENZOIC ACID

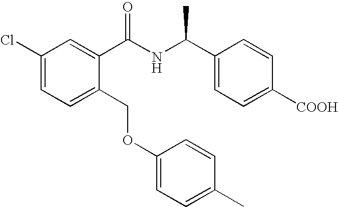

### STEP 1. Methyl 5-chloro-2-[(4-methylphenoxy)methyl]benzoate

The title compound was prepared according to the procedure described in step 1 of Example 1 from methyl 2-(bromoethyl)-5-chlorobenzoate and 4-methylphenol:

¹H-NMR (CDCl₃) δ 8.00 (1H, d, J=2.3 Hz), 7.71 (1H, d, J=8.4 Hz), 7.51 (1H, dd, J=8.4, 2.3 Hz), 7.10-7.07 (2H, m), 6.89-6.85 (2H, m), 5.43 (2H, s), 3.91 (3H, s), 2.29 (3H, s).

### STEP 2. 5-Chloro-2-[(4-methylphenoxy)methyl]benzoic acid

The title compound was prepared according to the procedure described in step 2 of Example 1 from methyl 5-chloro-2-[(4-methylphenoxy)methyl]benzoate (step 1):

¹H-NMR (DMSO-d₆) δ 7.85 (1H, m), 7.65-7.59 (2H, m), 7.09-7.06 (2H, m), 6.85-6.82 (2H, m), 5.37 (2H, s), 2.21 (3H, s), a peak of COOH was not observed.

### STEP 3. Methyl 4-[(1S)-1-({5-chloro-2-[(4-methylphenoxy)methyl]benzoyl}amino)ethyl]benzoate

The title compound was prepared according to the procedure described in step 6 of Example 1 from 5-chloro-2-[(4-methylphenoxy)methyl]benzoic acid (step 2) and methyl 4-[(1S)-1-aminoethyl]benzoate hydrochloride (step 5 of Example 1):

¹H-NMR (CDCl₃) δ 7.89-7.86 (2H, m), 7.68 (1H, br.s), 7.45-7.39 (2H, m), 7.29-7.26 (2H, m), 7.10-7.07 (2H, m), 7.01-6.99 (1H, m), 6.78-6.75 (2H, m), 5.33-5.22 (1H, m), 5.02-4.93 (2H, m), 3.91 (3H, s), 2.31 (3H, s), 1.42 (3H, d, J=6.9 Hz);

MS (ESI) m/z 438 (M+H)⁺, 436 (M−H)⁻.

### STEP 4. 4-[(1S)-1-({5-Chloro-2-[(4-methylphenoxy)methyl]benzoyl}amino)ethyl]benzoic acid

The title compound was prepared according to the procedure described in step 7 of Example 1 from methyl 4-[(1S)-1-({5-chloro-2-[(4-methylphenoxy)methyl]benzoyl}amino)ethyl]benzoate (step 3):

¹H-NMR (DMSO-d₆) δ 9.07-9.04 (1H, m), 7.86-7.83 (2H, m), 7.60-7.53 (3H, m), 7.49-7.46 (2H, m), 7.05-7.02 (2H, m), 6.74-6.71 (2H, m), 5.15-5.03 (3H, m), 2.22 (3H, s), 1.43 (3H, d, J=7.3 Hz), a peak of COOH was not observed;

MS (ESI) m/z 424 (M+H)⁺, 422 (M−H)⁻.

### Example 14

### 4-[(1S)-1-({5-CHLORO-2-{[(5-FLUOROPYRIDIN-3-YL)OXY]METHYL}BENZOYL)AMINO]ETHYL}BENZOIC ACID

### STEP 1. Methyl 5-chloro-2-{[(5-fluoropyridin-3-yl)oxy]methyl}benzoate

To a solution of 3-fluoro-5-hydroxypyridine (34 mg, 0.30 mmol) in dimethylformamide (3 mL) was added sodium hydride (60% dispersion in mineral oil, 12 mg, 0.30 mmol) at 0° C. and the mixture was stirred at room temperature for 15 minutes. To the mixture was added methyl 2-(bromomethyl)-5-chlorobenzoate (100 mg, 0.4 mmol) in dimethylformamide and the mixture was stirred at room temperature overnight. The mixture was quenched with water and extracted with ethyl acetate. The organic layer was washed with brine, dried over sodium sulfate and evaporated. The residue was purified by flash column chromatography on silica gel to afford 47 mg (53%) of the title compound:

¹H-NMR (CDCl₃) δ 8.25 (1H, d, J=1.5 Hz), 8.14 (1H, d, J=2.2 Hz), 8.05 (1H, d, J=2.2 Hz), 7.66 (1H, d, J=8.4 Hz), 7.57 (1H, dd, J=8.4, 2.2 Hz), 7.10-7.00 (1H, m), 5.51 (2H, s), 3.93 (3H, s).

### STEP 2. 5-Chloro-2-{[(5-fluoropyridin-3-yl)oxy]methyl}benzoic acid

The title compound was prepared according to the procedure described in step 2 of Example 1 from methyl 5-chloro-2-{[(5-fluoropyridin-3-yl)oxy]methyl}benzoate (step 1). The title compound was used in the next step without further purification:

MS (ESI) m/z 280 (M−H)⁻.

### STEP 3. Methyl 4-{(1S)-1-[(5-chloro-2-{[(5-fluoropyridin-3-yl)oxy]methyl}benzoyl)amino]ethyl}benzoate

The title compound was prepared according to the procedure described in step 6 of Example 1 from 5-chloro-2-{[(5-fluoropyridin-3-yl)oxy]methyl}benzoic acid (step 2) and methyl 4-[(1S)-1-aminoethyl]benzoate hydrochloride (step 5 of Example 1)

¹H-NMR (CDCl₃) δ 8.18-8.10 (2H, m), 7.95 (2H, d, J=8.3 Hz), 7.60-7.43 (3H, m), 7.38 (2H, d, J=8.3 Hz), 6.89 (1H, dt, J=10.0, 2.4 Hz), 6.49-6.42 (1H, m), 5.37-5.22 (1H, m), 5.16 (2H, s), 3.92 (3H, s), 1.55 (3H, d, J=7.0 Hz);

MS (ESI) m/z 443 (M+H)⁺.

### STEP 4. 4-{(1S)-1-[(5-Chloro-2-{[(5-fluoropyridin-3-yl)oxy]methyl}benzoyl)amino]ethyl}benzoic acid

The title compound was prepared according to the procedure described in step 7 of Example 1 from methyl 4-{(1S)-1-[(5-chloro-2-{[(5-fluoropyridin-3-yl)oxy]methyl}benzoyl)amino]ethyl}benzoate (step 3):

¹H-NMR (DMSO-d₆) δ 9.10 (1H, d, J=8.1 Hz), 8.23-8.09 (2H, m), 7.84 (2H, d, J=8.2 Hz), 7.60-7.50 (3H, m), 7.47 (2H, d, J=8.2 Hz), 7.38-7.28 (1H, m), 5.25 (2H, s), 5.10 (1H, dq, J=8.1, 6.9 Hz), 1.43 (3H, d, J=6.9 Hz);

MS (ESI) m/z 429 (M+H)⁺, 427 (M−H)⁻.

### Example 15

### 4-{(1S)-1-[(5-CHLORO-2-{[(5-CHLOROPYRIDIN-3-YL)OXY]METHYL}BENZOYL)AMINO]ETHYL}BENZOIC ACID

### STEP 1. Methyl 5-chloro-2-{[(5-chloropyridin-3-yl)oxy]methyl}benzoate

The title compound was prepared according to the procedure described in step 1 of Example 1 from methyl 2-(bromomethyl)-5-chlorobenzoate and 3-chloro-5-hydroxypyridine:

¹H-NMR (CDCl₃) δ 8.29 (1H, d, J=2.8 Hz), 8.22 (1H, d, J=2.0 Hz), 8.05 (1H, d, J=2.2 Hz), 7.66 (1H, d, J=8.4 Hz), 7.56 (1H, dd, J=8.4, 2.2 Hz), 7.35-7.25 (1H, m), 5.50 (2H, s), 3.92 (3H, s).

### STEP 2. 5-Chloro-2-{[(5-chloropyridin-3-yl)oxy]methyl}benzoic acid

The title compound was prepared according to the procedure described in step 2 of Example 1 from methyl 5-chloro-2-{[(5-chloropyridin-3-yl)oxy]methyl}benzoate (step 1): MS (ESI) m/z 296 (M−H)⁻.

### STEP 3. Methyl 4-{(1S)-1-[(5-chloro-2-{[(5-chloropyridin-3-yl)oxy]methyl}benzoyl)amino]ethyl}benzoate

The title compound was prepared according to the procedure described in step 6 of Example 1 from 5-chloro-2-{[(5-chloropyridin-3-yl)oxy]methyl}benzoic acid (step 2) and methyl 4-[(1S)-1-aminoethyl]benzoate hydrochloride (step 5 of Example 1)

¹H-NMR (CDCl₃) δ 8.23-8.15 (2H, m), 7.95 (2H, d, J=8.3 Hz), 7.57-7.54 (1H, m), 7.48 (2H, s), 7.38 (2H, d, J=8.3 Hz), 7.15 (1H, t, J=2.2 Hz), 6.45-6.35 (1H, m), 5.35-5.22 (1H, m), 5.15 (2H, s), 3.92 (3H, s), 1.56 (3H, d, J=7.0 Hz);

MS (ESI) m/z 459 (M+H)⁺.

### STEP 4. 4-{(1S)-1-[(5-Chloro-2-{[(5-chloropyridin-3-yl)oxy]methyl}benzoyl)amino]ethyl}benzoic acid

The title compound was prepared according to the procedure described in step 7 of Example 1 from 4-{(1S)-1-[(5-chloro-2-{[(5-chloropyridin-3-yl)oxy]methyl}benzoyl)amino]ethyl}benzoate (step 3):

¹H-NMR (DMSO-d₆) δ 9.09 (1H, d, J=8.6 Hz), 8.23-8.15 (2H, m), 7.84 (2H, d, J=8.2 Hz), 7.60 (3H, br.s), 7.50-7.40 (3H, m), 5.34-5.20 (2H, m), 5.19-5.00 (1H, m), 1.43 (3H, d, J=6.9 Hz);

MS (ESI) m/z 445 (M+H)⁺, 443 (M−H)⁻.

### Example 16

### 4-[(1S)-1-({5-CHLORO-2-[(CYCLOPENTYLOXY)METHYL]BENZOYL}AMINO)ETHYL]BENZOIC ACID

### STEP 1. 5-Chloro-2-[(cyclopentyloxy)methyl]benzoic acid

A mixture of methyl 2-(bromomethyl)-5-chlorobenzoate (200 mg, 0.80 mmol), cyclopentanol (379 mg 4.4 mmol), and potassium tert-butoxide (448 mg, 4.0 mmol) in tetrahydrofuran (8 mL) was stirred at room temperature for 3 hours. The mixture was acidified with 2 N hydrochloric acid and the acidic aqueous mixture was extracted with ethyl acetate. The organic layer was washed with brine, dried over sodium sulfate and evaporated. The residue was purified by flash column chromatography on silica gel eluting with hexane/ethyl acetate (1/1) to afford 100 mg (49%) of the title compound:

¹H-NMR (CDCl₃) δ 8.05 (1H, d, J=2.2 Hz), 7.58 (1H, d, J=8.4 Hz), 7.52 (1H, dd, J=8.4, 2.2 Hz), 4.78 (2H, s), 4.15-4.05 (1H, m), 1.90-1.50 (8H, m);

MS (ESI) m/z 253 (M−H)⁻.

### STEP 2. Methyl 4-[(1S)-1-({5-chloro-2-[(cyclopentyloxy)methyl]benzoyl}amino)ethyl]benzoate

The title compound was prepared according to the procedure described in step 6 of Example 1 from 5-chloro-2-[(cyclopentyloxy)methyl]benzoic acid (step 1) and methyl 4-[(1S)-1-aminoethyl]benzoate hydrochloride (step 5 of Example 1):

¹H-NMR (DMSO-d₆) δ 8.17-8.07 (1H, m), 8.03 (2H, d, J=8.4 Hz), 7.80 (1H, d, J=2.1 Hz), 7.47 (2H, d, J=8.4 Hz), 7.38 (1H, dd, J=8.1, 2.1 Hz), 7.25 (1H, d, J=8.1 Hz), 5.45-5.30 (1H, m), 4.50 (1H, d, J=11.7 Hz), 4.44 (1H, d, J=11.7 Hz), 3.98-3.87 (4H, m), 1.80-1.40 (11H, m);

MS (ESI) m/z 416 (M+H)⁺, 414 (M−H)⁻.

### STEP 3. 4-[(1S)-1-({5-Chloro-2-[(cyclopentyloxy)methyl]benzoyl}amino)ethyl]benzoic acid

The title compound was prepared according to the procedure described in step 7 of Example 1 from methyl 4-[(1S)-1-({5-chloro-2-[(cyclopentyloxy)methyl]benzoyl}amino)ethyl]benzoate (step 2):

¹H-NMR (DMSO-d₆) δ 8.97 (1H, d, J=7.7 Hz), 7.93 (2H, d, J=7.9 Hz), 7.60-7.40 (5H, m), 5.22-5.04 (1H, m), 4.42 (2H, s), 3.90-3.80 (1H, br), 1.70-1.35 (11H, m);

MS (ESI) m/z 402 (M+H)⁺, 400 (M−H)⁻.

### Example 17

### 4-((1S)-1-{[5-CHLORO-2-(ISOBUTOXYMETHYL)BENZOYL]AMINO}ETHYL)BENZOIC ACID

### STEP 1. 5-Chloro-2-(isobutoxymethyl)benzoic acid

The title compound was prepared according to the procedure described in step 1 of Example 16 from methyl 2-(bromomethyl)-5-chlorobenzoate and 2-methylpropan-1-ol:

¹H-NMR (CDCl₃) δ 8.05 (1H, d, J=2.4 Hz), 7.61 (1H, d, J=8.4 Hz), 7.53 (1H, dd, J=8.4, 2.4 Hz), 4.82 (2H, s), 3.36 (2H, d, J=6.4 Hz), 2.05-1.88 (1H, m), 0.96 (6H, d, J=6.6 Hz);

MS (ESI) m/z 241 (M−H)⁻.

### STEP 2. Methyl 4-((1S)-1-{[5-chloro-2-(isobutoxymethyl)benzoyl]amino}ethyl)benzoate

The title compound was prepared according to the procedure described in step 6 of Example 1 from 5-chloro-2-(isobutoxymethyl)benzoic acid (step 1) and methyl 4-[(1S)-1-aminoethyl]benzoate hydrochloride (step 5 of Example 1):

MS (ESI) m/z 404 (M+H)⁺, 402 (M−H)⁻.

### STEP 3. 4-((1S)-1-{[5-Chloro-2-(isobutoxymethyl)benzoyl]amino}ethyl)benzoic acid

The title compound was prepared according to the procedure described in step 7 of Example 1 from methyl 4-((1S)-1-{[5-chloro-2-(isobutoxymethyl)benzoyl]amino}ethyl)benzoate (step 2):

¹H-NMR (DMSO-d₆) δ 8.97 (1H, d, J=8.1 Hz), 7.92 (2H, d, J=7.9 Hz), 7.55-7.45 (5H, m), 5.12 (1H, dq, J=8.1, 7.0 Hz), 4.49 (1H, d, J=13.0 Hz), 4.44 (1H, d, J=13.0 Hz), 3.09 (2H, d, J=6.2 Hz), 1.80-1.65 (1H, m), 1.44 (3H, d, J=7.0 Hz) 0.82 (6H, d, J=6.8 Hz);

MS (ESI) m/z 390 (M+H)⁺, 388 (M−H)⁻.

### Example 18

### 4-{[(1S)-1-[({5-CHLORO-2-[(4-CHLOROPHENOXY)METHYL]PYRIDIN-3-YL}CARBONYL)AMINO]ETHYL}BENZOIC ACID

### STEP 1. 3-Chlorofuro[3,4-b]pyridin-5(7H)-one

A mixture of crude methyl 5-chloro-2-methylnicotinate 1-oxide (Organic letters, 2001, 3, 209, 2.29 mmol) and trifluoroacetic acid (453 μL, 3.21 mmol) in dichloromethane (20 mL) was stirred at room temperature for 2 days and heated at 45° C. for 1 hour. The mixture was partitioned between sat. aqueous sodium hydrogen carbonate (50 mL) and ethyl acetate (50 mL). The organic layer was washed with brine (50 mL), dried (sodium sulfate), and evaporated. The residue was purified by flash column chromatography on silica gel eluting with hexane/ethyl acetate (1/1) to afford 225 mg of the title compound.

¹H-NMR (CDCl₃) δ 8.55 (1H, d, J=2.0 Hz), 8.19 (1H, d, J=2.0 Hz), 5.34 (2H, s).

### STEP 2. 5-Chloro-2-[(4-chlorophenoxy)methyl]nicotinic acid

A mixture of 3-chlorofuro[3,4-b]pyridin-5(7H)-one (step 1, 110 mg, 0.65 mmol) and 4-choloro phenol (416 mg, 3.24 mmol) was heated to 130° C. under N₂, then sodium methoxide (28% methanol solution, 250 mg, 1.30 mmol) was added dropwise to the mixture at 130° C. The mixture was heated at the same temperature for 4 hours. After cooling, to the mixture was added 10% aqueous citric acid and the mixture was extracted with ethyl acetate. The extracts were dried over sodium sulfate and evaporated. The residue was purified by flash column chromatography on silica gel to afford 113 mg of the title compound:

MS (ESI) m/z 298 (M+H)⁺, 296 (M−H)⁻.

### STEP 3. Methyl 4-{(1S)-1-[({5-chloro-2-[(4-chlorophenoxy)methyl]pyridin-3-yl}carbonyl)amino]ethyl}benzoate

The title compound was prepared according to the procedure described in step 6 of Example 1 from 5-chloro-2-[(4-chlorophenoxy)methyl]nicotinic acid (step 2) and methyl 4-[(1S)-1-aminoethyl]benzoate hydrochloride (step 5 of Example 1):

MS (ESI) m/z 459 (M+H)⁺, 457 (M−H)⁻

### STEP 4. 4-{(1S)-1-[({5-Chloro-2-[(4-chlorophenoxy)methyl]pyridin-3-yl}carbonyl)amino]ethyl}benzoic acid

The title compound was prepared according to the procedure described in step 7 of Example 1 from methyl 4-{(1S)-1-[({5-chloro-2-[(4-chlorophenoxy)methyl]pyridin-3-yl}carbonyl)amino]ethyl}benzoate (step 3):

¹H-NMR (DMSO-d₆) δ9.17 (1H, d, J=7.5 Hz), 8.72 (1H, s), 8.08 (1H, s), 7.85 (2H, d, J=7.9 Hz), 7.46 (2H, d, J=7.9 Hz), 7.26 (2H, d, J=7.5 Hz), 6.83 (2H, d, J=7.5 Hz), 5.23 (1H, d, J=11.9 Hz), 5.18 (1H, d, J=11.9 Hz), 5.13-5.15 (1H, m), 1.41 (3H, d, J=7.3 Hz);

MS (ESI) m/z 445 (M+H)⁺, 443 (M−H)⁻.

### Example 19

### 4-((1S)-1-{[5-CHLORO-2-({3-[(METHYLAMINO)CARBONYL]PHENOXY}METHYL)BENZOYL]AMINO}ETHYL)BENZOIC ACID

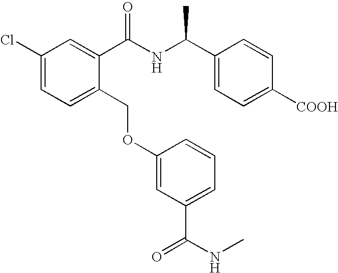

### STEP 1. Methyl 5-chloro-2-({3-[(methylamino)carbonyl]phenoxy}methyl)benzoate

The title compound was prepared according to the procedure described in step 1 of Example 1 from methyl 2-(bromoethyl)-5-chlorobenzoate and 3-hydroxy-N-methylbenzamide (WO 2003018566):

¹H-NMR (CDCl₃) δ 8.02 (1H, d, J=2.3 Hz), 7.68 (1H, d, J=8.4 Hz), 7.52 (1H, dd, J=8.4, 2.3 Hz), 7.43-7.29 (3H, m), 7.12-7.08 (1H, m), 5.49 (2H, s), 3.91 (3H, s), 3.01 (3H, d, J=4.9 Hz), a peak of NH was not observed;

MS (ESI) m/z 334 (M+H)⁺.

### STEP 2. 5-Chloro-2-({3-[(methylamino)carbonyl]phenoxy}methyl)benzoic acid

The title compound was prepared according to the procedure described in step 2 of Example 1 from methyl 5-chloro-2-({3-[(methylamino)carbonyl]phenoxy}methyl)benzoate (step 1):

¹H-NMR (DMSO-d₆) δ 8.43-8.41 (1H, m), 7.89 (1H, br.s), 7.70-7.63 (2H, m), 7.42-7.33 (3H, m), 7.12-7.09 (1H, m), 5.45 (2H, s), 2.75 (3H, d, J=4.5 Hz), a peak of COOH was not observed);

MS (ESI) m/z 320 (M+H)⁺, 318 (M−H)⁻.

### STEP 3. Methyl 4-((1S)-1-{[5-chloro-2-({3-[(methylamino)carbonyl]phenoxy}methyl)benzoyl]amino}ethyl)benzoate

The title compound was prepared according to the procedure described in step 6 of Example 1 from 5-chloro-2-({3-[(methylamino)carbonyl]phenoxy}methyl)benzoic acid (step 2) and methyl 4-[(1S)-1-aminoethyl]benzoate hydrochloride (step 3 of Example 1):

¹H-NMR (CDCl₃) δ 9.08 (1H, d, J=7.6 Hz), 8.40-8.39 (1H, m), 7.83 (2H, d, J=8.2 Hz), 7.61-7.54 (3H, m), 7.48 (2H, d, J=8.2 Hz), 7.42-7.38 (2H, m), 7.30 (1H, t, J=7.8 Hz), 6.97-6.94 (1H, m), 5.21-5.04 (3H, m), 3.81 (3H, s), 2.75 (3H, d, J=4.5 Hz), 1.41 (3H, d, J=7.1 Hz);

MS (ESI) m/z 481 (M+H)⁺, 479 (M−H)⁻.

### STEP 4. 4-((1S)-1-{[5-Chloro-2-({3-[(methylamino)carbonyl]phenoxy}methyl)benzoyl]amino}ethyl)benzoic acid

The title compound was prepared according to the procedure described in step 7 of Example 1 from methyl 4-((1S)-1-{[5-chloro-2-({3-[(methylamino)carbonyl]phenoxy}methyl)benzoyl]amino}ethyl)benzoate (step 3):

¹H-NMR (DMSO-d₆) δ 8.97-8.94 (1H, m), 8.31-8.29 (1H, m), 7.76-7.73 (2H, m), 7.47-7.14 (8H, m), 6.89-6.84 (1H, m), 5.08-4.94 (3H, m), 2.65 (3H, d, J=4.3 Hz), 1.31 (3H, d, J=7.0 Hz), a peak of COOH was not observed;

MS (ESI) m/z 467 (M+H)⁺, 465 (M−H)⁻.

### Example 20

### 4-{(1S)-1-[({5-CHLORO-2-[(3-CHLOROPHENOXY)METHYL]PYRIDIN-3-YL}CARBONYL)AMINO]ETHYL}BENZOIC ACID

### STEP 1. 5-Chloro-2-[(3-chlorophenoxy)methyl]nicotinic acid

The title compound was prepared according to the procedure described in step 2 of Example 18 from 3-chlorofuro[3,4-b]pyridin-5(7H)-one (Organic letters, 2001, 3, 209) and 3-choloro phenol:

MS (ESI) m/z 298 (M+H)⁺, 296 (M−H)⁻

### STEP 2. Methyl 4-{(1S)-1-[({5-chloro-2-[(3-chlorophenoxy)methyl]pyridin-3-yl}carbonyl)amino]ethyl}benzoate

The title compound was prepared according to the procedure described in step 3 of Example 1 from 5-chloro-2-[(3-chlorophenoxy)methyl]nicotinic acid (step 1) and methyl 4-[(1S)-1-aminoethyl]benzoate hydrochloride (step 5 of Example 1)

MS (ESI) m/z 459 (M+H)⁺, 457 (M−H)⁻

### STEP 3. 4-{(1S)-1-[({5-Chloro-2-[(3-chlorophenoxy)methyl]pyridin-3-yl}carbonyl)amino]ethyl}benzoic acid

The title compound was prepared according to the procedure described in step 3 of Example 1 from methyl 4-{(1S)-1-[({5-chloro-2-[(3-chlorophenoxy)methyl]pyridin-3-yl}carbonyl)amino]ethyl}benzoate (step 2):

¹H-NMR (DMSO-d₆) δ9.16 (1H, d, J=8.9 Hz), 8.73 (1H, s), 8.09 (1H, s), 7.86 (2H, d, J=8.1 Hz), 7.45 (2H, d, J=8.1 Hz), 7.25 (1H, t, J=7.6 Hz), 6.99 (1H, d, J=7.6 Hz), 6.93 (1H, s), 6.85-6.75 (1H, m), 5.29-5.22 (2H, m), 5.20-5.00 (1H, m), 1.42 (3H, d, J=7.2 Hz);

MS (ESI) m/z 445 (M+H)⁺, 443 (M−H)⁻.

### Example 21

### 4-{[(1S)-1-[({2-[(4-CHLOROPHENOXY)METHYL]-5-FLUOROPYRIDIN-3-YL}CARBONYL)AMINO]ETHYL}BENZOIC ACID

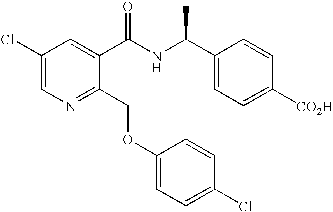

### STEP 1. Methyl 2-chloro-5-fluoronicotinate

To a solution of 2-chloro-5-fluoronicotinic acid (5.2 g, 30 mmol) in methanol (20 ml) was added conc. sulfuric acid (0.5 ml) and the reaction mixture was stirred at reflux for 30 hours. The reaction mixture was cooled to 0° C. and 0.5 N sodium hydroxide solution was added to the mixture. The whole was extracted with diethylether. The organic phase was washed with brine, dried (sodium sulfate), and concentrated to afford 3.2 g (25%) of the title compound: ¹H-NMR (CDCl₃) δ 8.41 (1H, d, J=3.0 Hz), 7.93 (1H, dd, J=3.0, 7.6 Hz), 3.98 (3H, s).

### STEP 2. Methyl 5-fluoro-2-methylnicotinate

A mixture of methyl 2-chloro-5-fluoronicotinate (step 1, 1.5 g, 7.91 mmol), tetrakis(triphenylphoshine)palladium (914 mg, 0.79 mmol), methyboronic acid (521 mg, 8.70 mmol) and potassium carbonate (3.28 g, 23.7 mmol) in 1,4-dioxane (20 ml) was heated at 110° C. for 20 h under nitrogen atmosphere. The reaction mixture was filtered through a pad of celite (Celite(trademark) (diatomaceous earth)) and the filtrate was concentrated. The residue was purified by flush column chromatography on silica gel eluting with hexane/ethyl acetate (20/1 to 4/1) to afford 936 mg (64%) of the title compound:

¹H-NMR (CDCl₃) δ 8.49 (1H, d, J=3.0 Hz), 7.93 (1H, dd, J=3.0, 8.7 Hz), 3.94 (3H, s), 2.81 (3H, s).

### STEP 3. Methyl 5-fluoro-2-methylnicotinate 1-oxide

To a cooled (0° C.) solution of methyl 5-fluoro-2-methylnicotinate (step 2, 936 mg, 5.53 mmol) in dichloromethane (100 ml) was added 3-chlorobenzenecarboperoxoic acid (2.38 g, 13.8 mmol) and the reaction suspension was stirred overnight at room temperature. The reaction was quenched by the addition of sat. sodium thiosulfate solution and sat. sodium bicarbonate solution was added. The whole mixture was extracted with dichloromethane. The organic phase was dried over sodium sulfate and concentrated to afford 1.12 g (quant.) of title compound:

¹H-NMR (CDCl₃) δ 8.40-8.03 (1H, m), 7.52 (1H, dd, J=2.3, 7.7 Hz), 3.96 (3H, s), 2.73 (3H, s).

### STEP 4. 3-Fluorofuro[3,4-b]pyridin-5(7H)-one

The title compound was prepared according to the procedure described in step 1 of Example 18 from methyl 5-fluoro-2-methylnicotinate 1-oxide (step 3):

¹H-NMR (CDCl₃) δ 8.80-8.74 (1H, m), 7.89 (1H, dd, J=2.6, 6.6 Hz), 5.35 (2H, s).

### STEP 5. 2-[(4-Chlorophenoxy)methyl]-5-fluoronicotinic acid

The title compound was prepared according to the procedure described in step 2 of Example 18 from 3-fluorofuro[3,4-b]pyridin-5(7H)-one (step 4) and 4-chlorophenol:

MS (ESI) m/z 282 (M+H)⁺, 280 (M−H)⁻.

### STEP 6. Methyl 4-{(1S)-1-[({2-[(4-Chlorophenoxy)methyl]-5-fluoropyridin-3-yl}carbonyl)amino]ethyl}benzoate

The title compound was prepared according to the procedure described in step 6 of Example 1 from 2-[(4-chlorophenoxy)methyl]-5-fluoronicotinic acid (step 5) and methyl 4-[(1S)-1-aminoethyl]benzoate hydrochloride (step 5 of Example 1):

¹H-NMR (CDCl₃) δ 8.54 (1H, d, J=3.0 Hz), 7.90 (2H, d, J=8.2 Hz), 7.80 (1H, dd, J=2.8, 8.2 Hz), 7.35-7.20 (5H, m), 6.83 (2H, d, J=9.1 Hz), 5.36-5.23 (1H, m), 5.17 (1H, d, J=10.1 Hz), 5.12 (1H, d, J=10.1 Hz), 3.93 (3H, s), 1.48 (3H, d, J=6.9 Hz).

### STEP 7. 4-{(1S)-1-[({2-[(4-Chlorophenoxy)methyl]-5-fluoropyridin-3-yl}carbonyl)amino]ethyl}benzoic acid

The title compound was prepared according to the procedure described in step 7 of Example 1 from methyl 4-{(1S)-1-[({2-[(4-chlorophenoxy)methyl]-5-fluoropyridin-3-yl}carbonyl)amino]ethyl}benzoate (step 6):

¹H-NMR (CDCl₃) δ 8.55 (1H, d, J=2.9 Hz), 7.97 (2H, d, J=8.2 Hz), 7.82 (1H, dd, J=2.9, 8.1 Hz), 7.38-7.20 (5H, m), 6.86 (2H, d, J=8.9 Hz), 5.36-5.25 (1H, m), 5.22-5.10 (2H, m), 1.49 (3H, d, J=6.9 Hz);

MS (ESI) m/z 429 (M+H)⁺, 427 (M−H)⁻.

### Example 22

### 4-{(1S)-1-[(5-CHLORO-2-{[(5-CHLOROPYRIDIN-2-YL)(METHYL)AMINO]METHYL}BENZOYL)AMINO]ETHYL}BENZOIC ACID

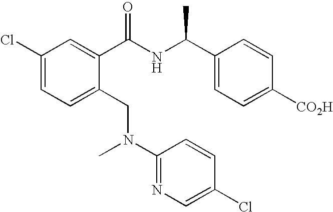

### STEP 1. Methyl 5-chloro-2-{[(5-chloropyridin-2-yl)(methyl)amino]methyl}benzoate

To s suspension of sodium hydride (60% dispersion in mineral oil, 46 mg, 1.1 mmol) in terahydrorofuran (4 ml) was added 5-chloro-N-methylpyridin-2-amine (128 mg, 1.14 mmol) in tetrahydrofuran (5 ml) at room temperature and stirred for 30 min. The mixture was added methyl 2-(bromomethyl)-5-chlorobenzoate (250 mg, 0.95 mmol) in tetrahydrofuran (5 ml) at room temperature and stirred at 80° C. for 8 hr. After cooling to room temperature, the mixture was added water and extracted with ethyl acetate. The organic layer was dried over sodium sulfate and evaporated. The residue was purified by flush column chromatography on silica gel eluting with hexane/ethyl acetate (10/1) to afford 102 mg (33%) of the title compound:

¹H-NMR (CDCl₃) δ 8.07 (1H, d, J=2.6 Hz), 7.99 (1H, d, J=2.2 Hz), 7.42-7.33 (2H, m), 7.09 (1H, d, J=8.3 Hz), 6.83 (1H, d, J=9.0 Hz), 5.07 (2H, s), 3.91 (3H, s), 3.11 (3H, s).

### STEP 2. 5-Chloro-2-{[(5-chloropyridin-2-yl)(methyl)amino]methyl}benzoic acid

The title compound was prepared according to the procedure described in step 2 of Example 1 from methyl 5-chloro-2-{[(5-chloropyridin-2-yl)(methyl)amino]methyl}benzoate (step 1):

¹H-NMR (CDCl₃) δ 8.02-7.97 (1H, m), 7.93-7.89 (1H, m), 7.46 (1H, dd, J=2.6, 9.2 Hz), 7.38 (1H, d, J=2.4, 8.3 Hz), 7.18 (1H, d, J=8.3 Hz), 6.51 (1H, d, J=9.2 Hz), 4.98-4.89 (2H, br.s), 3.29 (3H, s).

### STEP 3. Methyl 4-{(1S)-1-[(5-chloro-2-{[(5-chloropyridin-2-yl)(methyl)amino]methyl}benzoyl)amino]ethyl}benzoate

The title compound was prepared according to the procedure described in step 6 of Example 1 from 5-chloro-2-{[(5-chloropyridin-2-yl)(methyl)amino]methyl}benzoic acid (step 2) and methyl 4-[(1S)-1-aminoethyl]benzoate hydrochloride (step 5 of Example 1):

¹H-NMR (CDCl₃) δ 8.19 (1H, d, J=7.8 Hz), 8.02 (2H, d, J=8.2 Hz), 7.72 (1H, d, J=2.6 Hz), 7.49 (1H, d, J=2.1 Hz), 7.45 (2H, d, J=8.2 Hz), 7.40 (1H, dd, J=2.6, 9.1 Hz), 7.27 (1H, dd, J=2.1, 8.2 Hz), 7.15 (1H, d, J=8.2 Hz), 6.48 (1H, d, J=9.1 Hz), 5.42-5.27 (1H, m), 4.82 (1H, d, J=16.3 Hz), 4.69 (1H, d, J=16.3 Hz), 3.92 (3H, s), 3.20 (3H, s), 1.60 (3H, d, J=6.9 Hz).

### STEP 4. 4-{(1S)-1-[(5-Chloro-2-{[(5-chloropyridin-2-yl)(methyl)amino]methyl}benzoyl)amino]ethyl}benzoic acid

The title compound was prepared according to the procedure described in step 7 of Example 1 from methyl 4-{(1S)-1-[(5-chloro-2-{[(5-chloropyridin-2-yl)(methyl)amino]methyl}benzoyl)amino]ethyl}benzoate (step 3):

¹H-NMR (CDCl₃) δ 8.23 (1H, d, J=7.6 Hz), 8.07 (2H, d, J=8.2 Hz), 7.74 (1H, d, J=2.2 Hz), 7.54-7.44 (3H, m), 7.41 (1H, dd, J=2.6, 9.1 Hz), 7.31-7.25 (1H, m), 7.15 (1H, d, J=8.4 Hz), 6.49 (1H, d, J=9.1 Hz), 5.43-5.30 (1H, m), 4.84 (1H, d, J=16.5 Hz), 4.71 (1H, d, J=16.5 Hz), 3.21 (3H, s), 1.61 (3H, d, J=6.9 Hz);

MS (ESI) m/z 458 (M+H)⁺, 456 (M−H)⁻.

### Example 23

### 4-{(1S)-1-([{5-CHLORO-2-[(CYCLOHEXYLMETHOXY)METHYL]BENZOYL}AMINO)ETHYL}BENZOIC ACID

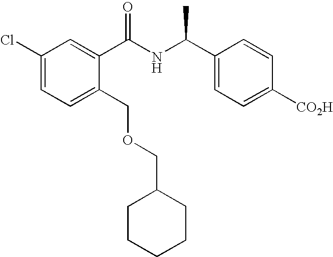

### STEP 1. Methyl 4-[(1S)-1-({5-chloro-2-[(cyclohexylmethoxy)methyl]benzoyl}amino)ethyl]benzoate

To a solution of potassium tert-butoxide (533 mg, 4.75 mmol) in tetrahydrofuran (10 ml) was added cyclohexylmethanol (594 mg, 5.20 mmol) in tetrahydrofuran (2.5 ml), methyl 2-(bromomethyl)-5-chlorobenzoate (250 mg, 0.95 mmol) in tetarahydrofuran (2.5 ml) at 0° C. The reaction mixture was stirred at room temperature for 2 hours. The resulting solution was acidified with 2 N hydrochloric acid solution (pH ca. 2) at 0° C. It was extracted with dichloromethane, the organic extracts were dried over sodium sulfate and concentrated to afford 302 mg of crude 5-chloro-2-[(cyclohexylmethoxy)methyl]benzoic acid. This carboxylic acid was converted into 132 mg (31%) of the title compound according to the procedure described in step 6 of Example 1:

¹H-NMR (CDCl₃) δ 8.13-8.03 (1H, m), 8.03 (2H, d, J=8.2 Hz), 7.81 (1H, d, J=2.2 Hz), 7.47 (2H, d, J=8.2 Hz), 7.38 (1H, dd, J=2.2, 8.1 Hz), 7.24 (1H, d, J=8.1 Hz), 5.48-5.29 (1H, m), 4.51 (1H, d, J=11.5 Hz), 4.44 (1H, d, J=11.5 Hz), 3.91 (3H, s), 3.26-3.10 (2H, m), 1.75-1.54 (6H, m), 1.59 (3H, d, J=7.1 Hz), 1.50-1.35 (1H, m), 1.23-1.05 (2H, m), 0.93-0.73 (2H, m).

### STEP 2. 4-{(1S)-1-({5-Chloro-2-[(cyclohexylmethoxy)methyl]benzoyl}amino)ethyl}benzoic acid

The title compound was prepared according to the procedure described in step 7 of Example 1 from methyl 4-[(1S)-1-({5-chloro-2-[(cyclohexyl methoxy)methyl]benzoyl}amino)ethyl]benzoate (step 1):

¹H-NMR (CDCl₃) δ 8.14 (1H, d, J=7.4 Hz), 8.09 (2H, d, J=8.2 Hz), 7.83 (1H, d, J=2.3 Hz), 7.50 (2H, d, J=8.2 Hz), 7.39 (1H, dd, J=2.3, 8.1 Hz), 7.25 (1H, d, J=8.1 Hz), 5.48-5.33 (1H, m), 4.53 (1H, d, J=11.5 Hz), 4.46 (1H, d, J=11.5 Hz), 3.29-3.12 (2H, m), 1.74-1.58 (6H, m), 1.61 (3H, d, J=6.9 Hz), 1.53-1.33 (1H, m), 1.26-1.08 (2H, m), 0.93-0.78 (2H, m);

MS (ESI) m/z 458 (M+H)⁺, 456 (M−H)⁻.

### Example 24

### 4-{(1S)-1-([{5-CHLORO-2-[(2,2-DIMETHYLPROPOXY)METHYL]BENZOYL}AMINO)ETHYL}BENZOIC ACID

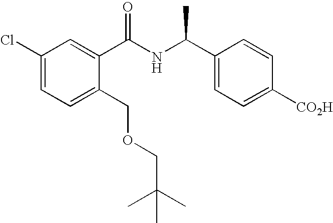

### STEP 1. Methyl 4-[(1S)-1-({5-chloro-2-[(2,2-dimethylpropoxy)methyl]benzoyl}amino)ethyl]benzoate

The title compound was prepared according to the procedure described in step 1 of Example 23 from methyl 2-(bromoethyl)-5-chlorobenzoate via 5-chloro-2-[(2,2-dimethylpropoxy)methyl]benzoic acid as an intermediate:

¹H-NMR (CDCl₃) δ 8.12-8.00 (1H, m), 8.03 (2H, d, J=8.4 Hz), 7.81 (1H, d, J=2.3 Hz), 7.48 (2H, d, J=8.4 Hz), 7.39 (1H, dd, J=2.3, 8.1 Hz), 7.26 (1H, d, J=8.1 Hz), 5.46-5.33 (1H, m), 4.53 (2H, s), 3.91 (3H, s), 3.10 (1H, d, J=8.6 Hz), 3.03 (1H, d, J=8.6 Hz), 1.59 (3H, d, J=7.1 Hz), 0.84 (9H, s).

### STEP 2. 4-{(1S)-1-({5-Chloro-2-[(2,2-dimethylpropoxy)methyl]benzoyl}amino)ethyl}benzoic acid

The title compound was prepared according to the procedure described in step 7 of Example 1 from methyl 4-[(1S)-1-({5-chloro-2-[(2,2-dimethylpropoxy)methyl]benzoyl}amino)ethyl]benzoate (step 1):

¹H-NMR (CDCl₃) δ 8.13-8.05 (3H, m), 7.84 (1H, d, J=2.2 Hz), 7.51 (2H, d, J=8.2 Hz), 7.40 (1H, dd, J=2.2, 8.1 Hz), 7.26 (1H, d, J=8.1 Hz), 5.49-5.34 (1H, m), 4.55 (2H, s), 3.11 (1H, d, J=8.6 Hz), 3.04 (1H, d, J=8.6 Hz), 1.61 (3H, d, J=6.9 Hz), 0.84 (9H, s);

MS (ESI) m/z 404 (M+H)⁺, 402 (M−H)⁻.

### Example 25

### 4-{(1S)-1-[(5-CHLORO-2-{[(5-FLUOROPYRIDIN-2-YL)(METHYL)AMINO]METHYL}BENZOYL)AMINO]ETHYL}BENZOIC ACID

### STEP 1. 5-Fluoro-N-methylpyridin-2-amine

To a suspension of sodium hydride (60% dispersion in mineral oil, 117.8 mg, 4.91 mmol) in tetrahydrofuran (25 ml) was added a solution of 5-fluoropyridin-2-amine (500 mg, 4.46 mmol) in tetrahydrofuran (25 ml) at room temperature and the reaction mixture was stirred at 40° C. for 30 min. Then to the reaction mixture was added methyl iodide (696.9 mg, 4.91 mmol) at −40° C. and the resulting mixture was stand at room temperature overnight with stirring. The reaction was quenched by the addition of water and whole mixture was extracted with ethyl acetate. The organic extracts were dried over sodium sulfate and concentrated. The residue was purified by flush column chromatography on silica gel eluting with hexane/ethyl acetate (4/1) to afford 129 mg (23%) of the title compound:

¹H-NMR (CDCl₃) δ 7.97 (1H, d, J=2.6 Hz), 7.28-7.17 (1H, m), 6.34 (1H, dd, J=3.5, 9.1 Hz), 2.90 (3H, d, J=5.1 Hz).

### STEP 2. Methyl 5-chloro-2-{[(5-fluoropyridin-2-yl)(methyl)amino]methyl}benzoate

The title compound was prepared according to the procedure described in step 1 of Example 22 from methyl 2-(bromoethyl)-5-chlorobenzoate and 5-fluoro-N-methylpyridin-2-amine (step 1):

¹H-NMR (CDCl₃) δ 8.00 (1H, d, J=3.3 Hz), 7.99 (1H, d, J=2.2 Hz), 7.39 (1H, dd, J=2.2, 8.4 Hz) 7.25-7.16 (1H, m), 7.13 (1H, d, J=8.4 Hz), 6.37 (1H, dd, J=3.3, 9.2 Hz), 5.05 (2H, s), 3.90 (3H, s), 3.11 (3H, s).

### STEP 3. Methyl 4-{(1S)-1-[(5-chloro-2-{[(5-fluoropyridin-2-yl)(methyl)amino]methyl}benzoyl)amino]ethyl}benzoate

The title compound was prepared according to the procedure described in step 2 and 6 of Example 1 from methyl 5-chloro-2-{[(5-fluoropyridin-2-yl)(methyl)amino]methyl}benzoate (step 2) via 5-chloro-2-{[(5-fluoropyridin-2-yl)(methyl)amino]methyl}benzoic acid as an intermediate:

¹H-NMR (CDCl₃) δ 8.32 (1H, d, J=7.3 Hz), 8.00 (2H, d, J=8.3 Hz), 7.71 (1H, d, J=2.9 Hz), 7.49 (1H, d, J=2.0 Hz), 7.43 (2H, d, J=8.3 Hz), 7.31-7.19 (2H, m), 7.16 (1H, d, J=8.3 Hz), 6.48 (1H, dd, J=3.3, 9.4 Hz), 5.43-5.25 (1H, m), 4.78 (1H, d, J=16.3 Hz), 4.67 (1H, d, J=16.3 Hz), 3.90 (3H, s), 3.17 (3H, s), 1.57 (3H, d, J=7.2 Hz).

### STEP 4. 4-{(1S)-1-[(5-Chloro-2-{[(5-fluoropyridin-2-yl)(methyl)amino]methyl}benzoyl)amino]ethyl}benzoic acid

The title compound was prepared according to the procedure described in step 7 of Example 1 from methyl 4-{(1S)-1-[(5-chloro-2-{[(5-fluoropyridin-2-yl)(methyl)amino]methyl}benzoyl)amino]ethyl}benzoate (step 3):

¹H-NMR (CDCl₃) δ 8.35 (1H, d, J=7.5 Hz), 8.06 (2H, d, J=8.3 Hz), 7.73 (1H, d, J=2.9 Hz), 7.52 (1H, d, J=2.2 Hz), 7.47 (2H, dd, J=8.3 Hz), 7.33-7.21 (2H, m), 7.18 (1H, d, J=8.3 Hz), 6.50 (1H, dd, J=3.3, 9.4 Hz), 5.42-5.32 (1H, m), 4.80 (1H, d, J=16.0 Hz), 4.69 (1H, d, J=16.0 Hz), 3.19 (3H, s), 1.59 (3H, d, J=7.2 Hz);

MS (ESI) m/z 442 (M+H)⁺, 440 (M−H)⁻.

### Example 26

### 4-{(1S)-1-[({5-CHLORO-2-[(3-FLUOROPHENOXY)METHYL]PYRIDIN-3-YL}CARBONYL)AMINO]ETHYL}BENZOIC ACID

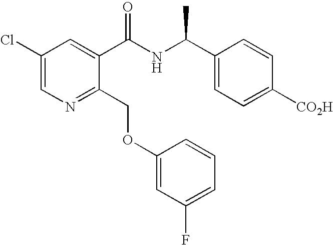

### STEP 1. Ethyl 2,5-dichloronicotinate

To a solution of 2,5-dichloronicotinic acid (30 g, 0.16 mol) in toluene (100 ml) was added ethanol (50 ml) and conc. sulfuric acid (1 ml). The reaction mixture was heated at 130° C. for 3 days with stirring. Then the reaction mixture was cooled and poured into sat. sodium bicarbonate solution. The whole mixture was extracted with ethyl acetate. The organic phase was washed with brine, dried (sodium sulfate), and concentrated to afford 34 g (quant.) of title compound:

¹H-NMR (CDCl₃) δ 8.48 (1H, d, J=2.6 Hz), 8.15 (1H, d, J=2.6 Hz), 4.44 (2H, dd, J=7.1, 14.3 Hz), 1.42 (3H, t, J=7.1 Hz).

### STEP 2. Ethyl 5-chloro-2-methylnicotinate

A mixture of ethyl 2,5-dichloronicotinate (step 1, 10 g, 0.045 mol), tetrakis(triphenylphoshine)palladium (5.2 g, 4.5 mmol), trimethylboroxine (5.65 g, 0.045 mmol) and potassium carbonate (18.66 g, 0.16 mmol) in 1,4-dioxane (contain 10% water, 100 ml) was refluxed for 7 h under nitrogen atmosphere. The reaction mixture was cooled to room temperature and poured into water. The aqueous mixture was extracted with ethyl acetate. The organic extracts were dried over sodium sulfate and concentrated. The residue was purified by flush column chromatography on silica gel eluting with hexane/ethyl acetate (50/1 to 20/1) to afford 3.41 g (38%) of the title compound:

¹H-NMR (CDCl₃) δ 8.57 (1H, d, J=2.5 Hz), 8.17 (1H, d, J=2.5 Hz), 4.39 (2H, dd, J=7.1, 14.2 Hz), 2.81 (3H, s), 1.41 (3H, t, J=7.1 Hz).

### STEP 3. Ethyl 5-chloro-2-methylnicotinate 1-oxide

The title compound was prepared according to the procedure described in step 3 of Example 21 from ethyl 5-chloro-2-methylnicotinate (step 2):

¹H-NMR (CDCl₃) δ 8.50 (1H, d, J=1.8 Hz), 7.74 (1H, d, J=1.8 Hz), 4.42 (2H, dd, J=7.1, 14.2 Hz), 2.75 (3H, s), 1.41 (3H, t, J=7.1 Hz).

### STEP 4. Ethyl 5-chloro-2-(hydroxymethyl)nicotinate

To a solution of ethyl 5-chloro-2-methylnicotinate 1-oxide (step 3, 4.1 g, 19 mmol) in dichloromethane (100 ml) was added trifluoromethane acetic acid anhydride (4 ml) at room temperature and the reaction mixture was stirred for 3 days. The reaction mixture was added 2 N hydrochloric acid solution (30 ml) with stirring. After 30 min, the whole mixture was extracted with dichloromethane. The organic phase was washed with water and brine, dried over sodium sulfate, and concentrated. The residue was purified by flush column chromatography on silica gel eluting with hexane/ethyl acetate (20/1 to 4/1) to afford 840 mg (20%) of the title compound:

¹H-NMR (CDCl₃) δ 8.69 (1H, d, J=2.3 Hz), 8.34 (1H, d, J=2.3 Hz), 5.06 (2H, s), 4.42 (2H, dd, J=7.1, 14.9 Hz), 1.42 (3H, t, J=7.1 Hz).

### STEP 5. Ethyl 5-chloro-2-[(3-fluorophenoxy)methyl]nicotinate

To a mixture of ethyl 5-chloro-2-(hydroxymethyl)nicotinate (step 4, 340 mg, 1.59 mmol), 3-fluorophenol (325 mg, 2.90 mmol), and triphenylphosphine (761 mg, 2.9 mmol) in tetrahydrofuran (10 ml) was added 40% solution of diethylazodicarboxylate in toluene (506 mg, 2.9 mmol) and the reaction mixture was stirred at room temperature for 5 hours. To the reaction mixture was added water and the whole mixture was extracted with ethyl acetate. The organic phase was dried over sodium sulfate and evaporated. The residue was purified by flush column chromatography on silica gel eluting with hexane/ethyl acetate (4/1) to afford 300 mg (56%) of the title compound:

¹H-NMR (CDCl₃) δ 8.69 (1H, d, J=2.3 Hz), 8.23 (1H, d, J=2.3 Hz), 7.27-7.15 (1H, m), 6.80-6.63 (3H, m), 5.49 (2H, s), 4.37 (2H, dd, J=7.1, 14.2 Hz), 1.33 (3H, t, J=7.1 Hz).

### STEP 6. 5-Chloro-2-[(3-fluorophenoxy)methyl]nicotinic acid

The title compound was prepared according to the procedure described in step 7 of Example 1 from ethyl 5-chloro-2-[(3-fluorophenoxy)methyl]nicotinate (step 5):

MS (ESI) m/z 282 (M+H)⁺, 280 (M−H)⁻.

### STEP 7. Methyl 4-{(1S)-1-[({5-chloro-2-[(3-fluorophenoxy)methyl]pyridin-3-yl}carbonyl)amino]ethyl}benzoate

The title compound was prepared according to the procedure described in step 6 of Example 1 from 5-chloro-2-[(3-fluorophenoxy)methyl]nicotinic acid (step 6) and methyl 4-[(1S)-1-aminoethyl]benzoate hydrochloride (step 5 of Example 1):

¹H-NMR (CDCl₃) δ 8.62 (1H, d, J=2.4 Hz), 8.00 (1H, d, J=2.4 Hz), 7.89 (2H, d, J=8.3 Hz), 7.34-7.14 (4H, m), 6.76-6.65 (2H, m), 6.65-6.56 (1H, m), 5.34-5.20 (1H, m), 5.17 (1H, d, J=10.5 Hz), 5.12 (1H, d, J=10.5 Hz), 3.91 (3H, s), 1.47 (3H, d, J=7.0 Hz).

### STEP 8. 4-{(1S)-1-[({5-chloro-2-[(3-fluorophenoxy)methyl]pyridin-3-yl}carbonyl)amino]ethyl}benzoic acid

The title compound was prepared according to the procedure described in step 7 of Example 1 from methyl 4-{(1S)-1-[({5-chloro-2-[(3-fluorophenoxy)methyl]pyridin-3-yl}carbonyl)amino]ethyl}benzoate (step 7):

¹H-NMR (DMSO-d₆) δ 9.19 (1H, d, J=7.7 Hz), 8.73 (1H, d, J=2.2 Hz), 8.10 (1H, d, J=2.2 Hz), 7.84 (2H, d, J=8.3 Hz), 7.47 (2H, d, J=8.3 Hz), 7.25 (1H, dd, J=8.1, 15.8 Hz), 6.83-6.60 (3H, m), 5.24 (1H, d, J=11.6 Hz), 5.18 (1H, d, J=11.6 Hz), 5.16-5.03 (1H, m), 1.41 (3H, d, J=7.0 Hz);

MS (ESI) m/z 429 (M+H)⁺, 427 (M−H)⁻.

### Example 27

### 4-{(1S)-1-[({5-CHLORO-2-[(4-FLUOROPHENOXY)METHYL]PYRIDIN-3-YL}CARBONYL)AMINO]ETHYL}BENZOIC ACID

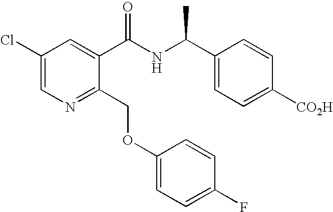

### STEP 1. 5-Chloro-2-[(4-fluorophenoxy)methyl]nicotinic acid

The title compound was prepared according to the procedure described in step 2 of Example 18 from 3-chlorofuro[3,4-b]pyridin-5(7H)-one (step 1 of Example 18) and 4-fluorophenol

¹H-NMR (CDCl₃) δ 8.73-8.62 (1H, brs), 8.30-8.19 (1H, brs), 6.98-6.80 (4H, m), 5.47 (2H, s).

### STEP 2. Methyl-4-{(1S)-1-[({5-chloro-2-[(4-fluorophenoxy)methyl]pyridin-3-yl}carbonyl)amino]ethyl}benzoate

The title compound was prepared according to the procedure described in step 6 of Example 1 from 5-chloro-2-[(4-fluorophenoxy)methyl]nicotinic acid (step 1) and methyl 4-[(1S)-1-aminoethyl]benzoate hydrochloride (step 5 of Example 1)

¹H-NMR (CDCl₃) δ 8.62 (1H, d, J=2.4 Hz), 8.05 (1H, d, J=2.4 Hz), 7.89 (2H, d, J=8.4 Hz), 7.34-7.27 (3H, m), 7.02-6.91 (2H, m), 6.87-6.78 (2H, m), 5.34-5.22 (1H, m), 5.14 (1H, d, J=10.1 Hz), 5.10 (1H, d, J=10.1 Hz), 3.92 (3H, s), 1.47 (3H, d, J=7.0 Hz).

### STEP 3. 4-{(1S)-1-[({5-Chloro-2-[(4-fluorophenoxy)methyl]pyridin-3-yl}carbonyl)amino]ethyl}benzoic acid

The title compound was prepared according to the procedure described in step 7 of Example 1 from methyl 4-{(1S)-1-[({5-chloro-2-[(4-fluorophenoxy)methyl]pyridin-3-yl}carbonyl)amino]ethyl}benzoate (step 2):

¹H-NMR (CDCl₃) δ 8.63 (1H, d, J=2.4 Hz), 8.07 (1H, d, J=2.4 Hz), 7.97 (2H, d, J=8.3 Hz), 7.40-7.28 (3H, m), 7.03-6.94 (2H, m), 6.92-6.83 (2H, m), 5.37-5.24 (1H, m), 5.17 (1H, d, J=10.3 Hz), 5.12 (1H, d, J=10.3 Hz), 1.48 (3H, d, J=7.0 Hz);

MS (ESI) m/z 429 (M+H)⁺, 427 (M−H)⁻.

The following examples illustrate the preparation of EP4-receptor antagonists described in U.S. 60/567,931:

### The Synthetic Procedure of Example 1-Example 6

The compounds disclosed hereinafter were prepared according to the following procedure:

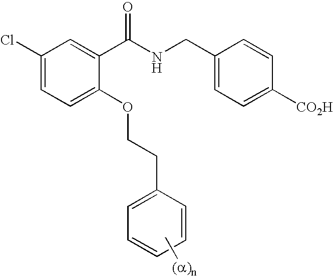

In the above structure, n represents 0, 1, 2, 3, 4 or 5.

### STEP 1. tert-Butyl 4-{[(5-chloro-2-hydroxybenzoyl)amino]methyl}benzoate

To a stirred solution of 5-chloro-2-hydroxybenzoic acid (0.57 g, 3.3 mmol) and tert-butyl 4-(aminomethyl)benzoate (0.72 g, 3.5 mmol) in dichloromethane (5 mL) were successively added 1-(3-dimethylaminopropyl)-3-ethylcarbodiimide hydrochloride (EDCl) (0.95 g, 5.0 mmol), 1-hydroxybenzotriazole hydrate (HOBT) (0.76 g, 5.0 mmol), and triethylamine (0.46 mL, 3.3 mmol). After being stirred overnight, the reaction mixture was poured into sodium bicarbonate aqueous solution (50 mL). The organic layer was separated, and the aqueous layer was extracted with dichloromethane (20 mL×2). The combined organic layers were washed with brine (50 mL), dried (magnesium sulfate), and evaporated. The residue was purified by flush column chromatography on silica gel eluting with hexane/ethyl acetate (10/1) to afford 0.57 g (48%) of the title compound as white solids:

¹H-NMR (CDCl₃) δ12.12 (1H, s), 7.99 (2H, d, J=7.9 Hz), 7.47-7.30 (4H, m), 6.97 (1H, d, J=8.4 Hz), 6.67-6.52 (1H, m), 4.68 (2H, d, J=5.7 Hz), 1.59 (9H, s).

### STEP 2. 4-[({[5-Fluoro-2-(substituted-phenoxy)pyridin-3-yl]carbonyl}amino)methyl]benzoic acid

To a solution of substituted-alcohol (0.10 mmol) were added a solution of tert-butyl 4-({[(5-chloro-2-hydroxybenzoyl)amino]methyl}benzoate (step 1, 0.05 mmol) in tetrahydrofuran (0.5 mL), triphenylphosphine on polystyrene (PS—PPh₃, 0.15 mmol), and di-tert-butyl azodicarboxylate (0.10 mmol) in tetrahydrofuran (0.2 mL). Then the mixture was agitated at room temperature overnight and filtered PS—PPh₃. The solvent was concentrated in vacuo and the residue was dissolved with ethyl acetate (0.65 mL), and then washed by water (0.45 mL). The organic layer was concentrated in vacuo. The crude product was purified by preparative LCMS (XTerra® C18, 20×50 mm) eluting with water/methanol/1% aqueous formic acid (90/5/5 to 0/95/5). After an addition of 1:1 mixture of trifluoroacetic acid and dichloroethane (0.6 mL) to the purified material, the mixture was stand at room temperature for 1 hour. Then the mixture was concentrated in vacuo to afford the desired product.

### Example 1

### 4-({[5-CHLORO-2-(2-PHENYLETHOXY)BENZOYL]AMINO}METHYL)BENZOIC ACID

Observed MS (ESI) m/z 409.99 (M+H)⁺

Exact Mass calcd for C₂₃H₂₀ClNO₄: m/z 409.11

### Example 2

### 4-[({5-CHLORO-2-[2-(2-CHLOROPHENYL)ETHOXY]BENZOYL}AMINO)METHYL]BENZOIC ACID

Observed MS (ESI) m/z 443.92 (M+H)⁺

Exact Mass calcd for C₂₃H₁₉Cl₂NO₄: m/z 443.07

### Example 3

### 4-[({5-CHLORO-2-[2-(4-FLUOROPHENYL)ETHOXY]BENZOYL}AMINO)METHYL]BENZOIC ACID

Observed MS (ESI) m/z 427.96 (M+H)⁺

Exact Mass calcd for C₂₃H₁₉ClFNO₄: m/z 427.10

### Example 4

### 4-[({5-CHLORO-2-[2-(4-CHLOROPHENYL)ETHOXY]BENZOYL}AMINO)METHYL]BENZOIC ACID

Observed MS (ESI) m/z 443.93 (M+H)⁺

Exact Mass calcd for C₂₃H₁₉Cl₂NO₄: m/z 443.07

### Example 5

### 4-[({5-CHLORO-2-(CYCLOHEXYLOXY)BENZOYL}AMINO)METHYL)BENZOIC ACID

Observed MS (ESI) m/z 387.98 (M+H)⁺

Exact Mass calcd for C₂₁H₂₂C1NO₄: m/z 387.12

### Example 6

### 4-[({5-CHLORO-2-[(4-CHLOROBENZYL)OXY]BENZOYL}AMINO)METHYL]BENZOIC ACID

Observed MS (ESI) m/z 429.91 (M+H)⁺

Exact Mass calcd for C₂₂H₁₇Cl₂NO₄: m/z 429.05

### Example 7

### 4-[({5-CHLORO-2-[2-(2-METHYLPHENYL)ETHOXY]BENZOYL}AMINO)METHYL]BENZOIC ACID

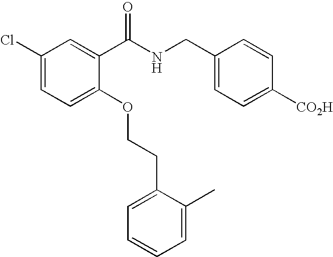

### STEP 1. tert-Butyl 4-[({5-chloro-2-[2-(2-methylphenyl)ethoxy]benzoyl}amino)methyl]benzoate

To a stirred solution of tert-butyl 4-{[(5-chloro-2-hydroxybenzoyl)amino]methyl}benzoate (step 1 of Example 1, 0.21 g, 0.58 mmol), 2-(2-methylphenyl)ethanol (0.16 g, 1.2 mmol) and triphenylphosphine (0.30 g, 1.2 mmol) in tetrahydrofuran (5 mL) was added di-tert-butyl azodicarboxylate (0.27 g, 1.2 mmol) at room temperature. After being stirred overnight, the reaction was quenched by the addition of sodium bicarbonate aqueous solution. The aqueous layer was extracted with ethyl acetate and the combined organic layers were washed with brine, dried (magnesium sulfate), and evaporated. The remaining residue was purified by flush column chromatography on silica gel eluting with hexane/ethyl acetate (5/1) to afford 0.21 g (76%) of the title compounds as a colorless oil:

¹H-NMR (CDCl₃) δ 8.20 (1H, d, J=2.8 Hz), 7.99 (1H, t, J=5.9 Hz), 7.91 (2H, d, J=8.2 Hz), 7.38 (1H, dd, J=8.7, 2.8 Hz), 7.25 (2H, d, J=8.2 Hz), 7.16-7.03 (4H, m), 6.93 (1H, d, J=8.7 Hz), 4.52 (2H, d, J=5.9 Hz), 4.34 (2H, t, J=6.8 Hz), 3.04 (2H, t, J=6.8 Hz), 2.25 (3H, s), 1.59 (9H, s);

MS (ESI) m/z 480 (M+H)⁺.

### STEP 2. 4-[({5-Chloro-2-[2-(2-methylphenyl)ethoxy]benzoyl}amino)methyl]benzoic acid

To a stirred solution of tert-butyl 4-[({5-chloro-2-[2-(2-methylphenyl)ethoxy]benzoyl}amino)methyl]benzoate (step 1, 0.21 g, 0.45 mmol) in dichloromethane (2 ml) was added trifluoroacetic acid (2 mL) at room temperature. The reaction mixture was stirred at room temperature for 1 h and then evaporated. The residual solid was washed with ether, and collected by filtration to afford 0.18 g (95%) of the title compound as white solids:

¹H-NMR (DMSO-d₆) δ 12.86 (1H, br.s), 8.60 (1H, t, J=6.1 Hz), 7.89 (2H, d, J=8.4 Hz), 7.66 (1H, d, J=2.8 Hz), 7.50 (1H, dd, J=8.9, 2.8 Hz), 7.40 (2H, d, J=8.4 Hz), 7.25 (1H, d, J=8.9 Hz), 7.22-7.04 (4H, m), 4.50 (2H, d, J=6.1 Hz), 4.32 (2H, t, J=6.9 Hz), 3.06 (2H, t, J=6.9 Hz), 2.26 (3H, s);

MS (ESI) m/z 424 (M+H)⁺, 422 (M−H)⁻.

### Example 8

### 4-[(1S)-1-({5-CHLORO-2-[2-(2,6-DIFLUOROPHENYL)ETHOXY]BENZOYL}AMINO)ETHYL]BENZOIC ACID

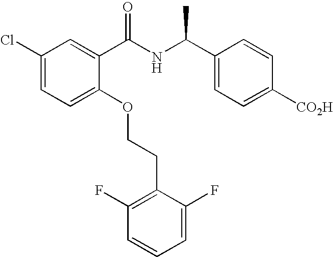

### STEP 1. tert-Butyl [(1S)-1-(4-bromophenyl)ethyl]carbamate

A mixture of [(1S)-1-(4-bromophenyl)ethyl]amine (10.00 g, 50.0 mmol) and di-tert-butyl dicarbonate (11.45 g, 52.5 mmol), triethylamine (7.66 mL, 55.0 mmol) in dichloromethane (200 mL) was stirred at room temperature for 1 hour. The mixture was diluted with dichloromethane (500 mL) and washed with 1 M hydrochloric acid (300 mL), saturated sodium hydrogen carbonate aqueous (300 mL), and brine (300 mL). The organic layer was dried over magnesium sulfate, and concentrated under reduced pressure. The residue was washed with cold hexane to afford 14.73 g (98%) of the title compounds as white solids:

¹H-NMR (CDCl₃) δ7.47-7.42 (2H, m), 7.18 (2H, d, J=8.4 Hz), 5.30 (2H, br.s), 1.41 (12H, br.s).

### STEP 2. Methyl 4-{(1S)-1-[(tert-butoxycarbonyl)amino]ethyl}benzoate

A mixture of tert-butyl [(1S)-1-(4-bromophenyl)ethyl]carbamate (step 1, 14.73 g, 49.1 mmol), 1,3-bis(diphenylphosphino)-propane (2.03 g, 4.91 mmol), palladium (II) acetate (1.10 g, 4.91 mmol), triethylamine (20.5 mL, 147 mmol), N,N-dimethylforamide (120 mL) and methanol (180 mL) was stirred at 80° C. for 16 h under carbon monoxide atmosphere. After cooling to room temperature, the mixture was diluted with ether (800 mL) and washed with water (500 mL×3). The organic layer was dried over magnesium sulfate and evaporated. The residue was purified by flush column chromatography on silica gel eluting with hexane/ethyl acetate (5/1) to afford 12.83 g (94%) of the title compounds as white solids:

¹H-NMR (CDCl₃) δ 8.02-7.99 (2H, m), 7.37 (2H, d, J=8.4 Hz), 4.83 (2H, br.s), 3.91 (3H, s), 1.46-1.42 (12H, m).

### STEP 3. Methyl 4-[(1S)-1-aminoethyl]benzoate hydrochloride

Methyl 4-{(1S)-1-[(tert-butoxycarbonyl)amino]ethyl}benzoate (step 2, 12.83 g, 45.9 mmol) was treated with trifluoroacetic acid (100 mL) and dichloromethane (100 mL) at room temperature for 16 hours. After removal of the solvent, the residue was diluted with 10% hydrogen chloride solution in methanol (100 mL). The mixture was concentrated under reduced pressure and the residue was washed with ethylacetate to give 9.40 g (95%) of the title compounds as white solids:

¹H-NMR (DMSO-d₆) δ 8.67 (2H, br.s), 8.01 (2H, d, J=8.4 Hz), 7.68 (2H, d, J=8.4 Hz), 4.49 (1H, q, J=6.9 Hz), 3.87 (3H, s), 1.53 (3H, d, J=6.9 Hz).

### STEP 4. Methyl 4-{(1S)-1-[(5-chloro-2-hydroxybenzoyl)amino]ethyl}benzoate

To a stirred solution of 5-chloro-2-hydroxybenzoic acid (1.2 g, 7.0 mmol) and methyl 4-[(1S)-1-aminoethyl]benzoate hydrochloride (step 3, 1.5 g, 7.0 mmol) in dichloromethane (18 mL) were successively added 1-(3-dimethylaminopropyl)-3-ethylcarbodiimide hydrochloride (EDCl) (2.0 g, 10 mmol), 1-hydroxybenzotriazole hydrate (HOBT) (1.6 g, 10 mmol), and triethylamine (1.0 mL, 7.3 mmol). After being stirred for 5 h, the reaction mixture was poured into sodium bicarbonate aqueous solution (100 mL). The organic layer was separated, and the aqueous layer was extracted with dichloromethane (50 mL×2). The combined organic layers were washed with brine (100 mL), dried (magnesium sulfate), and evaporated. The residue was purified by flush column chromatography on silica gel eluting with hexane/ethyl acetate (5/1) to afford 1.8 g (76%) of the title compounds as white solids:

¹H-NMR (CDCl₃) δ 12.08 (1H, s), 8.03 (2H, d, J=8.4 Hz), 7.47-7.30 (4H, m), 6.93 (1H, d, J=8.7 Hz), 6.59 (1H, d, J=7.3 Hz), 5.12 (1H, dq, J=7.3, 6.9 Hz), 3.92 (3H, s), 1.63 (3H, d, J=6.9 Hz);

MS (ESI) m/z 334 (M+H)⁺, 332 (M−H)⁻.

### STEP 5. Methyl 4-[(1S)-1-({5-chloro-2-[2-(2,6-difluorophenyl)ethoxy]benzoyl}amino)ethyl]benzoate

To a stirred solution of methyl 4-{(1S)-1-[(5-chloro-2-hydroxybenzoyl)amino]ethyl}benzoate (step 4, 0.12 g, 0.36 mmol), 2-(2,6-difluorophenyl)ethanol (0.12 g, 0.78 mmol) and triphenylphosphine (0.19 g, 0.72 mmol) in tetrahydrofuran (2 mL) was added di-tert-butyl azodicarboxylate (0.17 g, 0.72 mmol) at room temperature. After being stirred overnight, the reaction mixture was concentrated. Trifluoroacetic acid (2 mL) and dichloromethane (2 mL) were added to the residue, the solution was stirred for 1 h, and evaporated. The remaining residue was purified by flush column chromatography on silica gel eluting with hexane/ethyl acetate (5/1) to afford 0.16 g (84%) of the title compound as white solids:

¹H-NMR (CDCl₃) δ 8.17 (1H, d, J=7.3 Hz), 8.15 (1H, d, J=2.8 Hz), 8.01 (2H, d, J=8.4 Hz), 7.41 (2H, d, J=8.4 Hz), 7.37 (1H, dd, J=8.7, 2.8 Hz), 7.28-7.15 (1H, m), 6.92 (1H, d, J=8.7 Hz), 6.91-6.80 (2H, m), 5.36 (1H, dq, J=7.3, 6.9 Hz), 4.34 (2H, t, J=7.1 Hz), 3.91 (3H, s), 3.19 (2H, t, J=7.1 Hz), 1.57 (3H, d, J=6.9 Hz);

MS (ESI) m/z 474 (M+H)⁺.

### STEP 6. 4-[(1S)-1-({5-Chloro-2-[2-(2,6-difluorophenyl)ethoxy]benzoyl}amino)ethyl]benzoic acid

To a stirred solution of methyl 4-[(1S)-1-({5-chloro-2-[2-(2,6-difluorophenyl)ethoxy]benzoyl}amino)ethyl]benzoate (step 5, 0.16 g, 0.34 mmol) in methanol (2 mL) and tetrahydrofuran (3 mL) was added 2 N sodium hydroxide aqueous solution (2 mL). The reaction mixture was stirred at room temperature for 3 h and then evaporated. The residue was partitioned between ethyl acetate (30 mL) and 10% citric acid aqueous solution (30 mL). The organic phase was separated and the aqueous phase was extracted with ethyl acetate (30 mL). The combined organic extracts were washed with brine (50 mL), dried (magnesium sulfate), and concentrated. The residual solids were washed with ether, and dried in vacuo to afford 0.10 g (65%) of the title compound as white solids:

¹H-NMR (DMSO-d₆) δ 8.53 (1H, d, J=7.6 Hz), 7.90 (2H, d, J=8.2 Hz), 7.54 (1H, d, J=2.8 Hz), 7.51-7.43 (3H, m), 7.42-7.30 (1H, m), 7.22 (1H, d, J=8.9 Hz), 7.14-7.01 (2H, m), 5.13 (1H, dq, J=7.6, 7.1 Hz), 4.30 (2H, t, J=6.9 Hz), 3.13 (2H, t, J=6.9 Hz), 1.41 (3H, d, J=7.1 Hz);

MS (ESI) m/z 460 (M+H)⁺, 458 (M−H)⁻.

### Example 9

### 4-{(1S)-1-[({5-CHLORO-2-[2-(4-FLUOROPHENYL)ETHOXY]PYRIDIN-3-YL}CARBONYL)AMINO]ETHYL}BENZOIC ACID

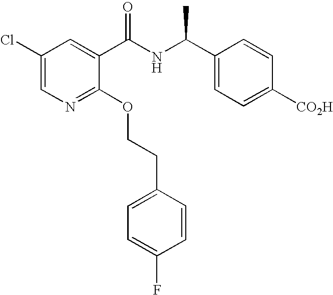

### STEP 1. 5-Chloro-2-[2-(4-fluorophenyl)ethoxy]nicotinic acid

A mixture of 2,5-dichloronicotinic acid (0.30 g, 1.6 mmol), 2-(4-fluorophenyl)ethanol (0.23 mL, 1.9 mmol), and sodium hydride in oil (0.15 g, 3.7 mmol) in N,N-dimethylforamide (2 mL) was heated at 90° C. in an oil bath for 3 hours. The reaction mixture was poured into 10% citric acid aqueous solution (50 mL), and extracted with ethyl acetate (50 mL×2). The combined organic layers were washed with water (50 mL×2) and brine (50 mL), dried (magnesium sulfate), and evaporated. The crude solids were washed with hexane/ether (10/1), and dried in vacuo to afford 0.31 g (68%) of the title compound as white solids:

¹H-NMR (CDCl₃) δ 8.41 (1H, d, J=2.8 Hz), 8.31 (1H, d, J=2.8 Hz), 7.27-7.20 (2H, m), 7.08-6.98 (2H, m), 4.78 (2H, t, J=6.8 Hz), 3.15 (2H, t, J=6.8 Hz);

MS (ESI) m/z 294 (M−H)⁻.

### STEP 2. Methyl 4-{(1S)-1-[({5-chloro-2-[2-(4-fluorophenyl)ethoxy]pyridin-3-yl}carbonyl)amino]ethyl}benzoate

The title compound was prepared according to the procedure described in step 4 of example 8 from 5-chloro-2-[2-(4-fluorophenyl)ethoxy]nicotinic acid (step 1):

¹H-NMR (CDCl₃) δ 8.45 (1H, d, J=2.8 Hz), 8.20 (1H, d, J=2.8 Hz), 8.03-7.94 (3H, m), 7.27 (2H, d, J=8.3 Hz), 7.18-7.08 (2H, m), 7.00-6.92 (2H, m), 5.25 (2H, t, J=7.2 Hz), 4.76 (1H, dq, J=6.8, 2.8 Hz), 3.92 (3H, s), 3.10 (2H, t, J=7.2 Hz), 1.39 (3H, d, J=6.8 Hz);

MS (ESI) m/z 457 (M+H)⁺.

### STEP 3. 4-{(1S)-1-[({5-Chloro-2-[2-(4-fluorophenyl)ethoxy]pyridin-3-yl}carbonyl)amino]ethyl}benzoic acid

The title compound was prepared according to the procedure described in step 6 of example 8 from methyl 4-{(1S)-1-[({5-chloro-2-[2-(4-fluorophenyl)ethoxy]pyridin-3-yl}carbonyl)amino]ethyl}benzoate (step 2):

¹H-NMR ((DMSO-d₆) δ8.54 (1H, d, J=7.7 Hz), 8.36 (1H, d, J=2.8 Hz), 8.04 (1H, d, J=2.8 Hz), 7.89 (2H, d, J=8.3 Hz), 7.43 (2H, d, J=8.3 Hz), 7.33-7.24 (2H, m), 7.12-7.02 (2H, m), 5.12 (1H, dq, J=7.7, 6.8 Hz), 4.60 (2H, t, J=6.6 Hz), 3.07 (2H, t, J=6.6 Hz), 1.37 (3H, d, J=6.8 Hz);

MS (ESI) m/z 443 (M+H)⁺, 441 (M−H)⁻.

### Example 10

### 4-[(1S)-1-({5-CHLORO-2-[2-(2-FLUOROPHENYL)ETHOXY]BENZOYL}AMINO)ETHYL]BENZOIC ACID

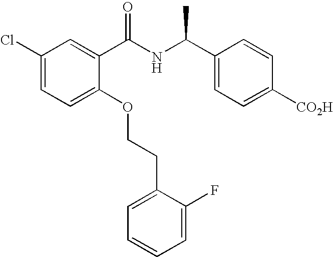

### STEP 1. Methyl 4-[(1S)-1-({5-chloro-2-[2-(2-fluorophenyl)ethoxy]benzoyl}amino)ethyl]benzoate

The title compound was prepared according to the procedure described in step 5 of Example 8 from methyl 4-{(1S)-1-[(5-chloro-2-hydroxybenzoyl)amino]ethyl}benzoate (step 4 of Example 8) and 2-(2-fluorophenyl)ethanol:

MS (ESI) m/z 456 (M+H)⁺.

### STEP 2. 4-[(1S)-1-({5-Chloro-2-[2-(2-fluorophenyl)ethoxy]benzoyl}amino)ethyl]benzoic acid

The title compound was prepared according to the procedure described in step 6 of Example 8 from methyl 4-[(1S)-1-({5-chloro-2-[2-(2-fluorophenyl)ethoxy]benzoyl}amino)ethyl]benzoate (step 1):

¹H-NMR (DMSO-d₆) [symbol] 8.49 (1H, d, J=7.5 Hz), 7.82 (2H, d, J=8.3 Hz), 7.57 (1H, d, J=2.8 Hz), 7.49 (1H, dd, J=9.0, 2.8 Hz), 7.43 (2H, d, J=8.3 Hz), 7.39-7.22 (3H, m), 7.21-7.08 (2H, m), 5.12 (1H, dq, J=7.5, 7.0 Hz), 4.36 (2H, t, J=6.8 Hz), 3.12 (2H, t, J=6.8 Hz), 1.36 (3H, d, J=7.0 Hz);

MS (ESI) m/z 442 (M+H)⁺, 440 (M−H)⁻.

### Example 11

### 4-[(1S)-1-({5-CHLORO-2-[2-(2-METHYLPHENYL)ETHOXY]BENZOYL}AMINO)ETHYL]BENZOIC ACID

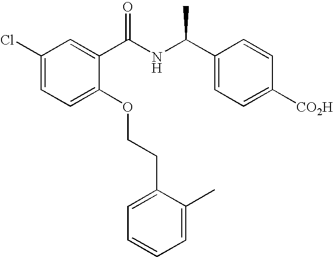

### STEP 1. Methyl 4-[(1S)-1-({5-chloro-2-[2-(2-methylphenyl)ethoxy]benzoyl}amino)ethyl]benzoate

The title compound was prepared according to the procedure described in step 5 of Example 8 from methyl 4-{(1S)-1-[(5-chloro-2-hydroxybenzoyl)amino]ethyl}benzoate (step 4 of Example 8) and 2-(2-methylphenyl)ethanol:

MS (ESI) m/z 452 (M+H)⁺.

### STEP 2. 4-[(1S)-1-({5-Chloro-2-[2-(2-methylphenyl)ethoxy]benzoyl}amino)ethyl]benzoic acid

The title compound was prepared according to the procedure described in step 6 of Example 8 from methyl 4-[(1S)-1-({5-chloro-2-[2-(2-methylphenyl)ethoxy]benzoyl}amino)ethyl]benzoate (step 1):

¹H-NMR (DMSO-d₆) [symbol] 8.50 (1H, d, J=7.6 Hz), 7.88 (2H, d, J=8.2 Hz), 7.58 (1H, d, J=2.8 Hz), 7.49 (1H, dd, J=8.9, 2.8 Hz), 7.42 (2H, d, J=8.2 Hz), 7.25 (1H, d, J=8.9 Hz), 7.24-7.06 (4H, m), 5.11 (1H, dq, J=7.6, 7.1 Hz), 4.34 (2H, t, J=6.9 Hz), 3.06 (2H, t, J=6.9 Hz), 2.28 (3H, s), 1.33 (3H, d, J=7.1 Hz);

MS (ESI) m/z 438 (M+H)⁺, 436 (M−H)⁻.

### Example 12

### 4-[(1S)-1-({5-CHLORO-2-[2-(4-METHYLPHENYL)ETHOXY]BENZOYL}AMINO)ETHYL]BENZOIC ACID

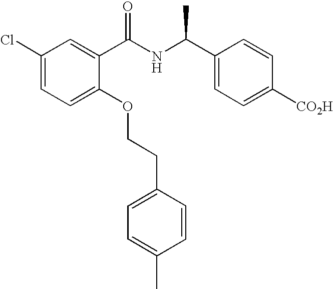

### STEP 1. Methyl 4-[(1S)-1-({5-chloro-2-[2-(4-methylphenyl)ethoxy]benzoyl}amino)ethyl]benzoate

The title compound was prepared according to the procedure described in step 5 of Example 8 from methyl 4-{(1S)-1-[(5-chloro-2-hydroxybenzoyl)amino]ethyl}benzoate (step 4 of Example 8) and 2-(4-methylphenyl)ethanol:

MS (ESI) m/z 452 (M+H)⁺.

### STEP 2. 4-[(1S)-1-({5-Chloro-2-[2-(4-methylphenyl)ethoxy]benzoyl}amino)ethyl]benzoic acid

The title compound was prepared according to the procedure described in step 6 of Example 8 from methyl 4-[(1S)-1-({5-chloro-2-[2-(4-methylphenyl)ethoxy]benzoyl}amino)ethyl]benzoate (step 1):

¹H-NMR (DMSO-d₆) [symbol] 8.44 (1H, d, J=7.6 Hz), 7.88 (2H, d, J=8.2 Hz), 7.60 (1H, d, J=2.8 Hz), 7.50 (1H, dd, J=8.9, 2.8 Hz), 7.40 (2H, d, J=8.2 Hz), 7.23 (1H, d, J=8.9 Hz), 7.14 (2H, d, J=8.1 Hz), 7.07 (2H, d, J=8.1 Hz), 5.12 (1H, dq, J=7.6, 6.9 Hz), 4.34 (2H, t, J=6.6 Hz), 3.04 (2H, t, J=6.6 Hz), 2.24 (3H, s), 1.36 (3H, d, J=6.9 Hz);

MS (ESI) m/z 438 (M+H)⁺, 436 (M−H)⁻.

### Example 13

### 4-((1S)-1-{[5-CHLORO-2-(CYCLOHEXYLOXY)BENZOYL]AMINO}ETHYL)BENZOIC ACID

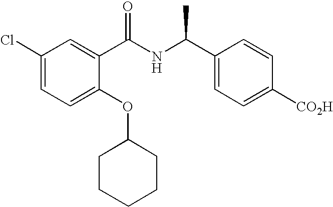

### STEP 1. Methyl 4-((1S)-1-{[5-chloro-2-(cyclohexyloxy)benzoyl]amino}ethyl)benzoate

The title compound was prepared according to the procedure described in step 5 of Example 8 from methyl 4-{(1S)-1-[(5-chloro-2-hydroxybenzoyl)amino]ethyl}benzoate (step 4 of example 8) and cyclohexanol:

MS (ESI) m/z 416 (M+H)⁺, 414 (M−H)⁻.

### STEP 2. 4-((1S)-1-{[5-Chloro-2-(cyclohexyloxy)benzoyl]amino}ethyl)benzoic acid

The title compound was prepared according to the procedure described in step 6 of Example 8 from methyl 4-((1S)-1-{[5-chloro-2-(cyclohexyloxy)benzoyl]amino}ethyl)benzoate (step 1):

¹H-NMR (DMSO-d₆) [symbol] 8.60 (1H, d, J=7.6 Hz), 7.91 (2H, d, J=8.2 Hz), 7.60 (1H, d, J=2.8 Hz), 7.51 (2H, d, J=8.2 Hz), 7.47 (1H, dd, J=8.9, 2.8 Hz), 7.23 (1H, d, J=8.9 Hz), 5.17 (1H, dq, J=7.6, 7.1 Hz), 4.59-4.44 (1H, m), 2.05-1.85 (2H, m), 1.70-1.10 (8H, m), 1.48 (3H, d, J=7.1 Hz);

MS (ESI) m/z 402 (M+H)⁺, 400 (M−H)⁻.

### Example 14

### 4-((1S)-1-{[5-CHLORO-2-(3-METHYLBUTOXY)BENZOYL]AMINO}ETHYL)BENZOIC ACID

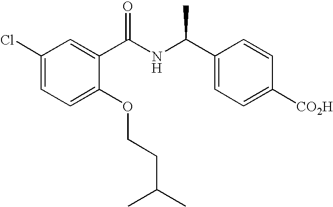

### STEP 1. Methyl 4-((1S)-1-{[5-chloro-2-(3-methylbutoxy)benzoyl]amino}ethyl)benzoate

The title compound was prepared according to the procedure described in step 5 of Example 8 from methyl 4-{(1S)-1-[(5-chloro-2-hydroxybenzoyl)amino]ethyl}benzoate (step 4 of Example 8) and 3-methylbutan-1-ol:

MS (ESI) m/z 404 (M+H)⁺.

### STEP 2. 4-((1S)-1-{[5-Chloro-2-(3-methylbutoxy)benzoyl]amino}ethyl)benzoic acid

The title compound was prepared according to the procedure described in step 6 of Example 8 from methyl 4-((1S)-1-{[5-chloro-2-(3-methylbutoxy)benzoyl]amino}ethyl)benzoate (step 1):

¹H-NMR (DMSO-d₆) [symbol] 8.57 (1H, d, J=7.6 Hz), 7.92 (2H, d, J=8.1 Hz), 7.58 (1H, d, J=2.6 Hz), 7.55-7.46 (3H, m), 7.20 (1H, d, J=8.9 Hz), 5.16 (1H, dq, J=7.6, 6.9 Hz), 4.09 (2H, t, J=6.3 Hz), 1.76-1.54 (3H, m), 1.46 (3H, d, J=6.9 Hz), 0.87 (3H, d, J=6.1 Hz), 0.86 (3H, d, J=6.1 Hz);

MS (ESI) m/z 390 (M+H)⁺, 388 (M−H)⁻.

### Example 15

### 4-{(1S)-1-[({5-CHLORO-2-[2-(4-CHLOROPHENYL)ETHOXY]PYRIDIN-3-YL}CARBONYL)AMINO]ETHYL}BENZOIC ACID

### STEP 1. 5-Chloro-2-[2-(4-chlorophenyl)ethoxy]nicotinic acid

The title compound was prepared according to the procedure described in step 1 of Example 9 from 2,5-dichloronicotinic acid and 2-(4-chlorophenyl)ethanol:

¹H-NMR (CDCl₃) δ8.38 (1H, d, J=2.8 Hz), 8.29 (1H, d, J=2.8 Hz), 7.35-7.12 (4H, m), 4.75 (2H, t, J=6.8 Hz), 3.13 (2H, t, J=6.8 Hz).

### STEP 2. Methyl 4-{(1S)-1-[({5-chloro-2-[2-(4-chlorophenyl)ethoxy]pyridin-3-yl}carbonyl)amino]ethyl}benzoate

The title compound was prepared according to the procedure described in step 4 of Example 8 from 5-chloro-2-[2-(4-chlorophenyl)ethoxy]nicotinic acid (step 1) and methyl 4-[(1S)-1-aminoethyl]benzoate hydrochloride (step 3 of Example 8):

¹H-NMR (CDCl₃) δ8.44 (1H, d, J=2.8 Hz), 8.19 (1H, d, J=2.8 Hz), 8.01-7.95 (2H, m), 7.95-7.88 (1H, m), 7.30-7.18 (4H, m), 7.12-7.04 (2H, m), 5.30-5.15 (1H, m), 4.84-4.67 (2H, m), 3.90 (3H, s), 3.08 (2H, t, J=6.6 Hz), 1.37 (3H, d, J=7.0 Hz).

### STEP 3. 4-{(1S)-1-[({5-Chloro-2-[2-(4-chlorophenyl)ethoxy]pyridin-3-yl}carbonyl)amino]ethyl}benzoic acid

The title compound was prepared according to the procedure described in step 6 of Example 8 from methyl 4-{(1S)-1-[({5-chloro-2-[2-(4-chlorophenyl)ethoxy]pyridin-3-yl}carbonyl)amino]ethyl}benzoate (step 2):

¹H-NMR (DMSO-d₆) δ 8.51 (1H, d, J=7.7 Hz), 8.34 (1H, d, J=2.8 Hz), 8.02 (1H, d, J=2.8 Hz), 7.88 (2H, d, J=8.4 Hz), 7.40 (2H, d, J=8.4 Hz), 7.30-7.20 (4H, m), 5.09 (1H, dq, J=7.7, 7.0 Hz), 4.59 (2H, t, J=6.4 Hz), 3.05 (2H, t, J=6.4 Hz), 1.35 (3H, d, J=7.0 Hz);

MS (ESI) m/z 459 (M+H)⁺, 457 (M−H)⁻.

### Example 16

### 4-{(1S)-1-[({5-CHLORO-2-[METHYL(2-PHENYLETHYL)AMINO]PYRIDIN-3-YL}CARBONYL)AMINO]ETHYL}BENZOIC ACID

### STEP 1. Methyl 4-((1s)-1-{[(2,5-dichloropyridin-3-yl)carbonyl]amino}ethyl)benzoate

To a stirred solution of 2,5-dichloronicotinic acid (Syn. Commun. 1989, 19, 553-9, 2.0 g, 10.4 mmol) and methyl 4-[(1S)-1-aminoethyl]benzoate hydrochloride (step 3 of Example 8, 2.35 g, 10.9 mmol) in dichloromethane (10 mL) was added 1,1′-carbonyldiimidazole (CDl) (1.77 g, 10.9 mmol) in small portions. After being stirred overnight, the reaction mixture was poured into water (80 mL). The precipitated solids were collected by the filtration and dried. The crude product was purified by flush column chromatography on silica gel (100 g) eluting with dichloromethane/ethyl acetate (20/1) to afford 3.4 g (93%) of the title compound as white solids:

¹H-NMR (CDCl₃) δ8.42 (1H, d, J=2.6 Hz), 8.10 (1H, d, J=2.6 Hz), 8.04 (2H, d, J=8.6 Hz), 7.46 (2H, d, J=8.6 Hz), 6.82 (1H, d, J=7.3 Hz), 5.40-5.30 (1H, m), 3.92 (3H, s), 1.64 (3H, d, J=7.0 Hz);

MS (ESI) m/z 353 (M+H)⁺, 351 (M−H)⁻.

### STEP 2. Methyl 4-{(1S)-1-[({5-chloro-2-[methyl(2-phenylethyl)amino]pyridin-3-yl}carbonyl)amino]ethyl}benzoate

A mixture of methyl 4-((1S)-1-{[(2,5-dichloropyridin-3-yl)carbonyl]amino}ethyl)benzoate (step 1, 150 mg, 0.43 mmol), [2-(4-chlorophenyl)ethyl]amine (64 mg, 0.47 mmol) and potassium carbonate (88 mg, 0.64 mmol) in N,N-dimethylformamide (1.5 mL) was heated at 100° C. for 18 hours. After cooling, the mixture was partitioned between ethyl acetate and water. The organic layer was separated, washed with brine, dried over sodium sulfate, and evaporated. The residue was purified by flash column chromatography on silica gel eluting with hexane/ethyl acetate (3/1) to afford 140 mg (72%) of the title compound:

¹H-NMR (CDCl₃) δ8.40-8.22 (2H, m), 8.17 (1H, d, J=2.8 Hz), 7.96 (2H, d, J=8.4 Hz), 7.30-7.00 (7H, m), 5.35-5.15 (1H, m), 3.90 (3H, s), 3.63-3.38 (2H, m), 2.80 (2H, t, J=7.0 Hz), 2.62 (3H, s), 1.33 (3H, d, J=6.9 Hz).

### STEP 3. 4-{(1S)-1-[({5-Chloro-2-[methyl(2-phenylethyl)amino]pyridin-3-yl}carbonyl)amino]ethyl}benzoic acid

The title compound was prepared according to the procedure described in step 6 of Example 8 from methyl 4-{(1S)-1-[({5-chloro-2-[methyl(2-phenylethyl)amino]pyridin-3-yl}carbonyl)amino]ethyl}benzoate (step 2):

¹H-NMR (DMSO-d₆) δ9.06 (1H, d, J=8.1 Hz), 8.15 (1H, d, J=2.6 Hz), 7.88 (2H, d, J=8.4 Hz), 7.55 (1H, d, J=2.6 Hz), 7.47 (2H, d, J=8.4 Hz), 7.30-7.10 (5H, m), 5.06 (1H, dq, J=8.1, 7.3 Hz), 3.64-3.44 (2H, m), 2.80-2.70 (5H, m), 1.40 (3H, d, J=7.3 Hz);

MS (ESI) m/z 438 (M+H)⁺, 436 (M−H)⁻.

### Example 17

### 4-[(1S)-1-({5-CHLORO-2-[(CIS-4-METHYLCYCLOHEXYL)OXY]BENZOYL}AMINO)ETHYL]BENZOIC ACID

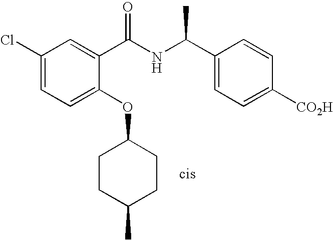

### STEP 1. Methyl 4-[(1S)-1-({5-chloro-2-[(cis-4-methylcyclohexyl)oxy]benzoyl}amino)ethyl]benzoate and methyl 4-[(1S)-1-({5-chloro-2-[(trans-4-methylcyclohexyl)oxy]benzoyl}amino)ethyl]benzoate

The title compounds were prepared according to the procedure described in step 5 of Example 8 from methyl 4-{(1S)-1-[(5-chloro-2-hydroxybenzoyl)amino]ethyl}benzoate (step 4 of example 8) and 4-methylcyclohexanol:

cis-isomer;

¹H-NMR (CDCl₃) δ8.43 (1H, d, J=7.4 Hz), 8.19 (1H, d, J=2.8 Hz), 8.00 (2H, d, J=8.4 Hz), 7.45 (2H, d, J=8.4 Hz), 7.43 (1H, dd, J=8.9, 2.8 Hz), 6.89 (1H, d, J=8.9 Hz), 5.40 (1H, dq, J=7.4, 7.0 Hz), 4.75-4.63 (1H, m), 3.89 (3H, s), 2.10-1.02 (9H, m), 1.61 (3H, d, J=7.0 Hz), 0.82 (3H, d, J=6.2 Hz);

MS (ESI) m/z 430 (M+H)⁺, 428 (M−H)⁻;

trans-isomer;

¹H-NMR (CDCl₃) δ8.46 (1H, d, J=7.4 Hz), 8.19 (1H, d, J=2.9 Hz), 8.02 (2H, d, J=8.3 Hz), 7.43 (2H, d, J=8.3 Hz), 7.34 (1H, dd, J=8.9, 2.9 Hz), 6.93 (1H, d, J=8.9 Hz), 5.33 (1H, dq, J=7.4, 6.8 Hz), 4.39-4.22 (1H, m), 3.91 (3H, s), 2.25-2.08 (2H, m), 1.87-1.72 (2H, m), 1.58 (3H, d, J=6.8 Hz), 1.50-1.26 (3H, m), 1.15-0.96 (2H, m), 0.93 (3H, d, J=6.4 Hz);

MS (ESI) m/z 430 (M+H)⁺, 428 (M−H)⁻.

### STEP 2. 4-[(1S)-1-({5-Chloro-2-[(cis-4-methylcyclohexyl)oxy]benzoyl}amino)ethyl]benzoic acid

The title compound was prepared according to the procedure described in step 6 of Example 8 from methyl 4-[(1S)-1-({5-chloro-2-[(cis-4-methylcyclohexyl)oxy]benzoyl}amino)ethyl]benzoate (step 1):

¹H-NMR (DMSO-d₆) [symbol] 8.58 (1H, d, J=7.4 Hz), 7.91 (2H, d, J=8.0 Hz), 7.62 (1H, d, J=2.6 Hz), 7.51 (2H, d, J=8.0 Hz), 7.49 (1H, dd, J=9.1, 2.6 Hz), 7.19 (1H, d, J=9.1 Hz), 5.20 (1H, dq, J=7.4, 6.9 Hz), 4.83-4.68 (1H, m), 1.98-1.82 (2H, m), 1.68-0.96 (7H, m), 1.49 (3H, d, J=6.9 Hz), 0.77 (3H, d, J=6.1 Hz);

MS (ESI) m/z 416 (M+H)⁺, 414 (M−H)⁻.

### Example 18

### 4-[(1S)-1-({5-CHLORO-2-[(TRANS-4-METHYLCYCLOHEXYL)OXY]BENZOYL}AMINO)ETHYL]BENZOIC ACID

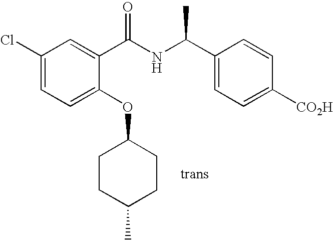

### STEP 2. 4-[(1S)-1-({5-Chloro-2-[(trans-4-methylcyclohexyl)oxy]benzoyl}amino)ethyl]benzoic acid

The title compound was prepared according to the procedure described in step 6 of Example 8 from methyl 4-[(1S)-1-({5-chloro-2-[(trans-4-methylcyclohexyl)oxy]benzoyl}amino)ethyl]benzoate (step 1 of example 17):

¹H-NMR (DMSO-d₆) [symbol] 8.58 (1H, d, J=7.6 Hz), 7.91 (2H, d, J=8.3 Hz), 7.60 (1H, d, J=2.8 Hz), 7.51 (2H, d, J=8.3 Hz), 7.46 (1H, dd, J=9.1, 2.8 Hz), 7.25 (1H, d, J=9.1 Hz), 5.15 (1H, dq, J=7.6, 6.9 Hz), 4.48-4.30 (1H, m), 2.18-1.98 (2H, m), 1.77-1.62 (2H, m), 1.47 (3H, d, J=6.9 Hz), 1.42-1.20 (3H, m), 1.15-0.95 (2H, m), 0.88 (3H, d, J=6.4 Hz);

MS (ESI) m/z 416 (M+H)⁺, 414 (M−H)⁻.

### Example 19

### 4-{(1S)-1-[({5-CHLORO-2-[2-(2-METHYLPHENYL)ETHOXY]PYRIDIN-3-YL}CARBONYL)AMINO]ETHYL}BENZOIC ACID

### STEP 1. 5-Chloro-2-[2-(2-methylphenyl)ethoxy]nicotinic acid

The title compound was prepared according to the procedure described in step 1 of Example 9 from 2,5-dichloronicotinic acid and 2-(2-methylphenyl)ethanol:

¹H-NMR (CDCl₃) δ8.42 (1H, d, J=2.8 Hz), 8.31 (1H, d, J=2.8 Hz), 7.25-7.15 (4H, m), 4.78 (2H, t, J=7.2 Hz), 3.19 (2H, t, J=7.2 Hz), 2.39 (3H, s);

MS (ESI) m/z 292 (M+H)⁺, 290 (M−H)⁻.

### STEP 2. Methyl 4-{(1S)-1-[({5-chloro-2-[2-(2-methylphenyl)ethoxy]pyridin-3-yl}carbonyl)amino]ethyl}benzoate

The title compound was prepared according to the procedure described in step 4 of Example 8 from 5-chloro-2-[2-(2-methylphenyl)ethoxy]nicotinic acid (step 1) and methyl 4-[(1S)-1-aminoethyl]benzoate hydrochloride (step 3 of Example 8):

¹H-NMR (CDCl₃) δ8.44 (1H, d, J=2.8 Hz), 8.20 (1H, d, J=2.8 Hz), 8.10-8.00 (1H, m), 7.96 (2H, d, J=8.4 Hz), 7.30-7.10 (6H, m), 5.30-5.10 (1H, m), 4.87-4.65 (2H, m), 3.96 (3H, s), 3.10 (2H, t, J=6.6 Hz), 2.30 (3H, s), 1.34 (3H, d, J=6.9 Hz).

### STEP 3. 4-{(1S)-1-[({5-Chloro-2-[2-(2-methylphenyl)ethoxy]pyridin-3-yl}carbonyl)amino]ethyl}benzoic acid

The title compound was prepared according to the procedure described in step 6 of Example 8 from methyl 4-{(1S)-1-[({5-chloro-2-[2-(2-methylphenyl)ethoxy]pyridin-3-yl}carbonyl)amino]ethyl}benzoate (step 2):

¹H-NMR (DMSO-d₆) δ 8.57 (1H, d, J=7.7 Hz), 8.33 (1H, d, J=2.8 Hz), 8.01 (1H, d, J=2.8 Hz), 7.87 (2H, d, J=8.3 Hz), 7.41 (2H, d, J=8.3 Hz), 7.20-7.05 (4H, m), 5.15-5.01 (1H, m), 4.57 (2H, t, J=6.8 Hz), 3.03 (2H, t, J=6.8 Hz), 2.25 (3H, s), 1.33 (3H, d, J=7.0 Hz);

MS (ESI) m/z 439 (M+H)⁺, 437 (M−H)⁻.

### Example 20

### 4-((1S)-1-{[5-CHLORO-2-(3-METHOXY-3-METHYLBUTOXY)BENZOYL]AMINO}ETHYL)BENZOIC ACID

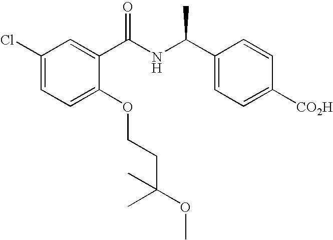

### STEP 1. Methyl 4-((1S)-1-{[5-chloro-2-(3-methoxy-3-methylbutoxy)benzoyl]amino}ethyl)benzoate

The title compound was prepared according to the procedure described in step 5 of Example 8 from methyl 4-{(1S)-1-[(5-chloro-2-hydroxybenzoyl)amino]ethyl}benzoate (step 4 of Example 8) and 3-methoxy-3-methylbutan-1-ol:

¹H-NMR (CDCl₃) δ 8.32 (1H, d, J=6.8 Hz), 8.14 (1H, d, J=2.8 Hz), 8.02 (2H, d, J=8.4 Hz), 7.45 (2H, d, J=8.4 Hz), 7.37 (1H, dd, J=8.9, 2.8 Hz), 6.93 (1H, d, J=8.9 Hz), 5.35 (1H, dq, J=6.9, 6.8 Hz), 4.25-4.10 (2H, m), 3.91 (3H, s), 3.15 (3H, s), 1.98-1.83 (2H, m), 1.59 (3H, d, J=6.9 Hz), 1.19 (3H, s), 1.18 (3H, s);

MS (ESI) m/z 434 (M+H)⁺, 432 (M−H)⁻.

### STEP 2. 4-((1S)-1-{[5-Chloro-2-(3-methoxy-3-methylbutoxy)benzoyl]amino}ethyl)benzoic acid

The title compound was prepared according to the procedure described in step 6 of Example 8 from methyl 4-((1S)-1-{[5-chloro-2-(3-methoxy-3-methyl butoxy)benzoyl]amino}ethyl)benzoate (step 1):

¹H-NMR (DMSO-d₆) δ 12.87 (1H, br.s), 8.60 (1H, d, J=7.5 Hz), 7.92 (2H, d, J=8.4 Hz), 7.56 (1H, d, J=2.8 Hz), 7.52 (2H, d, J=8.4 Hz), 7.48 (1H, dd, J=9.0, 2.8 Hz), 7.20 (1H, d, J=9.0 Hz), 5.16 (1H, dq, J=7.5, 7.0 Hz), 4.18-4.06 (2H, m), 3.06 (3H, s), 1.97-1.78 (2H, m), 1.46 (3H, d, J=7.0 Hz), 1.11 (3H, s), 1.10 (3H, s);

MS (ESI) m/z 420 (M+H)⁺, 418 (M−H)⁻.

### Example 21

### 4-((1S)-1-{[5-CHLORO-2-(2-ISOPROPOXYETHOXY)BENZOYL]AMINO}ETHYL)BENZOIC ACID

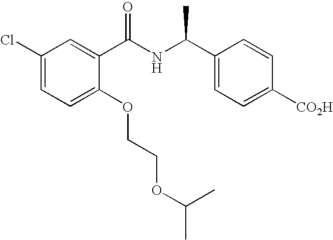

### STEP 1. Methyl 4-((1S)-1-{[5-chloro-2-(2-isopropoxyethoxy)benzoyl]amino}ethyl)benzoate

The title compound was prepared according to the procedure described in step 5 of Example 8 from methyl 4-{(1S)-1-[(5-chloro-2-hydroxybenzoyl)amino]ethyl}benzoate (step 4 of Example 8) and 2-isopropoxyethanol:

¹H-NMR (CDCl₃) δ 8.44 (1H, d, J=7.7 Hz), 8.15 (1H, d, J=2.8 Hz), 8.01 (2H, d, J=8.2 Hz), 7.47 (2H, d, J=8.2 Hz), 7.37 (1H, dd, J=8.9, 2.8 Hz), 6.90 (1H, d, J=8.9 Hz), 5.38 (1H, dq, J=7.7, 7.1 Hz), 4.28-4.20 (2H, m), 3.90 (3H, s), 3.82-3.75 (2H, m), 3.58 (1H, sep, J=6.1 Hz), 1.58 (3H, d, J=7.1 Hz), 1.13 (3H, d, J=6.1 Hz), 1.11 (3H, d, J=6.1 Hz);

MS (ESI) m/z 420 (M+H)⁺, 418 (M−H)⁻.

### STEP 2. 4-((1S)-1-{[5-Chloro-2-(2-isopropoxyethoxy)benzoyl]amino}ethyl)benzoic acid

The title compound was prepared according to the procedure described in step 6 of Example 8 from ethyl 4-((1S)-1-{[5-chloro-2-(2-isopropoxyethoxy)benzoyl]amino}ethyl)benzoate (step 1):

¹H-NMR (DMSO-d₆) δ 12.88 (1H, br.s), 8.60 (1H, d, J=7.7 Hz), 7.91 (2H, d, J=8.3 Hz), 7.69 (1H, d, J=2.8 Hz), 7.53 (1H, dd, J=9.0, 2.8 Hz), 7.50 (2H, d, J=8.3 Hz), 7.22 (1H, d, J=9.0 Hz), 5.19 (1H, dq, J=7.7, 7.1 Hz), 4.29-4.20 (2H, m), 3.78-3.70 (2H, m), 3.58 (1H, sep, J=6.1 Hz), 1.47 (3H, d, J=7.1 Hz), 1.04 (6H, d, J=6.1 Hz);

MS (ESI) m/z 406 (M+H)⁺, 404 (M−H)⁻.

### Example 22

### 4-[(1S)-1-({5-CHLORO-2-[(2-CHLOROBENZYL)OXY]BENZOYL}AMINO)ETHYL]BENZOIC ACID

### STEP 1. Methyl 4-[(1S)-1-({5-chloro-2-[(2-chlorobenzyl)oxy]benzoyl}amino)ethyl]benzoate

The title compound was prepared according to the procedure described in step 5 of Example 8 from methyl 4-{(1S)-1-[(5-chloro-2-hydroxybenzoyl)amino]ethyl}benzoate (step 4 of Example 8) and (2-chlorophenyl)methanol:

MS (ESI) m/z 458 (M+H)⁺, 456 (M−H)⁻.

### STEP 2. 4-[(1S)-1-({5-Chloro-2-[(2-chlorobenzyl)oxy]benzoyl}amino)ethyl]benzoic acid

The title compound was prepared according to the procedure described in step 6 of Example 8 from methyl 4-[(1S)-1-({5-chloro-2-[(2-chlorobenzyl)oxy]benzoyl}amino)ethyl]benzoate (step 1):

¹H-NMR (DMSO-d₆) δ 8.51 (1H, d, J=8.6 Hz), 7.78 (2H, d, J=8.3 Hz), 7.73-7.51 (4H, m), 7.50-7.30 (3H, m), 7.28 (2H, d, J=8.3 Hz), 5.29 (2H, s), 5.08 (1H, dq, J=8.6, 7.0 Hz), 1.26 (3H, d, J=7.0 Hz);

MS (ESI) m/z 444 (M+H)⁺, 442 (M−H)⁻.

### Example 23

### 4-[(1S)-1-({5-CHLORO-2-[(3-CHLOROBENZYL)OXY]BENZOYL}AMINO)ETHYL]BENZOIC ACID

### STEP 1. Methyl 4-[(1S)-1-({5-chloro-2-[(3-chlorobenzyl)oxy]benzoyl}amino)ethyl]benzoate

The title compound was prepared according to the procedure described in step 5 of Example 8 from methyl 4-{(1S)-1-[(5-chloro-2-hydroxybenzoyl)amino]ethyl}benzoate (step 4 of Example 8) and (3-chlorophenyl)methanol:

MS (ESI) m/z 458 (M+H)⁺, 456 (M−H)⁻.

### STEP 2. 4-[(1S)-1-({5-Chloro-2-[(3-chlorobenzyl)oxy]benzoyl}amino)ethyl]benzoic acid

The title compound was prepared according to the procedure described in step 6 of Example 8 from methyl 4-[(1S)-1-({5-chloro-2-[(3-chlorobenzyl)oxy]benzoyl}amino)ethyl]benzoate (step 1):

¹H-NMR (DMSO-d₆) δ 8.63 (1H, d, J=7.5 Hz), 7.80 (2H, d, J=8.4 Hz), 7.60-7.39 (5H, m), 7.34 (2H, d, J=8.4 Hz), 7.29 (2H, d, J=8.4 Hz), 5.22 (2H, s), 5.09 (1H, dq, J=7.5, 6.8 Hz), 1.33 (3H, d, J=6.8 Hz);

MS (ESI) m/z 444 (M+H)⁺, 442 (M−H)⁻.

### Example 24

### 4-[(1S)-1-({5-CHLORO-2-[(4-CHLOROBENZYL)OXY]BENZOYL}AMINO)ETHYL]BENZOIC ACID

### STEP 1. Methyl 4-[(1S)-1-({5-chloro-2-[(4-chlorobenzyl)oxy]benzoyl}amino)ethyl]benzoate

The title compound was prepared according to the procedure described in step 5 of Example 8 from methyl 4-{(1S)-1-[(5-chloro-2-hydroxybenzoyl)amino]ethyl}benzoate (step 4 of Example 8) and (4-chlorophenyl)methanol:

MS (ESI) m/z 458 (M+H)⁺, 456 (M−H)⁻.

### STEP 2. 4-[(1S)-1-({5-Chloro-2-[(4-chlorobenzyl)oxy]benzoyl}amino)ethyl]benzoic acid

The title compound was prepared according to the procedure described in step 6 of Example 8 from methyl 4-[(1S)-1-({5-chloro-2-[(4-chlorobenzyl)oxy]benzoyl}amino)ethyl]benzoate (step 1):

¹H-NMR (DMSO-d₆) δ 8.61 (1H, d, J=7.8 Hz), 7.81 (2H, d, J=8.4 Hz), 7.62-7.48 (4H, m), 7.44 (2H, d, J=8.4 Hz), 7.34 (2H, d, J=8.2 Hz), 7.29 (1H, d, J=8.9 Hz), 5.20 (2H, s), 5.09 (1H, dq, J=7.8, 6.8 Hz), 1.32 (3H, d, J=6.8 Hz);

MS (ESI) m/z 444 (M+H)⁺, 442 (M−H)⁻.

### Example 25

### 4-[(1S)-1-({5-CHLORO-2-[(4-FLUOROBENZYL)OXY]BENZOYL}AMINO)ETHYL]BENZOIC ACID

### STEP 1. Methyl 4-[(1S)-1-({5-chloro-2-[(4-fluorobenzyl)oxy]benzoyl}amino)ethyl]benzoate

The title compound was prepared according to the procedure described in step 5 of Example 8 from methyl 4-{(1S)-1-[(5-chloro-2-hydroxybenzoyl)amino]ethyl}benzoate (step 4 of Example 8) and (4-fluorophenyl)methanol:

MS (ESI) m/z 442 (M+H)⁺, 440 (M−H)⁻.

### STEP 2. 4-[(1S)-1-({5-Chloro-2-[(4-fluorobenzyl)oxy]benzoyl}amino)ethyl]benzoic acid

The title compound was prepared according to the procedure described in step 6 of Example 8 from methyl 4-[(1S)-1-({5-chloro-2-[(4-fluorobenzyl)oxy]benzoyl}amino)ethyl]benzoate (step 1):

¹H-NMR (DMSO-d₆) δ 8.58 (1H, d, J=7.5 Hz), 7.78 (2H, d, J=8.3 Hz), 7.60-7.50 (4H, m), 7.30 (3H, d, J=8.3 Hz), 7.20 (2H, t, J=8.8 Hz), 5.17 (2H, s), 5.07 (1H, dq, J=7.5, 7.0 Hz), 1.28 (3H, d, J=7.0 Hz);

MS (ESI) m/z 428 (M+H)⁺, 426 (M−H)⁻.

### Example 26

### 4-((1S)-1-{[5-CHLORO-2-(2-PHENOXYETHOXY)BENZOYL]AMINO}ETHYL)BENZOIC ACID

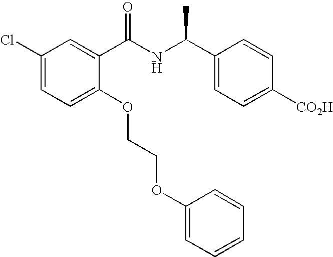

### STEP 1. Methyl 4-((1S)-1-{[5-chloro-2-(2-phenoxyethoxy)benzoyl]amino}ethyl)benzoate

The title compound was prepared according to the procedure described in step 5 of Example 8 from methyl 4-{(1S)-1-[(5-chloro-2-hydroxybenzoyl)amino]ethyl}benzoate (step 4 of Example 8) and 2-phenoxyethanol:

¹H-NMR (CDCl₃) [symbol] 8.32 (1H, d, J=7.6 Hz), 8.18 (1H, d, J=2.8 Hz), 7.85 (2H, d, J=8.4 Hz), 7.40 (1H, dd, J=8.7, 2.8 Hz), 7.35-7.26 (4H, m), 7.04-6.95 (1H, m), 6.92 (1H, d, J=8.7 Hz), 6.89-6.81 (2H, m), 5.31 (1H, dq, J=7.6, 6.9 Hz), 4.50-4.40 (2H, m), 4.37-4.26 (2H, m), 3.89 (3H, s), 1.38 (3H, d, J=6.9 Hz);

MS (ESI) m/z 454 (M+H)⁺, 452 (M−H)⁻.

### STEP 2. 4-((1S)-1-{[5-Chloro-2-(2-phenoxyethoxy)benzoyl]amino}ethyl)benzoic acid

The title compound was prepared according to the procedure described in step 6 of Example 8 from ethyl 4-((1S)-1-{[5-chloro-2-(2-phenoxyethoxy)benzoyl]amino}ethyl)benzoate (step 1):

¹H-NMR (DMSO-d₆) [symbol] 12.84 (1H, br.s), 8.59 (1H, d, J=7.5 Hz), 7.81 (2H, d, J=8.3 Hz), 7.69 (1H, d, J=2.8 Hz), 7.56 (1H, dd, J=8.8, 2.8 Hz), 7.39 (2H, d, J=8.3 Hz), 7.33-7.23 (3H, m), 6.99-6.90 (3H, m), 5.12 (1H, dq, J=7.5, 7.0 Hz), 4.54-4.45 (2H, m), 4.43-4.24 (2H, m), 1.30 (3H, d, J=7.0 Hz);

MS (ESI) m/z 440 (M+H)⁺, 438 (M−H)⁻.

### Example 27

### 4-((1S)-1-{[5-CHLORO-2-(2-METHOXY-2-PHENYLETHOXY)BENZOYL]AMINO}ETHYL)BENZOIC ACID

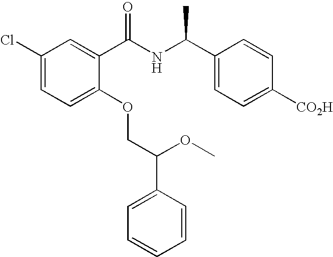

### STEP 1. Methyl 4-((1S)-1-{[5-chloro-2-(2-methoxy-2-phenylethoxy)benzoyl]amino}ethyl)benzoate

The title compound was prepared according to the procedure described in step 5 of Example 8 from methyl 4-{(1S)-1-[(5-chloro-2-hydroxybenzoyl)amino]ethyl}benzoate (step 4 of Example 8) and 2-methoxy-2-phenylethanol:

MS (ESI) m/z 468 (M+H)⁺, 466 (M−H)⁻.

### STEP 2. 4-((1S)-1-{[5-Chloro-2-(2-methoxy-2-phenylethoxy)benzoyl]amino}ethyl)benzoic acid

The title compound was prepared according to the procedure described in step 6 of Example 8 from methyl 4-((1S)-1-{[5-chloro-2-(2-methoxy-2-phenylethoxy)benzoyl]amino}ethyl)benzoate (step 1):

¹H-NMR (DMSO-d₆) [symbol] 12.88 (1H, br.s), 8.73-8.63 (1H, m), 7.99-7.89 (2H, m), 7.76-7.70 (1H, m), 7.56-7.46 (3H, m), 7.45-7.34 (5H, m), 7.26 (1H, d, J=9.0 Hz), 5.32-5.17 (1H, m), 4.77-4.65 (1H, m), 4.36-4.26 (2H, m), 3.13 and 3.12 (total 3H, each s), 1.55-1.45 (3H, m);

MS (ESI) m/z 454 (M+H)⁺, 452 (M−H)⁻.

### Example 28

### 4-[(1S)-1-({5-CHLORO-2-[2-(4-FLUOROPHENOXY)ETHOXY]BENZOYL}AMINO)ETHYL]BENZOIC ACID

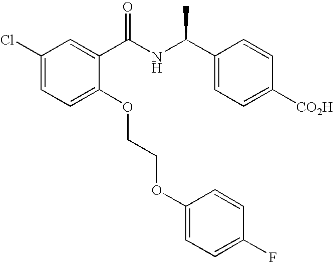

### STEP 1. Methyl 4-[(1S)-1-({5-chloro-2-[2-(4-fluorophenoxy)ethoxy]benzoyl}amino)ethyl]benzoate

The title compound was prepared according to the procedure described in step 5 of Example 8 from methyl 4-{(1S)-1-[(5-chloro-2-hydroxybenzoyl)amino]ethyl}benzoate (step 4 of Example 8) and 2-(4-fluorophenoxy)ethanol:

MS (ESI) m/z 472 (M+H)⁺, 470 (M−H)⁻.

### STEP 2. 4-[(1S)-1-({5-Chloro-2-[2-(4-fluorophenoxy)ethoxy]benzoyl}amino)ethyl]benzoic acid

The title compound was prepared according to the procedure described in step 6 of Example 8 from methyl 4-[(1S)-1-({5-chloro-2-[2-(4-fluorophenoxy)ethoxy]benzoyl}amino)ethyl]benzoate (step 1):

¹H-NMR (DMSO-d₆) [symbol] 8.58 (1H, d, J=7.4 Hz), 7.80 (2H, d, J=8.1 Hz), 7.68 (1H, d, J=2.8 Hz), 7.56 (1H, dd, J=8.9, 2.8 Hz), 7.40 (2H, d, J=8.1 Hz), 7.27 (1H, d, J=8.9 Hz), 7.15-7.05 (2H, m), 7.02-6.93 (2H, m), 5.12 (1H, dq, J=7.4, 6.9 Hz), 4.52-4.43 (2H, m), 4.40-4.31 (2H, m), 1.31 (3H, d, J=6.9 Hz);

MS (ESI) m/z 458 (M+H)⁺, 456 (M−H)⁻.

### Example 29

### 4-((1S)-1-{[5-CHLORO-2-(CYCLOBUTYLMETHOXY)BENZOYL]AMINO}ETHYL)BENZOIC ACID

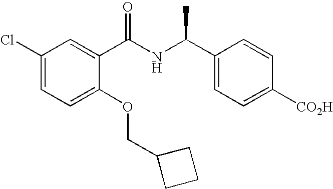

### STEP 1. Methyl 4-((1S)-1-{[5-chloro-2-(cyclobutylmethoxy)benzoyl]amino}ethyl)benzoate

The title compound was prepared according to the procedure described in step 5 of Example 8 from methyl 4-{(1S)-1-[(5-chloro-2-hydroxybenzoyl)amino]ethyl}benzoate (step 4 of Example 8) and cyclobutylmethanol:

¹H-NMR (CDCl₃) [symbol] 8.29 (1H, d, J=7.3 Hz), 8.17 (1H, d, J=2.8 Hz), 8.02 (2H, d, J=8.4 Hz), 7.43 (2H, d, J=8.4 Hz), 7.37 (1H, dd, J=8.9, 2.8 Hz), 6.90 (1H, d, J=8.9 Hz), 5.35 (1H, dq, J=7.3, 6.9 Hz), 4.07 (2H, d, J=7.1 Hz), 3.91 (3H, s), 2.86-2.66 (1H, m), 2.20-1.75 (6H, m), 1.57 (3H, d, J=6.9 Hz);

MS (ESI) m/z 402 (M+H)⁺, 400 (M−H)⁻.

### STEP 2. 4-((1S)-1-{[5-Chloro-2-(cyclobutylmethoxy)benzoyl]amino}ethyl)benzoic acid

The title compound was prepared according to the procedure described in step 6 of Example 8 from methyl 4-((1S)-1-{[5-chloro-2-(cyclobutylmethoxy)benzoyl]amino}ethyl)benzoate (step 1):

¹H-NMR (DMSO-d₆) [symbol] 12.87 (1H, br.s), 8.53 (1H, d, J=7.4 Hz), 7.92 (2H, d, J=8.2 Hz), 7.61 (1H, d, J=2.8 Hz), 7.55-7.46 (3H, m), 7.19 (1H, d, J=8.9 Hz), 5.16 (1H, dq, J=7.4, 6.9 Hz), 4.08 (2H, d, J=6.8 Hz), 2.80-2.65 (1H, m), 2.10-1.70 (6H, m), 1.47 (3H, d, J=6.9 Hz);

MS (ESI) m/z 388 (M+H)⁺, 386 (M−H)⁻.

### Example 30

### 4-{(1S)-1-[(5-CHLORO-2-ISOBUTOXYBENZOYL)AMINO]ETHYL}BENZOIC ACID

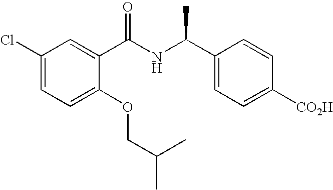

### STEP 1. Methyl 4-{(1S)-1-[(5-chloro-2-isobutoxybenzoyl)amino]ethyl}benzoate

The title compound was prepared according to the procedure described in step 5 of Example 8 from methyl 4-{(1S)-1-[(5-chloro-2-hydroxybenzoyl)amino]ethyl}benzoate (step 4 of Example 8) and 2-methylpropan-1-ol:

¹H-NMR (CDCl₃) [symbol] 8.31 (1H, d, J=7.3 Hz), 8.17 (1H, d, J=2.8 Hz), 8.01 (2H, d, J=8.4 Hz), 7.44 (2H, d, J=8.4 Hz), 7.36 (1H, dd, J=8.9, 2.8 Hz), 6.88 (1H, d, J=8.9 Hz), 5.37 (1H, dq, J=7.3, 6.9 Hz), 3.91 (3H, s), 3.87 (2H, d, J=6.3 Hz), 2.20-1.96 (1H, m), 1.59 (3H, d, J=6.9 Hz), 1.03 (3H, d, J=6.8 Hz), 0.99 (3H, d, J=6.8 Hz);

MS (ESI) m/z 390 (M+H)⁺, 388 (M−H)⁻.

### STEP 2. 4-{(1S)-1-[(5-Chloro-2-isobutoxybenzoyl)amino]ethyl}benzoic acid

The title compound was prepared according to the procedure described in step 6 of Example 8 from methyl 4-{(1S)-1-[(5-chloro-2-isobutoxybenzoyl)amino]ethyl}benzoate (step 1):

¹H-NMR (DMSO-d₆) [symbol] 12.88 (1H, br.s), 8.58 (1H, d, J=7.5 Hz), 7.91 (2H, d, J=8.0 Hz), 8.58 (1H, d, J=2.8 Hz), 7.54-7.44 (3H, m), 7.17 (1H, d, J=9.0 Hz), 5.17 (1H, dq, J=7.5, 7.0 Hz), 3.87 (2H, d, J=6.4 Hz), 2.15-1.95 (1H, m), 1.47 (3H, d, J=7.0 Hz), 0.95 (3H, d, J=7.0 Hz), 0.92 (3H, d, J=7.0 Hz);

MS (ESI) m/z 376 (M+H)⁺, 374 (M−H)⁻.

### Example 31

### 4-[(1S)-1-({[5-CHLORO-2-(3-METHYLBUTOXY)PYRIDIN-3-YL]CARBONYL}AMINO)ETHYL]BENZOIC ACID

### STEP 1. 5-Chloro-2-(3-methylbutoxy)nicotinic acid

The title compound was prepared according to the procedure described in step 1 of Example 9 from 2,5-dichloronicotinic acid and 3-methylbutan-1-ol:

¹H-NMR (CDCl₃) δ 8.43 (1H, d, J=2.8 Hz), 8.32 (1H, d, J=2.8 Hz), 4.62 (2H, t, J=6.6 Hz), 1.97-1.70 (3H, m), 1.00 (6H, d, J=6.4 Hz).

### STEP 2. Methyl 4-[(1S)-1-({[5-chloro-2-(3-methylbutoxy)pyridin-3-yl]carbonyl}amino)ethyl]benzoate

The title compound was prepared according to the procedure described in step 4 of Example 8 from 5-chloro-2-(3-methylbutoxy)nicotinic acid (step 1) and methyl 4-[(1S)-1-aminoethyl]benzoate hydrochloride (step 3 of Example 8):

¹H-NMR (CDCl₃) δ8.46 (1H, d, J=2.8 Hz), 8.40-8.28 (1H, m), 8.19 (1H, d, J=2.8 Hz), 8.04 (2H, d, J=8.4 Hz), 7.44 (2H, d, J=8.4 Hz), 5.45-5.25 (1H, m), 4.50 (2H, t, J=6.3 Hz), 3.91 (3H, s), 1.84-1.52 (6H, m), 0.94 (6H, d, J=5.6 Hz).

### STEP 3. 4-[(1S)-1-({[5-Chloro-2-(3-methylbutoxy)pyridin-3-yl]carbonyl}amino)ethyl]benzoic acid

The title compound was prepared according to the procedure described in step 6 of Example 8 from methyl 4-[(1S)-1-({[5-chloro-2-(3-methylbutoxy)pyridin-3-yl]carbonyl}amino)ethyl]benzoate (step 2):

¹H-NMR (DMSO-d₆) δ 8.66 (1H, d, J=7.5 Hz), 8.32 (1H, d, J=2.8 Hz), 8.00 (1H, d, J=2.8 Hz), 7.90 (2H, d, J=8.3 Hz), 7.50 (2H, d, J=8.3 Hz), 5.09 (1H, dq, J=7.5, 6.8 Hz), 4.35 (2H, t, J=6.3 Hz), 1.70-1.50 (3H, m), 1.40 (3H, d, J=6.8 Hz), 0.85 (6H, d, J=6.3 Hz);

MS (ESI) m/z 391 (M+H)⁺, 389 (M−H)⁻.

### Example 32

### 4-[(1S)-1-({5-CHLORO-2-[(2,5-DIFLUOROBENZYL)OXY]BENZOYL}AMINO)ETHYL]BENZOIC ACID

### STEP 1. Methyl 4-[(1S)-1-({5-chloro-2-[(2,5-difluorobenzyl)oxy]benzoyl}amino)ethyl]benzoate

A mixture of 4-{(1S)-1-[(5-chloro-2-hydroxybenzoyl)amino]ethyl}benzoate (step 4 of Example 8, 100 mg, 0.30 mmol), 2-(bromomethyl)-1,4-difluorobenzene (62 mg, 0.30 mmol), and potassium carbonate (83 mg, 0.60 mmol) in N,N-dimethylformamide was stirred at room temperature overnight. To the mixture was added water and the resulting mixture was extracted with ethyl acetate. The organic layer was washed with brine, dried over sodium sulfate, and evaporated. The residue was purified by flash column chromatography on silica gel eluting with hexane/ethyl acetate (3/1) to afford 130 mg (94%) of the title compound:

MS (ESI) m/z 460 (M+H)⁺, 458 (M−H)⁻.

### STEP 2. 4-[(1S)-1-({5-Chloro-2-[(2,5-difluorobenzyl)oxy]benzoyl}amino)ethyl]benzoic acid

The title compound was prepared according to the procedure described in step 6 of Example 8 from methyl 4-[(1S)-1-({5-chloro-2-[(2,5-difluorobenzyl)oxy]benzoyl}amino)ethyl]benzoate (step 1):

¹H-NMR (DMSO-d₆) δ 8.57 (1H, d, J=7.7 Hz), 7.78 (2H, d, J=8.1 Hz), 7.55 (1H, s), 7.51-7.43 (2H, m), 7.40-7.20 (5H, m), 5.23 (2H, s), 5.07 (1H, dq, J=7.7, 7.0 Hz), 1.30 (3H, d, J=7.0 Hz);

MS (ESI) m/z 446 (M+H)⁺, 444 (M−H)⁻.

### Example 33

### 4-[(1S)-1-({5-CHLORO-2-[(3,4-DIFLUOROBENZYL)OXY]BENZOYL}AMINO)ETHYL]BENZOIC ACID

### STEP 1. Methyl 4-[(1S)-1-({5-chloro-2-[(3,4-difluorobenzyl)oxy]benzoyl}amino)ethyl]benzoate

The title compound was prepared according to the procedure described in step 1 of Example 32 from 4-{(1S)-1-[(5-chloro-2-hydroxybenzoyl)amino]ethyl}benzoate (step 4 of Example 8) and 4-(bromomethyl)-1,2-difluorobenzene:

MS (ESI) m/z 460 (M+H)⁺, 458 (M−H)⁻.

### STEP 2. 4-[(1S)-1-({5-Chloro-2-[(3,4-difluorobenzyl)oxy]benzoyl}amino)ethyl]benzoic acid

The title compound was prepared according to the procedure described in step 6 of Example 8 from methyl 4-[(1S)-1-({5-chloro-2-[(3,4-difluorobenzyl)oxy]benzoyl}amino)ethyl]benzoate (step 1):

¹H-NMR (DMSO-d₆) δ 8.61 (1H, d, J=7.5 Hz), 7.76 (2H, d, J=8.3 Hz), 7.64-7.23 (8H, m), 5.15 (2H, s), 5.02 (1H, dq, J=7.5, 7.0 Hz), 1.31 (3H, d, J=7.0 Hz);

MS (ESI) m/z 446 (M+H)⁺, 444 (M−H)⁻.

### Example 34

### 4-[(1S)-1-({5-CHLORO-2-[2-(4-FLUOROPHENYL)ETHOXY]BENZOYL}AMINO)ETHYL]BENZOIC ACID

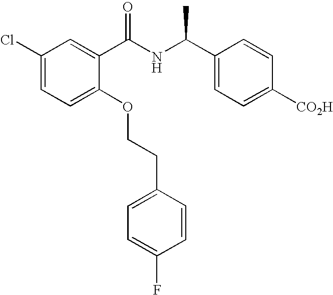

### STEP 1. Methyl 4-[(1S)-1-({5-chloro-2-[2-(4-fluorophenyl)ethoxy]benzoyl}amino)ethyl]benzoate

To a stirred solution of methyl 4-{(1S)-1-[(5-chloro-2-hydroxybenzoyl)amino]ethyl}benzoate (step 4 of example 8, 73 mg, 0.22 mmol), 2-(4-fluorophenyl)ethanol (46 mg, 0.33 mmol) and tributylphosphine (0.14 mL, 0.55 mmol) in tetrahydrofuran (2 mL) was added N,N,N′,N′-tetramethylazodicarboxamide (95 mg, 0.55 mmol) at room temperature. After being stirred 3 days, the reaction was quenched by the addition of sodium bicarbonate aqueous solution (30 mL). The aqueous layer was extracted with ethyl acetate (20 mL×2) and the combined organic layers were washed with brine, dried (magnesium sulfate), and evaporated. The remaining residue was purified by flush column chromatography on silica gel eluting with hexane/ethyl acetate (5/1) to afford a mixture (0.13 g) of the title compound and 2-(4-fluorophenyl)ethanol as a colorless oil:

MS (ESI) m/z 456 (M+H)⁺.

### STEP 2. 4-[(1S)-1-({5-Chloro-2-[2-(4-fluorophenyl)ethoxy]benzoyl}amino)ethyl]benzoic acid

The title compound was prepared according to the procedure described in step 3 of Example 8 from methyl 4-[(1S)-1-({5-chloro-2-[2-(4-fluorophenyl)ethoxy]benzoyl}amino)ethyl]benzoate (step 1):

¹H-NMR (DMSO-d₆) [symbol] 8.47 (1H, d, J=7.6 Hz), 7.89 (2H, d, J=8.1 Hz), 7.58 (1H, d, J=2.6 Hz), 7.49 (1H, dd, J=8.7, 2.6 Hz), 7.43 (2H, d, J=8.1 Hz), 7.33-7.17 (3H, m), 7.13-7.02 (2H, m), 5.12 (1H, dq, J=7.6, 6.9 Hz), 4.34 (2H, t, J=6.6 Hz), 3.07 (2H, t, J=6.6 Hz), 1.37 (3H, d, J=6.9 Hz);

MS (ESI) m/z 442 (M+H)⁺, 440 (M−H)⁻.

### Example 35

### 4-[(1S)-1-({5-CHLORO-2-[(2,4-DIFLUOROBENZYL)OXY]BENZOYL}AMINO)ETHYL]BENZOIC ACID

### STEP 1. Methyl 4-[(1S)-1-({5-chloro-2-[(2,4-difluorobenzyl)oxy]benzoyl}amino)ethyl]benzoate

The title compound was prepared according to the procedure described in step 1 of Example 32 from 4-{(1S)-1-[(5-chloro-2-hydroxybenzoyl)amino]ethyl}benzoate (step 4 of Example 8) and 1-(bromomethyl)-2,4-difluorobenzene:

MS (ESI) m/z 460 (M+H)⁺, 458 (M−H)⁻.

### STEP 2. 4-[(1S)-1-({5-Chloro-2-[(2,4-difluorobenzyl)oxy]benzoyl}amino)ethyl]benzoic acid

The title compound was prepared according to the procedure described in step 6 of Example 8 from methyl 4-[(1S)-1-({5-chloro-2-[(2,4-difluorobenzyl)oxy]benzoyl}amino)ethyl]benzoate (step 1):

¹H-NMR (DMSO-d₆) δ 8.50 (1H, d, J=7.7 Hz), 7.77 (2H, d, J=8.4 Hz), 7.64 (1H, q, J=6.6 Hz), 7.60-7.50 (2H, m), 7.38-7.24 (4H, m), 7.13-7.04 (1H, m), 5.21 (2H, s), 5.05 (1H, dq, J=7.7, 7.0 Hz), 1.27 (3H, d, J=7.0 Hz);

MS (ESI) m/z 446 (M+H)⁺, 444 (M−H)⁻.

### Example 36

### 4-[(1S)-1-({5-CHLORO-2-[(2-FLUOROBENZYL)OXY]BENZOYL}AMINO)ETHYL]BENZOIC ACID

### STEP 1. Methyl 4-[(1S)-1-({5-chloro-2-[(2-fluorobenzyl)oxy]benzoyl}amino)ethyl]benzoate

The title compound was prepared according to the procedure described in step 1 of Example 32 from 4-{(1S)-1-[(5-chloro-2-hydroxybenzoyl)amino]ethyl}benzoate (step 4 of Example 8) and 1-(bromomethyl)-2-fluorobenzene:

MS (ESI) m/z 442 (M+H)⁺, 440 (M−H)⁻.

### STEP 2. 4-[(1S)-1-({5-Chloro-2-[(2-fluorobenzyl)oxy]benzoyl}amino)ethyl]benzoic acid

The title compound was prepared according to the procedure described in step 6 of Example 8 from methyl 4-[(1S)-1-({5-chloro-2-[(2-fluorobenzyl)oxy]benzoyl}amino)ethyl]benzoate (step 1):

¹H-NMR (DMSO-d₆) δ 12.9-12.8 (1H, br.s), 8.53 (1H, d, J=7.4 Hz), 7.79 (2H, d, J=8.1 Hz), 7.67-7.51 (3H, m), 7.50-7.18 (6H, m), 5.29 (2H, s), 5.08 (1H, dq, J=7.4, 6.6 Hz), 1.28 (3H, d, J=6.6 Hz);

MS (ESI) m/z 428 (M+H)⁺, 426 (M−H)⁻.

### Example 37

### 4-[(1S)-1-({5-CHLORO-2-[(3,5-DIFLUOROBENZYL)OXY]BENZOYL}AMINO)ETHYL]BENZOIC ACID

### STEP 1. Methyl 4-[(1S)-1-({5-chloro-2-[(3,5-difluorobenzyl)oxy]benzoyl}amino)ethyl]benzoate

The title compound was prepared according to the procedure described in step 1 of Example 32 from 4-{(1S)-1-[(5-chloro-2-hydroxybenzoyl)amino]ethyl}benzoate (step 4 of Example 8) and 1-(bromomethyl)-3,5-difluorobenzene:

MS (ESI) m/z 460 (M+H)⁺, 458 (M−H)⁻.

### STEP 2. 4-[(1S)-1-({5-Chloro-2-[(3,5-difluorobenzyl)oxy]benzoyl}amino)ethyl]benzoic acid

The title compound was prepared according to the procedure described in step 6 of Example 8 from methyl 4-[(1S)-1-({5-chloro-2-[(3,5-difluorobenzyl)oxy]benzoyl}amino)ethyl]benzoate (step 1):

¹H-NMR (DMSO-d₆) δ 12.9-12.8 (1H, br.s), 8.67 (1H, d, J=7.6 Hz), 7.79 (2H, d, J=8.2 Hz), 7.60-7.48 (2H, m), 7.37 (2H, d, J=8.2 Hz), 7.30-7.13 (4H, m), 5.20 (2H, s), 5.05 (1H, dq, J=7.6, 7.1 Hz), 1.34 (3H, d, J=7.1 Hz);

MS (ESI) m/z 446 (M+H)⁺, 444 (M−H)⁻.

### Example 38

### 4-((1S)-1-{[2-(BENZYLOXY)-5-CHLOROBENZOYL]AMINO}ETHYL)BENZOIC ACID

### STEP 1. 4-((1S)-1-{[2-(Benzyloxy)-5-chlorobenzoyl]amino}ethyl)benzoic acid

The title compound was prepared according to the two steps procedure described in step 1 of Example 32 and step 6 of Example 8. Firstly, 4-{(1S)-1-[(5-chloro-2-hydroxybenzoyl)amino]ethyl}benzoate (step 4 of Example 8) was benzylated by benzyl bromide. Next, the crude product was hydrolyzed to the corresponding carboxylic acid:

¹H-NMR (DMSO-d₆) δ 12.9-12.8 (1H, br.s), 8.61 (1H, d, J=7.9 Hz), 7.78 (2H, d, J=8.4 Hz), 7.62-7.46 (4H, m), 7.45-7.25 (6H, m), 5.20 (2H, s), 5.07 (1H, dq, J=7.9, 6.9 Hz), 1.27 (3H, d, J=6.9 Hz);

MS (ESI) m/z 410 (M+H)⁺, 408 (M−H)⁻.

### Example 39

### 4-{(1S)-1-[({5-CHLORO-2-[(2-CHLOROBENZYL)OXY]PYRIDIN-3-YL}CARBONYL)AMINO]ETHYL}BENZOIC ACID

### STEP 1. 5-Chloro-2-[(2-chlorobenzyl)oxy]nicotinic acid

The title compound was prepared according to the procedure described in step 1 of Example 9 from 2,5-dichloronicotinic acid and (2-chlorophenyl)methanol:

MS (ESI) m/z 298 (M+H)⁺, 296 (M−H)⁻.

### STEP 2. Methyl 4-{(1S)-1-[({5-chloro-2-[(2-chlorobenzyl)oxy]pyridin-3-yl}carbonyl)amino]ethyl}benzoate

The title compound was prepared according to the procedure described in step 4 of Example 8 from 5-chloro-2-[(2-chlorobenzyl)oxy]nicotinic acid (step 1) and methyl 4-[(1S)-1-aminoethyl]benzoate hydrochloride (step 3 of Example 8):

¹H-NMR (CDCl₃) δ8.17 (1H, d, J=2.8 Hz), 8.02-7.95 (1H, m), 7.91 (2H, d, J=8.3 Hz), 7.43 (1H, dd, J=8.8, 2.8 Hz), 7.30-7.07 (5H, m), 7.04 (1H, d, J=8.6 Hz), 5.32-5.16 (3H, m), 3.91 (3H, s), 1.36 (3H, d, J=7.0 Hz);

MS (ESI) m/z 459 (M+H)⁺, 457 (M−H)⁻.

### STEP 3. 4-{(1S)-1-[({5-chloro-2-[(2-chlorobenzyl)oxy]pyridin-3-yl}carbonyl)amino]ethyl}benzoic acid

The title compound was prepared according to the procedure described in step 6 of Example 8 from methyl 4-{(1S)-1-[({5-chloro-2-[(2-chlorobenzyl)oxy]pyridin-3-yl}carbonyl)amino]ethyl}benzoate (step 2):

¹H-NMR (DMSO-d₆) δ 12.9-12.8 (1H, br.s), 8.70 (1H, d, J=7.7 Hz), 8.41 (1H, dd, J=2.8, 1.1 Hz), 8.08 (1H, dd, J=2.8, 1.1 Hz), 7.76 (2H, d, J=8.4 Hz), 7.64-7.58 (1H, m), 7.56-7.50 (1H, m), 7.45-7.30 (4H, m), 5.52 (2H, s), 5.12 (1H, dq, J=7.7, 7.0 Hz), 1.36 (3H, d, J=7.0 Hz);

MS (ESI) m/z 445 (M+H)⁺, 443 (M−H)⁻.

### Example 40

### 4-{(1S)-1-[({5-CHLORO-2-[(4-CHLOROBENZYL)OXY]PYRIDIN-3-YL}CARBONYL)AMINO]ETHYL}BENZOIC ACID

### STEP 1. 5-Chloro-2-[(4-chlorobenzyl)oxy]nicotinic acid

The title compound was prepared according to the procedure described in step 1 of Example 9 from 2,5-dichloronicotinic acid and (4-chlorophenyl)methanol:

MS (ESI) m/z 298 (M+H)⁺, 296 (M−H)⁻.

### STEP 2. Methyl 4-{(1S)-1-[({5-Chloro-2-[(4-chlorobenzyl)oxy]pyridin-3-yl}carbonyl)amino]ethyl}benzoate

The title compound was prepared according to the procedure described in step 4 of Example 8 from 5-chloro-2-[(4-chlorobenzyl)oxy]nicotinic acid (step 1) and methyl 4-[(1S)-1-aminoethyl]benzoate hydrochloride (step 3 of Example 8):

¹H-NMR (CDCl₃) δ8.49 (1H, d, J=2.8 Hz), 8.23 (1H, d, J=2.8 Hz), 8.18-8.08 (1H, m), 7.93 (2H, d, J=8.4 Hz), 7.39-7.30 (4H, m), 7.20 (2H, d, J=8.6 Hz), 5.42 (2H, s), 5.33-5.16 (1H, m), 3.93 (3H, s), 1.40 (3H, d, J=7.0 Hz);

MS (ESI) m/z 459 (M+H)⁺, 457 (M−H)⁻.

### STEP 3. 4-{(1S)-1-[({5-Chloro-2-[(4-chlorobenzyl)oxy]pyridin-3-yl}carbonyl)amino]ethyl}benzoic acid

The title compound was prepared according to the procedure described in step 6 of Example 8 from methyl 4-{(1S)-1-[({5-chloro-2-[(4-chlorobenzyl)oxy]pyridin-3-yl}carbonyl)amino]ethyl}benzoate (step 1):

¹H-NMR (DMSO-d₆) δ 8.74 (1H, d, J=7.3 Hz), 8.40-8.36 (1H, m), 8.07-8.03 (1H, m), 7.82 (2H, d, J=7.0 Hz), 7.52 (2H, d, J=7.3 Hz), 7.44 (2H, d, J=7.0 Hz), 7.37 (2H, d, J=7.3 Hz), 5.42 (2H, s), 5.11 (1H, dq, J=7.3, 7.0 Hz), 1.37 (3H, d, J=7.0 Hz);

MS (ESI) m/z 445 (M+H)⁺, 443 (M−H)⁻.

### Example 41

### 4-[(1S)-1-({5-CHLORO-2-[(2-CYANOBENZYL)OXY]BENZOYL}AMINO)ETHYL]BENZOIC ACID

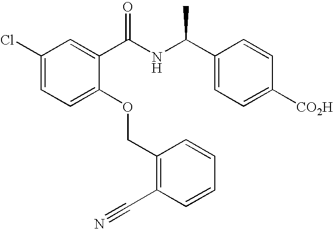

### STEP 1. Methyl 4-[(1S)-1-({5-chloro-2-[(2-cyanobenzyl)oxy]benzoyl}amino)ethyl]benzoate

The title compound was prepared according to the procedure described in step 1 of Example 32 from 4-{(1S)-1-[(5-chloro-2-hydroxybenzoyl)amino]ethyl}benzoate (step 4 of Example 8) and 2-(bromomethyl)benzonitrile:

¹H-NMR (CDCl₃) δ 8.13 (1H, d, J=2.8 Hz), 7.92-7.80 (3H, m), 7.74-7.68 (1H, m), 7.65-7.46 (3H, m), 7.42 (1H, dd, J=2.8, 8.8 Hz), 7.21 (2H, d, J=8.3 Hz), 7.04 (1H, d, J=8.8 Hz), 5.36-5.22 (3H, m), 3.91 (3H, s), 1.37 (3H, d, J=7.0 Hz).

### STEP 2. 4-[(1S)-1-({5-Chloro-2-[(2-cyanobenzyl)oxy]benzoyl}amino)ethyl]benzoic acid

The title compound was prepared according to the procedure described in step 6 of Example 8 from methyl 4-[(1S)-1-({5-chloro-2-[(2-cyanobenzyl)oxy]benzoyl}amino)ethyl]benzoate (step 1):

¹H-NMR (CDCl₃) δ8.14 (1H, d, J=2.8 Hz), 7.93 (2H, d, J=8.2 Hz), 7.86 (1H, d, J=7.3 Hz), 7.72 (1H, d, J=7.7 Hz), 7.67-7.48 (3H, m), 7.43 (1H, dd, J=2.8, 8.7 Hz), 7.27 (2H, d, J=8.2 Hz), 7.05 (1H, d, J=8.7 Hz), 5.39-5.22 (3H, m), 1.39 (3H, d, J=6.9 Hz);

MS (ESI) m/z 435 (M+H)⁺, 433 (M−H)⁻.

### Example 42

### 4-[(1S)-1-({5-CHLORO-2-[2-(TETRAHYDRO-2H-PYRAN-4-YL)ETHOXY]BENZOYL}AMINO)ETHYL]BENZOIC ACID

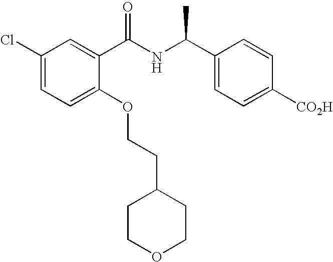

### STEP 1. Methyl 4-[(1S)-1-({5-chloro-2-[2-(tetrahydro-2H-pyran-4-yl)ethoxy]benzoyl}amino)ethyl]benzoate

The title compound was prepared according to the procedure described in step 5 of Example 8 from methyl 4-{(1S)-1-[(5-chloro-2-hydroxybenzoyl)amino]ethyl}benzoate (step 4 of Example 8) and 2-(tetrahydro-2H-pyran-4-yl)ethanol:

¹H-NMR (CDCl₃) [symbol] 8.23 (1H, d, J=7.4 Hz), 8.17 (1H, d, J=2.8 Hz), 8.02 (2H, d, J=8.4 Hz), 7.44 (2H, d, J=8.4 Hz), 7.37 (1H, dd, J=8.9, 2.8 Hz), 6.90 (1H, d, J=8.9 Hz), 5.37 (1H, dq, J=7.4, 7.1 Hz), 4.14 (2H, t, J=6.4 Hz), 3.98-3.88 (2H, m), 3.91 (3H, s), 3.38-3.22 (2H, m), 1.80-1.20 (7H, m), 1.59 (3H, d, J=7.1 Hz);

MS (ESI) m/z 446 (M+H)⁺, 444 (M−H)⁻.

### STEP 2. 4-[(1S)-1-({5-Chloro-2-[2-(tetrahydro-2H-pyran-4-yl)ethoxy]benzoyl}amino)ethyl]benzoic acid

The title compound was prepared according to the procedure described in step 6 of Example 8 from methyl 4-[(1S)-1-({5-chloro-2-[2-(tetrahydro-2H-pyran-4-yl)ethoxy]benzoyl}amino)ethyl]benzoate (step 1):

¹H-NMR (DMSO-d₆) [symbol] 12.86 (1H, br.s), 8.58 (1H, d, J=7.3 Hz), 7.92 (2H, d, J=8.3 Hz), 7.58 (1H, d, J=2.8 Hz), 7.52 (2H, d, J=8.4 Hz), 7.49 (1H, dd, J=8.8, 2.8 Hz), 7.19 (1H, d, J=8.8 Hz), 5.16 (1H, dq, J=7.3, 7.0 Hz), 4.12 (2H, t, J=5.7 Hz), 3.84-3.73 (2H, m), 3.25-3.10 (2H, m), 1.70-1.44 (5H, m), 1.47 (3H, d, J=7.0 Hz), 1.26-1.06 (2H, m);

MS (ESI) m/z 432 (M+H)⁺, 430 (M−H)⁻.

### Example 43

### 4-[(1S)-1-({5-CHLORO-2-[(3-FLUOROBENZYL)OXY]BENZOYL}AMINO)ETHYL]BENZOIC ACID

### STEP 1. Methyl 4-[(1S)-1-({5-chloro-2-[(3-fluorobenzyl)oxy]benzoyl}amino)ethyl]benzoate

The title compound was prepared according to the procedure described in step 1 of Example 32 from 4-{(1S)-1-[(5-chloro-2-hydroxybenzoyl)amino]ethyl}benzoate (step 4 of Example 8) and 1-(bromomethyl)-3-fluorobenzene:

¹H-NMR (CDCl₃) δ8.19 (1H, d, J=2.8 Hz), 8.16-8.05 (1H, m), 7.91 (2H, d, J=8.3 Hz), 7.46-7.33 (2H, m), 7.24-7.04 (5H, m), 7.00 (1H, d, J=8.6 Hz), 5.33-5.17 (1H, m), 5.11 (2H, s), 3.91 (3H, s), 1.33 (3H, d, J=7.0 Hz);

MS (ESI) m/z 442 (M+H)⁺, 440 (M−H)⁻.

### STEP 2. 4-[(1S)-1-({5-Chloro-2-[(3-fluorobenzyl)oxy]benzoyl}amino)ethyl]benzoic acid

The title compound was prepared according to the procedure described in step 6 of Example 8 from methyl 4-[(1S)-1-({5-chloro-2-[(3-fluorobenzyl)oxy]benzoyl}amino)ethyl]benzoate (step 1):

¹H-NMR (CDCl₃) δ 8.25-8.05 (2H, m), 7.97 (2H, d, J=8.4 Hz), 7.47-7.33 (2H, m), 7.33-7.06 (5H, m), 7.01 (1H, d, J=9.0 Hz), 5.40-5.20 (1H, m), 5.12 (2H, s), 1.33 (3H, d, J=7.0 Hz);

MS (ESI) m/z 428 (M+H)⁺, 426 (M−H)⁻.

### Example 44

### 4-[(1S)-1-({5-CHLORO-2-[(5-METHYLISOXAZOL-3-YL)METHOXY]BENZOYL}AMINO)ETHYL]BENZOIC ACID

### STEP 1. Methyl 4-[(1S)-1-({5-chloro-2-[(5-methylisoxazol-3-yl)methoxy]benzoyl}amino)ethyl]benzoate

The title compound was prepared according to the procedure described in step 1 of Example 32 from 4-{(1S)-1-[(5-chloro-2-hydroxybenzoyl)amino]ethyl}benzoate (step 4 of Example 8) and 3-(chloromethyl)-5-methyl isoxazole:

¹H-NMR (DMSO-d₆) [symbol] 8.35 (1H, d, J=7.3 Hz), 8.19 (1H, d, J=2.8 Hz), 7.98 (2H, d, J=8.4 Hz), 7.45-7.37 (3H, m), 6.99 (1H, d, J=8.8 Hz), 5.95 (1H, s), 5.35 (1H, dq, J=7.3, 7.0 Hz), 5.21 (2H, s), 3.90 (3H, s), 2.43 (3H, s), 1.54 (3H, d, J=7.0 Hz).

### STEP 2. 4-[(1S)-1-({5-Chloro-2-[(5-methylisoxazol-3-yl)methoxy]benzoyl}amino)ethyl]benzoic acid

The title compound was prepared according to the procedure described in step 6 of Example 8 from methyl 4-[(1S)-1-({5-chloro-2-[(5-methylisoxazol-3-yl)methoxy]benzoyl}amino)ethyl]benzoate (step 1):

¹H-NMR (DMSO-d₆) δ 13.0-12.8 (1H, br), 8.74 (1H, d, J=7.5 Hz), 7.84 (2H, d, J=8.3 Hz), 7.60 (1H, d, J=2.8 Hz), 7.55 (1H, dd, J=8.8, 2.8 Hz), 7.43 (2H, d, J=8.3 Hz), 7.32 (1H, d, J=8.8 Hz), 6.28 (1H, s), 5.28 (2H, s), 5.15 (1H, dq, J=7.5, 7.0 Hz), 2.40 (3H, s), 1.40 (3H, d, J=7.0 Hz);

MS (ESI) m/z 415 (M+H)⁺, 413 (M−H)⁻.

### Example 45

### 4-[(1S)-1-({5-CHLORO-2-[(4-CHLORO-2-FLUOROBENZYL)OXY]BENZOYL}AMINO)ETHYL]BENZOIC ACID

### STEP 1. Methyl 4-[(1S)-1-({5-chloro-2-[(4-chloro-2-fluorobenzyl)oxy]benzoyl}amino)ethyl]benzoate

The title compound was prepared according to the procedure described in step 5: of Example 8 from methyl 4-{(1S)-1-[(5-chloro-2-hydroxybenzoyl)amino]ethyl}benzoate (step 4 of Example 8) and (4-chloro-2-fluorophenyl)methanol:

¹H-NMR (CDCl₃) δ8.17 (1H, d, J=2.8 Hz), 8.03-7.95 (1H, m), 7.92 (2H, d, J=8.3 Hz), 7.46-7.07 (6H, m), 7.02 (1H, d, J=8.6 Hz), 5.32-5.17 (1H, m), 5.15 (2H, s), 3.92 (3H, s), 1.37 (3H, d, J=7.0 Hz).

### STEP 2. 4-[(1S)-1-({5-Chloro-2-[(4-chloro-2-fluorobenzyl)oxy]benzoyl}amino)ethyl]benzoic acid

The title compound was prepared according to the procedure described in step 6 of Example 8 from methyl 4-[(1S)-1-({5-chloro-2-[(4-chloro-2-fluorobenzyl)oxy]benzoyl}amino)ethyl]benzoate (step 1):

¹H-NMR (DMSO-d₆) δ 8.54 (1H, d, J=7.5 Hz), 7.80 (2H, d, J=8.1 Hz), 7.65-7.50 (4H, m), 7.37-7.28 (4H, m), 5.25 (2H, s), 5.08 (1H, dq, J=7.5, 6.8 Hz), 1.36 (3H, d, J=6.8 Hz);

MS (ESI) m/z 462 (M+H)⁺, 460 (M−H)⁻.

### Example 46

### 4-[(1S)-1-({5-CHLORO-2-[(2-CHLORO-4-FLUOROBENZYL)OXY]BENZOYL}AMINO)ETHYL]BENZOIC ACID

### STEP 1. Methyl 4-[(1S)-1-({5-chloro-2-[(2-chloro-4-fluorobenzyl)oxy]benzoyl}amino)ethyl]benzoate

The title compound was prepared according to the procedure described in step 5 of Example 8 from methyl 4-{(1S)-1-[(5-chloro-2-hydroxybenzoyl)amino]ethyl}benzoate (step 4 of Example 8) and (2-chloro-4-fluorophenyl)methanol:

¹H-NMR (CDCl₃) δ8.16 (1H, d, J=2.8 Hz), 8.10-7.98 (1H, m), 7.89 (2H, d, J=8.3 Hz), 7.48-7.36 (2H, m), 7.24-7.10 (3H, m), 7.08-6.95 (2H, m), 5.32-5.10 (3H, m), 3.92 (3H, s), 1.34 (3H, d, J=7.0 Hz);

MS (ESI) m/z 476 (M+H)⁺, 474 (M−H)⁻.

### STEP 2. 4-[(1S)-1-({5-Chloro-2-[(2-chloro-4-fluorobenzyl)oxy]benzoyl}amino)ethyl]benzoic acid

The title compound was prepared according to the procedure described in step 6 of Example 8 from methyl 4-[(1S)-1-({5-chloro-2-[(2-chloro-4-fluorobenzyl)oxy]benzoyl}amino)ethyl]benzoate (step 2):

¹H-NMR (DMSO-d₆) δ 8.49 (1H, d, J=7.5 Hz), 7.83-7.65 (3H, m), 7.64-7.50 (3H, m), 7.42-7.20 (4H, m), 5.25 (2H, s), 5.08 (1H, dq, J=7.5, 6.8 Hz), 1.28 (3H, d, J=6.8 Hz);

MS (ESI) m/z 462 (M+H)⁺, 460 (M−H)⁻.

### Example 47

### 4-[(1S)-1-({5-CHLORO-2-[(3-CHLOROPYRIDIN-2-YL)METHOXY]BENZOYL}AMINO)ETHYL]BENZOIC ACID

### STEP 1. Methyl 4-[(1S)-1-({5-chloro-2-[(3-chloropyridin-2-yl)methoxy]benzoyl}amino)ethyl]benzoate

The title compound was prepared according to the procedure described in step 5 of Example 8 from methyl 4-{(1S)-1-[(5-chloro-2-hydroxybenzoyl)amino]ethyl}benzoate (step 4 of Example 8) and (3-chloropyridin-2-yl)methanol:

MS (ESI) m/z 459 (M+H)⁺, 457 (M−H)⁻.

### STEP 2. 4-[(1S)-1-({5-Chloro-2-[(3-chloropyridin-2-yl)methoxy]benzoyl}amino)ethyl]benzoic acid

The title compound was prepared according to the procedure described in step 6 of Example 8 from methyl 4-[(1S)-1-({5-chloro-2-[(3-chloropyridin-2-yl)methoxy]benzoyl}amino)ethyl]benzoate (step 1):

¹H-NMR (DMSO-d₆) δ 9.26 (1H, d, J=7.7 Hz), 8.48 (1H, d, J=4.4 Hz), 8.05 (1H, d, J=8.1 Hz), 7.87-7.73 (3H, m), 7.59 (1H, dd, J=8.8, 2.8 Hz), 7.52-7.36 (4H, m), 5.55 (2H, s), 5.24 (1H, dq, J=7.7, 6.8 Hz), 1.45 (3H, d, J=6.8 Hz);

MS (ESI) m/z 445 (M+H)⁺, 443 (M−H)⁻.

### Example 48

### 4-{(1S)-1-[({5-CHLORO-2-[(2-CHLOROBENZYL)(METHYL)AMINO]PYRIDIN-3-YL}CARBONYL)AMINO]ETHYL}BENZOIC ACID

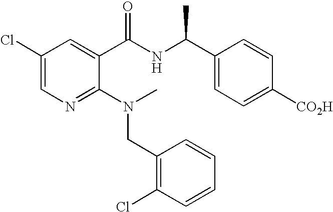

### STEP 1. Methyl 4-{(1S)-1-[({5-chloro-2-[(2-chlorobenzyl)(methyl)amino]pyridin-3-yl}carbonyl)amino]ethyl}benzoate

The title compound was prepared according to the procedure described in step 2 of Example 16 from methyl 4-{(1S)-1-[({5-chloro-2-[methyl(2-phenylethyl)amino]pyridin-3-yl}carbonyl)amino]ethyl}benzoate (step 1 of Example 16) and (2-chlorobenzyl)methylamine:

¹H-NMR (CDCl₃) δ 8.54 (1H, d, J=8.1 Hz), 8.30 (1H, d, J=2.7 Hz), 8.18 (1H, d, J=2.7 Hz), 7.88 (2H, J=8.3 Hz), 7.46-7.16 (6H, m), 5.38-5.25 (1H, m), 4.55 (1H, d, J=13.6 Hz), 4.40 (1H, d, J=13.6 Hz), 3.90 (3H, s), 2.53 (3H, s), 1.47 (3H, d, J=7.2 Hz).

### STEP 2. 4-{(1S)-1-[({5-Chloro-2-[(2-chlorobenzyl)(methyl)amino]pyridin-3-yl}carbonyl)amino]ethyl}benzoic acid

The title compound was prepared according to the procedure described in step 6 of Example 8 from ethyl 4-{(1S)-1-[({5-chloro-2-[(2-chlorobenzyl)(methyl)amino]pyridin-3-yl}carbonyl)amino]ethyl}benzoate (step 1):

¹H-NMR (CDCl₃) δ 8.59 (1H, d, J=8.1 Hz), 8.31 (1H, d, J=2.8 Hz), 8.19 (1H, d, J=2.8 Hz), 7.93 (2H, J=8.3 Hz), 7.43-7.36 (1H, m), 7.34-7.18 (5H, m), 5.40-5.25 (1H, m), 4.54 (1H, d, J=13.5 Hz), 4.42 (1H, d, J=13.5 Hz), 2.55 (3H, s), 1.48 (3H, d, J=7.0 Hz);

MS (ESI) m/z 458 (M+H)⁺, 456 (M−H)⁻.

### Example 49

### 4-((1S)-1-{[5-CHLORO-2-(TETRAHYDROFURAN-2-YLMETHOXY)BENZOYL]AMINO}ETHYL)BENZOIC ACID

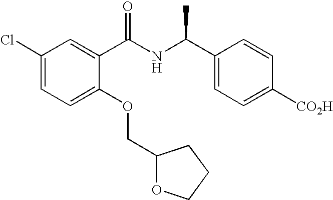

### STEP 1. Methyl 4-((1S)-1-{[5-chloro-2-(tetrahydrofuran-2-ylmethoxy)benzoyl]amino}ethyl)benzoate

The title compound was prepared according to the procedure described in step 5 of Example 8 from methyl 4-{(1S)-1-[(5-chloro-2-hydroxybenzoyl)amino]ethyl}benzoate (step 4 of Example 8) and tetrahydrofuran-2-ylmethanol:

¹H-NMR (CDCl₃) [symbol] 8.74-8.60 (1H, m), 8.15-8.11 (1H, m), 8.01 and 7.99 (total 2H, each d, J=8.3 Hz), 7.50 and 7.48 (total 2H, each d, J=8.3 Hz), 7.35 (1H, dd, J=8.8, 2.8 Hz), 6.89 and 6.87 (total 1H, each d, J=8.8 Hz), 5.38 (1H, dq, J=7.9, 7.0 Hz), 4.36-4.18 (2H, m), 3.94-3.70 (3H, m), 3.90 and 3.89 (total 3H, each s), 2.14-1.86 (3H, m), 1.70-1.55 (1H, m), 1.57 and 1.56 (total 3H, each d, J=7.0 Hz);

MS (ESI) m/z 418 (M+H)⁺, 416 (M−H)⁻.

### STEP 2. 4-((1S)-1-{[5-Chloro-2-(tetrahydrofuran-2-ylmethoxy)benzoyl]amino}ethyl)benzoic acid

The title compound was prepared according to the procedure described in step 6 of Example 8 from methyl 4-((1S)-1-{[5-chloro-2-(tetrahydrofuran-2-ylmethoxy)benzoyl]amino}ethyl)benzoate (step 1):

¹H-NMR (DMSO-d₆) [symbol] 8.82-8.72 (1H, m), 7.91 and 7.90 (total 2H, each d, J=8.3 Hz), 7.68 (1H, d, J=2.8 Hz), 7.60-7.46 (3H, m), 7.23 and 7.22 (total 1H, each d, J=8.8 Hz), 5.28-5.10 (1H, m), 4.32-4.16 (2H, m), 4.03-3.92 (1H, m), 3.83-3.60 (2H, m), 2.05-1.76 (3H, m), 1.70-1.55 (1H, m), 1.46 and 1.45 (total 3H, each d, J=6.9 Hz);

MS (ESI) m/z 404 (M+H)⁺, 402 (M−H)⁻.

### Example 50

### 4-{(1S)-1-[({5-CHLORO-2-[(2-FLUOROBENZYL)(METHYL)AMINO]PYRIDIN-3-YL}CARBONYL)AMINO]ETHYL}BENZOIC ACID

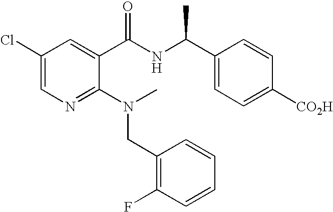

### STEP 1. Methyl 4-{(1S)-1-[({5-chloro-2-[(2-chlorobenzyl)(methyl)amino]pyridin-3-yl}carbonyl)amino]ethyl}benzoate

A mixture of methyl 4-((1S)-1-{[(2,5-dichloropyridin-3-yl)carbonyl]amino}ethyl)benzoate, (step 1 of Example 16, 204 mg, 0.58 mmol), (2-fluorobenzyl)methylamine (264 mg, 1.9 mmol), and diisoprpylethylamine (167 mg, 1.3 mmol) in dimethyl sulfoxide (5 mL) was heated at 150° C. in an oil bath for 24 hours. The reaction mixture was poured into water and the aqueous mixture was extracted with ethyl acetate. The organic extracts were washed with brine, dried (sodium sulfate), and concentrated. The residue was purified by flash column chromatography on silica gel eluting with hexane/ethyl acetate (4/1 to 2/1) to afford 222 mg (86%) of the title compound:

¹H-NMR (CDCl₃) δ 8.98 (1H, d, J=7.9 Hz), 8.30 (1H, d, J=2.6 Hz), 8.20 (1H, d, J=2.6 Hz), 7.93 (2H, d, J=8.3 Hz), 7.33 (2H, d, J=8.3 Hz), 7.40-7.14 (2H, m), 7.12-6.96 (2H, m), 5.40-5.20 (1H, m), 4.41 (2H, s), 3.89 (3H, s), 2.57 (3H, s), 1.49 (3H, d, J=7.0 Hz).

### STEP 2. 4-{(1S)-1-[({5-Chloro-2-[(2-fluorobenzyl)(methyl)amino]pyridin-3-yl}carbonyl)amino]ethyl}benzoic acid

The title compound was prepared according to the procedure described in step 6 of Example 8 from methyl 4-{(1S)-1-[({5-chloro-2-[(2-chlorobenzyl)(methyl)amino]pyridin-3-yl}carbonyl)amino]ethyl}benzoate (step 1):

¹H-NMR (CDCl₃) δ 9.08 (1H, d, J=7.5 Hz), 8.33 (1H, d, J=2.8 Hz), 8.25 (1H, d, J=2.8 Hz), 8.00 (2H, d, J=8.3 Hz), 7.37 (2H, d, J=8.3 Hz), 7.35-7.19 (2H, m), 7.12-6.99 (2H, m), 5.40-5.25 (1H, m), 4.43 (2H, s), 2.60 (3H, s), 1.51 (3H, d, J=7.0 Hz);

MS (ESI) m/z 442 (M+H)⁺, 440 (M−H)⁻.

### Example 51

### 4-{(1S)-1-[({5-CHLORO-2-[(4-CHLOROBENZYL)(METHYL)AMINO]PYRIDIN-3-YL}CARBONYL)AMINO]ETHYL}BENZOIC ACID

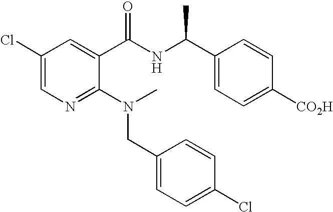

### STEP 1. Methyl 4-{(1S)-1-[({5-chloro-2-[(4-chlorobenzyl)(methyl)amino]pyridin-3-yl}carbonyl)amino]ethyl}benzoate

The title compound was prepared according to the procedure described in step 1 of Example 50 from methyl 4-{(1S)-1-[({5-chloro-2-[methyl(2-phenylethyl)amino]pyridin-3-yl}carbonyl)amino]ethyl}benzoate (step 1 of Example 16) and (4-chlorobenzyl)methylamine:

¹H-NMR (CDCl₃) δ8.69 (1H, d, J=7.7 Hz), 8.27 (1H, d, J=2.6 Hz), 8.16 (1H, d, J=2.6 Hz), 7.98 (2H, d, J=8.4 Hz), 7.35 (2H, d, J=8.4 Hz), 7.21 (2H, d, J=8.4 Hz), 7.06 (2H, d, J=8.4 Hz), 5.37-5.23 (1H, m), 4.34-4.29 (2H, brs), 3.91 (3H, s), 2.59 (3H, s), 1.53 (3H, d, J=7.0 Hz).

### STEP 2. 4-{(1S)-1-[({5-chloro-2-[(4-chlorobenzyl)(methyl)amino]pyridin-3-yl}carbonyl)amino]ethyl}benzoic acid

The title compound was prepared according to the procedure described in step 6 of Example 8 from methyl 4-{(1S)-1-[({5-chloro-2-[(4-chlorobenzyl)(methyl)amino]pyridin-3-yl}carbonyl)amino]ethyl}benzoate (step 1):

¹H-NMR (CDCl₃) δ 8.78 (1H, d, J=7.7 Hz), 8.31 (1H, d, J=2.6 Hz), 8.22 (1H, d, J=2.6 Hz), 8.06 (2H, d, J=8.2 Hz), 7.39 (2H, d, J=8.2 Hz), 7.24 (2H, d, J=8.3 Hz), 7.09 (2H, d, J=8.3 Hz), 5.37-5.27 (1H, m), 4.33 (2H, s), 2.62 (3H, s), 1.55 (3H, d, J=7.0 Hz);

MS (ESI) m/z 458 (M+H)⁺, 456 (M−H)⁻.

### Example 52

### 4-{(1S)-1-[({5-CHLORO-2-[(3-CHLOROBENZYL)(METHYL)AMINO]PYRIDIN-3-YL}CARBONYL)AMINO]ETHYL}BENZOIC ACID

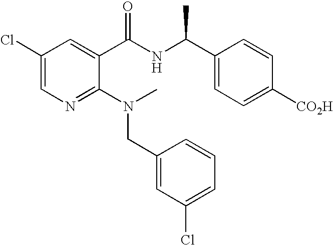

### STEP 1. Methyl 4-{(1S)-1-[({5-chloro-2-[(3-chlorobenzyl)(methyl)amino]pyridin-3-yl}carbonyl)amino]ethyl}benzoate

The title compound was prepared according to the procedure described in step 1 of Example 50 from methyl 4-{(1S)-1-[({5-chloro-2-[methyl(2-phenylethyl)amino]pyridin-3-yl}carbonyl)amino]ethyl}benzoate (step 1 of Example 16) and (3-chlorobenzyl)methylamine:

¹H-NMR (CDCl₃) δ 8.67 (1H, d, J=7.7 Hz), 8.28 (1H, d, J=2.6 Hz), 8.17 (1H, d, J=2.6 Hz), 7.96 (2H, d, J=8.3 Hz), 7.35 (2H, d, J=8.3 Hz), 7.29-7.16 (3H, m), 7.05 (1H, d, J=7.3 Hz), 5.40-5.24 (1H, m), 4.38 (1H, d, J=14.1 Hz), 4.31 (1H, d, J=14.1 Hz), 3.90 (3H, s), 2.58 (3H, s), 1.53 (3H, d, J=7.0 Hz).

### STEP 2. 4-{(1S)-1-[({5-Chloro-2-[(3-chlorobenzyl)(methyl)amino]pyridin-3-yl}carbonyl)amino]ethyl}benzoic acid

The title compound was prepared according to the procedure described in step 6 of Example 8 from methyl 4-{(1S)-1-[({5-chloro-2-[(3-chlorobenzyl)(methyl)amino]pyridin-3-yl}carbonyl)amino]ethyl}benzoate (step 1):

¹H-NMR (CDCl₃) δ 8.76 (1H, d, J=7.9 Hz), 8.31 (1H, d, J=2.6 Hz), 8.22 (1H, d, J=2.6 Hz), 8.04 (2H, d, J=8.3 Hz), 7.40 (2H, d, J=8.3 Hz), 7.32-7.18 (3H, m), 7.07 (1H, d, J=7.2 Hz), 5.39-5.26 (1H, m), 4.39 (1H, d, J=14.3 Hz), 4.34 (1H, d, J=14.3 Hz), 2.60 (3H, s), 1.55 (3H, d, J=6.8 Hz);

MS (ESI) m/z 458 (M+H)⁺, 456 (M−H)⁻.

### Example 53

### 4-{(1S)-1-[({5-CHLORO-2-[(3-FLUOROBENZYL)(METHYL)AMINO]PYRIDIN-3-YL}CARBONYL)AMINO]ETHYL}BENZOIC ACID

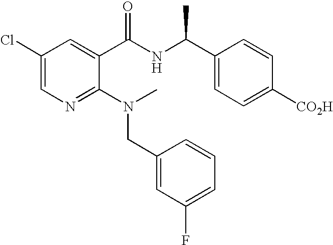

### STEP 1. Methyl 4-{(1S)-1-[({5-chloro-2-[(3-fluorobenzyl)(methyl)amino]pyridin-3-yl}carbonyl)amino]ethyl}benzoate

The title compound was prepared according to the procedure described in step 1 of Example 50 from methyl 4-{(1S)-1-[({5-chloro-2-[methyl(2-phenylethyl)amino]pyridin-3-yl}carbonyl)amino]ethyl}benzoate (step 1 of Example 16) and (3-fluorobenzyl)methylamine:

¹H-NMR (CDCl₃) δ 8.67 (1H, d, J=7.6 Hz), 8.29 (1H, d, J=2.3 Hz), 8.20-8.15 (1H, m), 7.98 (2H, d, J=8.2 Hz), 7.36 (2H, d, J=8.2 Hz), 7.24 (1H, dd, J=7.4, 13.8 Hz), 7.03-6.86 (3H, m), 5.40-5.25 (1H, m), 4.40 (1H, d, J=14.3 Hz), 4.34 (1H, d, J=14.3 Hz), 3.90 (3H, s), 2.60 (3H, s), 1.53 (3H, d, J=6.9 Hz).

### STEP 2. 4-{(1S)-1-[({5-Chloro-2-[(3-fluorobenzyl)(methyl)amino]pyridin-3-yl}carbonyl)amino]ethyl}benzoic acid

The title compound was prepared according to the procedure described in step 6 of Example 8 from methyl 4-{(1S)-1-[({5-chloro-2-[(3-fluorobenzyl)(methyl)amino]pyridin-3-yl}carbonyl)amino]ethyl}benzoate (step 1):

¹H-NMR (CDCl₃) δ 8.76 (1H, d, J=8.1 Hz), 8.31 (1H, d, J=2.6 Hz), 8.21 (1H, d, J=2.6 Hz), 8.04 (2H, d, J=8.3 Hz), 7.39 (2H, d, J=8.3 Hz), 7.32-7.20 (1H, m), 7.03-6.88 (3H, m), 5.40-5.28 (1H, m), 4.37 (2H, s), 2.62 (3H, s), 1.55 (3H, d, J=7.0 Hz);

MS (ESI) m/z 442 (M+H)⁺, 440 (M−H)⁻.

### Example 54

### 4-{(1S)-1-[({5-CHLORO-2-[(4-FLUOROBENZYL)(METHYL)AMINO]PYRIDIN-3-YL}CARBONYL)AMINO]ETHYL}BENZOIC ACID

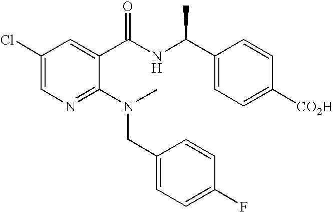

### STEP 1. Methyl 4-{(1S)-1-[({5-chloro-2-[(4-fluorobenzyl)(methyl)amino]pyridin-3-yl}carbonyl)amino]ethyl}benzoate

The title compound was prepared according to the procedure described in step 1 of Example 50 from methyl 4-{(1S)-1-[({5-chloro-2-[methyl(2-phenylethyl)amino]pyridin-3-yl}carbonyl)amino]ethyl}benzoate (step 1 of Example 16) and (4-fluorobenzyl)methylamine:

¹H-NMR (CDCl₃) δ 8.99 (1H, d, J=7.9 Hz), 8.30 (1H, d, J=2.6 Hz), 8.21 (1H, d, J=2.6 Hz), 7.93 (2H, d, J=8.3 Hz), 7.33 (2H, d, J=8.3 Hz), 7.30-7.16 (2H, m), 7.10-6.96 (2H, m), 5.40-5.20 (1H, m), 4.41 (2H, s), 3.89 (3H, s), 2.57 (3H, s), 1.49 (3H, d, J=7.0 Hz).

### STEP 2. 4-{(1S)-1-[({5-Chloro-2-[(4-fluorobenzyl)(methyl)amino]pyridin-3-yl}carbonyl)amino]ethyl}benzoic acid

The title compound was prepared according to the procedure described in step 6 of Example 8 from methyl 4-{(1S)-1-[({5-chloro-2-[(4-fluorobenzyl)(methyl)amino]pyridin-3-yl}carbonyl)amino]ethyl}benzoate (step 1):

¹H-NMR (CDCl₃) δ 8.89 (1H, d, J=7.5 Hz), 8.32 (1H, d, J=2.6 Hz), 8.23 (1H, d, J=2.6 Hz), 8.05 (2H, d, J=8.1 Hz), 7.38 (2H, d, J=8.3 Hz), 7.18-7.08 (2H, m), 7.02-6.90 (2H, m), 5.38-5.26 (1H, m), 4.33 (2H, s), 2.61 (3H, s), 1.54 (3H, d, J=7.0 Hz);

MS (ESI) m/z 442 (M+H)⁺, 440 (M−H)⁻.

Suitable alpha-2-delta ligand compounds of the present invention may be prepared as described herein below or in the aforementioned patent literature references, which are illustrated by the following non-limiting examples and intermediates.

The following examples and preparations illustrate the preparation of alpha-2-delta ligands disclosed in WO-A-2004/039367:

### Example 1

### (2S,4S)-4-(3-Chloro-phenoxy)-pyrrolidine-2-carboxylic acid

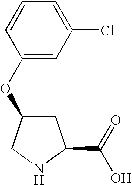

A solution of preparation 2 (29.25 mol) was dissolved in THF (20 L) & filtered. To this solution was added 4M HCl in dioxane (30 L) & stirred overnight. Tert-Butyl methyl ether (70 L) was added to the resultant suspension & the product was collected by filtration (7.06 kg, 86.7%).

¹H NMR (400 MHz, CD₃OD): δ=2.65 (m, 2H), 3.60 (dd, 1H), 3.70 (d, 1H), 4.60 (dd, 1H), 5.02 (m, 1H), 6.88 (m, 1H), 6.97 (s, 1H), 7.03 (d, 1H), 7.29 (dd, 1H).

LRMS (Electrospray [MH⁺] 242, [M−1] 240.

Microanalysis: Found, C, 46.97; H, 4.70; N, 4.90. C₁₁H₁₂ClNO₃.HCl.0.1H₂O requires C, 47.20; H, 4.75; N, 5.00.

### Example 2

### (2S,4S)-4-(3-Fluoro-benzyl)-pyrrolidine-2-carboxylic acid mono hydrochloride salt

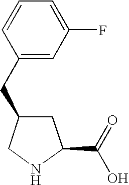

4-(3-Fluoro-benzyl)-pyrrolidine-1,2-dicarboxylic acid 1-tert-butyl ester 2-(2-isopropyl-5-methyl-cyclohexyl)ester (Preparation 3, 0.91 g, 1.96 mmol) was dissolved in toluene (2 ml). 6N hydrochloric acid (50 ml) was added and stirred at reflux for 18 h. The reaction mixture was cooled to room temperature and extracted with ethyl acetate (3×20 ml). The aqueous layer was concentrated by evaporated under reduced pressure to give the title compound (417 mg, 81%) as a white solid. ¹H-NMR showed a 7:1 ratio of cis:trans diastereoisomers so the product was recrystallised from isopropyl alcohol to give the title compound (170 mg, 65%) in a ratio of 14:1 cis:trans as determined by NMR.

¹H-NMR (400 MHz, CD₃OD): (mixture of diastereoisomers 2S,4S:2S,4R (14:1)): δ=1.85 (q, 1H), 2.51 (quin, 1H), 2.69-2.85 (m, 3H), 3.07 (t, 1H), 3.41 (dd, 1H), 4.38 and 4.48 (t, 1H), 6.90-7.04 (m, 3H), 7.32 (q, 1H).

LRMS (APCl): m/z [MH]⁺ 224.

[α]_{D}²⁵−1.27° (c=9.00 in methanol).

Microanalysis: Found C, 55.56; H, 5.81; N, 5.34%. C₁₂H₁₄FNO₂.HCl requires C, 55.50; H, 5.82; N, 5.39%.

### Preparation 1

### (2S,4S)-4-(3-Chloro-phenoxy)-pyrrolidine-1,2-dicarboxylic acid 1-tert-butyl ester 2-methyl ester

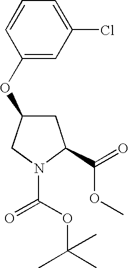

To a stirred solution of (2S,4R)-4-hydroxy-pyrrolidine-1,2-dicarboxylic acid 1-tert-butyl ester 2-methyl ester (CAS Reg 74844-91-0) (6.1 kg, 24.87 mol), 3-chlorophenol (3.52 kg, 27.39 mol) & triphenylphosphine (7.18 kg, 27.37 mol) in tert-butyl methyl ether (30.5 L) at 0° C. was added diisopropylazodicarboxylate (5.53 kg, 27.35 mol) in tert-butyl methyl ether (15 L) dropwise. The mixture was stirred overnight at 20° C. The reaction was filtered and the liquors washed with 0.5M sodium hydroxide (aq) (2×12.5 L) & water (12.2 L). The tert-butyl methyl ether solvent was replaced with n-heptane (42.7 L) by atmospheric pressure distillation & cooled to crystallise crude product, which was collected by filtration (11.1 kg, 125% contaminated with ca 35% reduced diisopropyl dicarboxylate & triphenylphosphine oxide−corrected yield=86%).

¹H NMR (400 MHz, CDCl₃): δ=1.46, 1.49 (2×s, 9H), 2.47 (2H, m), 3.71 (5H, m), 4.42 (1H, m), 4.42, 4.54 (1H, 2×m), 4.87 (1H, m), 6.68 (1H, m), 6.79 (1H, s), 6.92 (1H, m), 7.18 (1H, m).

LRMS (Electrospray): m/z 378 (MNa⁺).

### Preparation 2

### (2S,4S)-4-(3-Chloro-phenoxy)-pyrrolidine-1,2-dicarboxylic acid 1-tert-butyl ester

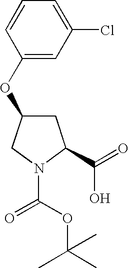

To the products of preparation 14 (11.1 kg, 20.28 mol) in THF (26.6 L) was added a solution of LiOH.H₂O (4.86 kg, 115.4 mol) in water (55.5 L). The mixture was stirred overnight at 25° C. The THF was removed by distillation & the resultant aqueous solution extracted with dichloromethane (33.3 L & 16.7 L). The combined dichloromethane layers were extracted with water (33 L & 16.7 L). The combined aqueous phases were adjusted to pH 3-3.5 with 1M hydrochloric acid (aq) & extracted with dichloromethane (2×22.2 L). The combined dichloromethane phases were replaced with toluene (33.3 L), which was cooled to crystallise the product, which was collected by filtration (6.1 kg, 98%).

¹H NMR (400 MHz, CDCl₃): δ=1.42, 1.48 (2×s, 9H), 2.30-2.70 (m, 2H), 3.60-3.80 (m, 2H), 4.40-4.60 (m, 1H), 4.86 (m, 1H), 6.71 (m, 1H), 6.82 (m, 1H), 6.94 (m, 1H), 7.16 (m, 1H).

LRMS (Electrospray): m/z [MNa⁺] 364, 340 [M−1] 340.

### Preparation 3

### 4-(3-Fluoro-benzyl)-pyrrolidine-1,2-dicarboxylic acid 1-tert-butyl ester 2-(2-isopropyl-5-methyl-cyclohexyl)ester

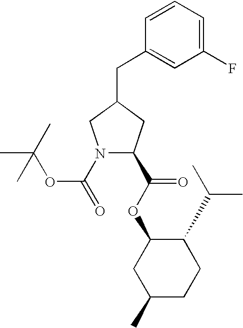

4-(3-fluoro-benzylidene)-pyrrolidine-1,2-dicarboxylic acid 1-tert-butyl ester 2-(2-isopropyl-5-methyl-cyclohexyl)ester (1.20 g, 2.61 mmol) was dissolved in ethyl acetate:toluene (1:1, 12 ml). The solution was submitted to hydrogenation on platinum oxide (120 mg, 10% by weight) at 25° C. and 15 psi for 1 hour. The reaction mixture was filtered through arbocel and the filtrate reduced under pressure. The residue was purified by flashmaster chromatography eluting with heptane:ethyl actetate (15:1) to yield the title compound as a colourless oil (1.11 g, 91%).

¹H-NMR (400 MHz, CD₃OD): δ=0.72-1.37 (m, 13H), 1.44 (d, 9H), 1.43-1.75 (m, 4H), 1.87-2.01 (m, 2H), 2.31-2.58 (m, 2H), 2.83 (d, 2H), 3.07 (t, 1H), 3.50-3.65 (m, 1H), 4.13-4.30 (dt, 1H), 4.71 (td, 1H), 6.90 (d, 2H), 7.00 (d, 1H), 7.30 (q, 1H).

LRMS (APCl): m/z [MH—BOC]⁺ 362.

1. A combination comprising an EP4-receptor antagonist and an alpha-2-delta ligand.

2. A combination according to claim 1, wherein the EP4-receptor antagonist is selected from 2-ethyl-4,6-dimethyl-1-(4-{2-[({[(4-methylphenyl)sulfonyl]amino}carbonyl)amino]ethyl}phenyl)-1H-imidazo[4,5-c]pyridine;
4-(6-chloro-2-ethyl-5-trifluoromethyl-1H-benzimidazol-1-yl)phenethyl-(4-methylphenyl)sulfonylcarbamate;
5-acetyl-2-ethyl-3-(4-{2-[({[(4-methylphenyl)sulfonyl]amino}carbonyl)amino]ethyl}phenyl)benzimidazole;
N-{[(2-{4-[2-ethyl-5-(1-hydroxy-1-methylethyl)-1H-benzimidazol-1-yl]phenyl}ethyl)amino]carbonyl}-4-methylbenzenesulfonamide;
2-{4-[6-chloro-2-ethyl-5-(trifluoromethyl)-1H-benzimidazol-1-yl]phenyl}ethyl (5-methyl-2-pyridinyl)sulfonylcarbamate;
2-{4-[6-chloro-2-(4-pyridinyl)-5-(trifluoromethyl)-1H-benzimidazol-1-yl]phenyl}ethyl (4-methylphenyl)sulfonylcarbamate;
2-{4-[5,7-dimethyl-2-(methylamino)-3H-imidazo[4,5-b]pyridin-3-yl]phenyl}ethyl (4-methylphenyl)sulfonylcarbamate;
N-{[(2-{4-[5,7-dimethyl-2-(methylamino)-3H-imidazo[4,5-b]pyridin-3-yl]phenyl}ethyl)amino]carbonyl}-4-methylbenzenesulfonamide;
2-{5-[6-chloro-2-ethyl-5-(trifluoromethyl)-1H-benzimidazol-1-yl]-2-pyridinyl}ethyl (4-methylphenyl)sulfonylcarbamate;
2-{4-[2-(1,1-dimethylethyl)-4,6-dimethyl-1H-imidazo[4,5-c]pyridin-1-yl]phenyl}ethyl (4-methylphenyl)sulfonylcarbamate;
6-chloro-2-ethyl-1-(4-{2-[methyl({[(4-methylphenyl)sulfonyl]amino}carbonyl)amino]ethyl}phenyl)-1H-benzimidazole-5-carboxamide;
4-[(1S)-1-({[5-chloro-2-(3-fluorophenoxy)pyridin-3-yl]carbonyl}amino)ethyl]benzoic acid;
4-((1S)-1-{[5-chloro-2-(3-fluorophenoxy)benzoyl]amino}ethyl)benzoic acid;
4-[(1S)-1-({[5-chloro-2-(3,4-difluorophenoxy)pyridin-3-yl]carbonyl}amino)ethyl]benzoic acid;
4-[(1S)-1-({[5-chloro-2-(4-fluorophenoxy)pyridin-3-yl]carbonyl}amino)ethyl]benzoic acid;
4-((1S)-1-{[5-chloro-2-(4-fluorophenoxy)benzoyl]amino}ethyl)benzoic acid;
4-[(1S)-1-({[5-chloro-2-(3-chlorophenoxy)pyridin-3-yl]carbonyl}amino)ethyl]benzoic acid;
4-[(1S)-1-({[5-chloro-2-(3-cyanophenoxy)pyridin-3-yl]carbonyl}amino)ethyl]benzoic acid;
4-[(1S)-1-({[5-chloro-2-(2,6-difluorophenoxy)pyridin-3-yl]carbonyl}amino)ethyl]benzoic acid;
4-((1S)-1-{[5-chloro-2-(3-chlorophenoxy)benzoyl]amino}ethyl)benzoic acid;
4-[(1S)-1-({[5-chloro-2-(2-chloro-4-fluorophenoxy)pyridin-3-yl]carbonyl}amino)ethyl]benzoic acid;
2-fluoro-N-{[(2-{4-[5-methyl-4-phenyl-3-(trifluoromethyl)-1H-pyrazol-1-yl]phenyl}ethyl)amino]carbonyl}benzenesulfonamide;
2,4-difluoro-N-{[(2-{4-[5-methyl-4-phenyl-3-(trifluoromethyl)-1H-pyrazol-1-yl]phenyl}ethyl)amino]carbonyl}benzenesulfonamide;
N-[({2-[4-(3,5-dimethyl-4-phenyl-1H-pyrazol-1-yl)phenyl]ethyl}amino)carbonyl]-4-methylbenzenesulfonamide;
2-[4-(3,5-dimethyl-4-phenyl-1H-pyrazol-1-yl)phenyl]ethyl [(4-methylphenyl)sulfonyl]carbamate;
N-[({2-[4-(3,5-dimethyl-4-phenyl-1H-pyrazol-1-yl)phenyl]ethyl}amino)carbonyl]-2-fluorobenzenesulfonamide;
N-[({2-[4-(3,5-dimethyl-4-phenyl-1H-pyrazol-1-yl)phenyl]ethyl}amino)carbonyl]-4-methoxybenzenesulfonamide;
N-[({2-[4-(3,5-dimethyl-4-phenyl-1H-pyrazol-1-yl)phenyl]ethyl}amino)carbonyl]-3,4-dimethoxybenzenesulfonamide;
N-{[(2-{4-[4-(4-ethoxyphenyl)-3,5-dimethyl-1H-pyrazol-1-yl]phenyl}ethyl)amino]carbonyl}-4-methylbenzenesulfonamide;
N-[({2-[4-(3,5-dimethyl-4-phenyl-1H-pyrazol-1-yl)phenyl]ethyl}amino)carbonyl]-2,4-difluorobenzenesulfonamide;
2-{4-[4-(4-fluorophenyl)-3,5-dimethyl-1H-pyrazol-1-yl]phenyl}ethyl [(4-methylphenyl)sulfonyl]carbamate;
2-[4-(2-isopropyl-4-phenyl-1H-imidazol-1-yl)phenyl]ethyl (2-chlorophenyl)sulfonylcarbamate;
2-[4-(2-ethyl-4-phenyl-1Himidazole-1-yl)phenyl]ethyl (4-methylphenyl)sulfonylcarbamate;
2-[4-(2-butyl-4-phenyl-1H-imidazol-1-yl)phenyl]ethyl (2-chlorophenyl)sulfonylcarbamate;
2-[4-(2-isobutyl-4-phenyl-1H-imidazol-1-yl)phenyl]ethyl (2-chlorophenyl)sulfonylcarbamate;
4-chloro-N-[({2-[4-(2-ethyl-4-phenyl-1H-imidazol-1-yl)phenyl]ethyl}amino)carbonyl]benzenesulfonamide;
2-[4-(2-amino-4,5-diphenyl-1H-imidazol-1-yl)phenyl]ethyl (4-methylphenyl)sulfonylcarbamate;
N-[({2-[4-(2-ethyl-4-phenyl-1H-imidazol-1-yl)phenyl]ethyl}amino)carbonyl]-4-methylbenzenesulfonamide;
2-chloro-N-[({2-[4-(2-ethyl-4-phenyl-1H-imidazol-1-yl)phenyl]ethyl}amino)carbonyl]benzenesulfonamide;
2-[4-(2-tert-butyl-4-phenyl-1H-imidazol-1-yl)phenyl]ethyl (2-chlorophenyl)sulfonylcarbamate; and
4-chloro-N-[({2-[4-(2-isopropyl-4-phenyl-1H-imidazol-1-yl)phenyl]ethyl}amino)carbonyl]benzenesulfonamide;
or a pharmaceutically acceptable salt thereof.

3. A combination according to claim 1, wherein the EP4-receptor antagonist is 2-ethyl-4,6-dimethyl-1-(4-{2-[({[(4-methylphenyl)sulfonyl]amino}carbonyl)amino]ethyl}phenyl)-1H-imidazo[4,5-q]pyridine or a pharmaceutically acceptable salt thereof.

4. A combination according to claim 1, wherein the EP4-receptor antagonist is 4-[(1S)-1-({[5-chloro-2-(3-fluorophenoxy)pyridin-3-yl]carbonyl}amino)ethyl]benzoic acid or a pharmaceutically acceptable salt thereof.

5. A combination according to claim 1, wherein the alpha-2-delta ligand is selected from gabapentin, pregabalin, [(1R,5R,6S)-6-(aminomethyl)bicyclo[3.2.0]hept-6-yl]acetic acid, 3-(1-aminomethyl-cyclohexylmethyl)-4H-[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)-acetic acid, (1α,3α,5α) (3-amino-methyl-bicyclo[3.2.0]hept-3-yl)-acetic acid, (3S,5R)-3-aminomethyl-5-methyl-octanoic acid, (3S,5R)-3-amino-5-methyl-heptanoic acid, (3S,5R)-3-amino-5-methyl-nonanoic acid, (3S,5R)-3-amino-5-methyl-octanoic acid, (2S,4S)-4-(3-chlorophenoxy)proline and (2S,4S)-4-(3-fluorobenzyl)proline or a pharmaceutically acceptable salts thereof.

6. A combination according to claim 1, wherein the alpha-2-delta ligand is gabapentin.

7. A combination according to claim 1, wherein the alpha-2-delta ligand is pregabalin.

8. A combination according to claim 1, wherein the alpha-2-delta ligand is (1α,3α,5α)(3-amino-methyl-bicyclo[3.2.0]hept-3-yl)-acetic acid or a pharmaceutically acceptable salt thereof.

9. A combination according to claim 1, wherein the alpha-2-delta ligand is (2S,4S)-4-(3-chlorophenoxy)proline or a pharmaceutically acceptable salt thereof.

10. A combination according to claim 1, wherein the alpha-2-delta ligand is (2S,4S)-4-(3-fluorobenzyl)proline or pharmaceutically acceptable salt thereof.

11. A pharmaceutical composition comprising a combination according to any one of claims 1 to 11, and a pharmaceutically acceptable excipient.

12. (canceled)

13. (canceled)

14. (canceled)

15. (canceled)

17. A method for the treatment of pain, comprising simultaneous, sequential or separate administration, in combination, of therapeutically effective amounts of an EP4-receptor antagonist or a pharmaceutically acceptable salt thereof, and an alpha-2-delta ligand or a pharmaceutically acceptable salt thereof, to a mammal in need of said treatment.

18. The method according to claim 18 wherein the pain is neuropathic pain.

19. The method according to claim 18 wherein the pain is inflammatory pain.
